(19) United States
(12) Patent Application Publication
(10) Pub. No.: US 2020/0405890 A1
(43) Pub. Date: Dec. 31, 2020

(54) POSITRON EMISSION TOMOGRAPHY IMAGING OF ACTIVATABLE BINDING POLYPEPTIDES AND RELATED COMPOSITIONS THEREOF

(71) Applicant: CytomX Therapeutics, Inc., South San Francisco, CA (US)

(72) Inventors: Olga VASILJEVA, Fremont, CA (US); Emma Geertruida Elisabeth DE VRIES, Groningen (NL); Marjolijn N. LUB-DE HOOGE, Groningen (NL); Annelies JORRITSMA-SMIT, Groningen (NL); Martin POOL, Groningen (NL); Danique GIESEN, Groningen (NL); Iris KOK, Groningen (NL); Linda BROER, Groningen (NL); Mark STROH, San Francisco, CA (US)

(21) Appl. No.: 16/971,671

(22) PCT Filed: Feb. 21, 2019
(86) PCT No.: PCT/US2019/019045

§ 371 Date: Aug. 20, 2020

Related U.S. Application Data
(60) Provisional application No. 62/680,416, filed on Jun. 4, 2018.
(60) Provisional application No. 62/656,752, filed on Apr. 12, 2018.
(60) Provisional application No. 62/633,536, filed on Feb. 21, 2018.

(51) Int. Cl. A61K 51/10; C07K 16/28

(52) U.S. Cl. CPC A61K 51/1027; A61K 51/1093; C07K 2317/515; C07K 2317/51; C07K 16/2827

(57) ABSTRACT

The present invention provides methods, compounds, and compositions useful for determining the biodistribution of an activated binding polypeptide in a mammalian subject. The present invention also provides methods for identifying mammalian subjects suitable for treatment with an activatable binding polypeptide.

### CROSS-REFERENCE TO RELATED APPLICATIONS

This application claims the benefit of provisional applications U.S. Ser. No. 62/633,536, filed Feb. 21, 2018, U.S. Ser. No. 62/656,752, filed Apr. 12, 2018, and U.S. Ser. No. 62/680,416, filed Jun. 4, 2018, pursuant 35 U.S.C. § 119(e), each of which is incorporated herein by reference in its entirety.

### FIELD OF THE INVENTION

The present invention relates to novel compounds, compositions, and related methods for detecting the in vivo distribution of activatable binding polypeptides in a subject, as well as identifying subjects suitable for treatment with an activatable binding polypeptide.

### REFERENCE TO SEQUENCE LISTING

The “Sequence Listing” submitted electronically concurrently herewith pursuant 37 C.F.R. § 1.821 in computer readable form (CRF) via EFS-Web as file name CYTX_047_PCT_ST25.txt is incorporated herein by reference. The electronic copy of the Sequence Listing was created on Feb. 21, 2019, and the size on disk is 708 kilobytes.

### BACKGROUND

Antibody-based therapies have proven to be effective in the treatment of several diseases, but in some cases, toxicities due to broad target expression have limited their therapeutic effectiveness. Other limitations such as rapid clearance from the circulation following administration further hinder their effective use as a therapy. Activatable antibodies are designed to selectively activate and bind when exposed to the microenvironment of a target tissue, thus potentially reducing toxicities associated with antibody binding to widely expressed binding targets.

Methods for assessing the potential therapeutic benefit of activatable antibodies are desired.

### SUMMARY OF THE INVENTION

In one aspect, the present invention is directed to a method for detecting an in vivo distribution of an activated binding polypeptide in a subject, the method comprising:

administrating to a mammalian subject a tracer dose of a radiolabeled activatable binding polypeptide,
wherein the radiolabeled activatable binding polypeptide comprises a radionuclide and an activatable binding polypeptide.
   wherein the activatable binding polypeptide comprises a prodomain and a binding moiety, wherein the prodomain comprises a masking moiety and a cleavable moiety.
   wherein, when the radiolabeled activatable binding polypeptide is activated, a radiolabeled activated binding polypeptide is generated that is capable of specifically binding, in vivo, a biological target; and

imaging the mammalian subject using positron emission tomography (PET) at a time point following administration of the tracer dose.

In one embodiment, the radionuclide is ⁸⁹Zr. In some embodiments, the activatable binding polypeptide is an activatable antibody.

In another aspect, the present invention further provides a method for identifying a mammalian subject suitable for treatment with an activatable binding polypeptide, the method comprising:

detecting the in vivo distribution of a radiolabeled activated binding polypeptide in a mammalian subject in accordance with the methods described herein, and

identifying the mammalian subject as being suitable for treatment with the corresponding unlabeled activatable binding polypeptide if the radionuclide is detectably present within the PET image of the tumor.

In a further aspect, the present invention provides a method of treating a mammalian subject with an activatable binding polypeptide, the method comprising:

identifying a mammalian subject suitable for treatment with an activatable binding polypeptide in accordance with the methods described herein; and

administering to the mammalian subject a therapeutically effective dose of the activatable binding polypeptide.

In a still further aspect, the present invention provides an 89Zr-conjugated activatable binding polypeptide,

wherein the ⁸⁹Zr-conjugated activatable binding polypeptide comprises 89Zr conjugated via a chelation moiety to an activatable binding polypeptide,

wherein the activatable binding polypeptide comprises a prodomain and a binding moiety, wherein the prodomain comprises a masking moiety and a cleavable moiety,

wherein, when the ⁸⁹Zr-conjugated activatable binding polypeptide is activated, an ⁸⁹Zr-conjugated activated binding polypeptide is generated that is capable of specifically binding, in vivo, a biological target.

In a further aspect, the present invention is directed to a stable composition comprising an ⁸⁹Zr-conjugated activatable binding polypeptide as described herein and a liquid phase carrier, wherein at least one property selected from the group consisting of percent (%) aggregates, concentration of the ⁸⁹Zr-conjugated activatable binding polypeptide, pH, and radiochemical purity is stable after storage at a temperature in the range of from about 2 to about 8° C. for a period of at least about 1 month, at least about 3 months, at least about 6 months, and at least about 12 months.

### BRIEF DESCRIPTION OF THE FIGURES

FIG. 1 provides a schematic overview of the protocol followed in the in vivo murine study described in Example 1.

FIG. 2A provides representative MicroPET images at 1 day (24 h), 3 days (72 h), and 6 days (144 h) post injection (p.i.) of 10 μg of ⁸⁹Zr-CX-072 (radiolabeled activatable antibody), ⁸⁹Zr-PBCtrl (radiolabeled non-binding control), and ⁸⁹Zr-CX-075 (radiolabeled parental antibody) in MDA-MB-231 xenograft bearing Balb-c/nude mice. Tracer uptake is presented as standardized uptake value (SUV). On the right, maximum intensity projections (MIPs) are presented at 6 days p.i. H: heart; T: tumor; S: spleen; L: lymph node. At 24 h, most uptake is in the heart (H) and other tissue for both tracers. Over time, relative uptake in the tumor (T) increases for ⁸⁹Zr-CX-072, but not for ⁸⁹Zr-PBCtrl.

FIGS. 2B, 2C, and 2D provide the quantification of ⁸⁹Zr-CX-072, ⁸⁹Zr-PbCtrl, and ⁸⁹Zr-CX-075 uptake, respectively, in MDA-MB-231 tumor, blood pool and spleen at 1, 3, and 6 days post injection (p.i.). The plots provide mean standardized uptake value (SUVₘₑₐₙ) on the left y-axis and tumor-to-blood ratio (TBR) on the right axis. Data is shown as mean 2 standard deviation.

FIG. 3A depicts tumor uptake of ⁸⁹Zr-CX-072 and ⁸⁹Zr-PBCtrl in MDA-MB-231 xenograft bearing Balb-c/nude mice 6 days (144 h) post-injection (dose) of ⁸⁹Zr-CX-072 and ⁸⁹Zr-PBCtrl for 10 μg supplemented with 0, 40, or 240 μg non-radiolabeled CX-072 or PBCtrl, resulting in a total protein dose of 10, 50, or 250 μg. The data is presented as mean % ID/g±SD, *: p<0.01.

FIG. 3B provides the quantification of ⁸⁹Zr-CX-072, ⁸⁹Zr-PbCtrl, and ⁸⁹Zr-CX-075 uptake 6 days p.i. in MDA-MB-231 tumor and blood pool at increasing total protein dose. Left: Tracer uptake is presented as mean standardized uptake value (SUVₘₑₐₙ). Right: Tracer uptake in tumor is presented as percentage of injected dose per gram tissue (% ID/g). Data is shown as mean±standard deviation (SD).

FIG. 3C depicts the ex vivo spleen uptake of ⁸⁹Zr-CX-072, ⁸⁹Zr-PbCtrl, and ⁸⁹Zr-CX-075 at increasing total protein dose. Tracer uptake is presented as % ID/g. Data is shown as mean±SD. **: p<0.01, *: p<0.05; ns: not significant

FIG. 4A depicts organ biodistribution of 10 μg ⁸⁹Zr-CX-072 and ⁸⁹Zr-PBCtrl in MDA-MB-231 xenograft bearing Balb-c/nude mice 6 days post-injection. Data is presented as mean % ID/g±SD and tumor-to-blood ratio (mean TBR)±SD. **: p<0.01.

FIG. 4B depicts the ex vivo biodistribution of 10 μg ⁸⁹Zr-CX-072, ⁸⁹Zr-CX-PbCtrl, and ⁸⁹Zr-CX-075 in MDA-MB-231 tumor-bearing mice at 6 days p.i. Tracer uptake per organ is presented as % ID/g. Data is shown as mean±SD. **: p<0.01, *: p<0.05

FIG. 4C depicts MDA-MB-231 tumor uptake of ⁸⁹Zr-CX072, ⁸⁹Zr-PbCtrl, and ⁸⁹Zr-CX-075 6 days p.i. Tracer uptake is presented as % ID/g. Data is shown as mean±SD. **: p<0.01, ns: not significant.

FIG. 4D provides a quantification of activated CX-072 in MDA-MB-231 tumor and spleen lysates in a plot of Concentration (ng/mL) (activated CX-072) vs. Total Protein Dose.

FIG. 4E shows activated CX-072 detected ex vivo in MDA-MB-231 tumor tissue and spleen by Western capillary electrophoresis. Data is shown as mean±SD.

FIG. 5A provides representative maximum intensity projections of ⁸⁹Zr-CX-072, ⁸⁹Zr-PbCtrl, and ⁸⁹Zr-CX-075 in MC38 tumor-bearing mice imaged at 6 days p.i. H: heart, T: tumor, S: spleen, L: lymph node.

FIG. 5B depicts organ biodistribution of 10 μg ⁸⁹Zr-CX-072 and ⁸⁹Zr-PBCtrl in MC38 xenograft bearing C57BL/6 mice. Data is presented as mean % ID/g±SD and tumor-to-blood ratio (mean TBR)±SD. *: p<0.05.

FIG. 5C depicts the quantification of ⁸⁹Zr-CX-072, ⁸⁹Zr-PbCtrl, and ⁸⁹Zr-CX-075 uptake in MC38 tumor, blood pool, and spleen at 6 days p.m. Tracer uptake is presented as mean standardized uptake value (SUVₘₑₐₙ) on the left y-axis. Tumor-to-blood ratio (TBR) is presented on the right y-axis. Data is shown as mean±standard deviation (SD).

FIG. 5D depicts the ex vivo biodistribution of ⁸⁹Zr-CX-072, ⁸⁹Zr-PbCtrl, and ⁸⁹Zr-CX-075 in MC38 tumor-bearing mice 6 days p.i. Tracer uptake per organ is presented as percentage of injected dose per gram tissue (% ID/g). Data is shown as mean±SD, *: p<0.05, **: p<0.01.

FIG. 6A depicts ex vivo uptake of ⁸⁹Zr-CX-072, ⁸⁹Zr-PbCtrl, ⁸⁹Zr-CX-075 in lymphoid tissues and MC38 tumor tissue at 6 days p.i. Tracer uptake per organ is presented as % ID/g. Data is shown as mean±SD. *: p<0.05, **: p<0.01, ns: not significant.

FIG. 6B depicts ex vivo uptake of ⁸⁹Zr-CX-072, ⁸⁹Zr-PbCtrl, ⁸⁹Zr-CX-075 in lymphoid tissues and MC38 tumor tissue at 6 days p.i. Tracer uptake per organ is presented as organ-to-blood ratio. Data is shown as mean±SD. *: p<0.05, **: p<0.01, ns: not significant.

FIG. 7A provides a plot of concentration of activated ⁸⁹Zr-CX-072 species detected in MDA-MB-231 tumor tissue and spleen as a function of protein dose.

FIG. 7B depicts the SDS-PAGE autoradiographs of intact (i.e., unactivated activatable antibody) ⁸⁹Zr-CX-072 and ⁸⁹Zr-PbCtrl in MC38 tumor lysates and plasma 6 days post-injection.

### DETAILED DESCRIPTION

The present invention provides novel compositions comprising radiolabeled activatable binding polypeptides and their use in assessing the biodistribution of the corresponding activated binding polypeptide in a mammalian subject. In one embodiment, the present invention provides a method for detecting an in vivo distribution of an activated binding polypeptide in a mammalian subject, the method comprising:

administrating to a mammalian subject a tracer dose of a radiolabeled activatable binding polypeptide,
wherein the radiolabeled activatable binding polypeptide comprises a radionuclide and an activatable binding polypeptide,
   wherein the activatable binding polypeptide comprises a prodomain and a binding moiety, wherein the prodomain comprises a masking moiety and a cleavable moiety,
   wherein, when the radiolabeled activatable binding polypeptide is activated, a radiolabeled activated binding polypeptide is generated that is capable of specifically binding, in vivo, a biological target; and

imaging the mammalian subject using positron emission tomography (PET) at a time point following administration of the tracer dose.

The term “radiolabeled activatable binding polypeptide” refers herein to a compound comprising a radionuclide and an activatable binding polypeptide. As used herein, the terms “activatable binding polypeptide” and “activatable BP” refer interchangeably to a compound that comprises a binding moiety (BM), linked either directly or indirectly, to a prodomain. The term “binding moiety” and “BM” are used interchangeably herein to refer to a polypeptide that is capable of specifically binding to a biological target. When in a form not modified by the presence of the prodomain, the BM is a polypeptide that specifically binds the biological target. The terms “biological target,” “binding target,” and “target” (when used in the context of binding) refer interchangeably herein to polypeptide that may be present in a mammalian subject. The terms “distribution” and “biodistribution” are used interchangeably herein to refer to the location of activated binding polypeptide in a mammalian subject.

As used herein, the term “prodomain” refers to a peptide, which comprises a masking moiety (MM) and a cleavable moiety (CM). The prodomain functions to mask the BM until the activatable binding polypeptide is exposed to an activation condition. As used herein, the terms “masking moiety” and “MM”, are used interchangeably herein to refer to a peptide that, when positioned proximal to the BM, interferes with binding of the BM to the biological target. The terms “cleavable moiety” and “CM” are used interchangeably herein to refer to a peptide that is susceptible to cleavage (e.g., an enzymatic substrate, and the like), bond reduction (e.g., reduction of disulfide bond(s), and the like), or other change in physical conformation. The CM is positioned relative to the MM and BM, such that cleavage, or other change in its physical conformation, causes release of the MM from its position proximal to the BM (also referred to herein as “unmasking”). The term “activation condition” refers to the condition that triggers unmasking of the BM, and results in generation of an “activated binding polypeptide” (or “activated BP”). Unmasking of the BM typically results in an activated binding polypeptide having greater binding affinity for the biological target as compared to the corresponding activatable binding polypeptide. Typically, the radiolabeled activatable binding polypeptide specifically binds, in vivo, a biological target. The terms “peptide,” “polypeptide,” and “protein” are used interchangeably herein to refer to a polymer comprising naturally occurring or non-naturally occurring amino acid residues or amino acid analogues.

Activatable binding polypeptides that are suitable for use in the practice of the present invention may comprise the BM and prodomain components, CM and MM, in a variety of linear or cyclic configurations (via, for example, a cysteine-cysteine disulfide bond), and may further comprise one or more optional linker moieties through which any two or more of the BM, CM, and/or MM moieties may be bound indirectly to each other. Linkers suitable for use in the activatable binding polypeptides employed in the practice of the invention may be any of a variety of lengths. Suitable linkers include those having a length in the range of from about 1 to about 20 amino acids, or from about 1 to about 19 amino acids, or from about 1 to about 18 amino acids, or from about 1 to about 17 amino acids, or from about 1 to about 16 amino acids, or from about 1 to about 15 amino acids, or from about 2 to about 15 amino acids, or from about 3 to about 15 amino acids, or from about 3 to about 14 amino acids, or from about 3 to about 13 amino acids, or from about 3 to about 12 amino acids. In some embodiments, the ABP comprises one or more linkers comprising 1, 2, 3, 4, 5, 6, 7, 8, 9, 10, 11, 12, 13, 14, 15, 16, 17, 18, 19, or 20 amino acids. Typically, the linker is a flexible linker. As used herein, the term “range” is intended to be inclusive of the endpoints which define the limits of the range.

Exemplary flexible linkers include glycine homopolymers (G)ₙ, (wherein n is an integer that is at least 1; in some embodiments, n is an integer in the range of from about 1 to about 30, or an integer in the range of from about 1 to about 25, or an integer in the range of from about 1 to about 20, or an integer in the range of from about 1 to about 20, or an integer in the range of from about 1 to about 15, or an integer in the range of from about 1 to about 10), glycine-serine polymers, including, for example, (GS)ₙ (wherein n is an integer that is at least 1), (GSGGS)ₙ (SEQ ID NO:68)(wherein n is an integer that is at least 1; in some embodiments, n is an integer in the range of from about 1 to about 30, or an integer in the range of from about 1 to about 25, or an integer in the range of from about 1 to about 20, or an integer in the range of from about 1 to about 20, or an integer in the range of from about 1 to about 15, or an integer in the range of from about 1 to about 10), (GGGS)ₙ (SEQ ID NO:69) (wherein n is an integer that is at least 1; in some embodiments, n is an integer in the range of from about 1 to about 30, or an integer in the range of from about 1 to about 25, or an integer in the range of from about 1 to about 20, or an integer in the range of from about 1 to about 20, or an integer in the range of from about 1 to about 15, or an integer in the range of from about 1 to about 10), GGSG (SEQ ID NO:70), GGSGG (SEQ ID NO:71), GSGSG (SEQ ID NO:72), GSGGG (SEQ ID NO:73), GGGSG (SEQ ID NO:74), GSSSG (SEQ ID NO:75), GSSGGSGGSGGSG (SEQ ID NO:76), GSSGGSGGSGG (SEQ ID NO:77), GSSGGSGGSGGS (SEQ ID NO:78), GSSGGSGGSGGSGGGS (SEQ ID NO:79), GSSGGSGGSG (SEQ ID NO:80), GSSGGSGGSGS (SEQ ID NO:81), GGGS (SEQ ID NO:69), GSSGT (SEQ ID NO:82), GSSG (SEQ ID NO:83), GGGSSGGSGGSGG (SEQ ID NO:173), GGS, and the like, and additionally, a glycine-alanine polymer, an alanine-serine polymer, and other flexible linkers known in the art.

Illustrative activatable binding polypeptide configurations include, for example, in either N- to C-terminal direction or C- to N-terminal direction:
(MM)-(CM)-(BM)
(BM)-(CM)-(MM)
(MM)-L₁-(CM)-(AB)
(MM)-L₁-(CM)-L₂-(AB)
cyclo[L₁-(MM)-L₂-(CM)-L₃-(AB)]
   wherein each of L₁, L₂, and L₃ is a linker peptide that may be identical or different.

An activatable binding polypeptide can also include a spacer located, for example, at the amino terminus of the prodomain. In some embodiments, the spacer is joined directly to the MM of the activatable binding polypeptide. In some embodiments, the spacer is joined directly to the MM of the activatable binding polypeptide in the structural arrangement from N-terminus to C-terminus of spacer-MM-CM-BM. An example of a spacer joined directly to the N-terminus of MM of the activatable antibody is selected from the group consisting of QGQSGS (SEQ ID NO: 157); GQSGS (SEQ ID NO: 158); QSGS (SEQ ID NO: 159); SGS; GS; S; QGQSGQG (SEQ ID NO: 160); GQSGQG (SEQ ID NO: 161); QSGQG (SEQ ID NO: 162); SGQG (SEQ ID NO: 163); GQG; QG; G; QGQSGQ (SEQ ID NO: 164); GQSGQ (SEQ ID NO: 165); QSGQ (SEQ ID NO: 166); SGQ; GQ; and Q.

In some embodiments, the spacer includes at least the amino acid sequence QGQSGS (SEQ ID NO: 157). In some embodiments, the spacer includes at least the amino acid sequence GQSGS (SEQ ID NO: 158). In some embodiments, the spacer includes at least the amino acid sequence QSGS (SEQ ID NO: 159). In some embodiments, the spacer includes at least the amino acid sequence SGS. In some embodiments, the spacer includes at least the amino acid sequence GS. In some embodiments, the spacer includes at least the amino acid sequence S. In some embodiments, the spacer includes at least the amino acid sequence QGQSGQG (SEQ ID NO: 160). In some embodiments, the spacer includes at least the amino acid sequence GQSGQG (SEQ ID NO: 161). In some embodiments, the spacer includes at least the amino acid sequence QSGQG (SEQ ID NO: 162). In some embodiments, the spacer includes at least the amino acid sequence SGQG (SEQ ID NO: 163). In some embodiments, the spacer includes at least the amino acid sequence GQG. In some embodiments, the spacer includes at least the amino acid sequence QG. In some embodiments, the spacer includes at least the amino acid sequence G. In some embodiments, the spacer includes at least the amino acid sequence QGQSGQ (SEQ ID NO: 164). In some embodiments, the spacer includes at least the amino acid sequence GQSGQ (SEQ ID NO: 165). In some embodiments, the spacer includes at least the amino acid sequence QSGQ (SEQ ID NO: 166). In some embodiments, the spacer includes at least the amino acid sequence SGQ. In some embodiments, the spacer includes at least the amino acid sequence GQ. In some embodiments, the spacer includes at least the amino acid sequence Q. In some embodiments, the activatable antibody does not include a spacer sequence.

Activatable binding polypeptides that are suitable for use in the radiolabeled binding polypeptide employed herein include any of the activatable binding polypeptides, modified antibodies, and activatable antibodies described in WO 2009/025846, WO 2010/096838, WO 2010/081173, WO 2013/163631, WO 2013/192546, WO 2013/192550, WO 2014/026136, WO 2014/052462, WO 2014/107599, WO 2014/197612, WO 2015/013671, WO 2015/048329, WO 2015/066279, WO 2015/116933, WO 2016/014974, WO 2016/118629, WO 2016/149201, WO 2016/179285, WO 2016/179257, WO 2016/179335, WO 2017/011580, PCT/US2017/059740, U.S. Provisional Application Ser. Nos. 62/469,429, 62/572,467, and 62/613,358, each of which is incorporated herein by reference in its entirety.

Typically, the prodomain is linked, either directly or indirectly, to the BM via the CM of the prodomain. The CM may be designed to be cleaved by upregulated proteolytic activity (i.e., the activation condition) in tissue, such as those present in many cancers. Thus, activatable binding polypeptides may be designed so they are predominantly activated at a target treatment site where proteolytic activity and the desired biological target are co-localized.

Cleavable moieties suitable for use in radiolabeled activatable binding polypeptides of the present invention include those that are a substrate for a protease. Usually, the protease is an extracellular protease. Suitable substrates may be readily identified using any of a variety of known techniques, including those described in U.S. Pat. Nos. 7,666,817, 8,563,269, PCT Publication No. WO 2014/026136, Boulware, et al., “Evolutionary optimization of peptide substrates for proteases that exhibit rapid hydrolysis kinetics,” Biotechnol. Bioeng. (2010) 106.3: 339-46, each of which is hereby incorporated by reference in its entirety. Exemplary substrates that are suitable for use as a cleavable moiety include, for example, those that are substrates cleavable by any one or more of the following proteases: an ADAM, an ADAM-like, or ADAMTS (such as, for example, ADAM8, ADAM9, ADAM10, ADAM12, ADAM15, ADAM17/TACE, ADAMDEC1, ADAMTS1, ADAMTS4, ADAMTS5); an aspartate protease (such as, for example, BACE, Renin, and the like); an aspartic cathepsin (such as, for example, Cathepsin D, Cathepsin E, and the like); a caspase (such as, for example, Caspase 1, Caspase 2, Caspase 3, Caspase 4, Caspase 5, Caspase 6, Caspase 7, Caspase 7, Caspase 8, Caspase 9, Caspase 10, Caspase 14, and the like); a cysteine proteinase (such as, for example, Cruzipain, Legumain, Otubain-2, and the like); a kallikrein-related peptidase (KLK) (such as, for example, KLK4, KLK5, KLK6, KLK7, KLK8, KLK10, KLK11, KLK13, KLK14, and the like); a metallo proteinase (such as, for example, Meprin, Neprilysin, prostate-specific membrane antigen (PSMA), bone morphogenetic protein 1 (BMP-1), and the like); a matrix metalloproteinase (MMP) (such as, for example, MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27, and the like); a serine protease (such as, for example, activated protein C, Cathepsin A, Cathepsin G, Chymase, a coagulation factor protease (such as, for example, FVIIa, FIXa, FXa, FXIa, FXIIa, and the like)); elastase, Granzyme B, Guanidinobenzoatase, HtrA1, Human Neutrophil Elastase, Lactoferrin, Marapsin, NS3/4A, PACE4, Plasmin, prostate-specific antigen (PSA), tissue plasminogen activator (tPA), Thrombin, Tryptase, urokinase (uPA), a Type II transmembrane Serine Protease (TTSP) (such as, for example, DESC1, DPP-4, FAP, Hepsin, Matriptase-2, MT-SP/Matriptase, TMPRSS2, TMPRSS3, TMPRSS4, and the like), and the like. Exemplary CMs that are suitable for use in the radiolabeled activatable binding polypeptides of the present invention include those described in, for example, WO 2010/081173, WO 2015/048329, WO 2015/116933, and WO 2016/118629, each of which is incorporated herein by reference in its entirety. Illustrative CMs are provided herein as SEQ ID NOs: 1-67. Thus, in some embodiments, the radiolabeled activatable binding polypeptide comprises (i.e., has a prodomain comprising) a CM that comprises an amino acid sequence selected from the group consisting of SEQ ID NOs:1-67. In some embodiments, the CM comprises an amino acid sequence corresponding to SEQ ID NO:24.

The MM is selected such that it reduces the ability of the BM to specifically bind the biological target. As such, the dissociation constant (Kd) of the activatable binding polypeptide toward the biological target is usually greater than the Kd of the corresponding activated binding polypeptide to the biological target. The MM can inhibit the binding of the activatable binding polypeptide to the biological target in a variety of ways. For example, the MM can bind to the BM thereby inhibiting binding of the activatable binding polypeptide to the biological target. The MM can allosterically or sterically inhibit binding of the activatable binding polypeptide to biological target. In some embodiments, the MM binds specifically to the BM. Suitable MMs may be identified using any of a variety of known techniques. For example, peptide MMs may be identified using the methods described in U.S. Patent Application Publication Nos. 2009/0062142 and 2012/0244154, and PCT Publication No. WO 2014/026136, each of which is hereby incorporated by reference in their entirety.

In some embodiments, the MM is selected such that binding of the activatable binding polypeptide to the biological target is reduced, relative to binding of the corresponding BM (i.e., without the prodomain) to the same target, by at least about 50%, or at least about 60%, or at least about 65%, or at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, and even 100%, for at least about 2 hours, or at least about 4 hours, or at least about 6 hours, or at least about 8 hours, or at least about 12 hours, or at least about 24 hours, or at least about 28 hours, or at least about 30 hours, or at least about 36 hours, or at least about 48 hours, or at least about 60 hours, or at least about 72 hours, or at least about 84 hours, or at least about 96 hours, or at least about 5 days, or at least about 10 days, or at least about 15 days, or at least about 30 days, or at least about 45 days, or at least about 60 days, or at least about 90 days, or at least about 120 days, or at least about 150 days, or at least about 180 days, or at least about 1 month, or at least about 2 months, or at least about 3 months, or at least about 4 months, or at least about 5 months, or at least about 6 months, or at least about 7 months, or at least about 8 months, or at least about 9 months, or at least about 10 months, or at least about 11 months, or at least about 12 months or more.

Typically, the MM is selected such that the Kd of the activatable binding polypeptide towards the biological target is at least about 2, about 3, about 4, about 5, about 10, about 25, about 50, about 100, about 250, about 500, about 1,000, about 2,500, about 5,000, about 10,000, about 100,000, about 500,000, about 1,000,000, about 5,000,000, about 10,000,000, about 50,000,000, or greater, or in the range of from about 5 to about 10, or from about 10 to about 100, or from about 10 to about 1,000, or from about 10 to about 10,000 or from about 10 to about 100,000, or from about 10 to about 1,000,000, or from about 10 to about 10 to about 10,000,000, or from about 100 to about 1,000, or from about 100 to about 10,000, or from about 100 to about 100,000, or from about 100 to about 1,000,000, or from about 100 to about 10,000,000, or from about 1,000 to about 10,000, or from about 1,000 to about 100,000, or from about 1,000 to about 1,000,000, or from about 1,000 to about 10,000,000, or from about 10,000 to about 100,000, or from about 10,000 to about 1,000,000, or from about 10,000 to about 10,000,000 or from about 100,000 to about 1,000,00, or 100,000 to about 10,000,000 times greater than the Kd of the BM (i.e., not modified with a prodomain).

Conversely, the MM is selected such that the Kd of the BM (i.e., not modified with a prodomain) towards the biological target is at least about 2, about 3, about 4, about 5, about 10, about 25, about 50, about 100, about 250, about 500, about 1,000, about 2,500, about 5,000, about 10,000, about 100,000, about 500,000, about 1,000,000, about 5,000,000, about 10,000,000, about 50,000,000, or more times lower than the binding affinity of the corresponding activatable binding polypeptide; or in the range of from about 5 to about 10, or from about 10 to about 100, or from about 10 to about 1,000, or from about 10 to about 10,000 or from about 10 to about 100,000, or from about 10 to about 1,000,000, or from about 10 to about 10 to about 10,000,000, or from about 100 to about 1,000, or from about 100 to about 10,000, or from about 100 to about 100,000, or from about 100 to about 1,000,000, or from about 100 to about 10,000,000, or from about 1,000 to about 10,000, or from about 1,000 to about 100,000, or from about 1,000 to about 1,000,000, or from about 1,000 to about 10,000,000, or from about 10,000 to about 100,000, or from about 10,000 to about 1,000,000, or from about 10,000 to about 10,000,000 or from about 100,000 to about 1,000,00, or 100,000 to about 10,000,000 times lower than the binding affinity of the corresponding activatable binding polypeptide.

In some embodiments, the Kd of the MM towards the BM is greater than the Kd of the BM towards the biological target. In these embodiments, the Kd of the MM towards the BM may be at least about 5, at least about 10, at least about 25, at least about 50, at least about 100, at least about 250, at least about 500, at least about 1,000, at least about 2,500, at least about 5,000, at least about 10,000, at least about 100,000, at least about 1,000,000, or even 10,000,000 times greater than the Kd of the BM towards the biological target.

Illustrative MMs include those provided as SEQ ID NOS:84-108 (for use in an anti-PDL-1 activatable antibody), as well as those disclosed in WO 2009/025846, WO 2010/096838, WO 2010/081173, WO 2013/163631, WO 2013/192546, WO 2013/192550, WO 2014/026136, WO 2014/052462, WO 2014/107599, WO 2014/197612, WO 2015/013671, WO 2015/048329, WO 2015/066279, WO 2015/116933, WO 2016/014974, WO 2016/118629, WO 2016/149201, WO 2016/179285, WO 2016/179257, WO 2016/179335, WO 2017/011580, PCT/US2017/059740, U.S. Provisional Application Ser. Nos. 62/469,429, 62/572,467, and 62/613,358, each of which is incorporated herein by reference in its entirety. In some embodiments, the radiolabeled activatable binding polypeptide comprises an anti-PDL-1 activatable antibody, where radiolabeled activatable binding polypeptide has an MM comprising an amino acid sequence selected from the group consisting of any of SEQ ID NOs:84-108. In certain of these embodiments, the MM comprises an amino acid sequence corresponding to SEQ ID NO: 90.

In some embodiments, the prodomain has an amino acid sequence that is a substantially lysine-depleted amino acid sequence. In certain embodiments, the prodomain has an amino acid sequence that is a substantially arginine-depleted amino acid sequence. In some of these embodiments, the prodomain has an amino acid sequence that is a substantially lysine- and arginine-depleted amino acid sequence.

As used herein, the term “substantially ‘X’-depleted” in connection with reference to the prodomain amino acid sequence, where “X” is an amino acid residue type, means that the amino acid sequence of the prodomain, inclusive of any linker(s) present that are proximal to any prodomain elements (i.e., masking moiety and cleavable moiety) comprises 10% or less of the specified amino acid residue type (i.e., “X”), on the basis of total number of amino acid residues in the prodomain, and if present, inclusive of any linker(s) present that are proximal to the prodomain elements (i.e., mask moiety and cleavable moiety). The amino acid sequence of the prodomain, and if present, any linker(s) present that are proximal to the prodomain elements, may be identified by first identifying the amino acid sequence of the binding moiety. The amino acid sequence that remains is considered the prodomain for the purpose of determining the basis on which to compute percentage of an amino acid type present in the prodomain. In some embodiments, when the activatable binding polypeptide is an activatable antibody, the prodomain, inclusive of any linker(s) present that are proximal to the prodomain elements, is located adjacent to (e.g., to the N-terminal side of) framework region 1 of a variable region of the antibody component. In some embodiments, the activatable binding polypeptide comprises

In some embodiments, the prodomain amino acid sequence is a substantially lysine-depleted prodomain amino acid sequence comprising lysine in a quantity that does not exceed 10% on the basis of total number of amino acid residue species in the prodomain amino acid sequence, as defined above. In certain embodiments, the prodomain amino acid sequence comprises lysine in a quantity that does not exceed 9%, or does not exceed 8%, or does not exceed 7%, or does not exceed 6%, or does not exceed 5%, or does not exceed 4%, or does not exceed 3%, or does not exceed 3%, or does not exceed 3%, or does not exceed 2%, or does not exceed 1% of the number of amino acid residues in the prodomain amino acid sequence, as defined above. In certain embodiments, prodomain amino acid sequence comprises from 0 to 5 lysine residues, or from 0 to 4 lysine residues, or from 0-3 lysine residues, or from 0-2 lysine residues, or from 0-1 lysine residues. In certain specific embodiments, the prodomain amino acid sequence comprises an amino acid sequence having no lysine residues present.

In some embodiments, the prodomain amino acid sequence is a substantially arginine-depleted prodomain amino acid sequence comprising arginine in a quantity that does not exceed 10% on the basis of total number of amino acid residue species in the prodomain amino acid sequence, as defined above. In certain embodiments, the prodomain amino acid sequence comprises arginine in a quantity that does not exceed 9%, or does not exceed 8%, or does not exceed 7%, or does not exceed 6%, or does not exceed 5%, or does not exceed 4%, or does not exceed 3%, or does not exceed 3%, or does not exceed 3%, or does not exceed 2%, or does not exceed 1% of the number of amino acid residues in the prodomain amino acid sequence, as defined above. In certain embodiments, the prodomain comprises an arginine-depleted amino acid sequence having no arginine residue present. In certain embodiments, the prodomain amino acid sequence comprises from 0 to 5 arginine residues, or from 0 to 4 arginine residues, or from 0-3 arginine residues, or from 0-2 arginine residues, or from 0-1 arginine residues. In certain specific embodiments, the prodomain amino acid sequence comprises an amino acid sequence having no arginine residues present.

In certain embodiments, the prodomain amino acid sequence is a lysine- and an arginine-depleted prodomain amino acid sequence comprising an amino acid

The binding moiety may be any of a variety of polypeptides that is capable of specifically binding a desired biological target. Illustrative classes of biological targets include cell surface receptors and secreted binding proteins (e.g., growth factors, and the like), soluble enzymes, structural proteins (e.g., collagen, fibronectin, and the like), and the like. Suitable biological targets include, for example, 1-92-LFA-3, α4-integrin, α-V-integrin, α4β1-integrin, AGR2, Anti-Lewis-Y, Apelin J receptor, APRIL, B7-H4, BAFF, BTLA, C5 complement, C-242, CA9, CA19-9 (Lewis a), carbonic anhydrase 9, CD2, CD3, CD6, CD9, CD11a, CD19, CD20, CD22, CD25, CD28, CD30, CD33, CD40, CD40L, CD41, CD44, CD44v6, CD47, CD51, CD52, CD56, CD64, CD70, CD71, CD74, CD80, CD81, CD86, CD95, CD117, CD125, CD132 (IL-2RG), CD133, CD137, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA-4, CTGF, CXCL10, CXCL13, CXCR1, CXCR2, CXCR4, CYR61, DL44, DLK, DLL4, DPP-4, DSG1, EGFR, EGFRviii, Endothelin B receptor (ETBR), ENPP3, EpCAM, EPHA2, ERBB3, F protein of RSV, FAP, FGF-2, FGF-8, FGFR1, FGFR2, FGFR3, FGFR4, Folate receptor, GAL3ST1, G-CSF, G-CSFR, GD2, GITR, GLUT1, GLUT4, GM-CSF, GM-CSFR, GP IIb/IIIa receptors, GP130, GPIIB/IIIA, GPNMB, GRP78, Her2/neu, HVEM, Hyaluronidase, ICOS, IFNα, IFNβHGF, hGH, hyaluronidase, ICOS, IFNα, IFNβ, IFNγ, IgE, IgE receptor (FceRI), IGF, IGF1R, IL1B, IL1R, IL2, IL11, IL12p40, IL-12R, IL-12Rβ1, IL13, IL13R, IL15, IL17, IL18, IL21, IL23, IL23R, IL27/IL27R (wsx1), IL29, IL-31R, IL31/IL31R, IL-2R, IL4, IL4-R, IL6, IL-6R, Insulin Receptor, Jagged Ligands, Jagged 1, Jagged 2, LAG-3, LIF-R, Lewis X, LIGHT, LRP4, LRRC26, MCSP, Mesothelin, MRP4, MUC1, Mucin-16 (MUC16, CA-125), Na/K ATPase, Neutrophil elastase, NGF, Nicastrin, Notch Receptors, Notch 1, Notch 2, Notch 3, Notch 4, NOV, OSM-R, OX-40, PAR2, PDGF-AA, PDGF-BB, PDGFRα, PDGFRβ, PD-1, PD-L1, PD-L2, Phosphatidylserine, P1GF, PSCA, PSMA, RAAG12, RAGE, SLC44A4, Sphingosine 1 Phosphate, STEAP1, STEAP2, TAG-72, TAPA1, TGFβ, TIGIT, TIM-3, TLR2, TLR6, TLR7, TLR8, TLR9, TMEM31, TNFα, TNFR, TNFRS12A, TRAIL-R1, TRAIL-R2, Transferrin, Transferrin receptor, TRK-A, TRK-B, uPAR, VAP1, VCAM-1, VEGF, VEGF-A, VEGF-B, VEGF-C, VEGF-D, VEGFR1, VEGFR2, VEGFR3, VISTA, WISP-1, WISP-2, WISP-3, and the like. In a specific embodiment, the binding target is PDL-1.

In some embodiments, the binding moiety comprises a non-antibody polypeptide, such as, for example, the soluble domain of a cell surface receptor, a secreted binding polypeptide, a soluble enzyme, a structural protein, and portions and variants thereof. As used herein, the term “non-antibody polypeptide” refers to a polypeptide that does not comprise the antigen binding domain of an antibody. Illustrative non-antibody polypeptides that are suitable for use as binding moieties in the radiolabeled activatable binding polypeptides employed herein include any of the biological targets listed above, as well as portions (e.g., soluble domains) and variants thereof.

In one embodiment, the activatable binding polypeptide is an activatable antibody. As used herein, the term “activatable antibody” refers to an activatable binding polypeptide in which the binding moiety comprises a full-length antibody or portion thereof. Typically, in these embodiments, the binding moiety comprises at least a portion of the antigen binding domain. The term “antigen binding domain” refers herein to the part of an immunoglobulin molecule that participates in antigen binding. The antigen binding site is formed by amino acid residues of the N-terminal variable (“V) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of an antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)); Chothia & Lesk, J. Mol. Biol. 196:901-917 (1987); Chothia, et al. Nature 342:878-883 (1989)).

Activatable antibodies may comprise, for example, one or more variable or hypervariable region of a light and/or heavy chain (VL and/or VH, respectively), variable fragment (Fv, Fab′ fragment, F(ab′)2 fragments, Fab fragment, single chain antibody (scab), single chain variable region (scFv), complementarity determining region (CDR), domain antibody (dAB), single domain heavy chain immunoglobulin of the BHH or BNAR type, single domain light chain immunoglobulins, or other polypeptide known to bind a biological target. In some embodiments, an activatable antibody comprises an immunoglobulin comprising two Fab regions and an Fc region. In some embodiments, an activatable antibody is multivalent, e.g., bivalent, trivalent, and so on. In some embodiments, the activatable antibody comprises a prodomain joined to the N-terminus of the VL domain of the antibody (or portion thereof) component of the activatable antibody (e.g., from N-terminus to C-terminus, MM-CM-VL, where each “-” refers to a direct or indirect linkage). In some embodiments, the activatable antibody comprises a prodomain joined to the N-terminus of the VH domain of the antibody (or portion thereof) component of the activatable antibody (e.g., from N-terminus to C-terminus, MM-CM-VH, where each “-” refers to a direct or indirect linkage).

Antibodies and portions thereof (including, for example, individual CDRs, as well as light and heavy chains) that are suitable for use in the radiolabeled activatable binding polypeptides employed herein, include, for example, any of those described in WO 2009/025846, WO 2010/096838, WO 2010/081173, WO 2013/163631, WO 2013/192546, WO 2013/192550, WO 2014/026136, WO 2014/052462, WO 2014/107599, WO 2014/197612, WO 2015/013671, WO 2015/048329, WO 2015/066279, WO 2015/116933, WO 2016/014974, WO 2016/118629, WO 2016/149201, WO 2016/179285, WO 2016/179257, WO 2016/179335, WO 2017/011580, PCT/US2017/059740/WO 2018/085555, WO 2018/165619, PCT/US2018/055733, PCT/US2018/055717, U.S. Provisional Application Ser. Nos. 62/469,429, 62/572,467, 62/613,358, each of which is incorporated herein by reference in its entirety. Illustrative specific sources of antibodies or portions thereof that may be employed in the practice of the present invention include, for example, bevacizumab (VEGF), ranibizumab (VEGF), cetuximab (EGFR), panitumumab (EGFR), infliximab (TNFα), adalimumab (TNFα), natalizumab (Integrin α4), basiliximab (IL2R), eculizumab (Complement C5), efalizumab (CD11a), tositumomab (CD20), ibritumomab tiuxetan (CD20), rituximab (CD20), ocrelizumab (CD20), ofatumamab (CD20), obinutuzumab (CD20), daclizumab (CD25), brentuximab vedotin (CD30), gemtuzumab (CD33), gemtuzumab ozogamicin (CD33), alemtuzumab (CD52), abiciximab (Glycoprotein receptor lib/IIIa), omalizumab (IgE), trastuzumab (Her2), trastuzumab emtansine (Her2), palivizumab (F protein of RSV), ipilimumab (CTLA-4), tremelimumab (CTLA-4), Hu5c8 (CD40L), pertuzumab (Her2-neu), ertumaxomab (CD3/Her2-neu), abatacept (CTLA-4), tanezumab (NGF), bavituximab (Phosphatidylserine), zalutumumab (EGFR), mapatumamab (EGFR), matuzumab (EGFR), nimotuzumab (EGFR), ICR62 (EGFR), mAB 528 (EGFR), CH806 (EGFR), MDX-447 (EGFR/CD64), edrecolomab (EpCAM), RAV12 (RAAG12), huJ591 (PSMA), etanercept (TNF-R), alefacept (1-92-LFA-3), ankinra IL-1Ra), GC1008 (TGFβ), adecatumumab (EpCAM), figitumamab (IGF1R), tocilizumab (IL-6 receptor), ustekinumab (IL-12/IL-23), denosumab (RANKL), nivolumab (PD1), pembrolizumab (PD1), pidilizumab (PD1), MEDI0680 (PD1), PDR001 (PD1), REGN2810 (PD1), BGB-A317 (PD1), BI-754091 (PD1), JNJ-63723283 (PD1), MGA012 (PD1), TSR042 (PD1), AGEN2034 (PD1), INCSHR-1210 (PD1), JS001 (PD1), durvalumab (PD-L1), atezolizumab (PD-L1), avelumab (PD-L1), FAZ053 (PD-L1), LY-3300054 (PD-L1), KN035 (PD-L1), and the like (with biological target indicated in parentheses).

In one embodiment, the BM comprises an anti-PDL1 antibody (i.e., full length antibody or portion thereof). Illustrative anti-PDL1 antibodies (i.e., full length antibodies or portions thereof), include, for example, those having all or a portion of a VL region of an anti-PDL-1 antibody (including, for example, those encoded by SEQ ID NO: 110 and SEQ ID NO:112 (encoded by polynucleotide sequences corresponding to SEQ ID NO:109 and SEQ ID NO:111, respectively)) and/or all or a portion of a VH region of an anti-PDL-1 antibody (including, for example, any of the VH domains encoded by SEQ ID NOs:114, 116, 118, 120, 122, 124, 126, 128, 130, 132, 134, 136, 138, 140, 142, 144, 146, 148, 150, 152, 154, and 156 (encoded by polynucleotide sequences corresponding to SEQ ID NOs:113, 115, 117, 119, 121, 123, 125, 127, 129, 131, 133, 135, 137, 139, 141, 143, 145, 147, 149, 151, 153, and 155, respectively). Illustrative activatable anti-PDL-1 antibodies include an activatable anti-PDL-1 antibody comprising a light chain having an amino acid sequence corresponding to SEQ ID NO:168 or SEQ ID NO:170, encoded by the polynucleotide sequence of SEQ ID NOs:167 and 169, respectively, and a heavy chain corresponding to SEQ ID NO:172 (encoded by the polynucleotide sequence of SEQ ID NO:171).

In some embodiments, the radiolabeled activatable binding polypeptide comprises an activatable anti-PDL-1 antibody having a variable heavy (VH) chain comprising an amino acid sequence selected from the group consisting of SEQ ID NOs:176, 177, 178, 179, 180, 181, 182, 183, 184, 185, and 186; and a variable light (VL) chain comprising an amino acid sequence selected from the group consisting of SEQ ID NOs:187, 188, 189, 190, 191, 192, 193, and 194. In other embodiments, the radiolabeled activatable anti-PDL-1 antibody has a VH chain comprising an amino acid sequence corresponding to SEQ ID NO:195 and a VL chain comprising an amino acid sequence corresponding to SEQ ID NO:196. In further embodiments, the radiolabeled activatable anti-PDL-1 antibody has a VH chain comprising an amino acid sequence selected from the group consisting of SEQ ID NOs: 197 and 198; and a VL chain comprising an amino acid sequence selected from the group consisting of SEQ ID NOs:199, 200, 201, 202, 203, 204, 205, 206, 207, 208, 209.210, 211, 212, 213, and 214. In still further embodiments, the radiolabeled activatable anti-PDL-1 antibody has a VH chain comprising an amino acid sequence selected from the group consisting of SEQ ID Nos:215, 177, 216, 179, 217, 181, 182, 183, 184, and 185; and a VL chain comprising an amino acid sequence selected from the group consisting of SEQ ID NOs:218, 187, 188, 189, 190, 191, 192, and 193 [[Group D]]. In other embodiments, the radiolabeled activatable anti-PDL-1 antibody has a VH chain comprising an amino acid sequence corresponding to SEQ ID NO:219 and a VL chain comprising an amino acid sequence corresponding to SEQ ID NO:220. In certain embodiments, the radiolabeled activatable anti-PDL-1 antibody has a VH chain comprising an amino acid sequence selected from the group consisting of SEQ ID NOs:221, 222, 223, 224, 225, 226, 227, 228, 229, 230, and 231; and a VL chain comprising an amino acid sequence selected from the group consisting of SEQ ID NOs:232, 233, 234, 235, 236, 237, 238, 239, 240, and 241. Instill further embodiments, the radiolabeled activatable anti-PDL-1 antibody has a VH chain comprising an amino acid sequence selected from the group consisting of SEQ ID NOs:242, 243, 244, 245, 246, 247, 248, 249, 250, 251, 252, 253, 254, and 255; and a VL chain comprising an amino acid sequence selected from the group consisting of SEQ ID NOs:256, 257, 258, 259, 260, 261, 262, 263, 264, 265, 266, 267, 268, and 269. In some embodiments, the radiolabeled activatable anti-PDL-1 antibody has a VH chain comprising an amino acid sequence selected from the group consisting of SEQ ID NOs:270, 271, 272, 273, and 274; and a VL chain comprising an amino acid sequence selected from the group consisting of SEQ ID NOs:275, 276, 277, and 278. In certain embodiments, the radiolabeled activatable anti-PDL-1 antibody has a VH chain comprising an amino acid sequence selected from the group consisting of SEQ ID NOs:279, 280, 281, 282, 283, 284, 285, 286, 287, 288, 289, 290, 291, 293, 294, 295, 296, 297, and 298; and a VL chain comprising an amino acid sequence selected from the group consisting of SEQ ID NOs:299, 300, 301, 302, 303, 304, 305, 306, 307, 308, 309, 310, 311, 312, 313, 314, 315, 316, 317, 318, 319, 320, 321, 322, 323, 324, 325, 326, and 327. In other embodiments, the radiolabeled activatable anti-PDL-1 antibody has a heavy chain comprising an amino acid sequence selected from the group consisting of SEQ ID NOs:328 and 329; and a light chain comprising an amino acid sequence selected from the group consisting of SEQ ID NOs:330 and 331. In further embodiments, the radiolabeled activatable anti-PDL-1 antibody has a VH chain comprising an amino acid sequence selected from the group consisting of SEQ ID NOs:332 and 333; and a VL chain comprising an amino acid sequence corresponding to SEQ ID NO:199. In some of these embodiments, the radiolabeled activatable anti-PDL-1 antibody comprises a heavy chain amino acid sequence comprising the amino acid sequence of SEQ ID NO:334, and/or a light chain amino acid sequence corresponding to SEQ ID NO:335. In other embodiments, the radiolableled activatable anti-PDL-1 antibody has a VH chain comprising an amino acid sequence selected from the group consisting of SEQ ID NOs:336, 337, 338, 339, 340, 341, 342, 343, 344, 345, 346, 347, 348, 349, 350, 351, 352, 353, 354, 355, 356, 357, 358, 359, 360, and 361; and a VL chain comprising an amino acid sequence selected from the group consisting of SEQ ID NOs:362, 363, 364, 365, 366, 367, 368, 369 370, 371, 372, 373, 374, 375, 376, and 377. In still other embodiments, the radiolabeled activatable anti-PDL-1 antibody has a VH chain comprising an amino acid sequence corresponding to SEQ ID NO:378 and a VL chain comprising an amino acid sequence corresponding to SEQ ID NO:379. In further embodiments, the radiolabeled activatable anti-PDL-1 antibody has a VH chain comprising an amino acid sequence selected from the group consisting of SEQ ID NOs:380, 381, 382, 383, 384, 385, 386, 387, 388, 389, 390, 391, 392, 393, 394, and 395; and a VL chain comprising an amino acid sequence selected from the group consisting of SEQ ID NOs:396, 397, 398, 399, 400, 401, 402, 403, 404, 405, 406, 407, 408, 409, 410, and 411. In certain embodiments, the radiolabeled activatable anti-PDL-1 antibody has a VH chain comprising an amino acid sequence corresponding to SEQ ID NO:412 and a VL chain comprising an amino acid sequence corresponding to SEQ ID NO:413.

In still further embodiments, the radiolabeled activatable anti-PDL-1 antibody comprises a VL chain having a CDR1 amino acid sequence comprising SEQ ID NO:414, a CDR2 amino acid sequence comprising SEQ ID NO:415, and a CDR3 amino acid sequence comprising SEQ ID NO:416; and a VH chain having a CDR1 amino acid sequence comprising SEQ ID NO:425, a CDR2 amino acid sequence comprising SEQ ID NO:426, and a CDR3 amino acid sequence comprising SEQ ID NO:427. In another embodiment, the radiolabeled activatable anti-PDL-1 antibody comprises a VL chain having a CDR1 amino acid sequence comprising SEQ ID NO:414, a CDR2 amino acid sequence comprising SEQ ID NO:417, and a CDR3 amino acid sequence comprising SEQ ID NO:418; and a VH chain having a CDR1 amino acid sequence comprising SEQ ID NO:425, a CDR2 amino acid sequence comprising SEQ ID NO:428, and a CDR3 amino acid sequence comprising SEQ ID NO:429. In a further embodiment, the radiolabeled activatable anti-PDL-1 antibody comprises a VL chain having a CDR1 amino acid sequence comprising SEQ ID NO:414, a CDR2 amino acid sequence comprising SEQ ID NO:419, and a CDR3 amino acid sequence comprising SEQ ID NO:420; and a VH chain having a CDR1 amino acid sequence comprising SEQ ID NO:425, a CDR2 amino acid sequence comprising SEQ ID NO:430, and a CDR3 amino acid sequence comprising SEQ ID NO:431. In yet another embodiment, the radiolabeled activatable anti-PDL-1 antibody comprises a VL chain having a CDR1 amino acid sequence comprising SEQ ID NO:414, a CDR2 amino acid sequence comprising SEQ ID NO:421, and a CDR3 amino acid sequence comprising SEQ ID NO:422; and a VH chain having a CDR1 amino acid sequence comprising SEQ ID NO:425, a CDR2 amino acid sequence comprising SEQ ID NO:432, and a CDR3 amino acid sequence comprising SEQ ID NO:433.

In a further embodiment, the radiolabeled activatable anti-PDL-1 antibody comprises a VL chain having a CDR1 amino acid sequence comprising SEQ ID NO:414, a CDR2 amino acid sequence comprising SEQ ID NO:423, and a CDR3 amino acid sequence comprising SEQ ID NO:424; and a VH chain having a CDR1 amino acid sequence comprising SEQ ID NO:425, a CDR2 amino acid sequence selected from the group consisting of SEQ ID NOs:434, 436, 443, 444, 445, 446, 447, 448, 449, 450, 451, and 452, and a CDR3 amino acid sequence selected from the group consisting of SEQ ID NOs:435, 437, 438, 439, 440, 441, and 442. In a still further embodiment, the radiolabeled activatable anti-PDL-1 antibody comprises a VL chain having a CDR1 amino acid sequence comprising SEQ ID NO:414, a CDR2 amino acid sequence comprising SEQ ID NO:417, and a CDR3 amino acid sequence comprising SEQ ID NO:424; and a VH chain having a CDR1 amino acid sequence comprising SEQ ID NO:425, a CDR2 amino acid sequence comprising SEQ ID NO:451, and a CDR3 amino acid sequence comprising SEQ ID NO:440.

In an additional embodiment, the radiolabeled activatable anti-PDL-1 antibody comprises a VL chain comprising a CDR1 amino acid sequence selected from the group consisting of SEQ ID NOs:491, 492, 493, 494, and 495, a CDR2 amino acid sequence selected from the group consisting of SEQ ID NOs:479, 417, 480, 481, 482, and a CDR3 amino acid sequence selected from the group consisting of SEQ ID NOs:463, 464, 465, 466, 467, 468, and 469; and a VH chain comprising a CDR1 amino acid sequence selected from the group consisting of SEQ ID NOs:483, 484, 485, 486, 487, 488, 489, and 490, a CDR2 amino acid sequence selected from the group consisting of SEQ ID NOs:470, 471, 472, 473, 474, 475, 476, 477, and 478, and a CDR3 amino acid sequence selected from the group consisting of SEQ ID NOs:453, 454, 455, 456, 457, 458, 459, 460, 461, and 462. In one embodiment, the radiolabeled activatable anti-PDL-1 antibody comprises a VL chain comprising a CDR1 amino acid sequence selected from the group consisting of SEQ ID NOs:499, 505, and 511, a CDR2 amino acid sequence selected from the group consisting of SEQ ID NOs:500, 506, and 512, and a CDR3 amino acid sequence selected from the group consisting of SEQ ID NOs:501, 507, and 513; and a VH chain comprising a CDR1 amino acid sequence selected from the group consisting of SEQ ID NOs:496, 502, and 508, a CDR2 amino acid sequence selected from the group consisting of SEQ ID NO:497, 503, and 509, and a CDR3 amino acid sequence selected from the group consisting of SEQ ID NOs:498, 504, and 510. In another embodiment, the radiolabeled activatable anti-PDL-1 antibody comprises a VL chain comprising a CDR1 amino acid sequence selected from the group consisting of SEQ ID NOs:514 and 520, and a CDR2 amino acid sequence selected from the group consisting of SEQ ID NOs:515 and 521, and a CDR3 amino acid sequence selected from the group consisting of SEQ ID NOs:516 and 522; and a VH chain comprising a CDR1 amino acid sequence selected from the group consisting of SEQ ID NOs:517 and 523, a CDR2 amino acid sequence selected from the group consisting of SEQ ID NOs:518 and 524, and a CDR3 amino acid sequence corresponding to SEQ ID NO:519. In a further embodiment, the radiolabeled activatable anti-PDL-1 antibody comprises a VL chain comprising a CDR1 amino acid sequence selected from the group consisting of SEQ ID NOs:525, 531, and 536, a CDR2 amino acid sequence corresponding to SEQ ID NO:526, and a CDR3 amino acid sequence selected from the group consisting of SEQ ID NOs:527, 532, and 537; and a VH chain comprising a CDR1 amino acid sequence selected from the group consisting of SEQ ID NOs:528, 533, 538, 541, and 542, a CDR2 amino acid sequence selected from the group consisting of SEQ ID NO:529, 534, and 539, and a CDR3 amino acid sequence selected from the group consisting of SEQ ID NO:530, 535, and 540.

In another embodiment, the radiolabeled activatable anti-PDL-1 antibody comprises a VL chain comprising a CDR1 amino acid sequence selected from the group consisting of SEQ ID NOs:543 and 549, a CDR2 amino acid sequence selected from the group consisting of SEQ ID NOs:544 and 550, and a CDR3 amino acid sequence selected from the group consisting of SEQ ID NOs:546 and 552; and a VH chain comprising a CDR1 amino acid sequence selected from the group consisting of SEQ ID NOs:547 and 553, a CDR2 amino acid sequence selected from the group consisting of SEQ ID NOs:547 and 553, and a CDR3 amino acid sequence selected from the group consisting of SEQ ID NOs:548 and 554. In certain embodiments, the radiolabeled activatable anti-PDL-1 antibody comprises a VH chain comprising a CDR1 amino acid sequence corresponding to SEQ ID NO:555, a CDR2 amino acid sequence corresponding to SEQ ID NO:556, and a CDR3 amino acid sequence corresponding to SEQ ID NO:557. In other embodiments, the radiolabeled activatable anti-PDL-1 antibody comprises a VL chain comprising a CDR1 amino acid sequence selected from the group consisting of SEQ ID NOs:558, 564, 569, 575, and 581, a CDR2 amino acid sequence selected from the group consisting of SEQ ID NOs:559, 565, 570, 576, and 526, and a CDR3 amino acid sequence selected from the group consisting of SEQ ID NOs:560, 566, 571, and 577; and a VH chain comprising a CDR1 amino acid sequence selected from the group consisting of SEQ ID NOs:561, 567, 572, 578, 582, and 584, and a CDR2 amino acid sequence selected from the group consisting of SEQ ID NOs:562, 568, 573, 579, and 585, and a CDR3 amino acid sequence selected from the group consisting of the sequence, GAL, and amino acid sequences corresponding to SEQ ID NOs:563, 574, 580, 583, and 586. In a further embodiment, the radiolabeled activatable anti-PDL-1 antibody comprises a VL chain comprising a CDR1 amino acid sequence selected from the group consisting of the amino acid sequence, YVS, and SEQ ID NOs:587, 592, 598, 604, 613, 619, 625, 630, 636, 642, 648, 652, and 656, a CDR2 amino acid sequence selected from the group consisting of SEQ ID NOs:588, 593, 599, 550, 480, 614, 620, 626, 631, 637, and 643, and a CDR3 amino acid sequence selected from the group consisting of SEQ ID NOs:589, 594, 600, 605, 609, 615, 621, 627, 632, 638, 644, 649, 653, 657, and 661; and a VH chain comprising a CDR1 amino acid sequence selected from the group consisting of SEQ ID NOs:488, 595, 601, 606, 610, 616, 622, 425, 633, 639, 645, 658, and 662, a CDR2 amino acid sequence selected from the group consisting of SEQ ID NOs:590, 596, 602, 607, 611, 617, 623, 628, 634, 640, 646, 650, 654, and 659, and a CDR3 amino acid sequence selected from the group consisting of SEQ ID NOs:591, 597, 603, 608, 612, 624, 629, 635, 641, 647, 651, 655, 660, and 663.

In some embodiments, the radiolabeled activatable anti-PDL-1 antibody comprises a VL chain having a CDR1 amino acid sequence comprising SEQ ID NO:664, a CDR2 amino acid sequence comprising SEQ ID NO:665, and a CDR3 amino acid sequence comprising SEQ ID NO:666; and a VH chain having a CDR1 amino acid sequence comprising SEQ ID NO:667, a CDR2 amino acid sequence comprising SEQ ID NO:668, and a CDR3 amino acid sequence comprising SEQ ID NO:669. In a further embodiment, the radiolabeled activatable anti-PDL-1 antibody comprises a VL chain having a CDR1 amino acid sequence comprising SEQ ID NO:520, a CDR2 amino acid sequence comprising SEQ ID NO:521, and a CDR3 amino acid sequence comprising SEQ ID NO:523; and a VH chain having a CDR1 amino acid sequence comprising SEQ ID NO:524, a CDR2 amino acid sequence comprising SEQ ID NO:525, and a CDR3 amino acid sequence comprising SEQ ID NO:518.

In certain embodiments, the radiolabeled activatable anti-PDL-1 antibody comprises a VL chain having a CDR1 amino acid sequence comprising an amino acid sequence selected from the group consisting of SEQ ID NOs:670, 675, 684, 689, 693, 698, 701, 1075, 706, 698, 718, 723, 728, and 698, a CDR2 amino acid sequence selected from the group consisting of KAS, TAS, AAS, KVS, KIS, VAS, GAS, and VVS, and a CDR3 amino acid sequence selected from the group consisting of SEQ ID NOs:671, 676, 680, 685, 694, 702, 694, 707, 711, 694, 719, 724, 729, 733, and 694; and a VH chain having a CDR1 amino acid sequence selected from the group consisting of SEQ ID NOs:672, 677, 681, 686, 690, 695, 703, 1076, 708, 712, 715, 720, 725, 730, 734, 737, 740, 742, 744, 747, 750, 753, 756, 759, and 762, a CDR2 amino acid sequence selected from the group consisting of SEQ ID NOs:673, 678, 682, 687, 691, 696, 699, 704, 1077, 709, 713, 716, 721, 726, 731, 735, 738, 704, 743, 745, 748, 751, 754, 757, and 760, and a CDR3 amino acid sequence selected from the group consisting of SEQ ID NOs:674, 679, 683, 688, 692, 697, 700, 705, 710, 714, 717, 722, 727, 732, 736, 739, 741, 746, 749, 752, 755, 758, 761, and 763. In other embodiments, the radiolabeled activatable anti-PDL-1 antibody comprises a VL chain having a CDR1 amino acid sequence comprising SEQ ID NO:764, a CDR2 amino acid sequence comprising SEQ ID NO:765, and a CDR3 amino acid sequence comprising SEQ ID NO:766; and a VH chain having a CDR1 amino acid sequence comprising SEQ ID NO:767, a CDR2 amino acid sequence comprising SEQ ID NO:768, and a CDR3 amino acid sequence comprising SEQ ID NO:769. In further embodiments, the radiolabeled activatable anti-PDL-1 antibody comprises a VL chain having a CDR1 amino acid sequence comprising SEQ ID NO:770, a CDR2 amino acid sequence comprising SEQ ID NO:771, and a CDR3 amino acid sequence comprising SEQ ID NO:772; and a VH chain having a CDR1 amino acid sequence comprising SEQ ID NO:773, a CDR2 amino acid sequence comprising SEQ ID NO:774, and a CDR3 amino acid sequence comprising SEQ ID NO:775.

In some embodiments, the radiolabeled activatable anti-PDL-1 antibody comprises a VL chain having a CDR1 amino acid sequence comprising SEQ ID NO:776, a CDR2 amino acid sequence comprising SEQ ID NO:777, and a CDR3 amino acid sequence comprising SEQ ID NO:778; and a VH chain having a CDR1 amino acid sequence comprising SEQ DI NO:779, a CDR2 amino acid sequence selected from the group consisting of SEQ ID NOs:780, 782, and 784, and a CDR3 amino acid sequence selected from the group consisting of SEQ ID NOs:781 and 783. In a still further embodiment, the radiolabeled activatable anti-PDL-1 antibody comprises a VL chain having a CDR1 amino acid sequence selected from the group consisting of SEQ ID NOs:785, 791, 793, 799, 803, 809, 815, 819, 824, and 830, a CDR2 amino acid sequence selected from the group consisting of SEQ ID NOs:786, 794, 800, 804, 810, 816, 786, 825, and 786, and a CDR3 amino acid sequence selected from the group consisting of SEQ ID NOs:787, 795, 805, 811, 817, 820, 826, and 787; and a VH chain having a CDR1 amino acid sequence selected from the group consisting of SEQ ID NOs:788, 796, 801, 806, 812, 821, 827, and 788, a CDR2 amino acid sequence selected from the group consisting of SEQ ID NOs:789, 792, 797, 802, 807, 813, 818, 822, 828, and 831, and a CDR3 amino acid sequence selected from the group consisting of SEQ ID NOs:790, 798, 808, 814, 823, 829, and 832. In other embodiments, the radiolabeled activatable anti-PDL-1 antibody comprises a VH chain comprising a CDR1 amino acid sequence selected from the group consisting of SEQ ID NOs:833, 834, 835, 836, 837, 838, 839, 840, 841, 842, 843, 844, 845, 846, 847, 848, 849, 850, 851, 852, 853, 854, 855, 856, and 857.

Exemplary combinations of CDR amino acid sequences in radiolabeled activatable anti-PDL-1 antibodies employed in the embodiments of the present invention are provided in Table 1, below.

TABLE 1
Exemplary CDR combinations for a Radiolabeled Activatable Anti-PDL-1 Antibody

| VL | | | VH | | |
|---|---|---|---|---|---|
| VL CDR1 | VL CDR2 | VL CDR3 | VH CDR1 | VH CDR2 | VH CDR3 |
| (SEQ ID NO:) | (SEQ ID NO:) | (SEQ ID NO:) | (SEQ ID NO:) | (SEQ ID NO:) | (SEQ ID NO:) |
| 414 | 415 | 416 | 425 | 426 | 427 |
| 414 | 417 | 418 | 425 | 428 | 429 |
| 414 | 419 | 420 | 425 | 430 | 431 |
| 414 | 421 | 422 | 425 | 432 | 433 |
| 414 | 423 | 424 | 425 | 434 | 435 |
| 414 | 423 | 424 | 425 | 436 | 437 |
| 414 | 423 | 424 | 425 | 436 | 438 |
| 414 | 423 | 424 | 425 | 436 | 439 |
| 414 | 423 | 424 | 425 | 436 | 440 |
| 414 | 423 | 424 | 425 | 436 | 441 |
| 414 | 423 | 424 | 425 | 436 | 442 |
| 414 | 423 | 424 | 425 | 443 | 435 |
| 414 | 423 | 424 | 425 | 444 | 435 |
| 414 | 423 | 424 | 425 | 445 | 435 |
| 414 | 423 | 424 | 425 | 446 | 435 |
| 414 | 423 | 424 | 425 | 447 | 435 |
| 414 | 423 | 424 | 425 | 448 | 435 |
| 414 | 423 | 424 | 425 | 449 | 435 |
| 414 | 423 | 424 | 425 | 450 | 435 |
| 414 | 423 | 424 | 425 | 443 | 435 |
| 414 | 423 | 424 | 425 | 446 | 435 |
| 414 | 423 | 424 | 425 | 451 | 440 |
| 414 | 423 | 424 | 425 | 451 | 441 |
| 414 | 423 | 424 | 425 | 451 | 442 |
| 414 | 423 | 424 | 425 | 452 | 440 |
| 414 | 423 | 424 | 425 | 452 | 441 |
| 414 | 423 | 424 | 425 | 452 | 442 |
| 414 | 417 | 424 | 425 | 451 | 440 |

Additional examples of combinations of CDR amino acid sequences suitable for use in radiolabeled activatable anti-PDL-1 antibodies used in the embodiments of the present invention are provided in Table 2.

TABLE 2
Exemplary CDR combinations for a Radiolabeled Activatable anti-PDL-1 Antibody

| VL | | | VH | | |
|---|---|---|---|---|---|
| VL CDR1 | VL CDR2 | VL CDR3 | VH CDR1 | VH CDR2 | VH CDR3 |
| (SEQ ID NO:) | (SEQ ID NO: /*) | (SEQ ID NO:) | (SEQ ID NO:) | (SEQ ID NO:) | (SEQ ID NO: /*) |
| 491 | 479 | 463 | 483 | 470 | 453 |
| 492 | 417 | 464 | 484 | 471 | 454 |
| 493 | 480 | 465 | 485 | 472 | 455 |
| 494 | 481 | 466 | 486 | 473 | 456 |
| 495 | 482 | 467 | 487 | 474 | 457 |
| | | 468 | 488 | 475 | 458 |
| | | 469 | 489 | 476 | 459 |
| | | | 490 | 477 | 460 |
| | | | | 478 | 461 |
| 499 | 500 | 501 | 496 | 497 | 498 |
| 505 | 506 | 507 | 502 | 503 | 504 |
| 511 | 512 | 513 | 508 | 509 | 510 |
| 514 | 515 | 516 | 517 | 518 | 519 |
| 520 | 521 | 522 | 523 | 524 | |
| 525 | 526 | 527 | 528 | 529 | 530 |
| 531 | | 532 | 533 | 534 | 535 |
| 536 | | 537 | 538 | 539 | 540 |
| 543 | 544 | 545 | 546 | 547 | 548 |
| 549 | 550 | 551 | 552 | 553 | 554 |
| | | | 555 | 556 | 557 |
| 558 | 559 | 560 | 561 | 562 | 563 |
| 564 | 565 | 566 | 567 | 568 | GAL |
| 569 | 570 | 571 | 572 | 573 | 574 |
| 575 | 576 | 577 | 578 | 579 | 580 |
| 581 | 526 | | 582 | | 583 |
| | | | 584 | 585 | 586 |
| 587 | 588 | 589 | 488 | 590 | 591 |
| 592 | 593 | 594 | 595 | 596 | 597 |
| 598 | 599 | 600 | 601 | 602 | 603 |
| 604 | 550 | 605 | 606 | 607 | 608 |
| YVS | 480 | 609 | 610 | 611 | 612 |
| 613 | 614 | 615 | 616 | 617 | 618 |
| 619 | 620 | 621 | 622 | 623 | 624 |
| 625 | 626 | 627 | 425 | 628 | 629 |
| 630 | 631 | 632 | 633 | 634 | 635 |
| 636 | 637 | 638 | 639 | 640 | 641 |
| 642 | 643 | 644 | 645 | 646 | 647 |
| 648 | | 649 | | 650 | 651 |
| 652 | | 653 | | 654 | 655 |
| 656 | | 657 | 658 | 659 | 660 |
| | | 661 | 662 | | 663 |
| 664 | 665 | 666 | 667 | 668 | 669 |
| 520 | 521 | 523 | 524 | 525 | 518 |
| 670 | KAS | 671 | 672 | 673 | 674 |
| 675 | TAS | 676 | 677 | 678 | 679 |
| 675 | AAS | 680 | 681 | 682 | 683 |
| 684 | KVS | 685 | 686 | 687 | 688 |
| 689 | VAS | 694 | 690 | 691 | 692 |
| 693 | VAS | 694 | 695 | 696 | 697 |
| 698 | AAS | 702 | 695 | 699 | 700 |
| 701 | AAS | 694 | 703 | 704 | 705 |
| 1075 | GAS | 707 | 1076 | 1077 | 705 |
| 706 | AAS | 711 | 708 | 709 | 710 |
| 698 | AAS | 694 | 712 | 713 | 714 |
| 698 | AAS | 719 | 715 | 716 | 717 |
| 718 | AAS | 724 | 720 | 721 | 722 |
| 723 | AAS | 729 | 725 | 726 | 727 |
| 728 | VVS | 733 | 730 | 731 | 732 |
| 698 | AAS | 694 | 734 | 735 | 736 |
| | | 740 | 737 | 738 | 739 |
| | | 742 | 740 | 704 | 741 |
| | | 744 | 742 | 743 | 741 |
| | | 747 | 744 | 745 | 746 |
| | | 750 | 747 | 748 | 749 |
| | | 753 | 750 | 751 | 752 |
| | | 756 | 753 | 754 | 755 |
| | | 759 | 756 | 757 | 758 |
| | | 762 | 759 | 760 | 761 |
| | | | 762 | | 763 |
| 764 | 765 | 766 | 767 | 768 | 769 |
| 770 | 771 | 772 | 773 | 774 | 775 |
| 776 | 777 | 778 | 779 | 780 | 781 |
| | | | | 782 | 783 |
| 705 | 786 | 787 | 788 | 789 | 790 |
| 791 | 786 | 787 | 788 | 792 | 790 |
| 793 | 794 | 795 | 796 | 797 | 798 |
| 799 | 800 | 795 | 801 | 802 | 798 |
| 803 | 804 | 805 | 806 | 807 | 808 |
| 809 | 810 | 811 | 812 | 813 | 814 |
| 815 | 816 | 817 | 801 | 818 | 798 |
| 819 | 786 | 820 | 821 | 822 | 823 |
| 824 | 825 | 826 | 827 | 828 | 829 |
| 830 | 786 | 787 | 788 | 831 | 832 |

*Or amino acid sequence if fewer than 4 amino acid residues in amino acid sequence

In certain embodiments, the activatable anti-PDL-1 antibody employed in the radiolabeled activatable binding polypeptide has: (A) alight chain sequence that comprises (i) a MM comprising an amino acid sequence selected from the group consisting of SEQ ID NOs:84, 85, 86, 87, 88, 89, 90, 91, 92, 93, 94, 95, 96, 97, 98, 99, 100, 101, 102, 103, 104, 105, 106, 107, and 108; (ii) a CM comprising an amino acid sequence selected from the group consisting of SEQ ID NOs:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, and 67; and (iii) a VL amino acid sequence comprising an amino acid sequence selected from the group consisting of SEQ ID NO: 110 and 112; and (B) a VH amino acid sequence comprising an amino acid sequence selected from the group consisting of SEQ ID NOs:114, 116, 118, 120, 122, 124, 126, 128, 130, 132, 134, 136, 140, 142, 144, 146, 148, 150, 152, 154, and 156. In some of these embodiments, the radiolabeled activatable binding polypeptide employed in the practice of the present invention comprises: (a) a light chain sequence that comprises (i) an MM that comprises an amino acid sequence corresponding to SEQ ID NO:90; (ii) a CM that comprises an amino acid sequence corresponding to SEQ ID NO:24; and (iii) a VL amino acid sequence comprising an amino acid sequence corresponding to SEQ ID NO: 112; and (B) a VH amino acid sequence comprising an amino acid sequence corresponding to SEQ ID NO:146.

In some embodiments, the activatable anti-PDL-1 antibody employed in the radiolabeled activatable binding polypeptide has a LC that comprises an amino acid sequence selected from the group consisting of SEQ ID NOs: 168, 170, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991 (which are encoded by polynucleotide sequences corresponding to SEQ ID NOs:858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, and 990, respectively); and a VH amino acid sequence that comprises the amino acid sequence of SEQ ID NO:146. In some embodiments, the activatable anti-PDL-1 antibody comprises a HC amino acid sequence comprising the amino acid sequence of SEQ ID NO:172. In certain embodiments, the LC has an amino acid sequence selected from the group consisting of SEQ ID NOs:992, 993, 994, and 995; and a VH amino acid sequence that comprises the amino acid sequence of SEQ ID NO:146. In other embodiments, the LC has an amino acid sequence selected from the group consisting of SEQ ID NOs:997, 999, 1001, 1003, 1005, 1007, 1009, 1011, 1013, 1015, 1017, and 1019 (which are encoded by polynucleotide sequences corresponding to SEQ ID NOs:996, 998, 1000, 1002, 1004, 1006, 1008, 1010, 1012, 1014, 1016, 1018, and 1020, respectively); and a VH amino acid sequence that comprises the amino acid sequence of SEQ ID NO:146. In further embodiments, the LC comprises an amino acid sequence selected from the group consisting of SEQ ID NOs:1021, 1022, 1023, 1024, 1025, 1026, 1027, 1028, 1028, 1029, 1029, 1030, 1031, 1032, 1033, 1034, 1036, 1037, 1038, 1040, 1041, 1042, 1043, 1044, 1045, 1046, 1047, 1048, 1049, 1050, 1051, 1052, 1053, 1054, 1055, 1056, 1057, 1058, and 1059; and a VH amino acid sequence that comprises the amino acid sequence of SEQ ID NO:146.

In some embodiments, the radiolabeled activatable anti-PDL-1 antibody is a single-chain variable fragment comprising an amino acid sequence selected from the group consisting of SEQ ID NOs:1061, 1063, 1065, 1067, and 1069 (encoded by the polynucleotide sequence corresponding to SEQ ID NOs:1060, 1062, 1064, 1066, and 1068, respectively).

The VH amino acid sequences described herein can be combined with human immunoglobulin heavy chain constant domains to yield, e.g., human IgG1 (SEQ ID NO:1071), a mutated human IgG4, e.g., human IgG4 S228P (SEQ ID NO:172), or mutated human IgG1 N2971 (SEQ ID NO:1074).

In some embodiments, the radiolabeled activatable anti-PDL-1 antibody comprises:

(a) a variable heavy chain complementarity determining region 1 (VH CDR1) comprising the amino acid sequence of SEQ ID NO:425;

(b) a variable heavy chain complementarity determining region 2 (VH CDR2) comprising an amino acid sequence selected from the group consisting of SEQ ID NOs: 436, 428, 430, 432, 434, 436, and 443-452; and

(c) a variable heavy chain complementarity determining region 3 (VH CDR3) comprising an amino acid sequence selected from the group consisting of SEQ ID NOs: 427, 429, 431, 433, 435, 437, and 438-442. In these embodiments, the radiolabeled activatable anti-PDL-1 antibody often further comprises:

(d) a variable light chain complementarity determining region 1 (VL CDR1) comprising the amino acid sequence of SEQ ID NO:414;

(e) a variable light chain complementarity determining region 2 (VL CDR2) comprising an amino acid sequence selected from the group consisting of SEQ ID NOs:415, 417, 419, 421, and 423; and

(f) a variable light chain complementarity determining region 3 (VL CDR3) comprising an amino acid sequence selected from the group consisting of SEQ ID NOs:416, 418, 420, 422, and 424. In certain of these embodiments, the VL CDR2 comprises the amino acid sequence of SEQ ID NO:417, the VL CDR3 comprises the amino acid sequence of SEQ ID NO:424, the VH CDR2 comprises the amino acid sequence of SEQ ID NO: 451, and the VH CDR3 comprises the amino acid sequence of SEQ ID NO: 440. Sometimes, the VL CDR2 comprises the amino acid sequence of SEQ ID NO:423, the VL CDR3 comprises the amino acid sequence of SEQ ID NO:424, the VH CDR2 comprises the amino acid sequence of SEQ ID NO:451, and the VH CDR3 comprises the amino acid sequence of SEQ ID NO:440. In some embodiments, the radiolabeled activatable anti-PDL-1 antibody comprises a variable light chain comprising the amino acid sequence of SEQ ID NO:112 and a variable heavy chain comprising the amino acid sequence of SEQ ID NO:146. The prodomain employed in these embodiments, may comprise an MM comprising an amino acid sequence selected from the group consisting of SEQ ID NOs:84-108. In certain embodiments, the MM comprises the amino acid sequence of SEQ ID NO:90. Often, the CM comprises the amino acid sequence of SEQ ID NO:24. In some embodiments, the radiolabeled activatable anti-PDL-1 antibody comprises a light chain amino acid sequence comprising the amino acid sequence of SEQ ID NO:971, or SEQ ID NO:969, or SEQ ID NO:170, or SEQ ID NO:168, or SEQ ID NO:146. In some of these embodiments, the radiolabeled activatable anti-PDL-1 antibody comprises a heavy chain amino acid sequence comprising the amino acid sequence of SEQ ID NO:172.

In some embodiments, the radiolabeled activatable anti-PDL-1 antibody comprises a light chain amino acid sequence comprising the amino acid sequence of SEQ ID NO:168 and a heavy chain amino acid sequence comprising the amino acid sequence of SEQ ID NO: 172. In other embodiments, the radiolabeled activatable anti-PDL-1 antibody comprises a light chain amino acid sequence comprising the amino acid sequence of SEQ ID NO:169 and a heavy chain amino acid sequence comprising the amino acid sequence of SEQ ID NO:172.

Additional activatable anti-PDL-1 antibodies, and portions thereof, that are suitable for use in the practice of the present invention include those described in WO 2016/149201, which is incorporated herein by reference in its entirety.

The activatable binding polypeptide may further comprise additional moieties conjugated thereto that impart an additional property or function to the corresponding activated binding polypeptide, such as, for example, extended half-life (by conjugation to a polyethylene glycol (PEG) moiety, a human serum albumin (HSA) moiety, and the like), cytotoxicity (by conjugation to all or part of a toxin, such as, for example, a dolastin or derivative thereof (e.g., auristatin E, AFP, MMAF, MMAE, MMAD, DMAF, DMAE, and the like, and derivatives thereof); a maytansinoid or derivative thereof; DM1; DM4, a duocarmycin or derivative thereof; a calicheamicin or derivative thereof; a pyrrolobenzodiazepine or derivative or dimer thereof; a heavy metal (e.g., barium, gold, platinum, and the like), a pseudomonas toxin A variant (e.g., PE38, ZZ-PE38, and the like), ZJ-101, OSW-1, a 4-nitrobenzyloxycarbonyl derivative of 06-benzylguanine, a topoisomerase inhibitor, hemiasterlin, cephalotaxine, homoharringonine, a pyrrolobenzodiazepine dimer, a pyrrolobenzodiazepene, a functionalized pyrrolobenzodiazepene, a functionalized pyrrolobenzodiazepene dimer, a calicheamicin, a podophyllotoxin, a taxane, a vinca alkaloid, and the like)), as well as any of a variety of other known cytotoxic agents; anti-viral activity (e.g., by conjugation to all or a portion of Acyclovir, Vira A, Symetrel, Turbostatin, a Phenstatin, Hydroxyphenstatin, Spongistatin 5, Spongistatin 7, Halistatin 1, Halistatin 2, Halistatin 3, a modified bryostatin, a halocomstatin, pyrrolobenzimadazole, cibrostatin6, doxaliform, an anthracycline analogue, a cemadotin analogue (e.g., CemCH2-SH)); antifungal activity (e.g., Nystatin, and the like); anti-neoplastic activity (e.g., by conjugation to Adriamycin, cerubidine, bleomycin, alkeran, velban, oncovin, fluorouracil, methotrexate, thiotepa, bisantrene, novantrone, thioguanine, procarabizine, cytarabine, and the like); anti-bacterial activity (e.g., by conjugation to an aminoglycoside, streptomycin, neomycin, kanamycin, amikacin, gentamicin, tobramycin, Streptomycin B, spectinomycin, ampicillin, sulfanilamide, polymyxin, chloramphenicol, and the like), anti-mycoplasmal activity (e.g., by conjugation to tylosine, spectinomycin, and the like); and other desirable other additional properties and functions. Moieties that impart such desired properties and functions can be readily conjugated to the BP using methods and linkers that are known in the art. Radionuclides that are suitable for use in the radiolabeled activatable binding polypeptides employed herein include any that are suitable for use in positron emission tomography. These include, for example, ¹¹¹In (half-life 67.3 hours), ¹³¹I (half-life 192.5 hours), ¹²³I (half-life 13.2 hours), ^{99m}Tc (half-life 6.0 hours), ¹⁷⁷Lu (half-life 159.5 hours), ⁸⁹Zr (half-life 78.4 hours), ¹²⁴I (half-life 100.2 hours), ⁶⁴Cu (half-life 12.7 hours), ⁸⁶Y (half-life 14.7 hours), ⁷⁰Br (half-life 16.1 hours), ¹⁸F (half-life 1.83 hours), ⁶⁸Ga (half-life 1.13 hours), and the like, corresponding to an ¹¹¹In-conjugated activatable binding polypeptide, an ¹³¹I-conjugated activatable binding polypeptide, an ¹²³I-conjugated activatable binding polypeptide, a ^{99m}Tc-conjugated activatable binding polypeptide, a ¹⁷⁷Lu-conjugated activatable binding polypeptide, a ⁸⁹Zr-conjugated activatable binding polypeptide, an ¹²⁴I-conjugated activatable binding polypeptide, a ⁶⁴Cu-conjugated activatable binding polypeptide, a ⁸⁶Y-conjugated activatable binding polypeptide, a ⁷⁰Br-conjugated activatable polypeptide, a ¹⁸F-conjugated activatable binding polypeptide, and a ⁶⁸Ga-conjugated activatable polypeptide, respectively. In some embodiments, the radionuclide is ⁸⁹Zr.

The radionuclide is often present in the activatable binding polypeptide at a radionuclide:activatable binding polypeptide conjugation ratio in the range of from about 0.5 to about 3.0, or from about 0.5 to about 2.0, or from about 0.5 to about 1.5. The radiolabeled activatable binding polypeptide is often prepared by reacting a conjugated activatable binding polypeptide intermediate with the radionuclide to thereby label the activatable antibody. As used herein, the term “conjugated activatable binding polypeptide intermediate” refers to an activatable binding polypeptide that has conjugated thereto a labeling moiety that is capable of forming a bond with the radionuclide. Typically, conjugation of the labeling moiety to the activatable binding polypeptide is via a covalent bond. Usually, the labeling moiety and thus, the radionuclide, is conjugated to the activatable binding polypeptide at an amino acid residue within the portion of the activatable binding polypeptide that is conserved in the corresponding activated binding polypeptide. In some embodiments, the labeling moiety is conjugated to the activatable binding polypeptide at an amino acid residue in a region selected from the group consisting of a variable region and a constant region of the activatable binding polypeptide. Often, the labeling moiety is conjugated to the activatable binding polypeptide via a linkage selected from the group consisting of an amide linkage and an ester linkage. In some embodiments, the labeling moeity is conjugated to a lysine residue and/or arginine residue. Often, the reactive moiety is conjugated to a lysine residue.

In an exemplary embodiment, the labeling moiety comprises a chelation moiety. The term “chelation moiety” refers to a moiety that is capable of forming one or more bonds with the radionuclide. In these embodiments, the radiolabeled activatable binding polypeptide further comprises a chelation moiety to which the radionuclide is chelated. When a chelation moiety is employed, it is conjugated to an amino acid residue in the activatable antibody. The chelation moiety may comprise a further substituent to facilitate and direct conjugation to the activatable binding polypeptide. In some embodiments, the further substituent comprises a succinyl substituent (i.e., the chelation moiety comprises succinyldeferoxamine (also referred to as “succinyldesferal”)). In some embodiments, the conjugated activatable binding polypeptide intermediate is an N-succinyldesferal activatable binding polypeptide. The present invention further provides conjugated activatable binding polypeptide intermediates N-succinyldesferoxamine-Fe (prepared by reacting N-succinyldesferal with Fe (III)) and 2,3,5,6-tetrafluorophenol (TFP)-N-succinyldesferal-Fe (prepared by reacting tetrafluorophenol with N-succinyldesferoxamine-Fe). The type of bond through which conjugation occurs will often depend on the nature of the chelation moiety and the amino acid residue targeted for conjugation.

Exemplary conjugated activatable binding polypeptide that comprise chelation moieties include those which result from reaction of the activatable binding polypeptide with chelation agents such as, for example, diethylenetriaminepentaacetic acid (DTPA), ethylenediaminetetraacetic acid (EDTA), 1,4,7,10-tetraacetic acid (DOTA), deferoxamine (DFO, sold under the brand name, DESFERAL (deferoxamine mesylate (i.e., N′[(Acetyl-hydroxy-amino)pentyl]-N-[5-[3-(5-aminopentyl-hydroxy-carbamoyl)propanoylamino]pentyl]-N-hydroxy-butane diamide), and the like. Thus, the structure of the chelation moiety corresponds to the structure of the structure of the chelation agent with the exception of the portion of the chelation agent that is conjugated to the amino acid residue of the activatable binding polypeptide. Thus, in some embodiments, the chelation moiety may comprise a structure corresponding to a chelation agent selected from the group consisting of diethylenetraminepentaacetic acid, ethylenediaminetetraacetic acid, 1,4,7,10-tetraacetic acid, and deferoxamine. Often, the radiolabeled activatable binding polypeptide comprises a chelation moiety comprising a structure corresponding to deferoxamine.

Known methods for preparing radiolabeled antibodies using chelation agents are suitable for preparing the radiolabeled activatable binding polypeptides employed herein. These methods are described in, for example, Chan, et al., Pharmaceuticals (2012) 5:79-91, van de Watering, et al., BioMed Research International Vol. 2014, Article ID 203601 (2014), Zhang, et al., Curr. Radiopharm. (2011) 4(2):131-139, and LeBeau, et al., Cancer Res. (2015) 75(7):1225-1235, Verl, et al., J. Nucl. Med. (2003) 44:1271-1281, Vosjan, et al., Eur. J. Nucl. Med. Mol. Imaging (2011) 38:753-763, each of which is incorporated herein by reference in their entireties.

The present invention further provides a method of making a radiolabeled activatable binding polypeptide comprising reacting a radionuclide with an activatable binding polypeptide or conjugated activatable binding polypeptide intermediate under conditions sufficient to form a bond between the radionuclide and the activatable binding polypeptide or labeling moiety. In one embodiment, the radiolabeled activatable binding polypeptide comprises a labeling moiety that comprises deferoxamine. In another embodiment, the method further comprises complexing the deferoxamine component of the labeling moiety with Fe (III) prior to the step of reacting a radionuclide with the activatable polypeptide or conjugated activatable binding polypeptide intermediate.

In one embodiment, the radiolabeled activatable binding polypeptide (and chelation moiety) comprises a radiolabeled N-succinyldesferal activatable binding polypeptide (i.e., comprises an N-succinyldesferal (N-sucDf) moiety chelated to the radionuclide, wherein the N-succinyldesferal moiety is conjugated to the activatable binding polypeptide. In a specific embodiment, the present invention provides a radiolabeled N-succinimidyl desferal activatable binding polypeptide. In certain embodiments, the radiolabeled activatable binding polypeptide is an ⁸⁹-conjugated N-succinimidyl desferal activatable binding polypeptide, such as, for example, an ⁸⁹Zr-conjugated N-succinimidyl desferal activatable antibody.

In some embodiments, the radiolabeled activatable binding polypeptide comprises an N-succinyldesferal-⁸⁹Zr substituent. An exemplary method for carrying out the conjugation of a monoclonal antibody with ⁸⁹Zr via a desferal and N-succinyldesferal-Fe synthetic route is described in Veral, et al., “⁸⁹Zr Immuno-PET: Comprehensive Procedures for the Production of ⁸⁹Zr-Labeled Monoclonal Antibodies,” J. Nucl. Med. (2003) 44(8): 1271.

During the course of manufacture of radiolabelled activatable binding polypeptide, it may be desired to produce and store conjugation intermediates prior to labeling the conjugation intermediate with the radiolabel, or, alternatively, carry out the labeling of the conjugation intermediate at a different facility. In this regard, the present invention provides a stable conjugation intermediate comprising an activatable binding polypeptide having conjugated thereto a chelation moiety. The dose of a radiolabeled activatable binding polypeptide (i.e., the “tracer” dose) is often administered in the form of a composition comprising a radiolabeled activatable binding polypeptide and one or more of a suitable carrier, an excipient, and/or other agent(s) that are incorporated into pharmaceutical formulations to provide improved transfer, delivery, tolerance, stability, and the like. In some embodiments, the carrier is a physiological saline solution (i.e., 0.9% NaCl), a saccharide solution (e.g., dextrose, and the like), an alcohol (e.g., ethanol), a polyol (e.g., a polyalcohol, such as, for example, mannitol, sorbitol, and the like), a glycol, such as ethylene glycol, propylene glycol, PEG, a coating agent, an isotonic agent, such as mannitol or sorbitol, an organic ester, such as ethyoleate, an absorption-delaying agent, such as aluminum monostearate and gelatins and the like. The composition can be in the form of a stable, aqueous solution. The aqueous solution may comprise an isotonic vehicle such as sodium chloride, Ringer's injection solution, dextrose, lactated Ringer's injection solution, or equivalent delivery vehicle (e.g., sodium chloride/dextrose injection solution). The composition may comprise aqueous and non-aqueous, isotonic sterile injection solutions, which can include solvents, co-solvents, antioxidants, reducing agents, chelating agents, buffers, bacteriostats, antimicrobial preservatives and solutes that render the composition isotonic with the blood of the intended recipient (e.g., PBS and/or saline solutions, such as 0.1 M NaCl) and aqueous and non-aqueous sterile suspensions that can include suspending agents, solubilizers, thickening agents, emulsifying agents, stabilizer, preservatives, and the like. Suitable agents can be found in Remington's Pharmaceutical Science (15th ed. Mack Publishing Company, Easton, Pa. (1975)), which is incorporated herein by reference in its entirety.

In some embodiments, the tracer dose comprises about 5 MBq or less of the radiolabeled activatable binding polypeptide. In other embodiments the dose comprises a quantity of radiolabeled activatable binding polypeptide corresponding to a radiation activity in the range of from about 1 MBq to about 5 MBq, or from about 1 MBq to about 4.5 MBq, or from about 1 MBq to about 4 MBq, or from about 2 MBq to about 4 MBq. In certain embodiments, the tracer dose comprises a quantity of radiolabeled activatable binding polypeptide corresponding to a radiation activity of about 3.7 MBq (100 μCi). The tracer dose is typically administered in the form of a composition comprising the radiolabeled activatable binding polypeptide and a carrier. The carrier in the composition of the tracer dose (i.e., “tracer dose composition”) is typically a liquid phase carrier. Typically, the mammalian subject is a human or non-human mammal suspected of having a disease or disorder. Usually the suspected disease or disorder is a cancer, as described in more detail hereinbelow.

In some embodiments, administration of the dose of radiolabeled activatable binding polypeptide is accompanied by administration of a blocking dose of corresponding non-radiolabeled (or “cold”) activatable binding polypeptide. The doses of radiolabeled and non-radiolabeled activatable binding polypeptide may be administered as a single dose of a composition comprising both radiolabeled and non-radiolabeled activatable binding polypeptide, or may be administered in two steps as a dose of cold activatable binding polypeptide and a dose of radiolabeled activatable binding polypeptide. When a blocking dose is administered, it is usually administered prior to administering the dose of radiolabeled activatable binding polypeptide to pre-block non-specific antigen sinks.

In some embodiments, the blocking dose comprises cold activatable binding polypeptide in quantity that is in the range of from about 0.1 mg/Kg to about 10 mg/Kg, or may be in the range of from about 0.2 mg/Kg to about 10 mg/Kg, or from about 0.3 mg/Kg to about 10 mg/Kg, or from about 0.01 mg/Kg to about 0.3 mg/Kg, or from about 0.01 mg/Kg to about 0.2 mg/Kg, or from about 0.01 mg/Kg to about 0.1 mg/Kg. In some embodiments, the blocking dose comprises the cold activatable binding polypeptide in a quantity that is less than a therapeutic dose. In some embodiments, the blocking dose comprises a fixed dose of about 5 mg or a dose of about 0.07 mg/Kg.

As used herein, the term “therapeutic dose” refers to a quantity of cold activatable binding polypeptide that lessens one or more symptoms of the disease or disorder. In certain embodiments, the blocking dose comprises the cold activatable binding polypeptide in a quantity that is about 0.1 mg/Kg, or about 0.2 mg/Kg, or about 0.3 mg/Kg, or about 1 mg/Kg, or about 3 mg/Kg, or about 10 mg/Kg. In some embodiments, the blocking dose comprises the cold activatable binding polypeptide in a quantity that is less than about 0.3 mg/Kg, or less than about 0.2 mg/Kg, or less than about 0.1 mg/Kg, but greater than about 0.01 mg/Kg.

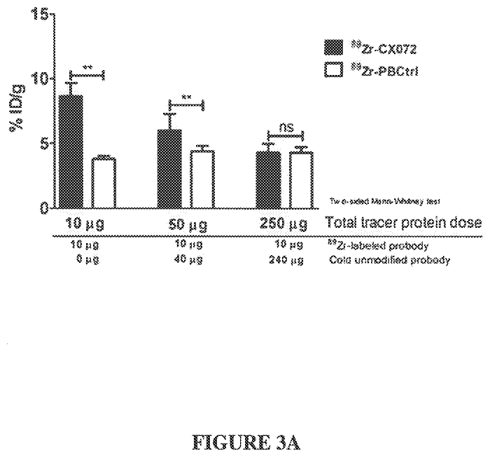

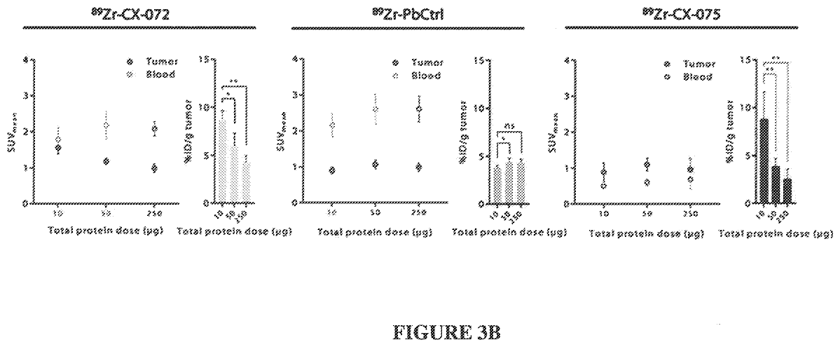

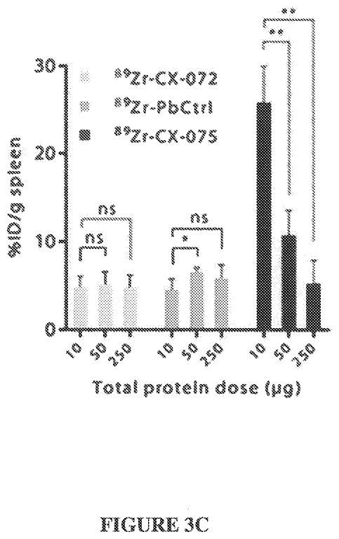

In some embodiments, no blocking dose or a de minimus quantity of the corresponding cold activatable binding polypeptide is administered to the mammalian subject. The term a “de minimis quantity of the corresponding cold activatable binding polypeptide” refers to a quantity of the corresponding cold activatable binding polypeptide that results in no detectable difference in resulting PET image when compared to the situation where no blocking dose is administered to the subject. Administration of a relatively small blocking dose, or omission of a blocking dose, may lead to greater uptake of activated binding polypeptide in the target organ or tissue. As depicted in FIG. 3A (Example 1), tumor uptake of an ⁸⁹Zr-labeled activatable binding polypeptide in a mouse model was greatest when no corresponding unlabeled activatable binding polypeptide was administered.

Treated subjects are typically subjected to positron emission tomography (PET) scanning at one or more time-points in the period of from about 1 day to about 10 days post tracer dose administration. In some embodiments, the treated subject is subjected to PET scanning at a time point in the period of from about 2 days to about 10 days post tracer dose administration, or in the period of from about 2 days to about 9 days post tracer dose administration, or in the period of from about 2 days to about 8 days post tracer dose administration, or in the period of from about 2 days to about 7 days post tracer dose administration, or in the period of from about 3 days to about 10 days post tracer dose administration, or in the period of from about 3 days to about 9 days post tracer dose administration, or in the period of from about 3 days to about 8 days post tracer dose administration. In certain embodiments, the treated subject is subjected to PET scanning at day 2, and/or day 4, and/or day 7 post tracer dose administration. In other embodiments, the treated subject is subjected to PET scanning at day 1, and/or day 3, and/or day 6 post tracer dose administration.

Typically, the resulting PET scan covers an area that includes one or more organs or tissue corresponding to the heart, blood, lung, liver, kidney, pancreas, stomach, ilium, colon, muscle, bone, skin, brain, thymus, brown adipose tissue (BAT), spleen, and/or tumor. Usually the PET scan covers an area that includes all or a portion of a tumor. In some embodiments, the PET scan covers an area that includes all or a portion of a tumor and all or a portion of at least one other organ or tissue type.

Detection of radionuclide in the PET scan indicates the presence of activated binding polypeptide and the location and thus the in vivo biodistribution of activated binding polypeptide in the mammalian subject. Detection of activated binding polypeptide indicates not only that the administered activatable binding polypeptide was activated, e.g., by proteases in the target microenvironment, but that the biological target was also present.

The method may be further used to identify subjects more likely to benefit from treatment with a particular activatable binding polypeptide. For example, if the biodistribution indicates the presence of activated binding polypeptide in a tumor, the subject may be more likely to benefit from the administration of an activatable binding polypeptide designed to treat the associated cancer. Thus, the present invention provides a method for identifying a mammalian subject suitable for treatment with an activatable binding polypeptide, the method comprising:

detecting the in vivo distribution of an activated binding polypeptide in a mammalian subject in accordance with the method of detecting the in vivo distribution of an activated binding polypeptide, as described herein, and

identifying the mammalian subject as being suitable for treatment with the activatable binding polypeptide if the radionuclide is detectably present within the PET image of the tumor. In some embodiments, the method further comprises obtaining a tumor tissue sample from the subject.

In one embodiment, the mammalian subject has been previously diagnosed with a disease or disorder. Often, the disease or disorder is a cancer. Exemplary types of cancer, include, for example, an advanced, unresectable solid tumor or lymphoma (e.g., a PDL1-responsive tumor type); a carcinoma such as, for example, carcinoma squamous cell carcinoma, an anal squamous cell carcinoma, gastric carcinoma, bowel carcinoma (such as, for example, small bowel carcinoma or small bowel adenocarcinoma), hepatocellular carcinoma, or a basal cell carcinoma; bladder cancer; bone cancer; breast cancer, such as, for example, triple negative breast cancer (TNBC) or estrogen receptor positive breast cancer; a carcinoid; castration-resistant prostate cancer (CRPC), cervical carcinoma, colon cancer (such as, for example, a colon adenocarcinoma); cutaneous squamous cell carcinoma, colorectal cancer (CRC), endometrial cancer, esophageal cancer, gastroesophageal junction cancer, glioblastoma/mixed glioma, glioma, head and neck cancer, hematologic malignancy, such as, for example, a lymphoma (such as, for example, a B-cell lymphoma, a T-cell lymphoma, Hodgkin's lymphoma, an EBV lymphoma, or a primary mediastinal B-cell lymphoma) or a leukemia; liver cancer, lung cancer (such as, for example, non-small cell lung cancer (NSCLC) (such as, for example, non-squamous NSCLC or squamous NSCLC) or small cell lung cancer); melanoma, Merkel cell carcinoma, multiple myeloma, nasopharyngeal cancer, osteosarcoma, ovarian cancer, pancreatic cancer, peritoneal carcinoma, undifferentiated pleomorphic sarcoma, prostate cancer (such as, for example, small cell neuroendocrine prostate cancer); rectal carcinoma, renal cancer (such as, for example, a renal cell carcinoma or a renal sarcoma); sarcoma, salivary gland carcinoma, squamous cell carcinoma, stomach cancer, testicular cancer, thymic carcinoma, thymic epithelial tumor, thymoma, thyroid cancer, urogenital cancer, urothelial cancer, uterine carcinoma, uterine sarcoma, and the like. In some embodiments, the cancer is a High Tumor Mutational Burden (hTMB) cancer.

Often, the mammalian subject has been previously diagnosed as having melanoma. In carrying out the practice of the present invention, some mammalian subjects have been previously diagnosed as having a cancer selected from the group consisting of undifferentiated pelomorphic sarcoma, small bowel adenocarcinoma, Merkel cell carcinoma, thymic carcinoma, anal squamous cell carcinoma, cutaneous squamous cell carcinoma, and triple negative breast cancer.

In a further embodiment, the present invention provides a method of treating a mammalian subject in need thereof with an activatable binding polypeptide, the method comprising:

identifying a mammalian subject suitable for treatment with an activatable binding polypeptide in accordance with the methods of the present invention; and

administering to the mammalian subject a therapeutically effective dose of the activatable binding polypeptide.

In carrying out the methods described herein, typically, the mammalian subjects are human. As used herein, the term, “therapeutically effective dose” refers to a quantity of activatable binding polypeptide effective in alleviating a symptom of a disease or disorder when administered either once, or in a series over a period of time. Therapeutically effective doses for anti-PDL-1 activatable antibodies can be found, for example, in WO 2018/222949, which is incorporated herein by reference. For example, when the activatable binding polypeptide is an activatable anti-PDL-1 antibody, the therapeutically effective dose may be in a range of from about 0.3 mg/kg to about 15 mg/kg (e.g., human), or in the range of from about 0.3 mg/kg to about 10 mg/kg, or in the range of from about 3 mg/kg to about 15 mg/kg, or in the range of from about 3 mg/kg to about 10 mg/kg (e.g., human). In some embodiments, the therapeutically effective dose is about 0.3 mg/kg, or is about 1 mg/kg, or is about 3 mg/kg, or is about 6 mg/kg (e.g., human).

### Compounds and Compositions

In another aspect, the present invention provides an ⁸⁹Zr-conjugated activatable binding polypeptide that is a useful as a tracer in connection with PET imaging a tumor in a mammalian subject. In some embodiments the ⁸⁹Zr-conjugated activatable binding polypeptide is an ⁸⁹Zr-conjugated activatable antibody, which may comprise any of the activatable anti-PDL-1 antibodies (including portions thereof) described herein. In a specific embodiment, the ⁸⁹Zr-conjugated activatable binding polypeptide is a ⁸⁹Zr-conjugated N-succinimidyl desferal activatable anti-PDL-1 antibody, which may comprise any of the activatable anti-PDL-1 antibodies (including portions thereof) described herein.

In a further embodiment, the present invention provides a composition comprising a radiolabeled activatable binding polypeptide and a carrier, wherein the radiolabeled activatable binding polypeptide comprises a radionuclide and an activatable binding polypeptide, wherein the activatable binding polypeptide comprises a binding moiety and a prodomain, wherein the prodomain comprises a masking moiety and a cleavable moiety. Radiolabeled activatable binding polypeptides that are suitable for use in the compositions of the present invention include any of those described hereinabove. Carriers that may be employed include any known in the art that are suitable for use in pharmaceutical products, and include those described hereinabove. The compositions may further include pharmaceutically acceptable excipients and additives. Carriers, excipients, and agents that may be employed in the practice of the present invention may be found in Remington's Pharmaceutical Science (15th ed. Mack Publishing Company, Easton, Pa. (1975)), which is incorporated herein by reference in its entirety. The compositions may further comprise a corresponding non-radiolabeled activatable binding polypeptide.

In one embodiment, the composition comprises the radiolabeled activatable binding polypeptide and a solid phase carrier. In these embodiments, the composition is typically in lyophilized form. Prior to administering the radiolabeled activatable binding polypeptide to the mammalian subject, the composition is reconstituted to a solution form by addition of a liquid to form the tracer dose composition, where the tracer dose composition comprises the radiolabeled activatable binding polypeptide at the desired quantity in the tracer dose. Typically, the liquid is physiological saline (0.9% NaCl). The term “tracer dose composition” refers to the composition of the tracer dose that is administered to the mammalian subject. In other embodiments, the composition comprises the radiolabeled activatable binding polypeptide and a liquid phase carrier. This composition may be the tracer dose composition, or it may be a composition that is diluted by addition of a liquid, e.g., physiological saline (0.9% NaCl), to a tracer dose composition comprising the radiolabeled activatable binding polypeptide at the desired quantity in the tracer dose.

In a further embodiment, the present invention provides a composition that is stable after storage at a temperature in the range of from about 2 to about 8° C. for a time period of at least about 1 month, or at least about 3 months, or at least about 6 months, or at least about 12 months, with respect to one or more properties selected from the group consisting of concentration of aggregates, concentration of radiolabeled activatable binding polypeptide, pH, and radiochemical purity. Often, the time period is at least about 6 months. In some embodiments, the composition is stable with respect to one or more of the above-described properties after a period of at least about 12 months. As used, herein, the term “stable” means that a metric associated with the specified property has not changed more than 20% from a measurement of the metric taken at an initial time point, just prior to implementation of the storage conditions. In some embodiments, the property remains within about 15%, or within about 14%, or within about 13%, or within about 12%, or within about 11%, or within about 10%, or within about 9%, or within about 8%, or within about 7%, or within about 6%, or within about 5%, or within about 4%, or within about 3%, or within about 2% or within about 1% of the same property at an initial time point. Concentration of aggregates is measured by Size Exclusion (SE)-HPLC measured at 280 nm. Concentration of radiolabeled activatable binding polypeptide may be determined by UV spectrophotometry. Radiochemical purity is determined by TCA assay. Often, the stable composition comprises an ⁸⁹Zr-conjugated N-succinimidyl desferal activatable binding polypeptide, such as, for example an ⁸⁹Zr-conjugated N-succinimidyl desferal activatable anti-PDL-1 antibody (including portions thereof) in accordance with any of the embodiments described herein, having a radionuclide:activatable binding polypeptide conjugation ratio in the range of from about 0.5 to about 3.0, or from about 0.5 to about 2.0, or from about 0.5 to about 1.5. In a specific embodiment, the stable composition comprises an ⁸⁹Zr-conjugated N-succinimidyl desferal activatable anti-PDL-1 antibody comprising a light chain sequence comprising the amino acid sequence of SEQ ID NO:168 and a heavy chain amino acid sequence comprising the amino acid sequence of SEQ ID NO:172.

Often, the concentration of aggregates remains at a level of less than 5% of the composition after the storage period of about 6 or 12 months, under the storage conditions described hereinabove. The concentration of radiolabeled activatable binding polypeptide in the composition often remains within 15%, or within 10%, or within 5% of the initial concentration of the radiolabeled activatable binding polypeptide, after a period of about 6 or 12 months, under the storage conditions described hereinabove. The pH of the composition often remains within 5%, or within 4%, or within 3%, or within 2%, or within 1% of an initial pH, after a period of about 6 or 12 months, under the storage conditions described hereinabove. The radiochemical purity of the composition often is at least 95%, or at least 96%, or at least 97%, or at least 98%, or at least 99% of an initial radiochemical purity, after a period of about 5 or 12 months, under the storage conditions described hereinabove.

### SPECIFIC EMBODIMENTS OF THE PRESENT INVENTION

Embodiments of the invention include the following:

1. A method for detecting an in vivo distribution of an activated binding polypeptide in a subject, the method comprising:

administrating to a mammalian subject a tracer dose of a radiolabeled activatable binding polypeptide,
wherein the radiolabeled activatable binding polypeptide comprises a radionuclide and an activatable binding polypeptide,
   wherein the activatable binding polypeptide comprises a prodomain and a binding moiety, wherein the prodomain comprises a masking moiety and a cleavable moiety,
   wherein, when the radiolabeled activatable binding polypeptide is activated, a radiolabeled activated binding polypeptide is generated that is capable of specifically binding, in vivo, a biological target; and

imaging the mammalian subject using positron emission tomography (PET) at a time point following administration of the tracer dose.

2. The method of embodiment 1, wherein the radionuclide is selected from the group consisting of ¹¹¹In, ¹³¹I, ¹²³I, ^{99m}Tc, ¹⁷⁷Lu, ⁸⁹Zr, ¹²⁴I, ⁶⁴Cu, ⁸⁶Y, ⁷⁰Br, ¹⁸F, and ⁶⁸Ga.

3. The method of embodiment 3, wherein the radionuclide is ⁸⁹Zr.

4. The method of any of embodiments 1-3, wherein the tracer dose comprises a quantity of the radiolabeled activatable binding polypeptide corresponding to a radiation activity in the range of from about 1 MBq to about 5 MBq, or from about 1 MBq to about 4.5 MBq, or from about 1 MBq to about 4 MBq, or from about 2 MBq to about 4 MBq.

5. The method of embodiment 4, wherein the tracer dose comprises a quantity of the radiolabeled activatable binding polypeptide corresponding to a radiation activity of about 3.7 MBq.

6. The method of any of embodiments 1-5, further comprising administration of a blocking dose to the mammalian subject, wherein the blocking dose comprising a corresponding non-radiolabeled activatable binding polypeptide.

7. The method of embodiment 6, wherein administration of the blocking dose precedes administration of the tracer dose.

8. The method of embodiment 6, wherein the blocking dose and tracer dose are administered as a single composition comprising the radiolabeled activatable binding polypeptide and the corresponding non-radiolabeled activatable binding polypeptide.

9. The method of any of embodiments 6-8, wherein the blocking dose comprises a quantity of the corresponding non-radiolabeled activatable binding polypeptide in the range of from about 0.1 mg/Kg to about 10 mg/Kg, and may be in the range of from about 0.2 mg/Kg to about 10 mg/Kg, or from about 0.3 mg/Kg to about 10 mg/Kg.

10. The method of any of embodiments 6-8, wherein the blocking dose comprises about 0.1 mg/Kg. or about 0.2 mg/Kg, or about 0.3 mg/Kg, or about 1 mg/Kg, or about 3 mg/Kg, or about 10 mg/Kg of the corresponding non-radiolabeled activatable binding polypeptide.

11. The method of any of embodiments 6-8, wherein the blocking dose comprises the corresponding non-radiolabeled activatable binding polypeptide in a quantity that is less than about 0.3 mg/Kg, or less than about 0.2 mg/Kg, or less than about 0.1 mg/Kg, but greater than about 0.01 mg/Kg.

12. The method of any of embodiments 1-11, wherein the imaging step occurs at a time point in the period of from about 1 day to about 10 days post tracer dose administration, or at a time point in the period of from about 2 days to about 10 days post tracer dose administration, or in the period of from about 2 days to about 9 days post tracer dose administration, or in the period of from about 2 days to about 8 days post tracer dose administration, or in the period of from about 2 days to about 7 days post tracer dose administration, or in the period of from about 3 days to about 10 days post tracer dose administration, or in the period of from about 3 days to about 9 days post tracer dose administration, or in the period of from about 3 days to about 8 days post tracer dose administration.

13. The method of any of embodiments 1-12, wherein the mammalian subject is subjected to PET scanning at a time point corresponding to day 2, and/or day 4, and/or day 7 post tracer dose administration.

14. The method of any of embodiments 1-13, wherein the imaging step results in a resulting PET scan that covers an area that includes one or more organs or tissue corresponding to the heart, blood, lung, liver, kidney, pancreas, stomach, ilium, colon, muscle, bone, skin, brain, thymus, brown adipose tissue (BAT), spleen, and/or tumor.

15. The method of embodiment 14, wherein the PET scan covers an area that includes all or a portion of a tumor.

16. The method of embodiment 15, wherein the PET scan covers an area that further covers at least all or a portion of one additional organ or tissue.

17. The method of any of embodiments 1-16, wherein the activatable binding polypeptide is an activatable antibody.

18. A composition comprising a radiolabeled activatable binding polypeptide and a carrier, wherein the radiolabeled activatable binding polypeptide comprises a radionuclide and an activatable binding polypeptide, wherein the activatable binding polypeptide comprises a binding moiety and a prodomain, wherein the prodomain comprises a masking moiety and a cleavable moiety.

19. The composition of embodiment 18, wherein the radionuclide is selected from the group consisting of ¹¹¹In, ¹³¹I, ¹²³I, ^{99m}Tc, ¹⁷⁷Lu, ⁸⁹Zr, ¹²⁴I, ⁶⁴Cu, ⁸⁶Y, ⁷⁰Br, ¹⁸F, and ⁶⁸Ga.

### ACKNOWLEDGMENTS

The radiolabelling and PET imaging studies described herein were conducted at and in collaboration with the University Medical Center Groningen (UMCG), Hanzeplein 1, 9700 RB Groningen, The Netherlands.

The following examples further illustrate the invention, but should not be construed as limiting its scope in any way.

### EXAMPLES

### Example 1

### Biodistribution of a Radiolabeled Activatable Antibody

In this study, CX-072, an activatable anti-PD-L1 antibody corresponding to SEQ ID NO:168 (light chain sequence encoded by the polynucleotide sequence of SEQ ID NO:167) and SEQ ID NO:172 (heavy chain sequence encoded by the polynucleotide sequence of SEQ ID NO:171), a non-specific (non-binding) activatable antibody control (PbCtrl), and CX-075 (⁸⁹Zr-PDL1-Ab (having a heavy chain sequence corresponding to SEQ ID NO:174, and a light chain sequence corresponding to SEQ ID NO:175), were radiolabeled with 500 MBq/mg ⁸⁹Zr using the bifunctional chelator N-succinyldesferrioxamine-B-tetrafluorphenol (“desferal-N-suc-TFP” or “Df-suc-N-TFP”, ABX Gmbh). CX-072 was prepared as described in WO 2016/149201, which is incorporated herein by reference in its entirety. CX-072-N-sucDf, PbCtrl-N-sucDf, and CX-075-N-sucDf were purified using a Vivaspin-2 concentrator, aliquoted and stored at −80° C. Concentration and purity were determined by a Waters size exclusion high-performance liquid chromatography (SE-HPLC) system equipped with a dual-wavelength absorbance detector (280 nm versus 430 nm), in-line radioactivity detector and TSK-Gel SW column G3000SWXL 5 μm, 7.8 mm (joint Analytical Systems; mobile phase: phosphate buffered saline (PBS; 9.0 nM sodium phosphate, 1.3 mM potassium phosphate, 140 mM sodium chloride, pH 7.2) (Hospital Pharmacy UMCG); flow: 0.7 mL/min).

CX-072-N-sucDf, PbCtrl-N-sucDf and CX-075-N-sucDf were radiolabeled with clinical grade ⁸⁹Zr (Perkin Elmer) using the method described in Nagengast, et al., J. Nucl. Med. 48:1313-1310 (2007).

Immunoreactivity to PD-L1 of CX-072 and CX-075 after conjugation to TFP-N-sucDf was assessed by an indirect enzyme-linked immunosorbent assay (ELISA). 96-well plates (Nunc Maxisorp) were coated with 1 μg/mL human extracellular PD-L1 domain (R&D Systems; 156-B7-100) diluted in PBS (Givco; 0.7 mM sodium phosphate, 1.5 mM potassium phosphate, 154 mM sodium chloride, pH 7.2) and incubated overnight at 4° C. Wells were blocked for 2 hours at room temperature (RT) with 1% bovine serum albumin (Sigma-Adrich), 0.05% Tween 20 in PBS. After blocking, plates were incubated with either unconjugated CX-072, PbCtrl or CX-075 or their respective N-sucDf-conjugates in a concentration ranging from 0.00914 to 600 nM for 60 minutes at RT. Plates were subsequently washed with 0.05% Tween 20 in PBS and incubated with horseradish peroxidase-labeled anti-human IgG antibody (Sigma-Aldrich; A0293) for 60 minutes at RT. Detection was performed with single-component TMB peroxidase substrate (BioRad) and optical density read-out was performed at 450 nm using a micro plate-reader. Immunoreactivity to PD-L1 was expressed as the effective concentration needed for 50% of receptor occupation (EC50).

Immunoreactivity was determined by ELISA. The results showed that immunoreactivity to PD-L1 was preserved for CX-072-N-sucDf and CX-075 N-sucDf.

### Evaluation in MDA-MB-231 Tumor Model

For in vivo studies, PD-L1 expressing MDA-MB-231 triple negative human breast cancer cells (MD Anderson Cancer Center (Houston, Tex.) were subcutaneously (sc) engrafted in Balb/c nude mice. To assess tracer protein dose dependency of the tumor uptake (indicative of antigen-dependency of ⁸⁹Zr-CX-072 tumor uptake and potential for antigen saturation), mice received 10 μg ⁸⁹Zr-CX-072, 89Zr-PbCtrl, or CX-075 (˜5 MBq) supplemented with 0, 40, or 240 μg of non-radiolabeled CX-072, PbCtrl, or CX-075, respectively.

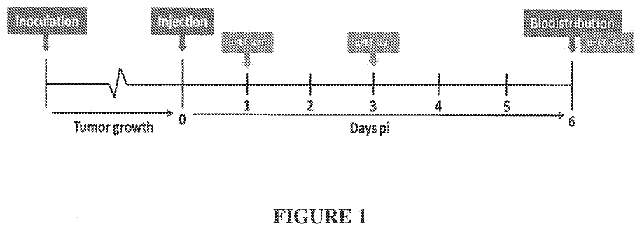

To evaluate ⁸⁹Zr-CX-072 biodistribution in an immune-competent setting, C57BL6 mice were implanted subcutaneously (sc) with low PD-L1 expressing MC38 syngeneic murine colon adenocarcinoma cells. All mice underwent serial in vivo PET imaging 1, 3 and 6 days post injection (pi), followed by tissue collection for ex vivo biodistribution. MicroPET images were quantified by mean standardized uptake value (SUVmean). A schematic depicting the in vivo study design is provided in FIG. 1. Activated antibody species were detected by Western capillary electrophoresis (Wes™ System, ProteinSimple). Tracer integrity in tumor lysates and plasma was assessed by SDS-PAGE. Autoradiography, PD-L1 immunofluorescence (IF) and PD-L1 immunohistochemistry (IHC) were performed on formalin-fixed paraffin-embedded 4 μm tumor slides.

All animal experiments were approved by the institutional animal care and use committee of the University of Groningen, and were performed in accordance with their guidelines. In vivo imaging and biodistribution experiments with 89Zr-CX-072 and 89Zr-PbCtrl were conducted in 5-7 week old male Balb-c/Ola HSD-fox nude (Balb-c/nude) or C57BL/6JOlaHsd (C57BL/6) mice obtained from Envigo. Male Balb-c/nude mice were injected subcutaneously (sc) on the right flank with 5.0×106 MDA-MB-231 cells in 0.3 mL PBS mixed equally with 0.3 mL Matrigel™ matrix (Corning). Male C57BL/6 mice were injected sc on the right flank with 1.5×106 MC38 cells (cell line derived from murine colon adenocarcinoma cells) mixed equally with 0.2 ml PBS. Animals were used for in vivo studies when the tumor volume measured≥200 mm3, 6-8 mm in diameter, approximately 4-5 weeks after inoculation.

Animals used for imaging and biodistribution studies were injected intravenously into the penile vein with 150 μl tracer solution, containing 10 μg ⁸⁹Zr-CX-072. 10 μg 89Zr-labeled non-binding isotype activatable antibody control (⁸⁹Zr-PBCtrl), or 10 g ⁸⁹Zr-CX-075 (5 MB1±0.5 MBq, 10 μg supplemented with 0, 40, 240 μg non-radiolabeled CX-072 or non-radiolabeled PBCtrl) resulting in total protein doses of 10, 50, 250 μg). Mice were subsequently scanned after 24, 72, and 144 h (i.e., 1 day, 3 days, and 6 days, respectively) post-injection (p.i.) using a Focus 220 microPET (CTI Molecular Imaging, Inc.) and subsequently sacrificed after the final scan. Organs of interest were excised, cleaned from blood and weighed. Samples and primed standards were counted in a calibrated well-type gamma-counter for radioactivity, and results expressed as percentage of injected dose per gram tissue (% ID/g).

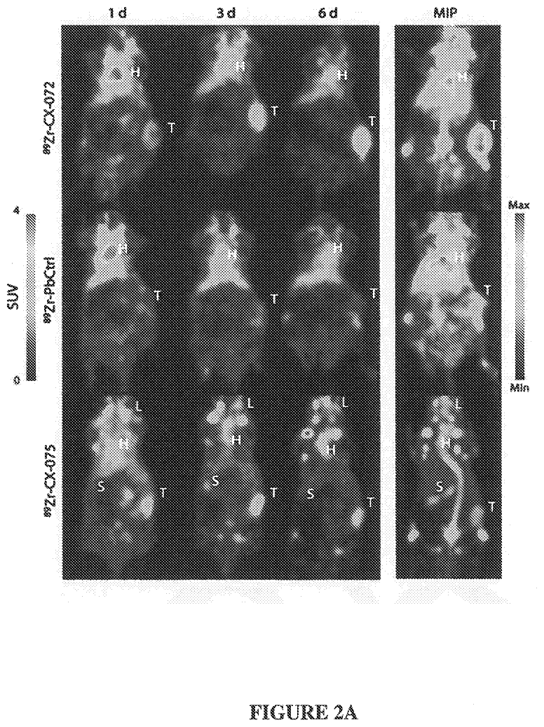

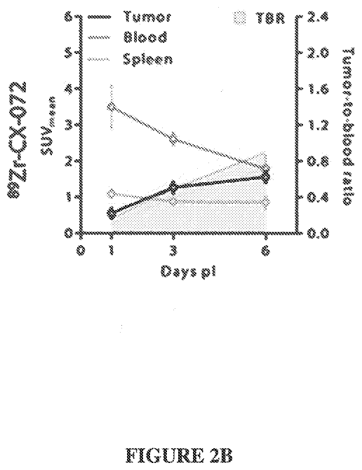

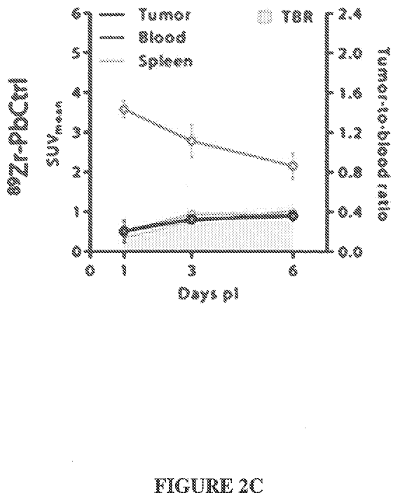

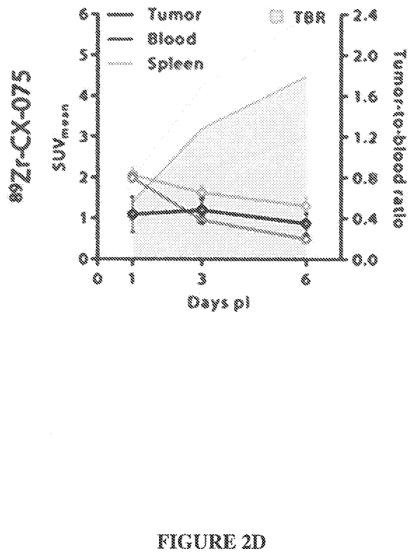

MicroPET scans indicated that tumor uptake of ⁸⁹Zr-CX-072 in MDA-MB-231 xenograft bearing Balb-c/nude mice increased over time with maximal tumor uptake at 6 days (144 h) post injection, as shown in FIG. 2A. FIG. 2A provides a representative set of MicroPET images taken at 1 day (24 h), 3 days (72 h), and 6 days (144 h), post injection (p.i.) for 10 μg of ⁸⁹Zr-CX-072, ⁸⁹Zr-PBCtrl, and ⁸⁹Zr-CX-075 in MDA-MB-231 xenograft bearing Balb-c/nude mice.

Comparison of ⁸⁹Zr-CX-072 and ⁸⁹Zr-PBCtrl: PET imaging at 1 day (24 h) p.i. revealed high uptake by the heart (H) and other tissues for both tracers. In time, relative uptake in the tumor (T) increases for ⁸⁹Zr-CX-072, but not for ⁸⁹Zr-PBCtrl. Tracer blood pool decreased over time, while ⁸⁹Zr-CX-072, but not ⁸⁹Zr-PBCtrl, showed tracer tumor accumulation. Uptake of ⁸⁹Zr-CX-072 in MDA-MB-231 tumor and blood pool was quantified by SUVmean at 1, 3, and 6 days p.i. Tumor uptake was highest (SUVₘₑₐₙ 1.5±0.2) for 10 μg ⁸⁹Zr-CX-072 at 6 days p.i. ⁸⁹Zr-CX-072 tumor uptake in MDA-MB-231 xenografts appeared to be protein dose (target binding) dependent, as demonstrated by decreasing tumor ⁸⁹Zr-CX-072 uptake with increasing cold CX-072 dose, as shown in FIG. 3A (at 144 h post dose). In contrast, tumor uptake of ⁸⁹Zr-PBCtrl was low and not affected by the presence of unlabeled PBCtrl (FIG. 3A). The 10 μg total tracer protein dose of ⁸⁹Zr-CX-072 provided the highest contrast in tumor uptake, when compared to ⁸⁹Zr-PBCtrl, and was therefore considered the optimal tracer protein dose.

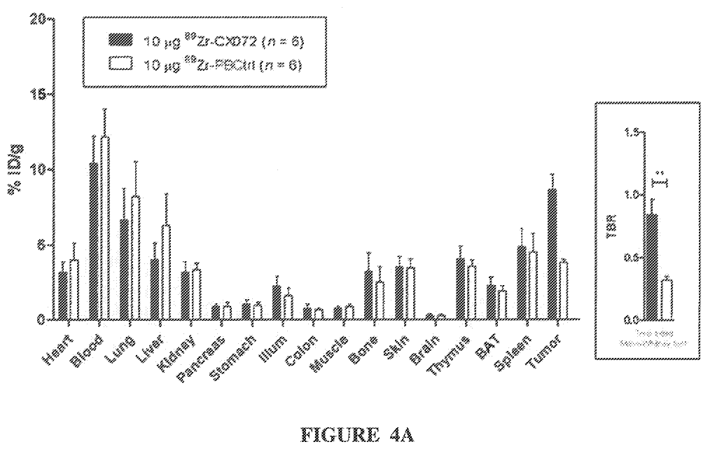

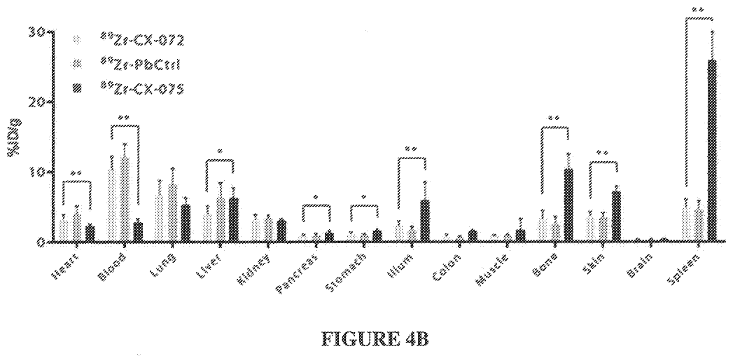

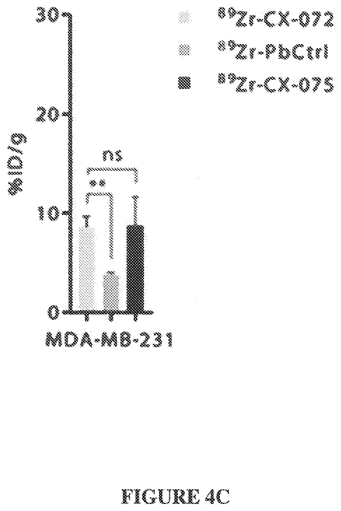

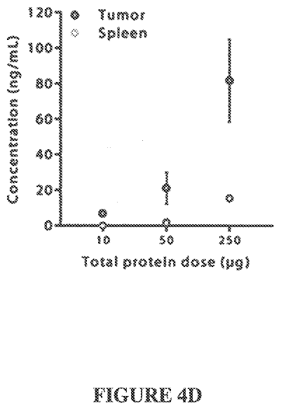

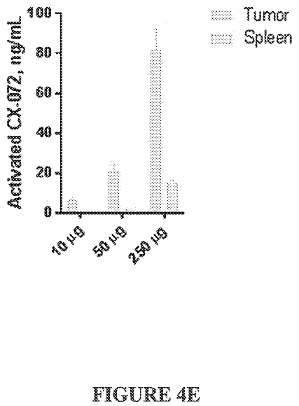

Uptake in other organs showed no difference between dose groups for both ⁸⁹Zr-CX-072 and ⁸⁹Zr-PBCtrl, as shown in FIG. 4A. ⁸⁹Zr-CX-072 tumor-to blood ratio (TBR) was significantly higher when compared to ⁸⁹Zr-PbCtrl (with a maximum TBR of 0.8 vs. 0.3 at 10 μg tracer protein dose), demonstrating target-specific tumor uptake of ⁸⁹Zr-CX-072 (FIG. 4A, insert).

Comparison of ⁸⁹Zr-CX-072, ⁸⁹Zr-PBCtrl, and ⁸⁹Zr-CX-075: PET imaging on day 1, 3, and 6 post intravenous injection (pi) revealed tumor accumulation over time for ⁸⁹Zr-CX-072 and ⁸⁹Zr-CX-075, but not for ⁸⁹Zr-PbCtrl as shown in FIG. 2A. As shown in FIG. 2A, tracer radioactivity in the blood pool decreased over time, resulting in increasing tumor to blood ratios for ⁸⁹Zr-CX-072 and ⁸⁹Zr-CX-075 from day 1 to 6 p.i. with highest tumor uptake at day 6 p.i.

⁸⁹Zr-CX-075 showed clear uptake in spleen and lymph nodes on PET images, which was not visible for ⁸⁹Zr-CX-072 and ⁸⁹Zr-PbCtrl (FIG. 2A). PET quantification revealed an 1.5-fold higher spleen uptake for ⁸⁹Zr-CX-075 than for ⁸⁹Zr-CX-072 at day 6 p.i. (p<0.01) (FIGS. 2B-2D). ⁸⁹Zr-CX-075 spleen uptake was higher than blood pool levels, supporting that this uptake is PD-L1-mediated (FIGS. 2B-2D).

⁸⁹Zr-CX-072 in the circulation remained intact at 6 days p.i., as confirmed by sodium dodecylsulphate polyacrylamide gel electrophoresis (SDS-PAGE).

Ex vivo analysis revealed decreasing ⁸⁹Zr-CX-072 tumor uptake from 8.7±1.0% ID/g at the 10 μg total protein dose to 6.0±1.3% ID/g and 4.3±0.7% ID/g for the 50 μg and 250 μg dose groups respectively indicating competition of tracer with the unlabeled CX-072 binding to PD-L1 receptor (FIG. 3B). Similarly, ⁸⁹Zr-CX-075 tumor uptake was reduced by unlabeled antibody (FIG. 3B). ⁸⁹Zr-PbCtrl tumor uptake was independent of total protein dose, confirming its non-specificity for PD-L1 target binding (FIG. 3B).

Although immune-compromised mice were used for this model, specific spleen uptake was observed for ⁸⁹Zr-CX-075, as demonstrated by decreased spleen uptake from 25.8±4.1% ID/g at the 10 μg total protein dose to 10.8±2.8% ID/g and 5.3±2.6% ID/g for the 50 μg and 250 g dose groups respectively. ⁸⁹Zr-CX-072 and ⁸⁹Zr-PbCtrl did not show dose-dependent spleen uptake, suggesting the CX-072 is not activated in this tissue which otherwise could lead to accumulation in this PD-L1 expressing spleen tissue (FIG. 3C).

Except for tumor, similar ex vivo biodistribution results were found for ⁸⁹Zr-CX-072 and ⁸⁹Zr-PbCtrl in other normal tissues (FIG. 4B). ⁸⁹Zr-CX-075 blood pool levels and uptake in the heart, however, were lower, while liver, pancreas, stomach, ilium, bone, skin and spleen uptake were higher compared to ⁸⁹Zr-CX-072. ⁸⁹Zr-CX-072 and ⁸⁹Zr-CX-075 showed comparable tumor uptake of 8.7±1.0% ID/g and 8.8±2.9% ID/g, respectively, for the 10 μg ⁸⁹Zr-PbCtrl (FIG. 4C). This suggests that the prodomain architecture affects biodistribution but not its tumor-targeting properties. Highest tumor uptake was found for 10 μg of ⁸⁹Zr-CX-072, therefore this total protein dose was selected for further in vivo studies.

To investigate whether CX-072 is activated by proteases in the tumor microenvironment and peripheral PD-L1-expressing organs, MDA-MB-231 tumor and spleen lysates were analyzed for the presence of activated CX-072 (FIG. 4D). MDA-MB-231 tumor lysates contained 6.9 ng/ml activated CX-072 species at the 10 μg total protein dose, 21.2 ng/ml at the 50 g total protein dose and highest concentration of 81.7 ng/ml was found for the 250 μg dose group (FIG. 4E). There was a 5.3-fold lower level of activated CX-072 detected in spleen at the 250 μg/total protein dose (p<0.05). This suggests that the activatable binding polypeptide is specifically activated in tumor tissue and remains predominantly within the tumor microenvironment.

Ex vivo macroscopic tracer visualization in paraffin-embedded formalin-fixed (FFPE) tumor tissue slices using autoradiography revealed a heterogeneous distribution pattern for ⁸⁹Zr-CX-072 and ⁸⁹Zr-CX-075. Immunohistochemistry showed PD-L1 staining in viable tumor tissue and to a lesser extent in necrotic tumor tissue, correlating to regions showing high uptake of ⁸⁹Zr-CX-072 on autoradiography. In contrast, ⁸⁹Zr-PbCtrl distributed to non-tumor tissue areas while PD-L1 expression was present in viable tumor indicating observed uptake is not PD-L1 specific. ⁸⁹Zr-CX-075 distributed mostly to PD-L1 expressing tumor, however, uptake in non-PD-L1 expressing, necrotic tumor tissue was also observed.

### Evaluation in Immune Competent Mouse Model Bearing MC38 Syngeneic Tumors

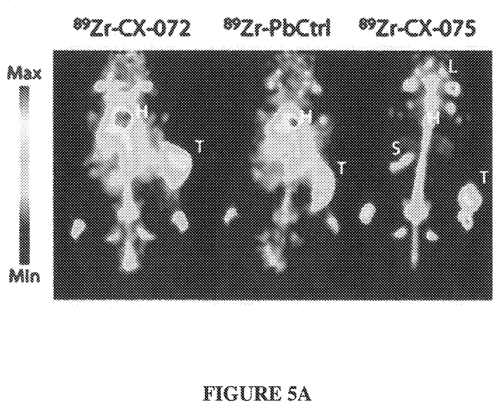

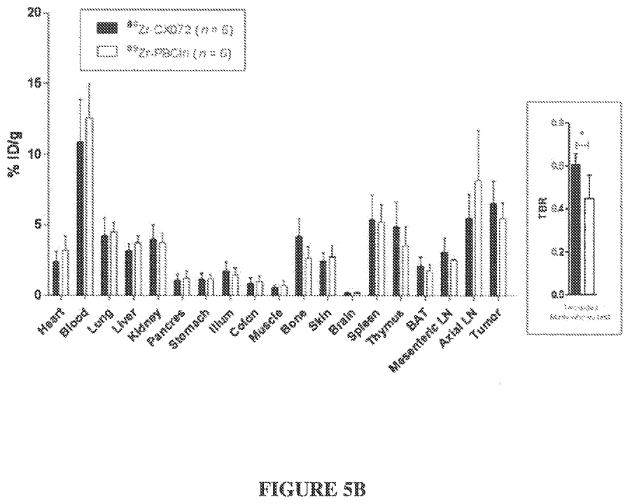

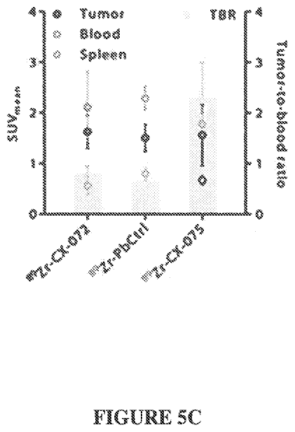

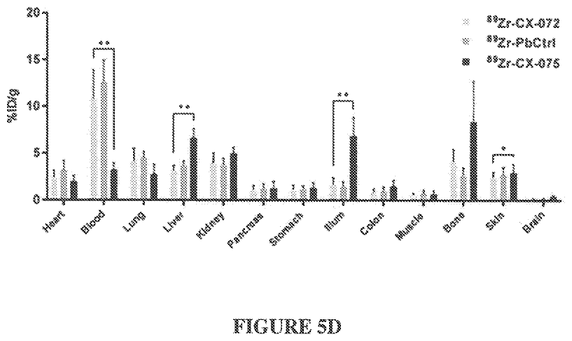

The biodistribution of ⁸⁹Zr-CX-072, ⁸⁹Zr-PBCtrl, and CX-075 (10 μg total tracer protein dose was evaluated by PET imaging in fully immune-competent MC38 xenograft bearing Cs57Bl/6 mice, in accordance with the method of the present invention. The MC38 cells were obtained from the University of Pittsburgh. FIG. 5A depicts representative maximum intensity projections of ⁸⁹Zr-CX-072, ⁸⁹Zr-CX-PbCtrl, and ⁸⁹Zr-CX-075 in the MC38 tumor-bearing mice imaged at 6 days p.i. H; heart, T: tumor, S: spleen, L: lymph node. FIGS. 5B and 5C depict ex vivo biodistribution of ⁸⁹Zr-CX-072 and ⁸⁹Zr-PbCtrl, and ⁸⁹Zr-CX-072, ⁸⁹Zr-PbCtrl, and ⁸⁹Zr-CX-075, respectively. As shown in FIG. 5B, ⁸⁹Zr-CX-072 showed significantly higher TBR at 144 h post-injection when compared to ⁸⁹Zr-PBCtrl (FIG. 5B, insert), however, the difference is smaller compared to the MDA-MB-231 xenograft model. ⁸⁹Zr-CX-072 and ⁸⁹Zr-CX-075 showed comparable tumor uptake at 6 days p.i., which 3.1-fold higher spleen uptake was observed for ⁸⁹Zr-CX-075 compared to ⁸⁹Zr-CX-072 (p<0.01) (FIG. 5C).

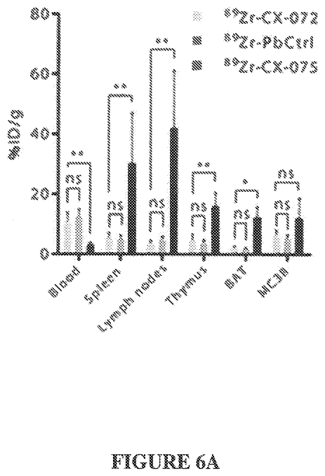

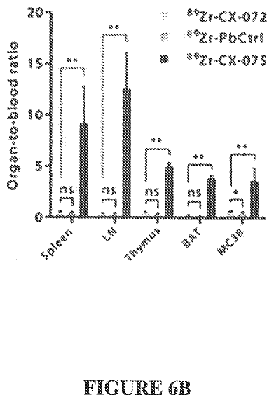

As shown in FIG. 5B (showing a comparison of tissue uptake for tracers ⁸⁹Zr-CX0-072 and ⁸⁹Zr-CX-PBCtrl) and 5D (showing a comparison of tissue tracer uptake for all three tracers, ⁸⁹Zr-CX-072, ⁸⁹Zr-PbCtrl, and ⁸⁹Zr-CX-075), uptake of ⁸⁹Zr-CX-072 by lymphoid tissues (e.g., spleen, lymph nodes, thymus) detected in immune-competent C57BL/6 mice was similar to (i.e., not significantly different than) that in the non-binding isotype control ⁸⁹Zr-PBCtrl Blood pool levels of ⁸⁹Zr-CX-075 were lower, while uptake was higher in liver, ilium and brain compared to ⁸⁹Zr-CX-072 (FIG. 5D). The organ-to-blood ratio of ⁸⁹Zr-CX-072, ⁸⁹Zr-PbCtrl, and ⁸⁹Zr-CX-075 in lymphoid tissues of the MC38 tumor bearing syngeneic mice 6 days p.i. is provided in FIG. 6B (i.e. spleen, mesenteric and axial lymph nodes (LN), thymus, brown adipose tissue (BAT), and MC38 tumor tissue). High ⁸⁹Zr-CX-075 uptake was also found in lymphoid tissues including spleen, mesenteric and axial lymph nodes, thymus and BAT (FIGS. 6A and 6B). In contrast, minor ⁸⁹Zr-CX-072 uptake was observed in these tissues, comparable with ⁸⁹Zr-PbCtrl. Thus, the results from these in vivo studies suggest that ⁸⁹Zr-CX-072 accumulates more in PD-L1 expressing tumor tissues than in lymphoid tissues. In addition, residual radioactivity measured in MDA-MB0231 and MC38 tumor-bearing mice at 1, 3, and 6 days p.i. suggests faster elimination of ⁸⁹Zr-CX-075 compared to ⁸⁹Zr-CX-072.

The results further showed that no significant target-mediated deposition of 89Zr-CX-072 was detected in C57BL/6 mouse lymphoid tissues, in contrast to results obtained for the corresponding parental antibody, CX-075.

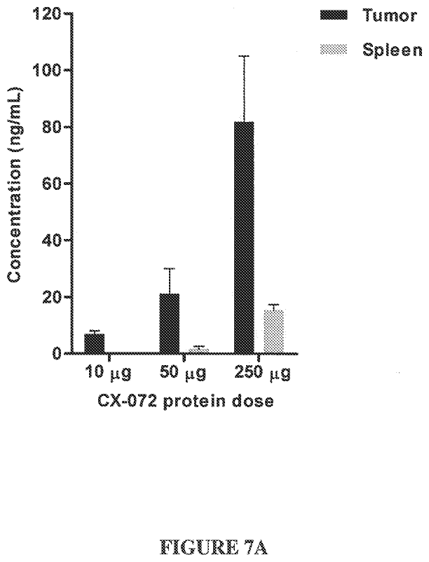

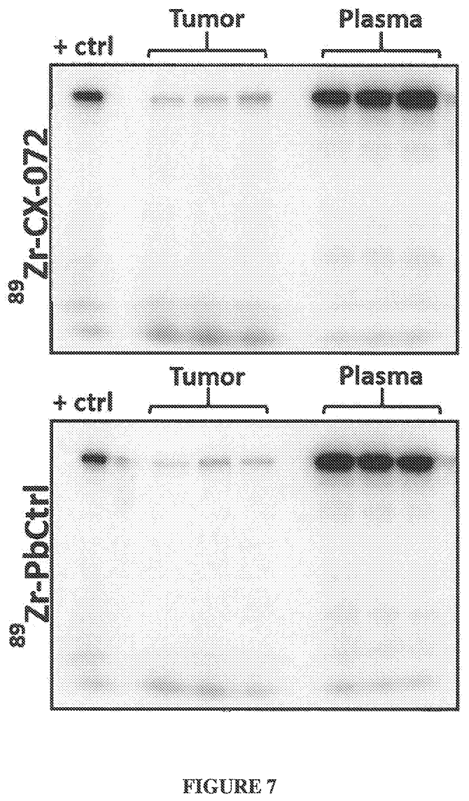

Tracer integrity in tumor lysates and plasma was assessed by Western Capillary Electrophoresis (WES). FIG. 7A depicts the concentration of activated ⁸⁹Zr-CX-072 species detected in MDA-MB-231 tumor tissue and spleen by WES. FIG. 7B depicts an SDS-PAGE autoradiograph of ⁸⁹Zr-CX-072 and ⁸⁹Zr-PbCtrl in MC38 tumor lysates and plasma 6 days p.i. The results indicate that activated activatable antibody species is predominantly detected in tumor tissue. Intact (unactivated activatable antibody) tracer appeared to be present in both tumor and plasma.

Ex vivo autoradiography was conducted on the ⁸⁹Zr-CX-072 and ⁸⁹Zr-PbCtrl in MDA-MB-231 tumor tissue, in conjunction with PD-L1 immunofluorescence and PD-L1 immunohistochemistry (IHC). The results showed uptake of ⁸⁹Zr-CX-072 in PD-L1 expressing tumor tissue, and as a comparison, limited uptake of ⁸⁹Zr-PbCtrl in non-tumor tissue.

The data obtained from these experiments indicate that ⁸⁹Zr-CX-072 accumulates in tumor over time, but not in spleen, and that ⁸⁹Zr-CX-072 biodistribution in healthy tissues is similar to ⁸⁹Zr-PbCtrl. Therefore, ⁸⁹Zr-CX-072 tumor uptake appears to be PD-L1 specific, in contrast to spleen uptake. ⁸⁹Zr-CX-072 appeared to be preferentially activated in PDL-1-expressing tumor, but not in PDL-1 expressing spleen. It appeared that no PDL-1 mediated uptake of ⁸⁹Zr-CX-072 occurred in lymphoid tissues. Thus, the results obtained by in vivo PET imaging showing accumulation of the ⁸⁹Zr-CX-072 in tumor tissue were consistent with the results obtained from the ex vivo biodistribution studies, and therefore indicate that in vivo distribution of an activated binding polypeptide in a mammalian subject can be ascertained via PET imaging, as described herein.

### Example 2

### Conjugation of Activatable Antibody with Df-Suc-N-TFP and Radiolabelling with Zr⁸⁹

Conjugation with Df-Suc-N-TFP. The bifunctional chelator N-succinyldesferrioxamine-B-tetrafluorphenol (“desferal-N-suc-TFP” or “Df-suc-N-TFP”, ABX GmbH), which is the active tetrafluorophenol (TFP) ester of the succinylated form of desferal, was used to conjugate the succinylated form of desferal to the activatable antibody CX-072. For each conjugation, 60 mg of CX-072 was used. Before the start of the conjugation procedure, buffer exchange was performed on the CX-072 starting material using centrifugation with a 30 kDa filter (Vivaspin-2 Centrifugal Concentrator, Vivaproducts, Inc.). This step was performed two times until the buffer was partially replaced by water for injections and the desired volume of retentate was obtained. Next, conjugation was performed with the chelator Df-suc-N-TFP (7.5 mol/μl) at pH 8.5 and room temperature. The achieved desferal:activatable antibody ratio was determined by SE-HPLC. Subsequently, the protective iron (II) in the desferal moiety was removed with an excess of EDTA at pH 4.0-4.5. The intermediate Df-Suc-N-CX-072 was purified using centrifugation with a 30 kDa filter (Vivaspin-2), which was performed five times. The purified product was then diluted to a concentration of 10 mg/ml in Water for Injection (WFI), followed by sterile filtration. Df-Suc-N-CX-072 was stored at <−70° C. In each batch, 60 mg CX-072 was modified with Df-Suc-N-CX-072, and 25 mg aliquots made.

The conjugation process (up until the sterile filtration) was performed in a class A downflow cabinet in a class C background environment. The sterile filtration was performed in a closed glove-box (class) with a class B transfer chamber in a class C background environment. Environmental monitoring of the rooms was performed by continuous monitoring of the air pressure hierarchy and by measurement of microorganism and particulate levels.

Three independent 60 mg batches of Df-Suc-N-CX-072 were produced at yields of greater than 90%.

Df-Suc-N-CX-PbCtrl and Df-Suc-N-CX-075 were similarly prepared.

Radiolabeling of CX-072, PbCtrl CX-075. CX-072, PbCtrl and CX-075 (CytomX Therapeutics Inc.) were allowed to react with an 1:2 molar excess of TFP-N-sucDf (ABX GmbH) in accordance with the method for conjugating antibodies with ⁸⁹Zr described in Verel, et al., J. Nucl. Med. 44:1271-1281 (2003). CX-072-N-sucDf, PbCtrl-N-sucDf and CX-075-N-sucDf were purified using a Vivaspin-2 concentrator, aliquoted and stored at −80° C. Concentration and purity were determined by a Waters size exclusion high-performance liquid chromatography (SE-HPLC) system equipped with a dual-wavelength absorbance detector (280 nm versus 430 nm), in-line radioactivity detector and TSK-Gel SW column G3000SWXL 5 μm, 7.8 mm (Joint Analytical Systems; mobile phase: phosphate buffered saline (PBS; 9.0 mM sodium phosphate, 1.3 mM potassium phosphate, 140 mM sodium chloride, pH 7.2) (Hospital Pharmacy UMCG); flow: 0.7 mL/min).

Radiochemical purity was assessed by a trichloroacetic acid precipitation assay using methods described in Nagengast, et al., J. Nucl. Med. 48: 1313-1319 (2007).

### Example 3 cGMP Labeling of Df-Suc-N-CX-072 with Zirconium-89

Df-Suc-N-CX-072 aliquots were thawed and radiolabeled with a known volume and radioactive dose of clinical grade ⁸⁹Zr. The ⁸⁹Zr was obtained as a solution in 1 M oxalic acid (PerkinElmer Nederland B.V. in accordance with cGMP, activity between 740 and 1850 MBq/ml, with >99.9% radionuclide purity). The product was purified using centrifugation with a 30 kDa filter (Vivaspin-2) and the amount of radioactivity was determined in the filter, filtrate, and the retentate. The labeling process was performed in a closed Glove-box (class A) with a class B transfer chamber in a class C background environment. Environmental monitoring of the rooms was performed by continuous monitoring of the air pressure hierarchy and by measurement of microorganism and particulate levels. Three independent batches of ⁸⁹Zr-N-Suc-Df-CX-072 (each of batch size 2.5 mg/37 MBq) were prepared. The radiochemical purity pre-purification of the three batches was 97.0% or greater. The radiochemical purity post-purification of the three batches was greater than 99%. The yields were 51.63 MBq, 79.63 MBq, and 62.87 MBq.

### Example 4

### Stability Testing of ⁸⁹Zr-Activatable Binding Polypeptide

Three batches of GMP compliant CX-072-N-sucDf intermediate were produced and radiolabeled with ⁸⁹Zr as described above, followed by purification, dilution and sterile filtration. These batches were characterized on conjugation efficiency/ratio, yield, aggregates, concentration, pH, and radiochemical purity. The results are shown below in Table 3.

TABLE 3

| Test | Method | Batch 1 | Batch 2 | Batch3 |
|---|---|---|---|---|
| Appearance | Visual | Colorless | Colorless | Colorless |
| | Inspection | | | |
| Conjugation | SE-HPLC | 1.28 | 1.34. | 1.27 |
| Ratio | | | | |
| Purity - | SE-HPLC | 0.6% | 0.8% | 1.9% |
| Aggregates | at 280 nm | | | |
| Concentration | UV | 10.87 mg/ml | 9.32 mg/ml | 9.86 mg/ml |
| | spectrophotometry | | | |
| pH | European | 5.24 | 5.0 | 5.16 |
| | Pharmacopoeia | | | |
| Radiochemical | TCA | 99.5% | 99.0% | 99.2% |
| purity | Assay | | | |

The CX-072-N-sucDf intermediate was stored in sterile vials (Biopure) at −80° C. Stability of CX-072-N-sucDf batch 1 was analyzed at 0, 1, 3, 6 and 12 months after production. Data were analyzed for statistical significance in GraphPad Prism (v7.0) using the Mann-Whitney U test for non-parametric data followed by Bonferroni post-test correction for comparison of more than two groups. Immunoreactivity was analysed by nonlinear regression Log(agonist) vs. response in Graphpad Prism (v7.0). Experiments were performed at least three times. P values<0.05 were considered significant. The results are shown in Table XX below.

TABLE 4
Stability Testing after Radiolabeling with ⁸⁹Zr

| Test | t = 0 | t = 6 months | T = 12 months |
|---|---|---|---|
| Appearance | Colorless | Colorless | Colorless |
| Purity | ≤5% | ≤5% | ≤5% |
| Concentration | 10.87 mg/ml | | 10.25 mg/ml |
| pH | 5.24 | | 5.19 |
| Radiochemical | 99.5% | | 99.0% |
| purity | | | |

### Example 5

### Biodistribution of ⁸⁹Zr-Activatable Binding Polypeptide in a Human Subject

This is a study designed to evaluate the whole body distribution of ⁸⁹Zr-CX-072 in human subjects with locally advanced or metastatic solid tumors prior to treatment with standard CX-072.

The human subjects eligible for the studies are those having advanced or metastatic solid tumors and who have at least 1 tumor site that is accessible and safe to biopsy. Additional inclusion criteria include the following:
1. PD-L1 status:
   At least 14 of 21 subjects have documented PD-L1 expression in 25% tumor cells by 22C3 PharmDx (DAKO) assay; and
   Up to 7 subjects with unknown PD-L1 status or documented PD-L1 negativity may be enrolled.
2. Measurable disease, as defined by standard Response Evaluation Criteria in Solid Tumors (RECIST) v1.1. Metastatic lesion(s) (21 cm) of which a histological biopsy can safely be obtained according to standard clinical care procedures.
   Subjects who fulfill any of the following criteria will be excluded:

1. Signs or symptoms of infection 2 weeks prior to ⁸⁹ZR-CX-072 injection.

2. Ionizing radiation exposure in the last 12 months.

3. Inability to comply with any additional requirement of the substudy protocol.

The study is divided into 2 parts. Part A is the dose-finding part of the substudy, performed to assess the optimal protein dose of CX-072 and the optimal interval between ⁸⁹Zr-CX-072 injection and scanning. A fixed dose of 37 MBq ⁸⁹Zr-CX-072 combined with an escalating dose of unlabeled CX-072 will be administered by IV infusion over 60 minutes for doses of 0.3, 1, 3, and 10 mg/kg. CX-072 will be supplied as a sterile, preservative-free solution in 100 mg vials at a concentration of 10 mg/mL and diluted to the following dose levels: 0.03 mg/kg; 0.1 mg/kg, 0.3 mg/kg. Unlabeled CX-072 will be administered by IV infusion followed by injection of the labeled ⁸⁹Zr-CX-072 dose. The cold dose is used to pre-block the non-specific antigen sinks, thus allowing for better imaging resolution. All infusions will be administered through a non-pyrogenic, low protein binding in-line filter (pore size of 0.2 μm). Following completion of the infusion, flush with an adequate amount of normal saline for infusion.

A maximum of 3 ⁸⁹Zr-CX-072-PET scans will be performed on Days 2 (48 [±6] h), 4 (96 [±6] h), and 7 (168 [±6] h) after ⁸⁹Zr-CX-072 administration. All scans will be obtained in total body mode (trajectory feet-skull vertex), using low-dose (LD) computed tomography (CT) for attenuation correction and localization purposes. For all PET scans, acquisition will comprise approximately 14 bed positions. The maximum total acquisition time, including LD-CT, will be approximately 90 minutes (approximately 50 minutes for PET scans post-injection on Days 2 and 4 and approximately 90 minutes for PET scans post-injection on Day 7). For ⁸⁹Zr-CX-072 imaging, the harmonization procedures, comparable to the European Association of Nuclear Medicine (EANM) Research Limited PET/CT accreditation and EANM guidelines, as described by Makris et al (Makris et al, 2014) will be applied. The imagine schedule is set forth in Table 5 below.

TABLE 5
Part A: Imaging Dose and Schedule Finding

| Part A | Day 0 | Day 2 | Day 4 | Day 7 | |
|---|---|---|---|---|---|
| ⁸⁹Zr-CX- | X | | | | Initiate standard |
| 072 | | | | | CX-072 treatment |
| ⁸⁹Zr-PET | X | X | X | X | |
| scan | | | | | |

After completion of Part A of the study, all subjects will receive standard CX-072 treatment.

The purpose of Part B is to evaluate the whole body distribution of ⁸⁹Zr-CX-072 in subjects with locally advanced or metastatic solid tumors. In Part B, subjects will undergo 1 PET scan according to the optimal scanning schedule determined in Part A. A maximum of 3 ⁸⁹Zr-CX-072-PET scans will be performed on Days 2 (48 [±6] h), 4 (96 [±6] h), and 7 (168 [±6]h) after ⁸⁹Zr-CX-072 administration.

Blood samples for PK will be drawn before ⁸⁹Zr-CX-072 injection (2×5 mL, 1×10 mL) and 60 (10) minutes (1×10 mL) after administration of the ⁸⁹Zr-CX-072 dose, and on Day 2 (T=48 [±6] hours), Day 4 (T=96 [±6] hours), and Day 7 (T=168 [±6] hours). If a PET scan is scheduled on the same day, blood sampling will be performed a maximum of 60 minutes before or after the PET scan procedure. The imaging schedule is set forth in Table 6, below

TABLE 6
Implementation of Imaging

| Part B | Day 0 | Day 2, 4, or 7 | |
|---|---|---|---|
| ⁸⁹Zr-CX- | X | | Initiate standard |
| 072 | | | CX-072 treatment |
| ⁸⁹Zr-PET | | X | |
| scan | | | |
| Biopsy | | X | |

Whole body ⁸⁹Zr-CX-072 distribution is determined by measuring the SUV on the ⁸⁹Zr-CX-072-PET scans. Quantification of ⁸⁹Zr-CX-072 distribution will be performed using AMIDE software (Stanford University, Palo Alto, Calif., USA). ⁸⁹Zr-CX-072 uptake will be corrected for body weight and injected dose and be quantitatively assessed as SUV, which is calculated using the formula: [tissue activity concentration (MBq/g)]/[(injected dose (MBq)/body weight (g)]. The SUV of all tumor lesions and relevant normal tissues will be calculated on all PET-CT scans. The in vivo PK of ⁸⁹Zr-CX-072 will be evaluated using summary statistics of SUV by organ and imaging time point.

Observations to Date:

The uptake of ⁸⁹Zr-CX-072 in tumor lesions was detected by PET imaging in multiple human patients.

TABLE 7
Table of Sequences

| SEQ | | |
|---|---|---|
| ID | | |
| NG: | NAME | SEQUENCE |
| 1 | CM | LSGRSDNH |
| 2 | CM | TGRGPSWV |
| 3 | CM | PLTGRSGG |
| 4 | CM | TARGPSFK |
| 5 | CM | NTLSGRSENHSG |
| 6 | CM | NTLSGRSGNHGS |
| 7 | CM | TSTSGRSANPRG |
| 8 | CM | TSGRSANP |
| 9 | CM | VHMPLGFLGP |
| 10 | CM | AVGLLAPP |
| 11 | CM | AQNLLGMV |
| 12 | CM | QNQALRMA |
| 13 | CM | LAAPLGLL |
| 14 | CM | STFPFGMF |
| 15 | CM | ISSGLLSS |
| 16 | CM | PAGLWLDP |
| 17 | CM | VAGRSMRP |
| 18 | CM | VVPEGRRS |
| 19 | CM | ILPRSPAF |
| 20 | CM | MVLGRSLL |
| 21 | CM | QGRAITFI |
| 22 | CM | SPRSIMLA |
| 23 | CM | SMLRSMPL |
| 24 | CM | ISSGLLSGRSDNH |
| 25 | CM | AVGLLAPPGGLSGRSDNH |
| 26 | CM | ISSGLSSGGSGGLSLSGRSDNH |
| 27 | CM | LSGRSGNH |
| 28 | CM | SGRSANPRG |
| 29 | CM | LSGRSDDH |
| 30 | CM | LSGRSDIH |
| 31 | CM | LSGRSDQH |
| 32 | CM | LSGRSDTH |
| 33 | CM | ISGRSDYH |
| 34 | CM | LSGRSDNP |
| 35 | CM | LSGRSANP |
| 36 | CM | LSGRSANI |
| 37 | CM | LSGRSDNI |
| 38 | CM | MIAPVAYR |
| 39 | CM | RPSPMWAY |
| 40 | CM | WATPRPMR |
| 41 | CM | FRLLDWQW |
| 42 | CM | ISSGL |
| 43 | CM | ISSGLLS |
| 44 | CM | ISSGLL |
| 45 | CM | ISSGLLSGRSANPRG |
| 46 | CM | AVGLLAPPTSGPSANPRG |
| 47 | CM | AVGLLAPPSGRSANPRG |
| 48 | CM | ISSGLLSGRSDDH |
| 49 | CM | ISSGLLSGRSDIH |
| 50 | CM | ISSGLLSGRSDQH |
| 51 | CM | ISSGLLSGRSDTH |
| 52 | CM | ISSGLLSGRSDYH |
| 53 | CM | ISSGLLSGRSDNP |
| 54 | CM | ISSGLLSGRSANP |
| 55 | CM | ISSGLLSGRSANI |
| 56 | CM | AVGLLAPPGGLSGRSDDH |
| 57 | CM | AVGLLAPPGGLSGRSDIH |
| 58 | CM | AVGLLAPPGGLSGRSDQH |
| 59 | CM | AVGLLAPPGGLSGRSDTH |
| 60 | CM | AVGLLAPPGGLSGRSDYH |
| 61 | CM | AVGLLAPPGGLSGRSDNP |
| 62 | CM | AVGLLAPPGGLSGRSANP |
| 63 | CM | AVGLLAPPGGLSGRSANI |
| 64 | CM | ISSGLLSGRSDNI |
| 65 | CM | AVGLLAPPGGLSGRSDNI |
| 66 | CM | GLSGRSDNHGGAVGLLAPP |
| 67 | CM | GLSGRSDNHGGVHMPLGFLGP |
| 68 | Linker | GSGGS |
| 69 | Linker | GGGS |
| 70 | Linker | GGSG |
| 71 | Linker | GGSGG |
| 72 | Linker | GSGSG |
| 73 | Linker | GSGGG |
| 74 | Linker | GGGSG |
| 75 | Linker | GSSSG |
| 76 | Linker | GSSGGSGGSGGSG |
| 77 | Linker | GSSGGSGGSGG |
| 78 | Linker | GSSGGSGGSGGS |
| 79 | Linker | GSSGGSGGSGGSGGGS |
| 80 | Linker | GSSGGSGGSG |
| 81 | Linker | GSSGGSGGSGS |
| 82 | Linker | GSSGT |
| 83 | Linker | GSSG |
| 84 | Mask | YCEVSELFVLPWCMG |
| | Moiety | |
| | PL01 | |
| 85 | Mask | SCLMHPHYAHDYCYV |
| | Moiety | |
| | PL02 | |
| 86 | Mask | LCEVLMLLQHPWCMG |
| | Moiety | |
| | PL03 | |
| 87 | Mask | IACRHFMEQLPFCHH |
| | Moiety | |
| | PL04 | |
| 88 | Mask | FGPRCGEASTCVPYE |
| | Moiety | |
| | PL05 | |
| 89 | Mask | ILYCDSWGAGCLTRP |
| | Moiety | |
| | PL06 | |
| 90 | Mask | GIALCPSHFCQLPQT |
| | Moiety | |
| | PL07 | |
| 91 | Mask | DGPRCFVSGECSPIG |
| | Moiety | |
| | PL08 | |
| 92 | Mask | LCYKLDYDDRSYCHI |
| | Moiety | |
| | PL09 | |
| 93 | Mask | PCHPHPYDARPYCNV |
| | Moiety | |
| | PL10 | |
| 94 | Mask | PCYWHPFFAYRYCNT |
| | Moiety | |
| | PL11 | |
| 95 | Mask | VCYYMMVLGRNWCSS |
| | Moiety | |
| | PL12 | |
| 96 | Mask | LCDLFKLREFPYCMG |
| | Moiety | |
| | PL13 | |
| 97 | Mask | YLPCHFVPIGACNNK |
| | Moiety | |
| | PL14 | |
| 98 | Mask | IFCHMGVVVPQCANY |
| | Moiety | |
| | PL15 | |
| 99 | Mask | ACHPHPYDARPYCNV |
| | Moiety | |
| | PL16 | |
| 100 | Mask | PCHPAPYDARPYCNV |
| | Moiety | |
| | PL17 | |
| 101 | Mask | PCHPHAYDARPYCNV |
| | Moiety | |
| | PL18 | |
| 102 | Mask | PCHPHPADARPYCNV |
| | Moiety | |
| | PL19 | |
| 103 | Mask | PCHPHPYAARPYCNV |
| | Moiety | |
| | PL20 | |
| 104 | Mask | PCHPHPYDAAPYCNV |
| | Moiety | |
| | PL21 | |
| 105 | Mask | PCHPHPYDARPACNV |
| | Moiety | |
| | PL22 | |
| 106 | Mask | PCHPHPYDARPYCAV |
| | Moiety | |
| | PL23 | |
| 107 | Mask | PCHAHPYDARPYCNV |
| | Moiety | |
| | PL24 | |
| 108 | Mask | PCHPHPYDARAYCNV |
| | Moiety | |
| | PL25 | |
| 109 | VL domain | GACATCCAGATGACCCAGTCTCCATCCTCCCTGTCTGC |
| | of anti- | ATCTGTAGGAGACAGAGTCACCATCACTTGCCGGGCA |
| | PDL1 | AGTCAGAGCATTAGCAGCTATTTAAATTGGTATCAGC |
| | activatable | AGAAACCAGGGAAAGCCCCTAAGCTCCTGATCTATTA |
| | antibody | TGCATCCACTTTGCAAAGTGGGGTCCCATCAAGGTTC |
| | | AGTGGCAGTGGATCTGGGACAGATTTCACTCTCACCA |
| | | TCAGCAGTCTGCAACCTGAAGATTTTGCAACTTACTA |
| | | CTGTCAACAGGATAATGGTTATCCTTCTACGTTCGGCC |
| | | AAGGGACCAAGGTGGAAATCAAACGG |
| 110 | VL domain | DIQMTQSPSSLSASVGDRVTITCRASQSISSYLNWYQQKP |
| | of anti- | GKAPKLLIYYASTLQSGVPSRFSGSGSGTDFTLTISSLQPE |
| | PDL1 | DFATYYCQQDNGYPSTFGQGTKVEIKR |
| | activatable | |
| | antibody | |
| 111 | VL domain | GATATTCAGATGACCCAGAGCCCGAGCAGCCTGAGCG |
| | of anti- | CGAGCGTGGGCGATCGCGTGACCATTACCTGCCGCGC |
| | PDL1 | GAGCCAGAGCATTAGCAGCTATCTGAACTGGTATCAG |
| | activatable | CAGAAACCGGGCAAAGCGCCGAAACTGCTGATTTATG |
| | antibody | CGGCGAGCAGCCTGCAGAGCGGCGTGCCGAGCCGCTT |
| | | TAGCGGCAGCGGCAGCGGCACCGATTTTACCCTGACC |
| | | ATTAGCAGCCTGCAGCCGGAAGATTTTGCGACCTATT |
| | | ATTGCCAGCAGGATAACGGCTATCCGAGCACCTTTGG |
| | | CGGCGGCACCAAAGTGGAAATTAAACGC |
| 112 | VL domain | DIQMTQSPSSLSASVGDRVTITCRASQSISSYLNWYQQKP |
| | of anti- | GKAPKLLIYAASSLQSGVPSRFSGSGSGTDFTLTISSLQPE |
| | PDL1 | DFATYYCQQDNGYPSTFGGGTKVEIKR |
| | activatable | |
| | antibody | |
| 113 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTATTCTACTGGTGGTGCTACAGCTTACGCAGA |
| | | CTCCGTGAAGGGCCGGTTCACCATCTCCAGAGACAAT |
| | | TCCAAGAACACGCTGTATCTGCAAATGAACAGCCTGA |
| | | GAGCCGAGGACACGGCCGTATATTACTGTGCGAAATC |
| | | TTCTGCTGGTAGTCGGCCGGGTTTTGACTACTGGGGC |
| | | CAGGGAACCCTGGTCACCGTCTCGAGC |
| 114 | VH domain | EVQLLESGGGLVQPGGSLRLSCAASGFTFSSYAMSWVR |
| | of anti- | QAPGKGLEWVSSIYSTGGATAYADSVKGRFTISRDNSK |
| | PDL1 | NTLYLQMNSLRAEDTAVYYCAKSSAGQSRPGFDYWGQ |
| | activatable | GTLVTVSS |
| | antibody | |
| 115 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTATTCTACTGGTGGTGCTACAGCTTACGCAGA |
| | | CTCCGTGAAGGGCCGGTTCACCATCTCCAGAGACAAT |
| | | TCCAAGAACACGCTGTATCTGCAAATGAACAGCCTGA |
| | | GAGCCGAGGACACGGCCGTATATTACTGTGCGAAATC |
| | | TTCTGCTGGTTCGTGGCCGGGTTTTGACTACTGGGGCC |
| | | AGGGAACCCTGGTCACCGTCTCGAGC |
| 116 | VH domain | EVQLLESGGGLVQPGGSLRLSCAASGFTFSSYAMSWVR |
| | of anti- | QAPGKGLEWVSSIYSTGGATAYADSVKGRFTISRDNSK |
| | PDL1 | NTLYLQMNSLRAEDTAVYYCAKSSAGQSWPGFDYWGQ |
| | activatable | GTLVTVSS |
| | antibody | |
| 117 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTATTCTACTGGTGGTGCTACAGCTTACGCAGA |
| | | CTCCGTGAAGGGCCGGTTCACCATCTCCAGAGACAAT |
| | | TCCAAGAACACGCTGTATCTGCAAATGAACAGCCTGA |
| | | GAGCCGAGGACACGGCCGTATATTACTGTGCGAAATC |
| | | TTCTGCTGGTCAGTCGTTTCCGGGTTTTGACTACTGGG |
| | | GCCAGGGAACCCTGGTCACCGTCTCGAGC |
| 118 | VH domain | EVQLLESGGGLVQPGGSLRLSCAASGFTFSSYAMSWVR |
| | of anti- | QAPGKGLEWVSSIYSTGGATAYADSVKGRFTISRDNSK |
| | PDL1 | NTLYLQMNSLRAEDTAVYYCAKSSAGQSFPGFDYWGQ |
| | activatable | GTLVTVSS |
| | antibody | |
| 119 | VH domain | GAGGTGCAGCTGTTGGAGTCTGGGGGAGGCTTGGTAC |
| | of and- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTATTCTACTGGTGGTGCTACAGCTTACGCAGA |
| | | CTCCGTGAAGGGCCGGTTCACCATCTCCAGAGACAAT |
| | | TCCAAGAACACGCTGTATCTGCAAATGAACAGCCTGA |
| | | GAGCCGAGGACACGGCCGTATATTACTGTGCGAAATG |
| | | GTCTGCTGCTTTTGACTACTGGGGCCAGGGAACCCTG |
| | | GTCACCGTCTCGAGC |
| 120 | VH domain | EVQLLESGGGLVQPGGSLRLSCAASGFTFSSYAMSWVR |
| | of anti- | QAPGKGLEWVSSIYSTGGATAYADSVKGRFTISRDNSK |
| | PDL1 | NTLYLQMNSLRAEDTAVYYCAKWSAAFDYWGQGTLV |
| | activatable | TVSS |
| | antibody | |
| 121 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTATTCTACTGGTGGTGCTACAGCTTACGCAGA |
| | | CTCCGTGAAGGGCCGGTTCACCATCTCCAGAGACAAT |
| | | TCCAAGAACACGCTGTATCTGCAAATGAACAGCCTGA |
| | | GAGCCGAGGACACGGCCGTATATTACTGTGCGAAATG |
| | | GTCTGCTGCTTTTGACTACTGGGGCCAGGGAACCCTG |
| | | GTCACCGTCTCGAGC |
| 122 | VH domain | EVQLLESGGGLVQPGGSLRLSCAASGFTFSSYAMSWVR |
| | of anti- | QAPGKGLEWVSSIYSTGGATAYADSVKGRFTISRDNSK |
| | PDL1 | NTLYLQMNSLRAEDTAVYYCAKWSAGYDYWGQGTLV |
| | activatable | TVSS |
| | antibody | |
| 123 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTATTCTACTGGTGGTGCTACAGCTTACGCAGA |
| | | CTCCGTGAAGGGCCGGTTCACCATCTCCAGAGACAAT |
| | | TCCAAGAACACGCTGTATCTGCAAATGAACAGCCTGA |
| | | GAGCCGAGGACACGGCCGTATATTACTGTGCGAAATG |
| | | GTCTAAGGGTTTTGACTACTGGGGCCAGGGAACCCTG |
| | | GTCACCGTCTCGAGC |
| 124 | VH domain | EVQLLESGGGLVQPGGSLRLSCAASGFTFSSYAMSWVR |
| | of anti- | QAPGKGLEWVSSIYSTGGATAYADSVKGRFTISRDNSK |
| | PDL1 | NTLYLQMNSLRAEDTAVYYCAKWSKGFDYWGQGTLV |
| | activatable | TVSS |
| | antibody | |
| 125 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTGGAAGTAGGGTATTGTGACAGTGAGCTTAC |
| | | GCAGACTCCGTGAAGGGCCGGTTCACCATCTCCAGAG |
| | | ACAATTCCAAGAACACGCTGTATCTGCAAATGAACAG |
| | | CCTGAGAGCCGAGGACACGGCCGTATATTACTGTGCG |
| | | AAATCTTCTGCTGGTTTTGACTACTGGGGCCAGGGAA |
| | | CCCTGGTCACCGTCTCGAGC |
| 126 | VH domain | EVQLLESGGGLVQPGGSLRLSCAASGFTFSSYAMSWVR |
| | of anti- | QAPGKGLEWVSSIWKQGIVTVYDSVKGRFTISRDNSKN |
| | PDL1 | TLYLQMNSLRAEDTAVYYCAKSSAGFDYWGQGTLVTV |
| | activatable | |
| | antibody | |
| 127 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTGGCGGAATGGTATTGTTACAGTTAGCTTAC |
| | | GCAGACTCCGTGAAGGGCCGGTTCACCATCTCCAGAG |
| | | ACAATTCCAAGAACACGCTGTATCTGCAAATGAACAG |
| | | CCTGAGAGCCGAGGACACGGCCGTATATTACTGTGCG |
| | | AAATCTTCTGCTGGTTTTGACTACTGGGGCCAGGGAA |
| | | CCCTGGTCACCGTCTCGAGC |
| 128 | VH domain | EVQLLESGGGLVQPGGSLRLSCAASGFTFSSYAMSWVR |
| | of anti- | QAPGKGLEWVSSIWRNGIVTVYDSVKGRFTISRDNSKNT |
| | PDL1 | LYLQMNSLRAEDTAVYYCAKSSAGFDYWGQGTLVTVS |
| | activatable | S |
| | antibody | |
| 129 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | GATATTTGGAAGTAGGGTATGGTTACAGTGAGCTTAC |
| | | GCAGACTCCGTGAAGGGCCGGTTCACCATCTCCAGAG |
| | | ACAATTCCAAGAACACGCTGTATCTGCAAATGAACAG |
| | | CCTGAGAGCCGAGGACACGGCCGTATATTACTGTGCG |
| | | AAATCTTCTGCTGGTTTTGACTACTGGGGCCAGGGAA |
| | | CCCTGGTCACCGTCTCGAGC |
| 130 | VH domain | EVQLLESGGGLVQPGGSLRLSCAASGFTFSSYAMSWVR |
| | of anti- | QAPGKGLEWVSDIWKQGMVTVYDSVKGRFTISRDNSK |
| | PDL1 | NTLYLQMNSLRAEDTAVYYCAKSSAGFDYWGQGTLVT |
| | activatable | VSS |
| | antibody | |
| 131 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCAT |
| | antibody | CGATTTGGAGGTAGGGTCTGGCGACAGCGAGCTTACG |
| | | CAGACTCCGTGAAGGGCCGGTTCACCATCTCCAGAGA |
| | | CAATTCCAAGAACACGCTGTATCTGCAAATGAACAGC |
| | | CTGAGAGCCGAGGACACGGCCGTATATTACTGTGCGA |
| | | AATCTTCTGCTGGTTTTGACTACTGGGGCCAGGGAAC |
| | | CCTGGTCACCGTCTCGAGC |
| 132 | VH domain | EVQLLESGGGLVQPGGSLRLSCAASGFTFSSYAMSWVR |
| | of anti- | QAPGKGLEWVSSIWRQGLATAYDSVKGRFTISRDNSKN |
| | PDL1 | TLYLQMNSLRAEDTAVYYCAKSSAGFDYWGQGTLVTV |
| | activatable | SS |
| | antibody | |
| 133 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | GAGATTGTGGCTACTGGTATTTTGACAAGTAGCTTAC |
| | | GCAGACTCCGTGAAGGGCCGGTTCACCATCTCCAGAG |
| | | ACAATTCCAAGAACACGCTGTATCTGCAAATGAACAG |
| | | CCTGAGAGCCGAGGACACGGCCGTATATTACTGTGCG |
| | | AAATCTTCTGCTGGTTTTGACTACTGGGGCCAGGGAA |
| | | CCCTGGTCACCGTCTCGAGC |
| 134 | VH domain | EVQLLESGGGLVQPGGSLRLSCAASGFTFSSYAMSWVR |
| | of anti- | QAPGKGLEWVSEIVATGILTSYDSVKGRFTISRDNSKNT |
| | PDL1 | LYLQMNSLRAEDTAVYYCAKSSAGFDYWGQGTLVTVS |
| | activatable | S |
| | antibody | |
| 135 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatab1e | CGCCAGGCTCCAGGGAAGGGGCTGGAGTGGGTCTCAT |
| | antibody | CGATTGGTCGGTAGGGTTTGATTACAGTTAGCTTACG |
| | | CAGACTCCGTGAAGGGCCGGTTCACCATCTCCAGAGA |
| | | CAATTCCAAGAACACGCTGTATCTGCAAATGAACAGC |
| | | CTGAGAGCCGAGGACACGGCCGTATATTACTGTGCGA |
| | | AATCTTCTGCTGGTTTTGACTACTGGGGCCAGGGAAC |
| | | CCTGGTCACCGTCTCGAGC |
| 136 | VH domain | EVQLLESGGGLVQPGGSLRLSCAASGFTFSSYAMSWVR |
| | of anti- | QAPGKGLEWVSSIGRQGLITVYDSVKGRFTISRDNSKNT |
| | PDL1 | LYLQMNSLRAEDTAVYYCAKSSAGFDYWGQGTLVTVS |
| | activatable | S |
| | antibody | |
| 137 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCAT |
| | antibody | CTATTTGGTATTAGGGTCTGGTGACAGTTAGCTTACGC |
| | | AGACTCCGTGAAGGGCCGGTTCACCATCTCCAGAGAC |
| | | AATTCCAAGAACACGCTGTATCTGCAAATGAACAGCC |
| | | TGAGAGCCGAGGACACGGCCGTATATTACTGTGCGAA |
| | | ATCTTCTGCTGGTTTTGACTACTGGGGCCAGGGAACC |
| | | CTGGTCACCGTCTCGAGC |
| 138 | VH domain | EVQLLESGGGLVQPGGSLRLSCAASGFTFSSYAMSWVR |
| | of anti- | QAPGKGLEWVSSIWYQGLVTVYDSVKGRFTISRDNSKN |
| | PDL1 | TLYLQMNSLRAEDTAVYYCAKSSAGFDYWGQGTLVTV |
| | activatab1e | SS |
| | antibody | |
| 139 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | GATATTTGGAAGTAGGGTTTTGCTACAGCGAGCTTAC |
| | | GCAGACTCCGTGAAGGGCCGGTTCACCATCTCCAGAG |
| | | ACAATTCCAAGAACACGCTGTATCTGCAAATGAACAG |
| | | CCTGAGAGCCGAGGACACGGCCGTATATTACTGTGCG |
| | | AAATCTTCTGCTGGTTTTGACTACTGGGGCCAGGGAA |
| | | CCCTGGTCACCGTCTCGAGC |
| 140 | VH domain | EVQLLESGGGLVQPGGSLRLSCAASGFTFSSYAMSWVR |
| | of anti- | QAPGKGLEWVSDIWKQGFATADSVKGRFTISRDNSKNT |
| | PDL1 | LYLQMNSLRAEDTAVYYCAKSSAGFDYWGQGTLVTVS |
| | activatable | S |
| | antibody | |
| 141 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTGGAAGTAGGGTATTGTGACAGTGAGCTTAC |
| | | GCAGACTCCGTGAAGGGCCGGTTCACCATCTCCAGAG |
| | | ACAATTCCAAGAACACGCTGTATCTGCAAATGAACAG |
| | | CCTGAGAGCCGAGGACACGGCCGTATATTACTGTGCG |
| | | AAATCTTCTGCTGGTTTTGACTACTGGGGCCAGGGAA |
| | | CCCTGGTCACCGTCTCGAGC |
| 142 | VH domain | EVQLLESGGGLVQPGGSLRLSCAASGFTFSSYAMSWVR |
| | of anti- | QAPGKGLEWVSSIWKQGIVTVYDSVKGRFTTSRDNSKN |
| | PDL1 | TLYLQMNSLRAEDTAVYYCAKSSAGFDYWGQGTLVTV |
| | activatable | SS |
| | antibody | |
| 143 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCAT |
| | antibody | CGATTTGGAGGTAGGGTCTGGCGACAGCGAGCTTACG |
| | | CAGACTCCGTGAAGGGCCGGTTCACCATCTCCAGAGA |
| | | CAATTCCAAGAACACGCTGTATCTGCAAATGAACAGC |
| | | CTGAGAGCCGAGGACACGGCCGTATATTACTGTGCGA |
| | | AATCTTCTGCTGGTTTTGACTACTGGGGCCAGGGAAC |
| | | CCTGGTCACCGTCTCGAGC |
| 144 | VH domain | EVQLLESGGGLVQPGGSLRLSCAASGFTFSSYAMSWVR |
| | of anti- | QAPGKGLEWVSSIWRQGLATAYDSVKGRFTISRDNSKN |
| | PDL1 | TLYLQMNSLRAEDTAVYYCAKSSAGFDYWGQGTLVTV |
| | activatable | SS |
| | antibody | |
| 145 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatab1e | CGCCAGGCTCCAGGGAAGGGGCTGGAGTGGGTCTCA |
| | antibody | AGTATTTGGCGGAATGGTATTGTTACAGTTTACGCAG |
| | | ACTCCGTGAAGGGCCGGTTCACCATCTCCAGAGACAA |
| | | TTCCAAGAACACGCTGTATCTGCAAATGAACAGCCTG |
| | | AGAGCCGAGGACACGGCCGTATATTACTGTGCGAAAT |
| | | GGTCTGCTGCTTTTGACTACTGGGGCCAGGGAACCCT |
| | | GGTCACCGTCTCGAGC |
| 146 | VH domain | EVQLLESGGGLVQPGGSLRLSCAASGFTFSSYAMSWVR |
| | of anti- | QAPGKGLEWVSSIWRNGIVTVYADSVKGRFTISRDNSK |
| | PDL1 | NTLYLQMNSLRAEDTAVYYCAKWSAAFDYWGQGTLV |
| | activatable | TVSS |
| | antibody | |
| 147 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTGGCGGAATGGTATTGTTACAGTTTACGCAG |
| | | ACTCCGTGAAGGGCCGGTTCACCATCTCCAGAGACAA |
| | | TTCCAAGAACACGCTGTATCTGCAAATGAACAGCCTG |
| | | AGAGCCGAGGACACGGCCGTATATTACTGTGCGAAAT |
| | | GGTCTGCTGGTTATGACTACTGGGGCCAGGGAACCCT |
| | | GGTCACCGTCTCGAGC |
| 148 | VH domain | EVQLLESGGGLVQPGGSLRLSCAASGFTFSSYAMSWVR |
| | of anti- | QAPGKGLEWVSSIWRNGIVTVYADSVKGRFTISRDNSK |
| | PDL1 | NTLYLQMNSLRAEDTAVYYCAKWSAGYDYWGQGTLV |
| | activatable | TVSS |
| | antibody | |
| 149 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTGGCGGAATGGTATTGTTACAGTTTACGCAG |
| | | ACTCCGTGAAGGGCCGGTTCACCATCTCCAGAGACAA |
| | | TTCCAAGAACACGCTGTATCTGCAAATGAACAGCCTG |
| | | AGAGCCGAGGACACGGCCGTATATTACTGTGCGAAAT |
| | | GGTCTAAGGGTTTTGACTACTGGGGCCAGGGAACCCT |
| | | GGTCACCGTCTCGAGC |
| 150 | VH domain | EVQLLESGGGLVQPGGSLRLSCAASGFTFSSYAMSWVR |
| | of anti- | QAPGKGLEWVSSIWRNGIVTVYADSVKGRFTISRDNSK |
| | PDL1 | NTLYLQMNSLRAEDTAVYYCAKWSKGFDYWGQGTLV |
| | activatable | TVSS |
| | antibody | |
| 151 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTAGCAGCTATGCCATGAGCTGGGTC |
| | activatable | CGCCAGGCTCCAGGGAAGGGGCTGGAGTGGGTCTCAT |
| | antibody | CTATTTGGTATCAGGGTCTGGTGACAGTTTACGCAGA |
| | | CTCCGTGAAGGGCCGGTTCACCATCTCCAGAGACAAT |
| | | TCCAAGAACACGCTGTATCTGCAAATGAACAGCCTGA |
| | | GAGCCGAGGACACGGCCGTATATTACTGTGCGAAATG |
| | | GTCTGCTGCTTTTGACTACTGGGGCCAGGGAACCCTG |
| | | GTCACCGTCTCGAGC |
| 152 | VH domain | EVQLLESGGGLVQPGGSLRLSCAASGFTFSSYAMetSWV |
| | of anti- | RQAPGKGLEWVSSIWYQGLVTVYADSVKGRFTISRDNS |
| | PDL1 | KNTLYLQMetNSLRAEDTAVYYCAKWSAAFDYWGQGT |
| | activatable | LVTVSS |
| | antibody | |
| 153 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTGGCGGAATGGTATTGTTACAGTTTACGCAG |
| | | ACTCCGTGAAGGGCCGGTTCACCATCTCCAGAGACAA |
| | | TTCCAAGAACACGCTGTATCTGCAAATGAACAGCCTG |
| | | AGAGCCGAGGACACGGCCGTATATTACTGTGCGAAAT |
| | | GGTCTGCTGGTTATGACTACTGGGGCCAGGGAACCCT |
| | | GGTCACCGTCTCGAGC |
| 154 | VH domain | EVQLLESGGGLVQPGGSLRLSCAASGFTFSSYAMSWVR |
| | of anti- | QAPGKGLEWVSSIWYQGLVTVYADSVKGRFTISRDNSK |
| | PDL1 | NTLYLQMNSLRAEDTAVYYCAKWSAGYDYWGQGTLV |
| | activatable | TVSS |
| | antibody | |
| 155 | VH domain | GAGGTGCAGCTGTTGGAGTCTGGGGGAGGCTTGGTAC |
| | of anti- | AGCCTGGGGGGTCCCTGAGACTCTCCTGTGCAGCCTC |
| | PDL1 | TGGATTCACCTTTAGCAGCTATGCCATGAGCTGGGTC |
| | activatable | CGCCAGGCTCCAGGGAAGGGGCTGGAGTGGGTCTCA |
| | antibody | AGTATTTGGCGGAATGGTATTGTTACAGTTTACGCAG |
| | | ACTCCGTGAAGGGCCGGTTCACCATCTCCAGAGACAA |
| | | TTCCAAGAACACGCTGTATCTGCAAATGAACAGCCTG |
| | | AGAGCCGAGGACACGGCCGTATATTACTGTGCGAAAT |
| | | GGTCTAAGGGTTTTGACTACTGGGGCCAGGGAACCCT |
| | | GGTCACCGTCTCGAGC |
| 156 | VH domain | EVQLLESGGGLVQPGGSLRLSCAASGFTFSSYAMSWVR |
| | of anti- | QAPGKGLEWVSSIWYQGLVTVYADSVKGRFTISRDNSK |
| | PDL1 | NTLYLQMNSLRAEDTAVYYCAKWSKGFDYWGQGTLV |
| | activatable | TVSS |
| | antibody | |
| 157 | Spacer | QGQSGS |
| 158 | Spacer | GQSGS |
| 159 | Spacer | QSGS |
| 160 | Spacer | QGQSGQG |
| 161 | Spacer | GQSGQG |
| 162 | Spacer | QSGQG |
| 163 | Spacer | SGQG |
| 164 | Spacer | QGQSGQ |
| 165 | Spacer | GQSGQ |
| 166 | Spacer | QSGQ |
| 167 | CX-072 | CAGGGCCAGTCCGGCTCATATCTGCCCTGCCACTTCG |
| | light chain | TGCCAATCGGGGCCTGTACAATAAGGGCGGTGGATC |
| | with spacer | TAGTGGTGGCTCAGGCGGGTCTGGCGGCATTTCCAGT |
| | | GGACTCTTGTCAGGACGATCCGATAATCATGGCGGGT |
| | | CCGACATCCAGATGACACAGAGCCCTTCTTCCCTCTC |
| | | CGCAAGCGTTGGCGACAGGGTCACCATTACCTGTAGG |
| | | GCTTCTCAGAGCATCTCAAGCTATCTGAACTGGTACC |
| | | AGCAGAAACCTGGAAAGGCTCCAAAACTGCTGATTTA |
| | | CGCTGCCTCCAGTCTTCAGTCAGGCGTCCCCTCCAGAT |
| | | TTAGCGGATCAGGTAGTGGAACTGATTTTACCCTTAC |
| | | AATATCTTCTCTGCAGCCAGAGGACTTCGCCACATAC |
| | | TATTGTCAGCAAGACAATGGTTACCCCAGTACATTTG |
| | | GCGGAGGGACAAAGGTCGAGATCAAAAGGACCGTAG |
| | | CAGCACCAAGCGTCTTTATTTTCCCCCCCAGTGACGA |
| | | ACAGCTGAAGAGCGGAACAGCTTCAGTGGTGTGTCTC |
| | | CTGAATAACTTCTATCCACGCGAGGCAAAGGTGCAGT |
| | | GGAAGGTGGACAATGCACTGCAGTCTGGTAATTCCCA |
| | | AGAAAGTGTTACTGAGCAGGATTCCAAGGATTCAACT |
| | | TACTCTCTGTCTAGCACCCTGACTCTTTCTAAAGCAGA |
| | | TTATGAGAAGCATAAGGTCTACGCTTGCGAGGTGACC |
| | | CACCAGGGGCTTTCCTCTCCAGTTACCAAGTCATTCA |
| | | ACCGGGGTGAGTGTTGATGAGAATTC |
| 168 | Light chain | QGQSGSGIALCPSHFCQLPQTGGGSSGGSGGSGGISSGLL |
| | with spacer | SGRSDNHGGSDIQMTQSPSSLSASVGDRVTITCRASQSIS |
| | | SYLNWYQQKPGKAPKLLIYAASSLQSGVPSRFSGSGSGT |
| | | DFTLTISSLQPEDFATYYCQQDNGYPSTFGGGTKVEIKRT |
| | | VAAPSVFIFPPSDEQLKSGTASVVCLLNNFYPREAKVQW |
| | | KVDNALQSGNSQESVTEQDSKDSTYSLSSTLTLSKADYE |
| | | KHKVYACEVTHQGLSSPVTKSFNRGEC |
| 169 | Light chain | TATCTGCCCTGCCACTTCGTGCCAATCGGGGCCTGTA |
| | without | ACAATAAGGGCGGTGGATCTAGTGGTGGCTCAGGCG |
| | spacer | GGTCTGGCGGCATTTCCAGTGGACTCTTGTCAGGACG |
| | | ATCCGATAATCATGGCGGGTCCGACATCCAGATGACA |
| | | CAGAGCCCTTCTTCCCTCTCCGCAAGCGTTGGCGACA |
| | | GGGTCACCATTACCTGTAGGGCTTCTCAGAGCATCTC |
| | | AAGCTATCTGAACTGGTACCAGCAGAAACCTGGAAA |
| | | GGCTCCAAAACTGCTGATTTACGCTGCCTCCAGTCTTC |
| | | AGTCAGGCGTCCCCTCCAGATTTAGCGGATCAGGTAG |
| | | TGGAACTGATTTTACCCTTACAATATCTTCTCTGCAGC |
| | | CAGAGGACTTCGCCACATACTATTGTCAGCAAGACAA |
| | | TGGTTACCCCAGTACATTTGGCGGAGGGACAAAGGTC |
| | | GAGATCAAAAGGACCGTAGCAGCACCAAGCGTCTTTA |
| | | TTTTCCCCCCCAGTGACGAACAGCTGAAGAGCGGAAC |
| | | AGCTTCAGTGGTGTGTCTCCTGAATAACTTCTATCCAC |
| | | GCGAGGCAAAGGTGCAGTGGAAGGTGGACAATGCAC |
| | | TGCAGTCTGGTAATTCCCAAGAAAGTGTTACTGAGCA |
| | | GGATTCCAAGGATTCAACTTACTCTCTGTCTAGCACCC |
| | | TGACTCTTTCTAAAGCAGATTATGAGAAGCATAAGGT |
| | | CTACGCTTGCGAGGTGACCCACCAGGGGCTTTCCTCT |
| | | CCAGTTACCAAGTCATTCAACCGGGGTGAGTGTTGAT |
| | | GAGAATTC |
| 170 | Light chain | GIALCPSHFCQLPQTGGGSSGGSGGSGGISSGLLSGRSDN |
| | without | HGGSDIQMTQSPSSLSASVGDRVTITCRASQSISSYLNWY |
| | spacer | QQKPGKAPKLLIYAASSLQSGVPSRFSGSGSGTDFTLTIS |
| | | SLQPEDFATYYCQQDNGYPSTFGGGTKVEIKRTVAAPSV |
| | | FIFPPSDEQLKSGTASVVCLLNNFYPREAKVQWKVDNA |
| | | LQSGNSQESVTEQDSKDSTYSLSSTLTLSKADYEKHKVY |
| | | ACEVTHQGLSSPVTKSFNRGEC |
| 171 | Heavy chain | GAAGTGCAGCTGCTCGAAAGCGGCGGAGGCTTGGTG |
| | | CAGCCAGGAGGGAGCCTGCGACTGTCTTGCGCAGCCA |
| | | GCGGATTCACTTTCTCTTCCTATGCCATGAGCTGGGTT |
| | | CGACAGGCACCCGGCAAAGGTCTCGAGTGGGTGTCTA |
| | | GCATCTGGCGAAACGGAATAGTTACAGTGTATGCCGA |
| | | TAGCGTGAAGGGTCGCTTTACTATTTCACGGGATAAT |
| | | TCTAAGAACACCCTCTACCTGCAAATGAATAGCCTTA |
| | | GGGCAGAAGATACCGCCGTGTACTACTGTGCCAAATG |
| | | GTCCGCAGCCTTTGACTACTGGGGCCAGGGGACACTG |
| | | GTGACCGTGTCCTCTGCATCAACCAAGGGGCCATCAG |
| | | TGTTCCCACTCGCCCCATGTAGCAGATCAACATCTGA |
| | | ATCCACCGCAGCCCTTGGCTGCCTTGTTAAGGACTATT |
| | | TCCCAGAACCCGTGACCGTAAGTTGGAACTCTGGCGC |
| | | CCTTACTTCTGGGGTGCACACCTTCCCAGCAGTGTTGC |
| | | AGTCCAGTGGCCTTTACTCTCTGTCTAGTGTAGTGACT |
| | | GTGCCTTCCTCTAGTCTCGGTACCAAGACCTATACCTG |
| | | CAACGTAGATCATAAGCCCAGCAATACAAAGGTTGAT |
| | | AAGAGAGTAGAGTCAAAGTACGGCCCACCCTGCCCA |
| | | CCTTGTCCAGCTCCCGAGTTCCTGGGCGGACCCTCAG |
| | | TCTTTCTCTTCCCACCTAAACCCAAGGATACCCTTATG |
| | | ATCTCCAGGACTCCTGAGGTGACCTGCGTTGTGGTCG |
| | | ACGTGTCACAAGAGGACCCTGAGGTACAGTTTAACTG |
| | | GTACGTGGACGGTGTGGAGGTACATAACGCTAAGACT |
| | | AAGCCACGAGAGGAGCAATTTAACTCCACTTACAGGG |
| | | TGGTCAGCGTCCTGACCGTTCTCCATCAGGACTGGCT |
| | | GAACGGGAAGGAATATAAGTGTAAGGTTAGCAACAA |
| | | AGGTCTGCCCAGTTCTATCGAGAAGACAATCAGCAAG |
| | | GCAAAAGGGCAGCCTCGGGAACCTCAGGTCTACACCC |
| | | TCCCTCCTAGCCAGGAAGAGATGACAAAGAACCAGG |
| | | TCTCTCTCACCTGCCTGGTGAAAGGCTTCTATCCATCT |
| | | GACATTGCTGTGGAGTGGGAATCCAACGGCCAGCCTG |
| | | AAAATAATTATAAGACCACACCCCCCGTCCTTGATTC |
| | | CGATGGATCTTTCTTCCTGTACAGTCGCCTCACCGTCG |
| | | ACAAATCACGGTGGCAGGAAGGTAACGTGTTCAGCTG |
| | | TTCTGTCATGCATGAGGCTCTGCATAACCATTACACA |
| | | CAAAAGTCTTTGTCATTGTCTCTCGGATGATGAGAATT |
| | | CATTGATCATAATCAGCCATACCAC |
| 172 | Heavy chain | EVQLLESGGGLVQPGGSLRLSCAASGFTGSSYAMSWVR |
| | | QAPGKGLEWVSSIWRNGIVTVYADSVKGRFTISRDNSK |
| | | NTLYLQMNSLRAEDTAVYYCAKWSAAFDYWGQGTLV |
| | | TVSSASTKGPSVFPLAPCSRSTSESTAALGCLVKDYFPEP |
| | | VTVSWNSGALTSGVHTFPAVLQSSGLYSLSSVVTVPSSS |
| | | LGTKTYTCNVDHKPSNTKVDKRVESKYGPPCPPCPAPEF |
| | | LGGPSVFLFPPKPKDTLMISRTPEVTCVVVDVSQEDPEV |
| | | QFNWYVDGVEVHNAKTKPREEQFNSTYRVVSVLTVLH |
| | | QDWLNGKEYKCKVSNKGLPSSIEKTISKAKGQPREPQV |
| | | YTLPPSQEEMTKNQVSLTCLVKGFYPSDIAVEWESNGQP |
| | | ENNYKTTPPVLDSDGSFFLYSRLTVDKSRWQEGNVFSCS |
| | | VMHEALHNHYTQKSLSLSLG |
| 173 | Linker | GGGSSGGSGGSGG |
| 174 | PDL1-Ab | DIQMTQSPSSLSASVGDRVTITCRASQSISSYLNWYQQKP |
| | Antibody | GKAPKLLIYAASSLQSGVPSRFSGSGSGTDFTLTISSLQPE |
| | Light Chain | DFATYYCQQDNGYPSTFGGGTKVEIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| 175 | PDL1-Ab | EVQLLESGGGLVQPGGSLRLSCAASGFTFSSYAMSWVR |
| | Antibody | QAPGKGLEWVSSIWRNGIVTVYADSVKGRFTISRDNSK |
| | Heavy | NTLYLQMNSLRAEDTAVYYCAKWSAAFDYWGQGTLV |
| | Chain | TVSSASTKGPSVFPLAPCSRSTSESTAALGCLVKDYFPEP |
| | | VTVSWNSGALTSGVHTFPAVLQSSGLYSLSSVVTVPSSS |
| | | LGTKTYTCNVDHKPSNTKVDKRVESKYGPPCPPCPAPEF |
| | | LGGPSVFLFPPKPKDTLMISRTPEVTCVVVDVSQEDPEV |
| | | QFNWYVDGVEVHNAKTKPREEQFNSTYRVVSVLTVLH |
| | | QDWLNGKEYKCKVSNKGLPSSIEKTISKAKGQPREPQV |
| | | YTLPPSQEEMTKNQVSLTCLVKGFYPSDIAVEWESNGQP |
| | | ENNYKTTPPVLDSDGSFFLYSRLTVDKSRWQEGNVFSCS |
| | | VMHEALHNHYTQKSLSLSLG |
| 176 | VH | QVQLVQSGAEVKKPGASVKVSCKASGYTFTDYGFSWV |
| | | RQAPGQGLEWMGWITAYNGNTNYAQKLQGRVTMTTD |
| | | TSTSTVYMELRSLRSDDTAVYYCARDYFYGMDVWGQG |
| | | TTVTVSS |
| 177 | VH | QVQLVQSGAEVKKPGSSVKVSCKTSGDTFSTYAISWVR |
| | | QAPGQGLEWMGGIIPIFGKAHYAQKFQGRVTITADESTS |
| | | TAYMELSSLRSEDTAVYFCARKFHFVSGSPFGMDVWGQ |
| | | GTTVTVSS |
| 178 | VH | QVQLVQSGAEVKKPGASVKVSCKASGYTFTSYDVHWV |
| | | RQAPGQRLEWMGWLHADTGITKFSQKFQGRVTITRDTS |
| | | ASTAYMELSSLRSEDTAVYYCARERIQLWFDYWGQGT |
| 179 | VH | QVQLVQSGAEVKKPGSSVKVSCKVSGGIFSTYAINWVR |
| | | QAPGQGLEWMGGIIPIFGTANHAQKFQGRVTITADESTS |
| | | TAYMELSSLRSEDTAVYYCARDQGIAAALFDYWGQGT |
| | | LVTVSS |
| 180 | VH | EVQLVESGGGLVQPGRSLRLSCAVSGFTFDDYVVHWVR |
| | | QAPGKGLEWVSGNSGNIGYADSVKGRFTISRDNAKNSL |
| | | YLQMNSLRAEDTALYYCAVPFDYWGQGTLVTVSS |
| 181 | VH | QVQLVQSGAEVKKPGSSVKVSCKTSGDTFSSYAISWVR |
| | | QAPGQGLEWMGGLIPIFGRAHYAQKFQGRVTITADESTS |
| | | TAYMELSSLRSEDTAVYFCARKFHFVSGSPFGMDVWGQ |
| | | GTTVTVSS |
| 182 | VH | QVQLVQSGAEVKKPGSSVKVSCKTSGGTFSSYAISWVR |
| | | QAPGQGLEWMGGIIPIFGKAHYAQKFQGRVTITADESTT |
| | | TAYMELSSLRSEDTAVYYCARKYDYVSGSPFGMDVWG |
| | | QGTTVTVSS |
| 183 | VH | QVQLVQSGAEVKKPGSSVKVSCKASGGTFSSYAINWVR |
| | | QAPGQGLEWMGGIIPIFGSANYAQKFQDRVTITADESTS |
| | | AAYMELSSLRSEDTAVYYCARDSSGWSRYYMDVWGQ |
| | | GTTVTVSS |
| 184 | VH | QVQLVQSGAEVKEPGSSVKVSCKASGGTFNSYAISWVR |
| | | QAPGQGLEWMGGIIPLFGIAHYAQKFQGRVTITADESTN |
| | | TAYMDLSSLRSEDTAVYYCARKYSYVSGSPFGMDVWG |
| | | QGTTVTVSS |
| 185 | VH | EVQLVESGGGLVQPGRSLRLSCAASGITFDDYGMHWVR |
| | | QAPGKGLEWVSGISWNRGRIEYADSVKGRFTISRDNAK |
| | | NSLYLQMNSLRAEDTALYYCAKGRFRYFDWFLDYWGQ |
| | | GTLVTVSS |
| 186 | VH | QMQLVQSGGGLVQPGGSLRLSCAASGFTFSSYWMSWV |
| | | RQAPGKGLEWVANIKQDGSEKYYVDSVKGRFTISRDNA |
| | | KNSLYLQMNSLRAEDTAVYYCARDYFWSGFSAFDIWG |
| | | KGTLVTVS |
| 187 | VL | EIVLTQSPATLSLSPGERATLSCRASQSVSSYLVWYQQK |
| | | PGQAPRLLIYDASNRATGIPARFSGSGSGTDFTLTISSLEP |
| | | EDFAVYYCQQRSNWPRTFGQGTKVEIK |
| 188 | VL | EIVLTQSPATLSLSPGERATLSCRASQSVSSYLAWYQQK |
| | | PGQAPRLLIYDASNRATGIPARFSGSGSGTDFTLTISSLEP |
| | | EDFAVYYCQQRSNWPTFGQGTKVEIK |
| 189 | VL | DIQMTQSPSSLSASVGDRVTITCRASQGISSWLAWYQQK |
| | | PEKAPKSLIYAASSLQSGVPSRFSGSGSGTDFTLTISSLQP |
| | | EDFATYYCQQYNSYPYTFGQGTKLEIK |
| 190 | VL | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQYGSSPWTFGQGTKVEIK |
| 191 | VL | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQYGSSPFGGGTKVEIK |
| 192 | VL | EIVLTQSPATLSLSPGERATLSCRASQSVSSYLAWYQQK |
| | | PGQAPRLLIYDASNRATGIPARFSGSGSGTDFTLTISSLEP |
| | | EDFAVYYCQQRSNWPTFGQGTRLEIK |
| 193 | VL | AIQLTQSPSSLSASVGDRVTITCRASQGISSALAWYQQKP |
| | | GKARKLLIYDASSLESGVPSRFSGSGSGTDFTLTISSLQPE |
| | | DFATYYCQQFNSYPFTFGPGTKVDIK |
| 194 | VL | DIVMTQSPSTLSASVGDRVTITCRASQGISSWLAWYQQK |
| | | PGRAPKVLIYKASTLESGVPSRFSGSGSGTDFTLTISSLQP |
| | | EDFATYYCQQSYSTPWTFGQGTKLEIK |
| 195 | VH | EVQLVESGGGLVQPGGSLRLSCAASGFTFSRYWMSWVR |
| | | QAPGKGLEWVANIKQDGSEKYYVDSVKGRFTISRDNAK |
| | | NSLYLQMNSLRAEDTAVYYCAREGGWFGELAFDYWG |
| | | QGTLVTVSS |
| 196 | VL | EIVLTQSPGTLSLSPGERATLSCRASQRVSSSYLAWYQQ |
| | | KPGQAPRLLIYDASSRATGIPDRFSGSGSGTDFTLTISRLE |
| | | PEDFAVYYCQQYGSLPWTFGQGTKVEIK |
| 197 | VH | EVQLVESGGGLVQPGGSLRLSCAASGFTFSDSWIHWVR |
| | | QAPGKGLEWVAWISPYGGSTYYADSVKGRFTISADTSK |
| | | NTAYLQMNSLRAEDTAVYYCARRHWPGGFDYWGQGT |
| | | LVTVSA |
| 198 | VH | EVQLVESGGGLVQPGGSLRLSCAASGFTFSGSWIHWVR |
| | | QAPGKGLEWVAWILPYGGSSYYADSVKGRFTISADTSK |
| | | NTAYLQMNSLRAEDTAVYYCARRHWPGGFDYWGQGT |
| | | LVTVSA |
| 199 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQYLYHPATFGQGTKVEIKR |
| 200 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQYYNVPWTFGQGTKVEIKR |
| 201 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQYYAPPWTFGQGTKVEIKR |
| 202 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQYYTVPWTFGQGTKVEIKR |
| 203 | VL | DIQMTQSPSSLSASVGDRVTITCRASQVINTFLAWYQQK |
| | | PGKAPKLLIYSASTLASGVPSRFSGSGSGTDFTLTISSLQP |
| | | EDFATYYCQQYYTVPRTFGQGTKVEIKR |
| 204 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQGYGVPRTFGQGTKVEIKR |
| 205 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQYLFTPPTFGQGTKVEIKR |
| 206 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQYFITPTTFGQGTKVEIKR |
| 207 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQYYYTPPTFGQGTKVEIKR |
| 208 | VL | DIQMTQSPSSLSASVGDRVTITCRASQDVSTAVAWYQQ |
| | | KPGKAPKLLIYSASFLYSGVPSRFSGSGSGTDFFLTISSLQ |
| | | PEDFATYYCQQFFYTPPTFGQGTKVEIKR |
| 209 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQSLFTPPTFGQGTKVEIKR |
| 210 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQSLYTPPTFGQGTKVEIKR |
| 211 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQSWYHPPTFGQGTKVEIKR |
| 212 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQYFYIPPTFGQGTKVEIKR |
| 213 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQYWYTPTTFGQGTKVEIKR |
| 214 | VL | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQSYFIPPTFGQGTKVEIKR |
| 215 | VH | METGLRWLLLVAVLKGVQCLSVEESGGRLVTPGTPLTL |
| | | TCTASGFTITNYHMFWVRQAPGKGLEWIGVITSSGIGSSS |
| | | TTYYATWAKGRFTISKTSTTVNLRITSPTTEDTATYFCAR |
| | | DYFTNTYYALDIWGPGTLVTVSS |
| 216 | VH | QVQLVQSGAEVKKPGASVKVSCKASGYTFTSYDVHWV |
| | | RQAPGQRLEWMGWLHADTGITKFSQKFQGRVTITRDTS |
| | | ASTAYMELSSLRSEDTAVYYCARERIQLWFDYWGQGTL |
| | | VTVSS |
| 217 | VH | EVQLVESGGGLVQPGRSLRLSCAVSGFTFDDYVVHWVR |
| | | QAPGKGLEWVSGISGNSGNIGYADSVKGRFTISRDNAK |
| | | NSLYLQMNSLRAEDTALYYCAVPFDYWGQGTLVTVSS |
| 218 | VL | MDTRAPTQLLGLLLLWLPGARCALVMTQTPSSTSTAVG |
| | | GTVTIKCQASQSISVYLAWYQQKPGQPPKLLIYSASTLA |
| | | SGVPSRFKGSRSGTEYTLTISGVQREDAATYYCLGSAGS |
| 219 | VH | EVQLLESGGGLVQPGGSLRLSCAASGFTFSSYIMMWVR |
| | | QAPGKGLEWVSSIYPSGGITFYADTVKGRFTISRDNSKN |
| | | TLYLQMNSLRAEDTAVYYCARIKLGTVTTVDYWGQGT |
| | | LVTVSS |
| 220 | VL | QSALTQPASVSGSPGQSITISCTGTSSDVGGYNYVSWYQ |
| | | QHPGKAPKLMIYDVSNRPSGVSNRFSGSKSGNTASLTIS |
| | | GLQAEDEADYYCSSYTSSSTRVFGTGTKVTVL |
| 221 | VH | EVKLQESGPSLVKPSQTLSLTCSVTGYSITSDYWNWIRK |
| | | FPGNKLEYVGYISYTGSTYYNPSLKSRISITRDTSKNQYY |
| | | LQLNSVTSEDTATYYCARYGGWLSPFDYWGQGTTLTVS |
| | | S |
| 222 | VH | EVQLQESGPGLVAPSQSLSITCTVSGFSLTTYSINWIRQPP |
| | | GKGLEWLGVMWAGGGTNSNSVLKSRLIISKDNSKSQVF |
| | | LKMNSLQTDDTARYYCARYYGNSPYYAIDYWGQGTSV |
| 223 | VH | EVKLQESGPSLVKPSQTLSLTCSVTGYSIISDYWNWIRKF |
| | | PGNKLEYLGYISYTGSTYYNPSLKSRISITRDTSKNQYYL |
| | | QLNSVTTEDTATYYCARRGGWLLPFDYWGQGTTLTVSS |
| 224 | VH | EVKLQESGPSLVKPGASVKLSCKASGYTFTSYDINWVK |
| | | QRPGQGLEWIGWIFPRDNNTKYNENFKGKATLTVDTSS |
| | | TTAYMELHSLTSEDSAVYFCTKENWVGDFDYWGQGTT |
| | | LTLSS |
| 225 | VH | EVQLQQSGPDLVTPGASVRISCQASGYTFPDYYMNWVK |
| | | QSHGKSLEWIGDIDPNYGGTTYNQKFKGKAILTVDRSSS |
| | | TAYMELRSLTSEDSAVYYCARGALTDWGQGTSLYVSS |
| 226 | VH | EIVLTQSPATLSLSPGERATLSCRASSSVSYIYWFQQKPG |
| | | QSPRPLIYAAFNRATGIPARFSGSGSGTDYTLTISSLEPED |
| | | FAVYYCQQWSNNPLTFGQGTKVEIK |
| 227 | VH | QVQLVQSGAEVKKPGASVKVSCKASGYTFPDYYMNWV |
| | | RQAPGQGLEWMGDIDPNYGGTNYAQKFQGRVTMTRDT |
| | | SISTAYMELSRLRSDDTAVYYCARGALTDWGQGTMVT |
| | | VSS |
| 228 | VH | QVQLVQSGAEVKKPGASVKVSCKASGYTFPDYYMNWV |
| | | RQAPGQGLEWMGDIDPNYGGTNYAQKFQGRVTMTRDT |
| | | SISTAYMELSRLRSDDTAVYYCARGALTDWGQGTMVT |
| | | VSS |
| 229 | VH | EVQLVQSGAEVKKPGASVKVSCKASGYTFPDYYMNWV |
| | | RQAPGQSLEWMGDIDPNYGGTNYNQKFQGRVTMTVDR |
| | | SSSTAYMELSRLRSDDTAVYYCARGALTDWGQGTMVT |
| | | VSS |
| 230 | VH | EVQLVESGGGLVQPGRSLRLSCTASGYTFPDYYMNWVR |
| | | QAPGKGLEWVGDIDPNYGGTTYAASVKGRFTISVDRSK |
| | | SIAYLQMSSLKTEDTAVYYCTRGALTDWGQGTMVTVSS |
| 231 | VH | EVQLVESGGGLVQPGRSLRLSCTASGYTFPDYYMNWVR |
| | | QAPGKGLEWVGDIDPNYGGTTYNASVKGRFTISVDRSK |
| | | SIAYLQMSSLKTEDTAVYYCARGALTDWGQGTMVTVS |
| | | S |
| 232 | VL | DIVMTQSHKLMSTSVGDRVSITCKASQDVGTAVAWYQ |
| | | QKPGQSPKLLIYWASTRHTGVPDRFTGSGSGTDFTLTISN |
| | | VQSEDLADYFCQQDSSYPLTFGAGTKVELK |
| 233 | VL | DIVTTQSHKLMSTSVGDRVSITCKASQDVGTAVAWYQQ |
| | | KPGQSPKLLIYWASTRHTGVPDRFTGSGSGTDFTLTISNV |
| | | QSEDLADYFCQQDSSYPLTFGAGTKVELK |
| 234 | VL | DIVMTQSPSSLAVSVGEKVSMGCKSSQSLLYSSNQKNSL |
| | | AWYQQKPGQSPKLLIDWASTRESGVPDRFTGSGSGTDF |
| | | TLTISSVKAEDLAVYYCQQYYGYPLTFGAGTKLELK |
| 235 | VL | DIVMTQSPAIMSASPGEKVTMTCSASSSIRYMHWYQQK |
| | | PGTSPKRWISDTSKLTSGVPARFSGSGSGTSYALTISSME |
| | | AEDAATYYCHQRSSYPWTFGGGTKLEIK |
| 236 | VL | QIVLSQSPAILSASPGEKVTMTCRASSSVSYIYWFQQKPG |
| | | SSPKPWIYATFNLASGVPARFSGSGSGTSYSLTISRVETE |
| | | DAATYYCQQWSNNPLTFGAGTKLELK |
| 237 | VL | EIVLTQSPATLSLSPGERATLSCRASSSVSYIYWFQQKPG |
| | | QAPRLLIYAAFNRATGIPARFSGSGSGTDYTLTISSLEPED |
| | | FAVYYCQQWSNNPLTFGQGTKVEIK |
| 238 | VL | QIVLTQSPATLSLSPGERATLSCRASSSVSYIYWFQQKPG |
| | | QSPRPLIYATFNLASGIPARFSGSGSGTSYTLTISRLEPEDF |
| | | AVYYCQQWSNNPLTFGQGTKVEIK |
| 239 | VL | DIQLTQSPSSLSASVGDRVTITCRASSGVSYIYWFQQKPG |
| | | KAPKLLIYAAFNLASGVPSRFSGSGSGTEYTLTISSLQPE |
| | | DFATYYCQQWSNNPLTFGQGTKVEIK |
| 240 | VL | DIQLTQSPSSLSASVGDRVTITCRASSGVSYIYWFQQKPG |
| | | KAPKPLIYAAFNLASGVPSRFSGSGSGTEYTLTISSLQPE |
| | | DFATYYCQQWSNNPLTFGQGTKVEIK |
| 241 | VL | DIQLTQSPSILSASVGDRVTITCRASSSVSYIYWFQQKPG |
| | | KAPKPLIYATFNLASGVPSRFSGSGSGTSYTLTISSLQPED |
| | | FATYYCQQWSNNPLTFGQGTKVEIK |
| 242 | VH | QVQLVQSGAEVKKPGASVKVSCKASGYTFTSYGISWVR |
| | | QAPGQGLEWMGWISAYNGNTNYAQKLQGRVTMTTDT |
| | | STSTAYMELRSLRSDDTAVYYCARALPSGTILVGGWFD |
| | | PWGQGTLVTVSS |
| 243 | VH | EVQLVQSGGGVVQPGRSLRLSCAASGFTFSSYALSWVR |
| | | QAPGKGLEWVSAISGGGGSTYYADSVKGRFTISRDNSK |
| | | NTLYLQMNSLRAEDTAVYYCAKDVFPETFSMNYGMDV |
| | | WGQGTLVTVSS |
| 244 | VH | QVQLVQSGGGVVQPGGSLRLSCAASGFTFDDYAMHWV |
| | | RQAPGKGLEWVSLISGDGGSTYYADSVKGRFTISRDNSK |
| | | NSLYLQMNSLRTEDTALYYCAKVLLPCSSTSCYGSVGA |
| | | FDIWGQGTTVTVSS |
| 245 | VH | QVQLVQSGGSVVRPGESLRLSCVASGFIFDNYDMSWVR |
| | | QVPGKGLEWVSRVNWNGGSTTYADAVKGRFTISRDNT |
| | | KNSLYLQMNNLRAEDTAVYYCVREFVGAYDLWGQGT |
| | | TVTVSS |
| 246 | VH | QVQLVQSGAEVKKPGATVKVSCKVFGDTFRGLYIHWV |
| | | RQAPGQGLEWMGGIIPIFGTANYAQKFQGRVTITTDEST |
| | | STAYMELSSLRSEDTAVYYCASGLRWGIWGWFDPWGQ |
| | | GTLVTVSS |
| 247 | VH | EVQLVQSGAELKKPGSSVKVSCKAFGGTFSDNAISWVR |
| | | QAPGQGPEWMGGIIPIFGKPNYAQKFQGRVTITADESTS |
| | | TAYMVLSSLRSEDTAVYYCARTMVRGFLGVMDVWGQ |
| | | GTTVTVSS |
| 248 | VH | QVQLVQSGGGLVQPGGSLRLSCAASGFTFSSYAMSWVR |
| | | QAPGKGLEWVSAISGSGGSTYYADSVKGRFTISRDNSKN |
| | | TLYLQMNSLRAEDTAVYYCAKDQFVTIFGVPRYGMDV |
| | | WGQGTTVTVSS |
| 249 | VH | QVQLVQSGAEVKKPGSSVKVSCKASGGTFSSYAISWVR |
| | | QAPGQGLEWMGGIIPIFGTANYAQKFQGRVTITADKSTS |
| | | TAYMELSSLRSEDTAVYYCARGRQMFGAGIDFWGPGTL |
| | | VTVSS |
| 250 | VH | EVQLVESGAEVKKPGSSVKYSCKVSGGTFGTYALNWV |
| | | RQAPGQGLEWMGRIVPLIGLVNYAHNFEGRISITADKST |
| | | GTAYMELSNLRSDDTAVYYCAREVYGGNSDYWGQGTL |
| | | VTVSS |
| 251 | VH | QVQLVQSGGEVKKPGASVKVSCKASGYTLSSHGITWVR |
| | | QAPGQGLEWMGWISAHNGHASNAQKVEDRVTMTTDT |
| | | STNTAYMELRSLTADDTAVYYCARVHAALYYGMDVW |
| | | GQGTLVTVSS |
| 252 | VH | QVQLQESGGGVVQPGRSLRLSCSASGFTFSRHGMHWVR |
| | | QAPGKGLEWVAVISHDGSVKYYADSMKGRFSISRDNSN |
| | | NTLYLQMDSLRADDTAVYYCARGLSYQVSGWFDPWG |
| | | QGTLVTVSS |
| 253 | VH | NFMLTQPHSVSESPGKTVTISCTRSSGSIASNYVQWYQQ |
| | | RPGSSPTTVIYEDNQRPSGVPDRFSGSIDTSSNSASLTISG |
| | | LKTKDEADYYCQSYDGITVIFGGGTKLTVL |
| 254 | VH | NFMLTQPHSVSGSPGKTVTLPCTRSSGSIASHYVQWYQQ |
| | | RPGSAPTTVIYEDNKRPSGVPDRFSGSIDSSSNSASLSISG |
| | | LKTEDEADYYCQSYDSSNRWVFGGGTKLTVL |
| 255 | VH | LPVLTQPASLSASPGASASLTCTLRSGLNVGSYRIYWYQ |
| | | QKPGSRPQYLLNYKSDSNKQQASGVPSRFSGSKDASAN |
| | | AGILLISGLQSEDEADYYCMIWYSSAVVFGGGTKLTVL |
| 256 | VL | NFMLTQPHSVSESPGKTVTISCTRSSGNIASNYVQWYQQ |
| | | RPGSAPTTVIYEDNQRPSGVPDRFSGSIDSSSNSASLTISG |
| | | LKTEDEADYYCQSYDSSNLWVFGGGTKLTVL |
| 257 | VL | SSELTQDPAVSVALGQTVRITCQGDSLRSYYASWYQQK |
| | | PGQAPVLVIYGKNNRPSGIPDRFSGSSSGNTASLTITGAQ |
| | | AEDEADYYCNSRDSSGNHYVFGTGTKVTVL |
| 258 | VL | LPVLTQAPSVSVAPGKTARITCGGSDIGRKSVHWYQQKP |
| | | GQAPALVIYSDRDRPSGISERFSGSNSGNTATLTISRVEA |
| | | GDEADYYCQVWDNNSDHYVFGAGTELIVL |
| 259 | VL | QSALTQPASVSGSPGQSITISCTGTSSDVGGYNYVSWYQ |
| | | QHPGKAPKLMIYDVSNRPSGVSNRFSGSKSGNTASLTIS |
| | | GLQAEDEADYYCSSYTSSTLPFGGGTKLTVL |
| 260 | VL | EIVLTQSPATLSLSPGERATLSCRASQSIGNSLAWYQQKP |
| | | GQAPRLLMYGASSRATGIPDRFSGSGAGTDFTLTISSLEP |
| | | EDFATYYCQQHTIPTFSFGPGTKVEVK |
| 261 | VL | DIVMTQTPSFLSASIGDRVTITCRASQGIGSYLAWYQQRP |
| | | GEAPKLLIYAASTLQSGVPSRFSGSGSGTDFTLTISNLQPE |
| | | DFATYYCQQLNNYPITFGQGTRLEIK |
| 262 | VL | QSALTQPPSVSVSPGQTANIPCSGDKLGNKYAYWYQQK |
| | | PGQSPVLLIYQDIKRPSRIPERFSGSNSADTATLTISGTQA |
| | | MDEADYYCQTWDNSVVFGGGTKLTVL |
| 263 | VL | NFMLTQPHSVSESPGKTVTISCTRSSGSIDSNYVQWYQQ |
| | | RPGSAPTTVIYEDNQRPSGVPDRFSGSIDSSSNSASLTISG |
| | | LKTEDEADYYCDSYDSNNRHVIFGGGTKLTVL |
| 264 | VL | NFMLTQPHSVSESPGKTVTISCTRSSGNIGTNYVQWYQQ |
| | | RPGSAPVALIYEDYRRPSGVPDRFSGSIDSSSNSASLIISG |
| | | LKPEDEADYYCQSTHSSGWEFGGGTKLTVL |
| 265 | VL | QSVLTQPPSVSVAPGQTARITCGGNNIGSKGVHWYQQK |
| | | PGQAPVLVVYDDSDRPSGIPERFSGSNSGNTATLTISRVE |
| | | AGDEADYYCQVWDSSSDHWVFGGGTKLTVL |
| 266 | VL | NFMLTQPHSVSESPGKTVTISCTRSSGSIASNYVQWYQQ |
| | | RPGSAPTTVIYEDNQRPSGVPDRFSGSIDSSSNSASLTISG |
| | | LKTEDEADYYCQSYDSTTPSVFGGGTKLTVL |
| 267 | VL | QVQLVQSGAEVKKPGASVKVSCKASGYTFTSYGISWVR |
| | | QAPGQGLEWMGWTSPHNGLTAFAQILEGRVTMTTDTS |
| | | TNTAYMELRNLTFDDTAVYFCAKVHPVFSYALDVWGQ |
| | | GTLVTVSS |
| 268 | VL | EVQLVESGAEVMNPGSSVRVSCRGSGGDFSTYAFSWVR |
| | | QAPGQGLEWMGRIIPILGIANYAQKFQGRVTITADKSTS |
| | | TAYMELSSLRSDDTAVYYCARDGYGSDPVLWGQGTLV |
| | | TVSS |
| 269 | VL | EVQLVQSGAEVKKPGASVKVSCKASGYTFTNYGISWVR |
| | | QAPGQGLEWMGWISAYNGNTNYAQKVQGRVTMTTDT |
| | | STSTGYMELRSDDTAVYYCARGDFRKPFDYWGQG |
| | | TLVTVSS |
| 270 | VH | EVQLVQSGPELKKPGASVKMSCKASGYTFTSYVMHWV |
| | | KQAPGQRLEWIGYVNPFNDGTKYNEMFKGRATLTSDKS |
| | | TSTAYMELSSLRSEDSAVYYCARQAWGYPWGQGTLVT |
| | | VSS |
| 271 | VH | EVQLVQSGAEVKKPGASVKMSCKASGYTFSYVMHWV |
| | | KQAPGQRLEWIGYVNPFNDGTKYNEMFKGRATLTSDKS |
| | | TSTAYMELSSLRSEDSAVYYCARQAWGYPWGQGTLVT |
| | | VSS |
| 272 | VH | EVQLVQSGAEVKKPGASVKMSCKASGYTFTSYVMHWV |
| | | RQAPGQRLEWIGYVNPFNDGTKYNEMFKGRATLTSDKS |
| | | TSTAYMELSSLRSEDTAVYYCARQAWGYPWGQGTLVT |
| | | VSS |
| 273 | VH | EVQLVQSGAEVKKPGASVKMSCKASGYTFTSYVMHWV |
| | | RQAPGQRLEWIGYVNPFNDGTKYNEMFKGRATLTSDKS |
| | | TSTAYMELSSLRSEDTAVYYCARQAWGYPWGQGTLVT |
| | | VSS |
| 274 | VH | EVQLVQSGAEVKKPGASVKMSCKASGYTFTSYVMHWV |
| | | RQAPGQRLEWIGYVNPFNDGTKYNEMFKGRATLTSDKS |
| | | TSTAYMELSSLRSEDTAVYYCARQAWGYPWGQGTLVT |
| | | VSS |
| 275 | VL | DIVLTQSPASLALSPGERATLSCRATESVEYYGTSLVQW |
| | | YQQKPGQPPKLLIYAASSVDSGVPSRFSGSGSGTDFTLTI |
| | | NSLEEEDAAMYFCQQSRRVPYTFGQGTKLEIK |
| 276 | VL | DIVLTQSPATLSLSPGERATLSCRATESVEYYGTSLVQW |
| | | YQQKPGQPPKLLIYAASSVDSGVPSRFSGSGSGTDFTLTI |
| | | NSLEEEDAAMYFCQQSRRVPYTFGQGTKLEIK |
| 277 | VL | EIVLTQSPATLSLSPGERATLSCRATESVEYYGTSLVQW |
| | | YQQKPGQPPKLLIYAASSVDSGVPSRFSGSGSGTDFTLTI |
| | | NSLEEEDAAMYFCQQSRRVPYTFGQGTKLEIK |
| 278 | VL | DIVLTQSPATLSLSPGERATLSCRATESVEYYGTSLVQW |
| | | YQQKPGQPPKLLIYAASSVDSGVPSRFSGSGSGTDFTLTI |
| | | NSLEAEDAATYFCQQSRRVPYTFGQGTKLEIK |
| 279 | VH | EVQLVQSGAEVKKPGSSVKVSCKASGGTFSSYAISWVR |
| | | QAPGQGLEWMGGIIPIFGTANYAQKFQGRVTITADKSTS |
| | | TAYMELSSLRSEDTAVYYCAREGTIYDSSGYSFDYWGQ |
| | | GTLVTVSS |
| 280 | VH | EVQLVQSGAEVKKPGSSVKVSCKASGGTFSSYAISWVR |
| | | QAPGQGLEWMGIINPSGGSTSYAQKFQGRVSMTRDTST |
| | | STVYMELSSLTSEDTAVYYCARDLFPHIYGNYYGMDIW |
| | | GQGTTVTVSS |
| 281 | VH | QVQLVQSGAEVKKPGSSVKVSCKASGGTFSSYAISWVR |
| | | QAPGQGLEWMGGIIPIFGTANYAQKFQGRVTITADKSTS |
| | | TAYMELSSLRSEDTAVYYCARLAVPGAFDIWGQGTMV |
| | | TVSS |
| 282 | VH | EVQLVESGGGVVQPGRSLRLSCAASGFTFSSYAMHWVR |
| | | QAPGKGLAVISYDGSNKYYADSVKGRFTISRDNSKNTL |
| | | YLQMNSLRAEDTAVYYCARGQWLVTELDYWGQGTLV |
| | | TVSS |
| 283 | VH | EVQLVESGSEVEKPGSSVKVSCKASGGTFSDSGISWVRQ |
| | | APGQGLEWMGGIIPMFATPYYAQKFQDRVTITADESTST |
| | | VYMELSGLRSDDTAVFYCARDRGRGHLPWYFDLWGRG |
| | | TLVTVSS |
| 284 | VH | EVQLVESGAEVKKPGSSVKVSCKASGGTFSSYAISWVR |
| | | QAPGQGLEWMGGIIPIFGTANYAQKFQGRVTITADESTS |
| | | TAYMELSSLRSEDTAVYYCARAPYYYYYMDVWGQGTT |
| | | VTVSS |
| 285 | VH | EVQLLESGAEVKKPGSSVKVSCKASGGTLSRYALSWVR |
| | | QAPGQGPEWVGAIIPIFGTPHYSKKFQDRVTITVDTSTNT |
| | | AFMELSSLRFEDTALYFCARGHDEYDISGYHRLDYWGQ |
| | | GTLVTVSS |
| 286 | VH | QVQLVQSGSELKKPGSSVKVSCKASGYSFSGYYIHWVR |
| | | QAPGQGLEWMGWIDPNSGVTNYVRRFQGRVTMTRDTS |
| | | LSTAYMELSGLTADDTAVYYCARDENLWQFGYLDYWG |
| | | QGTLVTVSS |
| 287 | VH | QVQLVQSGAEVKKPGSSVKVSCKASGGTFSRYGVHWV |
| | | RQAPGQGLEWMGRLIPIVSMTNYAQKFQDRVSITTDKS |
| | | TGTAYMELRSLTSEDTALYYCASVGQQLPWVFFAWGQ |
| | | GTLVTVSS |
| 288 | VH | QVQLVESGGGVVQPGRSLRLSCAASGFTFSSYAMHWVR |
| | | QAPGKGLEWVAVISFDGSNKYYADSVRGRFTISRDNSK |
| | | NTLYLQMNSLRTEDTAVYYCARGWLDRDIDYWGQGTL |
| | | VTVSS |
| 289 | VH | EVQLVESGGGVVQPGRSLRLSCAASGFTFSSYAMHWVR |
| | | QAPGKGLEWVAVISFDGSNKYYADSVRGRFTISRDNSK |
| | | NTLYLQMNSLRTEDTAVYYCARGWLDRDIDYWGQGTL |
| | | VTVSS |
| 290 | VH | EVQLVQSGGGLVQPGGSLRLSCAASGFTFSDYGMHWV |
| | | RQPPGKGLEWLAVISYDGSYKIHADSVQGRFTISRDNAK |
| | | NSVFLQMNSLKTEDTAVYYCTTDRKWLAWHGMDVWG |
| | | QGTTVTVSS |
| 291 | VH | EVQLVQSGAEVKKPGSSVKVSCKASGGTFSSYAISWVR |
| | | QAPGQGLEWMGGIIPIFGTANYAQKFQGRVTITADESTS |
| | | TAYMELSSLRSEDTAVYYCARDGIVADFQHWGQGTLV |
| | | TVSS |
| 292 | VH | EVQLVESGAEVKKPGASVKVSCKASGDTFSRYGITWVR |
| | | QAPGRGLEWMGNIVPFFGATNYAQKFQGRLTITADKSS |
| | | YTSYMDLSSLRSDDTAVYYCARDHFYGSGGYFDYWGQ |
| | | GTLVTVSS |
| 293 | VH | EVQLLESGAEVKKPGASVKVSCKASGYTFNSYDINWVR |
| | | QAPGQGLEWMGGIIPVFGTANYAESFQGRVTMTADHST |
| | | STAYMELNNLRSEDTAVYYCARDRWHYESRPMDVWG |
| | | QGTTVTVSS |
| 294 | VH | EVQLVESGGGLVRPGGSLRLACAASGFSFSDYYMTWIR |
| | | QAPGRGLEWIAYISDSGQTVHYADSVKGRFTISRDNTKN |
| | | SLFLQVNTLRAEDTAVYYCAREDLLGYYLQSWGQGTL |
| | | VTVSS |
| 295 | VH | QVQLQQSGPGLVKPSQTLSLTCAISGDSVSSNSAAWNWI |
| | | RQSPSRGLEWLGRTYYRSKWYNDYAVSVKSRITINPDTS |
| | | KNQFSLQLNSVTPEDTAVYYCARDEPRAVAGSQAYYY |
| | | YGMDVWGQGTTVTVSS |
| 296 | VH | EVQLVQSGAEVKKPGASVKVSCKASGYTFTSYMHWV |
| | | RQAPGQGLEWMGIINPSDGSTSYAQKFQGRVTMTRDTS |
| | | TSTVHMELSSLRSEDTAVYYCARDLFPHIYGNYYGMDI |
| | | WGQTTVTVSS |
| 297 | VH | QMQLVQSGGGVVQPGRSLRLSCAASGFTFSSYAMHWV |
| | | RQAPGKGLEWVAVISFDGSNKYYADSVRGRFTISRDNS |
| | | KNTLYLQMNSLRTEDTAVYYCARGWLDRDIDYWGQGT |
| | | LVTVSS |
| 298 | VH | QMQLVQSGGGVVQPGRSLRLSCAASGFTFSSYAMHWV |
| | | RQAPGKGLEWVAVISFDGSNKYYADSVRGRFTISRDNS |
| | | KNTLYLQMNSLRTEDTAVYYCARGWLDRDIDYWGQGT |
| | | LVTVSS |
| 299 | VL | QSVLTQPPSVSAAYGQKVTISCSGNNSNIANNYVSWYQQ |
| | | LPGTAPKLLIYDNNYRPSGIPDRFSGSKSGTSATLDITGL |
| | | QTGDEADYYCGVWDGSLTTGVFGGGTKLTVL |
| 300 | VL | AIQMTQSPSSLSASVGDRVTITCRASQGISNYLAWYQQK |
| | | PGKVPKLLIYAASTLESGVPSRFSGSGSGTDFTLTISSLQP |
| | | EDLATYYCQQLHTFPLTFGGGTKVEIK |
| 301 | VL | QPVLTQPPSASGSPGQSVTISCTGTSSDVGAYNFVSWYR |
| | | QHPGKAPKLMIYEVNKRPSGVPDRFSGSKSGNTASLTVS |
| | | GLQAEDEADYYCSSYAGTNSLGIFGTGTKLTVL |
| 302 | VL | QSVVTQPPSVSAAPGQKVTISCSGSSSDIGNHYVSWYQQ |
| | | LPGTAPKLLIYDNNQRPSGIPDRFSGSKSGTSATLAITGL |
| | | QTGDEADYYCGTWDNSLSPHLLFGGGTKLTVL |
| 303 | VL | QSVLTQPPSVSAAPGQKVTISCSGSSSNMGNNYVSWYK |
| | | QVPGTAPKLLIYENDKRPSGIPDRFSGSKSGTSATLGITG |
| | | LQTGDEADYYCGTWDNSLSGFVFASGTKVTVL |
| 304 | VL | QSALTQPASVSGSLGQSVTISCTGSSSDVGSYNLVSWYQ |
| | | QHPGKAPNLMIYDVSKRSGVSNRFSGSKSGNTASLTISG |
| | | LQAEDEADYYCSSYTGISTVVFGGGTKLTVL |
| 305 | VL | QSALTQPASVSGSLGQSVTISCTGSSSDVGSYNLVSWYQ |
| | | QHPGKAPKLMIYEVSKRPSGVSNRFSGSKSGNTASLTIS |
| | | GLQAEDEADYYCSSYGGFNNLLFGGGTKLTVL |
| 306 | VL | DIVMTQSPSSLSASIGDRVTITCRASQRISAYVNWYQQKP |
| | | GKAPKVLIYAASSLRGVPSRFSGSGSGTDFTLTISSLQPE |
| | | DFATYYCQQTYSSPWTFGQGTKVEIK |
| 307 | VL | QSVLTQPPSASGSPGQSVTISCTGTSSDIGGYDSVSWYQQ |
| | | HPGKAPKLMIYDVSKRPSGVSNRFSGSKSGNTASLTISG |
| | | LQAEDEADYYCSSYTSSSTFFYVFGTGTKVTVL |
| 308 | VL | LPVLTQPASVSGSPGQSITISCTGTTSDIGGYDYVSWYQQ |
| | | HPGKAPKLMIYDVSKRPSGVSNRFSGSKSGNTASLTISG |
| | | LQAEDEADYYCSSYTSSSTHVFGTGTKLTVL |
| 309 | VL | QSALTQPASVSGSPGQSITISCTGTSSDVGGYNYVSWYQ |
| | | QHPGKAPKLMIYDVSNRPSGVSNRFSGSKSGNTASLTIS |
| | | GLQAEDEADYYCSSYRSSTLGPVFGGGTKLTVL |
| 310 | VL | QAGLTQPPSVSEAPRQRVTISCSGSSSNIGNNAVNWYQQ |
| | | LPGKAPKLLIYYDDLLPSGVSDRFSGSKSGTSASLAISGL |
| | | QSEDEADYYCAAWDDSLNGYVFGTGTKLTVL |
| 311 | VL | QSALTQPASVSGSPGQSITISCTGTSSDVGGYNYVSWYQ |
| | | QHPGKAPKLMIYDVSKRPSGVPDRFSGSKSGNTASLTIS |
| | | GLQAEDEADYYCSSYTSSTTHVFGTKVTVL |
| 312 | VL | QSVVTQPPSVSAAPGQKVTISCSGSSSNIGNNYVSWYQQ |
| | | LPGTAPKLLIYDNNKRPSGIPDRFSGSKSGTSATLGITGL |
| | | QTGDEADYYCGTWDSSLSVWVFGGGTQLTVL |
| 313 | VL | QSVLTQPASVSGSPGQSITISCTGTSSDVGGYNYVSWYQ |
| | | QHPGRAPRLMIYDVSNRPSGVSNRFSGSKSGNTASLTIS |
| | | GLQAEDEGDYYCSSYTSGGTLGPVFGGGTKLTVL |
| 314 | VL | QAGLTQPPSASGTPGQRVTISCSGSSSNIGSNTVNWYQQ |
| | | LPGTAPKLLIYSNNQRPSGVPDRFSGSKSGTSASLAISGL |
| | | QSEDEADYYCAAWDDSLNGWVFGGGTKLTVL |
| 315 | VL | AIRMTQSPSSLSASVGDRVTITCRASQSISNYLNWYQQR |
| | | PGKAPNLLIYAASSLQSGVPSRFSGSGSGTDFTLTISSLQP |
| | | EDFATYYCQQTYSTPYTFGQGTKLEIAK |
| 316 | VL | QSVLTQPASVSGSPGQSITISCTGTSSDVGGYNYVSWYR |
| | | QHPGKAPKLMIYDVSYRPSGVSNRFSGSKSGNTASLTIS |
| | | GLQAEDEADYYCSSYTDSSTRYVFGTGTKLTVL |
| 317 | VL | QPVLTQPPSASGTPGQRVAISCSGSRSNIEINSVNWYQQL |
| | | PGTAPKLLIYDNNKRPSGIPDRFSGSKSGTSATLGITGLQ |
| | | TGDEADYYCGSWDSSLSADVFGTGTKLTVL |
| 318 | VL | QSVLTQPPSVSAAPGKKVTISCSGSSSNIGNNYVSWYQQ |
| | | LPGTAPKLLIYRNNQRPSGVPDRFSGSKSGTSASLAISGL |
| | | QSEDEADYYCATWDDSLNGWVFGGGTKLTVL |
| 319 | VL | QSVVTQPPSVSGAPGQRVTISCTGSSSNIGAGYDVHWYQ |
| | | QLPGTAPKLLIYGNNNRHSGVPDRFSGSKSGTSASLAITG |
| | | LQAEDEAEFFCGTWDSRLTTYVFGSGTKLTVL |
| 320 | VL | QSVVTQPPSVSAAPGQKVTISCSGSSSNIGNNYVSWYQQ |
| | | LPGTAPKLLIYDNNKRPSGIPDRFSGSKSGTSATLGITGL |
| | | QTFGDEADYYCGTWDSSLSAVVFGGGTKLTVL |
| 321 | VL | VIWMTQSPSSLSASVGDRVTITCAASSLQSWYQQKPGK |
| | | APKLLIYEASTLESGVPSRFSGSGSGTEFTLTISSLQPEDF |
| | | ATYYCQQSYSTPYTFGQGTKLEIK |
| 322 | VL | QSVVTQPPSVSAAPGQKVTISCSGSSSNIGNNYVSWYQQ |
| | | VPGTAPKLLIYDNNKRPSGIPDRFSGSNSDTSATLGITGL |
| | | QTGDEADYYCGTWDSSLSAWVFGGGTKLTVL |
| 323 | VL | QSVVTQPPSVSAAPGQKVTISCSGSSSNIGNNYVSWYQQ |
| | | VPGTAPKLLIYDNNKRPSGIPDRFSGSNSDTSATLGITGL |
| | | QTGDEADYYCGTWDSSLSAGSVVFGGGTKLTVL |
| 324 | VL | SYELMQPPSVSVAPGKTATIACGGENIGRKTVHWYQQK |
| | | PGQAPVLVIYYDSDRPSGIPEPFSGSNSGNTATLTISRVE |
| | | AGDEADYYCLVWDSSSDHRIFGGGTKLTVL |
| 325 | VL | SYELMQPPSVSVAPGKTATIACGGENIGRKTVHWYQQK |
| | | PGQAPVLVIYYDSDRPSGIPEPFSGSNSGNTATLTISRVE |
| | | AGDEADYYCLVWDSSSDHRIFGGGTKLTVL |
| 326 | VL | SYELMQPPSVSVAPGKTATIACGGENIGRKTVHWYQQK |
| | | PGQAPVLVIYYDSDRPSGIPEPFSGSNSGNTATLTISRVE |
| | | AGDEADYYCLVWDSSSDHRIFGGGTKLTVL |
| 327 | VL | SYELMQPPSVSVAPGKTATIACGGENIGRKTVHWYQQK |
| | | PGQAPVLVIYYDSDRPSGIPEPFSGSNSGNTATLTISRVE |
| | | AGDEADYYCLVWDSSSDHRIFGGGTKLTVL |
| 328 | HC | QVQLVQSGVEVKKPGASVKVSCKASGYTFTNYYMYW |
| | | VRQAPGQGLEWMGGINPSNGGTNFNEKFKNRVTLTTDS |
| | | STTTAYMELKSLQFDDTAVYYCARRDYRFDMGFDYWG |
| | | QGTTVTVSSASTKGPSVFPLAPCSRSTSESTAALGCLVK |
| | | DYFPEPVTVSWNSGALTSGVHTFPAVLQSSGLYSLSSVV |
| | | TVPSSSLGTKTYTCNVDHKPSNTKVDKRVESKYGPPCPP |
| | | CPAPEFLGGPSVFLFPPKPKDTLMISRTPEVTCVVVDVSQ |
| | | EDPEVQFNWYVDGVEVHNAKTKPREEQFNSTYRVVSV |
| | | LTVLHQDWLNGKEYKCKVSNKGLPSSIEKTISKAKGQP |
| | | REPQVYTLPPSQEEMTKNQVSLTCLVKGFYPSDIAVEWE |
| | | SNGQPENNYKTPPPVLDSDGSFFLYSRLTVDKSRWQEG |
| | | NVFSCSVMHEALHNHYTQKSLSLSLGK |
| 329 | HC | QVQLVESGGGVVQPGRSLRLDCKASGITFSNSGMHWVR |
| | | QAPGKGLEWVAVIWYDGSKRYYADSVKGRFTISRDNS |
| | | KNTLFLQMNSLRAEDTAVYYCATNDDYWGQGTLVTVS |
| | | SASTKGPSVFPLAPCSRSTSESTAALGCLVKDYFPEPVTV |
| | | SWNSGALTSGVHTFPAVLQSSGLYSLSSVVTVPSSSLGT |
| | | KTYTCNVDHKPSNTKVDKRVESKYGPPCPPCPAPEFLG |
| | | GPSVFLFPPKPKDTLMISRTPEVTCVVVDVSQEDPEVQF |
| | | NWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGYPSDIAVEWESNGQPEN |
| | | NYKTTPPVLDSDGSFFLYSRLTVDKSRWQEGNVFSCSV |
| | | MHEALHNHYTQKSLSLSLGK |
| 330 | LC | EIVLTQSPATLSLSPGERATLSCRASKGVSTSGYSYLHW |
| | | YQQKPGQAPRLLIYLASYLESGVPARFSGSGSGTDFTLTI |
| | | SSLEPEDFAVYYCQHSRDLPLTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 331 | LC | EIVLTQSPATLSLSPGERATLSCRASQSVSSYLAWYQQK |
| | | PGQAPRLLIYDASNRATGIPARFSGSGSGTDFTLTISSLEP |
| | | EDFAVYYCQQSSNWPRTFGQGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 332 | VH | EVQLVESGGGLVQPGGSLRLSCAASGFTFSDSWIHWVR |
| | | QAPGKGLEWVAWISPYGGSTYYADSVKGRFTISADTSK |
| | | NTAYLQMNSLRAEDTAVYYCARRHWPGGFDYWGQGT |
| | | LVTVSSASTK |
| 333 | VH | EVQLVESGGGLVQPGGSLRLSCAASGFTFSDSWIHWVR |
| | | QAPGKGLEWVAWISPYGGSTYYADSVKGRFTISADTSK |
| | | NTAYLQMNSLRAEDTAVYYCARRHWPGGFDYWGQGT |
| | | LVTVSS |
| 334 | HC | EVQLVESGGGLVQPGGSLRLSCAASGFTFSDSWIHWVR |
| | | QAPGKGLEWVAWISPYGGSTYYADSVKGRFTISADTSK |
| | | NTAYLQMNSLRAEDTAVYYCARRHWPGGFDYWGQGT |
| | | LVTVSSASTKGPSVFPLAPSSKSTSGGTAALGCLVKDYF |
| | | PEPVTVSWNSGALTSGVHTFPAVLQSSGLYSLSSVVTVP |
| | | SSSLGTQTYICNVNHKPSNTKVDKKVEPKSCDKTHTCPP |
| | | CPAPELLGGPSVFLFPPKPKDTLMISRTPEVTCVVVDVSH |
| | | EDPEVKFNWYVDGVEVHNAKTKPREEQYASTYRVVSV |
| | | LTVLHQDWLNGKEYKCKVSNKALPAPIEKTISKAKGQP |
| | | REPQVYTLPPSREEMTKNQVSLTCLVKGFYPSDIAVEWE |
| | | SNGQPENNYKTTPPVLDSDGSFFLYSKLTVDKSRWQQG |
| | | NVFSCSVMHEALHNHYTQKSLSLSPG |
| 335 | LC | DIQMTQSPSSLSASVGDRVTITCRASQDVSTAVAWYQQ |
| | | KPGKAPKLLIYSASFLYSGVPSRFSGSGSGTDFTLTISSLQ |
| | | PEDFATYYCQQYLYHPATFGQGTKVEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 336 | VH | EVQLVESGGGLVQPGGSLRLSCAASGFTFSRFWMSWVR |
| | | QAPGKGLEWVANINQDGTEKYYVDSVKGRFTISRDNAK |
| | | NSLYLQMNSLRAGDTAVYYCANTYYDFWSGHFDYWG |
| | | QGTLVTVSS |
| 337 | VH | QEHLVESGGGVVQPGRSLRLSCEASGFTFSNFGMHWVR |
| | | QAPGKGLEWVAALWSDGSNKYYADSVKGRVTISRDNS |
| | | KNTLYLQMNSLRAEDTAVYYCARGRGAPGIPIFGYWGQ |
| | | GTLVTVSS |
| 338 | VH | EVQLVESGGGLVKPGGSLRLSCAASGFTFSNAWMSWV |
| | | RQAPGKGLEWVGRIKRKTDGGTTDYAAPVKGRFTISRD |
| | | DSKNTLHLQMNSLKTEDTAVYYCTTDDIVVVPAVMRE |
| | | YYFGMDVWGQGTTVTVSS |
| 339 | VH | QVQLVQSGAEVKKPGASVQVSCKASGYSFTGYYIHWV |
| | | RQAPGQGLEWMGWINPNSGTKKYAHKFQGRVTMTRD |
| | | TSIDTAYMILSSLISDDTAVYYCARDEDWNFGSWFDSW |
| | | GQGTLVTVSS |
| 340 | VH | QVHLVQSGAEVKKPGASVKVSCKASGYTFTGYYIHWV |
| | | RQAPGHGLEWMGWLNPNTGTTKYIQNFQGRVTMTRDT |
| | | SSSTAYMELTRLRSDDTAVYYCARDEDWNYGSWFDTW |
| | | GQGTLVTVSS |
| 341 | VH | EVQLVESGGGVVRPGGSLRLSCAASGFTFDDYGMTWV |
| | | RQAPGRGLEWVSGIHWHGKRTGYADSVKGRFTISRDNA |
| | | KKSLYLQMNSLKGEDTALYHCVRGGMSTGDWFDPWG |
| | | QGTLVIVSS |
| 342 | VH | EVQLVESGGGVVRPGGSLRLSCAASGFTFDDYGMTWV |
| | | RQVPGKGLEWVSGIHWSGRSTGYADSVKGRFTISRDNA |
| | | KNSLYLQMNSLRAEDTALYYCARGGMSTGDWFDPWG |
| | | QGTLVTVSS |
| 343 | VH | EVQLVESGGGLVQPGGSLRLSCAASGFTVGSNYMNWV |
| | | RQAPGKGLEWVSVIYSGGSTYYADSVKGRFTISRLTSKN |
| | | TLYLQMSSLRPEDTAVYYCARGIRGLDVWGQGTTVTVS |
| | | S |
| 344 | VH | EERLVESGGDLVQPGGSLRLSCAASGITVGTNYMNWVR |
| | | QAPGKGLEWVSVISSGGNTHYADSVKGRFIMSRQTSKN |
| | | TLYLQMNSLETEDTAVYYCARGIRGLDVWGQGTMVTV |
| | | SS |
| 345 | VH | QVQLVQSGAEVKMPGSSVRVSCKASGGIFSSSTISWVRQ |
| | | APGQGLEWMGEIIPVFGTVNYAQKFQDRVIFTADESTTT |
| | | AYMELSSLKSGDTAVYFCARNWGLGSFYIWGQGTMVT |
| | | VSS |
| 346 | VH | EVQLVESGGDLVHPGRSLRLSCAASGFPFDEYAMHWVR |
| | | QVPGKGLEWVSGISWSNNNIGYADSVKGRFTISRDNAK |
| | | NSLYLQMNSLRPEDTAFYYCAKSGIFDSWGQGTLVTVS |
| | | S |
| 347 | VH | EVQLVESGGGVVQPGRSLRLSCAASGFTFSSYGMHWVR |
| | | QAPGKGLEWVTLISYEGRNKYYADSVKGRFTISRDNSK |
| | | NTLYLQMNSLRAEDTAVYYCAKDRTLYGMDVWGQGT |
| | | TVTVSS |
| 348 | VH | QVTLRESGPALVKTTQTLTLTCTFSGFSLSTNRMCVTWI |
| | | RQPPGKALEWLARIDWDGVKYYNTSLKTRLTISKDTSK |
| | | NQVVLTMTNMDPVDTATFYCARSTSLTFYYFDYWGQG |
| | | TLVTVSS |
| 349 | VH | EVQLVESGGGLVQPGGSLRLSCAASEFTVGTNHMNWV |
| | | RQAPGKGLEWVSVIYSGGNTFYADSVKGRFTISRHTSKN |
| | | TLYLQMNSLTAEDTAVYYCARGLGGMDVWGQGTTVT |
| | | VSS |
| 350 | VH | EVQLVESGGGLVQRGESLRLYCAASGFTFSKYWMNWV |
| | | RQAPGKGLEWVANIKGDGSEKYYVDSVKGRFTISRDNA |
| | | KNSLYLQMNSLRAEDTAVYYCARDYWGSGYYFDFWG |
| | | QGTLVTVSS |
| 351 | VH | EVQLVESGGGLVQSGGSLRLSCAASGFTFSSYWMSWVR |
| | | QAPGKGLEWVANIKQDGSEKYYVDSVKGRFTISRDNAK |
| | | NSLYLQMNSLRADDTAVYYCARDDIVVVPAPMGYYYY |
| | | YFGMDVWGQGTTVTVSS |
| 352 | VH | EVQLVESGGGLVQPGRSLRLSCAASGFTFDDFAMHWVR |
| | | QAPGKGLEWVSGISWTGGNMDYANSVKGRFTISREDA |
| | | KNSLYLQMNSLRAADTALYYCVKDIRGIVATGGAFDIW |
| | | GRGTMVTVSS |
| 353 | VH | EVQLVESGGGLVQPGGSLRLSCAASGFTVGTNYMNWV |
| | | RQAPGKGLEWISVIYSGGSTFYADSVKGRFTISRQTSQN |
| | | TLYLQMNSLRPEDTAVYYCARGIRGFDIWGQGTMVTVS |
| | | S |
| 354 | VH | EVQLVESGGGLVQPGGSLRLSCAAGFTISTNYMNWVR |
| | | QAPGKGLEWVAVIYSSGSTYYIDSVKGRFTISRLTSKNT |
| | | VYLQMSSLNSEDTAVYYCARGIRGFDIWGQGTMVTVSS |
| 355 | VH | EVQLVESGGGLVQPGRSLRLSCAASGFTIDDSAMHWVR |
| | | QTPGKGLEWVSGISWKSGSIGYADSVRGRFTISRDNAKN |
| | | SLYLQMNSLRVEDTALYYCVKDIRGNWNYGGNWFDP |
| | | WGQGTLVTVSS |
| 356 | VH | EVQLVESGGGLVQPGGSLRLSCEASGFTVGVNHMNWV |
| | | RQAPGKGLEWVSVIFSSGRTFYGDYVKGRLTIFRQTSQN |
| | | TVYLQMNSLRSEDTAIYYCARGIGGLDIWGRGTMVTVS |
| | | S |
| 357 | VH | EVQLVESGGGLVQPGRSLRLSCAASGFTFDDYALHWVR |
| | | QAPGKGLEWVSGISWTGGTIDYADSVKGRFTISRDNAK |
| | | NSLYLQMSSLRTEDTAIYYCTRDIRGNWKYGGWFDPW |
| | | GQGTLVTVSS |
| 358 | VH | QVQLVQSGTEVKKPGASVKVSCKASGYTFTAYYMHWV |
| | | RQAPGQGLDWMGWISPNSGFTNYAQKFQGRVTMTRDT |
| | | SINTFYMELSGLRSDDTAVYYCAREGSTHHNSFDPWGQ |
| | | GTLVTVSS |
| 359 | VH | EVQLVESGGGLVQPGGSLRLSCAASGFTVGTNFMNWV |
| | | RQAPGKGLEWVSAIYSGGTANYADSVKGRFTISRDTSR |
| | | NTLYLQMNSLRTEDTAVYYCARGGGMDVWGQGTTVT |
| | | VSS |
| 360 | VH | QVQLVQSGAEVKKPGSSVKVSCKASGGTFNTYVLSWV |
| | | RQAPGQGLEWMGEIIPILGAANYAQNFQGRVTFTTDEST |
| | | NTAYMDLSSRsEDTAVYYCARDRTSGGFDPWGQGTL |
| | | VTVSS |
| 361 | VH | QVQLVQSGAEVEKPGASVKVSCKASGYIFTHYGISWVR |
| | | QAPGQGLEWVGWISPYNGYTDYAQKLQGRVTLTTDTS |
| | | TTTAYMELRNLRSDDTAMYYCSRGRGPYWSFDLWGRG |
| | | TLVTVSS |
| 362 | VL | DIQMTQSPSTLSASVGDRVTITCRASQSISNWLAWYQQK |
| | | PGKAPKLLIYKASSLESGVPSRFSGSGSGTEPTLTISSLQP |
| | | DDFATYYCQQYHSYSYTFGQGTKEIK |
| 363 | VL | DIQMTQSPSSLSASVGDRVTITCRASQGIRNDLGWYQQK |
| | | PGKAPKRLIYTASSLQSGVPSRFSGSGSGTEFTLTISSLQP |
| | | EDFATYYCLQHNSYPLTFGGGTKVAIK |
| 364 | VL | DIQMTQSPSSLSASVGDRVTITCRTSQGIRNDLGWYQQK |
| | | PGKAPKRLIYAASSLQSGVPSRFSGSGSGTEFTLTISSLQP |
| | | EDFATYYCLQHNNYPYTFGQGTKLEIK |
| 365 | VL | DIVMTQTPLSSPVTLGQPASISCRSSQTLVHGDGNTYLS |
| | | WIQQRPGQPPRLLIYKVSNQFSGVPDRFSGSGAGTDFTL |
| | | KISRVEAEDVGLYFCMQATHFPITFGQGTRLEIK |
| 366 | VL | DIVMTQTPLSSPVTLGQPASISCRSSPSLVHSDGNTYLSW |
| | | LQQRPGQPPRLLIYKISNRFSGVPDRFSGSGAGTTDFTLKIS |
| | | RVEAEDVGVYYCMQATHFPITFGQGTRLEIR |
| 367 | VL | DIQMTQSPSSLSASLGDRVTITCRASQSINSYLNWYQQK |
| | | PGKAPKLLIYVASSLQSGVPSRFSGSGSGTEFTLTISNLQP |
| | | EDFATYYCQQSYSTPPITFGQGTRLEIK |
| 368 | VL | DIQMTQSPSSLSASVGDRVTITCRASQSISSYLNWYQQKP |
| | | GKAPKLLIYVASSLQSGVPSRFSGSGSGTDFTLTISSLQPE |
| | | DFATYYCQQSYSTPPITFGQGTRLEIK |
| 369 | VL | DIQMTQSPSSLSASVGDRVTITCRASQTINIYLNWYQQKP |
| | | GRAPRLLIYAASSLQSGVPSRFSGSGSGTDFTLTISSLQPE |
| | | DFATYYCHQSYSTPPTIFGQGTRLEIK |
| 370 | VL | DIQMTQSPSSLSASVGDRVTITCRASQSMSSYLNWYQQK |
| | | PGRAPKLLIFAASSLQSGVPSRFSGSGSGTDFTLTISSLQP |
| | | EDFATYYCQQSYSTPPITFGQGTRLEIK |
| 371 | VL | EIVLTQSPGTLSLSPGERATLSCRASQSFNFNYLAWYQQ |
| | | KPGQAPRLLIYGASSRATGIPDRFSGSGSGTDFTLTINRLE |
| | | PEDFGVFYCQQYESAPWTFGQGTKVEIK |
| 372 | VL | DIQMTQSPSSLSASVGDRVTITCRASQSISSYLNWYQQKP |
| | | GKLLIYAASSLQSGVPSRFSGGGSGTDFTLTISSLRPEDFA |
| | | TYYCQQSYCTPPITFGQGTRLEIK |
| 373 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYSTPPITFGQGTRLEIK |
| 374 | VL | DRVTITCRASQVISNYLAWYQQKPGKVPRLLIYAASTLQ |
| | | SGVPSRFSGSGSGTDFTLTISSLQPEDVATYYCQKYNSAP |
| | | RTFGQGTKVEIK |
| 375 | VL | DIQMTQSPSSLSASVGDRVTITCRASQNINNYLNWYQQK |
| | | PGKAPKLLIYAASSFQNAVPSRFSGSGSGTDFTLTISSLQP |
| | | EDFATYYCQQSYNTPLTFGGGTKVEIK |
| 376 | VL | DIQMTQSPSSLSASVGDRVTITCRASQGIRNDLGWYQQK |
| | | PGKAPKRLIYAASSLQSGVPSRFSGSGSGTEFTLTISSLQP |
| | | EDFATYYCLQHNSYPYTFGQGTKLEIK |
| 377 | VL | DIQMTQSPSSLSASVGDRVTITCRASQSISSYLNWYQQKP |
| | | GKAPKILLYAASSLQSGVPSRFSGSGSGTDFTLTISSLQPE |
| | | DFATYYCQQSYSTPPITFGQGTRLEIK |
| 378 | VH | QSLEESGGRLVKPDETLTITCTVSGIDLSSNGLTWVRQAP |
| | | GEGLEWIGTINKDASAYYASWAKGRLTISKPSSTKVDLK |
| | | ITSPTTEDTATYFCGRIAFKTGTSIWGPGTLVTVSS |
| 379 | VL | AIVMTQTPSPVSAAVGGTVTINCQASESVYSNNYLSWFQ |
| | | QKPGQPPKLLIYLASTLASGVPSRFKGSGSGTQFTLTISG |
| | | VQCDDAATYYCIGGKSSSTDGNAFGGGTEVVVR |
| 380 | VH | QMQLVQSGAEVKKPGSSVKVSCKASGGTFSSYAISWVR |
| | | QAPGQGLEWMGGIIPIFGTANYAQKFQGRVTITADESTS |
| | | TAYMELSSLRSEDTAVYYCARGNIVATITPLDYWGQGT |
| | | LVTVSS |
| 381 | VH | QPVLTQPPSVSAAPGQKVTISCSGSSSNIANNYVSWYQQ |
| | | LPGTAPKLLIFANNKRPSGIPDRFSGSKSGTSGTSAALDITGL |
| | | QTGDEADYYCGTWDSDLRAGVFGGGTKLTVL |
| 382 | VH | EVQLVQSGAEVKKPGSSVKVSCKASGGTFSSYAISWVR |
| | | QAPGQGLEWMGGIIPIFGTANYAQKFQGRVTITADKSTS |
| | | TAYMELSSLRSEDTAVYYCAREGTIYDSSGYSFDYWGQ |
| | | GTLVTVSS |
| 383 | VH | QVQLVESGGGVVQPGRSLRLSCAASGFTFSSYAMHWVR |
| | | QAPGKGLEWVAVISFDGSNKYYADSVRGRFTISRDNSK |
| | | NTLYLQMNSLRTEDTAVYYCARGWLDRDIDYWGQGTL |
| | | VTVSS |
| 384 | VH | QVQLVESGGGVVQPGRSLRLSCAASGFTFSSYAMHWVR |
| | | QAPGKGLEWVAVISFDGSNKYYADSVRGRFTISRDNSK |
| | | NTLYLQMNSLRTEDTAVYYCARGWLDRDIDYWGQGTL |
| | | VTVSS |
| 385 | VH | QVQLVESGGGVVQPGRSLRLSCAASGFTFSSYAMHWVR |
| | | QAPGKGLEWVAVISFDGSNKYYADSVRGRFTISRDNSK |
| | | NTLYLQMNSLRTEDTAVYYCARGWLDRDIDYWGQGTL |
| | | VTVSS |
| 386 | VH | QVQLVESGGGVVQPGRSLRLSCAASGFTFSSYAMHWVR |
| | | QAPGKGLEWVAVISFDGSNKYYADSVRGRFTISRDNSK |
| | | NTLYLQMNSLRTEDTAVYYCARGWLDRDIDYWGQGTL |
| | | VTVSS |
| 387 | VH | QVQLVESGGGVVQPGRSLRLSCAASGFTFSSYAMHWVR |
| | | QAPGKGLEWVAVISFDGSNKYYADSVRGRFTISRDNSK |
| | | NTLYLQMNSLRTEDTAVYYCARGWLDRDIDYWGQGTL |
| | | VTVSS |
| 388 | VH | QVQLVESGGGVVQPGRSLRLSCAASGFTFSSYAMHWVR |
| | | QAPGKGLEWVAVISFDGSNKYYADSVRGRFTISRDNSK |
| | | NTLYLQMNSLRTEDTAVYYCARGWLDRDIDYWGQGTL |
| | | VTVSS |
| 389 | VH | QVQLVQSGAEVKKPGSSVKVSCKASGGTFSRYGVHWV |
| | | RQAPGQGLEWMGRLIPIVSMTNYAQKFQDRVSITTDKS |
| | | TGTAYMELRSLTSEDTALYYCASVGQQLPWVFFAWGQ |
| | | GTLVTVSS |
| 390 | VH | QMQLVQSGGGVVQPGRSLRLSCAASGFTFSSYAMHWV |
| | | RQAPGKGLEWVAVISFDGSNKYYADSVRGRFTISRDNS |
| | | KNTLNLQMNSLRTEDTAVYYCARGWLDRDIDYWGQGT |
| | | LVTVSS |
| 391 | VH | QVQLVQSGGGVVQPGRSLRLSCAASGFTFSSYAMHWV |
| | | RQAPGKGLEWVAVISFDGSNKYYADSVRGRFTISRDNS |
| | | KNTLYLQMNSLRTEDTAVYYCARGWLDRDIDYWGQGT |
| | | LVTVSS |
| 392 | VH | QMQLVQSGAEVKKPGSSVKVSCKASGGTFSSYAYSWV |
| | | RQAPGQGLEWMGGIIPSFGTANYAQKFQGRVTITADEST |
| | | STAYMELSSLRSEDTAVYYCARGPIVATITPLDYWGQGT |
| | | LVTVSS |
| 393 | VH | QMQLVQSGAEVKKPGSSVKVSCKASGGTFSSYAYSWV |
| | | RQAPGQGLEWMGGIIPSFGTANYAQKFQGRVTITADEST |
| | | STAYMELSSLRSEDTAVYYCARGPIVATITPLDYWGQGT |
| | | LVTVSS |
| 394 | VH | QMQLVQSGAEVKKPGSSVKVSCKASGGTFSSYAYSWV |
| | | RQAPGQGLEWMGGIIPSFGTANYAQKFQGRVTITADEST |
| | | STAYMELSSLRSEDTAVYYCARGPIVATITPLDYWGQGT |
| | | LVTVSS |
| 395 | VH | QMQLVQSGAEVKKPGSSVKVSCKASGGTFSSYAISWVR |
| | | QAPGQGLEWMGGIIPAFGTANYAQKFQGRVTITADESTS |
| | | TAYMELSSLRSEDTAVYYCARGPIVATITPLDYWGQGTL |
| | | VTVSS |
| 396 | VL | SYELMQPPSVSVAPGKTATIACGGENIGRKTVHWYQQK |
| | | PGQAPVLVIYYDSDRPSGIPERFSGSNSGNTATLTISRVE |
| | | AGDEADYYCQVWDSSSDHRIFGGGTKLTVL |
| 397 | VL | AIRMTQSPSSLSASVGDRVTITCRASQSISSYLNWYQQKP |
| | | GKAPKLLIYTTSSLKSGVPSRFSGSGSGTDFTLTISRLQPE |
| | | DFATYYCQQSYSSTWTFGRGTKVEIK |
| 398 | VL | QSVLTQPPSVSAAPGQKVTISCSGNNSNIANNYVSWYQQ |
| | | LPGTAPKLLIYDNNYRPSGIPDRFSGSKSGTSATLDITGL |
| | | QTGDEADYYCGVWDGSLTTGVFGGGTKLTVL |
| 399 | VL | LPVLTQPASVSGSPGQSITISCTGTTSDIGGYDYVSWYQQ |
| | | HPGKAPKLMIYDVSKRPSGVSNRFSGSKSGNTASLTISG |
| | | LQAEDEADYYCSSYTSSSTHVFGTGTKLTVL |
| 400 | VL | QSALTQPASVSGSPGQSITSCTGTSSDVGGYNYVSWYQ |
| | | QHPGKAPKLMIYDVSNRPSGVSNRFSGSKSGNTASLTIS |
| | | GLQAEDEADYYCSSYRSSTLGPVFGGGTKLTVL |
| 401 | VL | QAGLTQPPSVSEAPRQRVTISCSGSSSNIGNNAVNWYQQ |
| | | PGKAPKLLIYYDDLLPSGVSDRFSGSKSGTSASLAISGL |
| | | QSEDEADYYCAAWDDSLNGYVFGTGTKLTVL |
| 402 | VL | QSALTQPRSVSGSPGQSVTISCTGTSSDVGGYNYVSWYQ |
| | | QHPGKAPKLMIYDVSKRPSGVPDRFSGSKSGNTASLTIS |
| | | GLQAEDEADYYCSSYTSSTTHVFGTGTKVTVL |
| 403 | VL | QSVVTQPPSVSAAPGQKVTISCSGSSSNIGNNYVSWYQQ |
| | | LPGTAPKLLIYDNNKRPSGIPDRESGSKSGTSATLGITGL |
| | | QTGDEADYYCGTWDSSLSVWVFGGGTQLTVL |
| 404 | VL | QSVLTQPASVSGSPGQSITISCTGTSSDVGGYNYVSWYQ |
| | | QHPGRAPRLMIYDVSNRPSGVSNRFSGSKSGNTASLTIS |
| | | GLQAEDEGDYYCSSYTSGGTLGPVFGGGTKLTVL |
| 405 | VL | QSVVTQPPSVSAAPGQKVTISCSGSSSNIGNNYVSWYQQ |
| | | LPGTAPKLLIYDNNKRPSGIPDRFSGSKSGTSATLGITGL |
| | | QTGDEADYYCGTWDSSLSAVVFGGGTKLTVL |
| 406 | VL | QSVVTQPPSVSAAPGQKVTISCSGSSSNIGNNYVSWYQQ |
| | | VPGTAPKLLIYDNNKRPSGIPDRESGSNSDTSATLGITGL |
| | | QTGDEADYYCGTWDSSLSAWVFGGGTKLTVL |
| 407 | VL | QSVVTQPPSVSAAPGQKVTISCSGSSSNIGNNYVSWYQQ |
| | | LPGTAPKLLIYDNNKRPSGIPDRFSGSKSGTSATLGITGL |
| | | QTGDEADYYCGTWDSSLSAGSVVFGGGTKLTVL |
| 408 | VL | SYELMQPPSVSVAPGKTATIACGGENIGRKTVHWYQQK |
| | | PGQAPVLVIYYDSDRPSGIPERFSGSNSGNTATLTISRVE |
| | | AGDEADYYCLVWDSSSDHRIFGGGTKLTVL |
| 409 | VL | SYELMQPPSVSVAPGKTATIACGGENIGRKTVHWYQQK |
| | | PGQAPVLVIYYDSDRPSGIPERFSGSNSGNTATLTISRVE |
| | | AGDEADYYCQVWDSSSDHRIFGGGTKLTVL |
| 410 | VL | SYELMQPPSVSVAPGKTATIACGGENIGRKTVHWYQQK |
| | | PGQAPVLVIYYDSDRPSGIPERFSGSNSGNTATLTISRVE |
| | | AGDEADYYCQVWDSSSDHRIFGGGTKLTVL |
| 411 | VL | SYELMQPPSVSVAPGKTATIACGGENIGRKTVHWYQQK |
| | | PGQAPVLVIYYDSDRPSGIPERFSGSNSGNTATLTISRVE |
| | | AGDEADYYCQVWDSSSDHRIFGGGTKLTVL |
| 412 | VH | QVQLVQSGSEVKKSGSSVKVSCKTSGGTFSITNYAINWV |
| | | RQAPGQGLEWMGGILPIFGAAKYAQKFQDRVTITADES |
| | | TNTAYLELSSLTSEDTAMYYCARGKRWLQSDLQYWGQ |
| | | GTLVTVSS |
| 413 | VL | QPVLTQPASVSGSPGQSITISCTGSSSDVGSYDLVSWYQQ |
| | | SPGKVPKLLIYEGVKRPSGVSNRFSGSKSGNTASLTISGL |
| | | QAEDEADYYCSSYAGTRNFVFGGGTQLTVL |
| 414 | VL CDR1 | RASQSISSYLN |
| 415 | C8 VL | KASRLQS |
| | CDR2 | |
| 416 | C8 VL | RALKPVT |
| | CDR3 | |
| 417 | VL CDR2 | AASSLQS |
| 418 | C12 VL | SYSTPNT |
| | CDR3 | |
| 419 | C16 VL | SASQLQS |
| | CDR2 | |
| 420 | C16 VL | ANSRPST |
| | CDR3 | |
| 421 | C20 VL | NASSLQS |
| | CDR2 | |
| 422 | C20 VL | YPYGPG |
| | CDR3 | |
| 423 | VL CDR2 | YASTLQS |
| 424 | VL CDR3 | DNGYPST |
| 425 | VH CDR1 | SYAMS |
| 426 | VH CDR2 | DITASGQRTTYADS |
| 427 | VH CDR3 | SKAIFDY |
| 428 | VH CDR2 | SINKDGHYTSYADS |
| 429 | VH CDR3 | NIDEFDY |
| 430 | VH CDR2 | SIMATGAGTLYADS |
| 431 | VH CDR3 | DGAGFDY |
| 432 | VH CDR2 | TITSSGAATYYADS |
| 433 | VH CDR3 | NYTGFDY |
| 434 | VH CDR2 | SIYSTGGATAYADS |
| 435 | VH CDR3 | SSAGFDY |
| 436 | VH CDR2 | SSIYSTGGATAYADS |
| 437 | VH CDR3 | SSAGQSRPGFDY |
| 438 | VH CDR3 | SSAGQSWPGFDY |
| 439 | VH CDR3 | SSAGQSFPGFDY |
| 440 | VH CDR3 | WSAAFDY |
| 441 | VH CDR3 | WSAGYDY |
| 442 | VH CDR3 | WSKGFDY |
| 443 | VH CDR2 | SSIWKQGIVTVYDS |
| 444 | VH CDR2 | SSIWRNGIVTVYDS |
| 445 | VH CDR2 | SDIWKQGMVTVYDS |
| 446 | VH CDR2 | SSIWRQGLATAYDS |
| 447 | VH CDR2 | SEIVATGILTSYDS |
| 448 | VH CDR2 | SSIGRQGLITVYDS |
| 449 | VH CDR2 | SSIWYQGLVTVYDS |
| 450 | VH CDR2 | SDIWKQGFATADS |
| 451 | VH CDR2 | SSIWRNGIVTVYADS |
| 452 | VH CDR2 | SSIWYQGLVTVYADS |
| 453 | VH CDR3 | DYFYGMDV |
| 454 | VH CDR3 | KFHFVSGSPFGMDV |
| 455 | VH CDR3 | ERIQLWFDY |
| 456 | VH CDR3 | DQGIAAALFDY |
| 457 | VH CDR3 | PFDY |
| 458 | VH CDR3 | KYDYVSGSPFGMDV |
| 459 | VH CDR3 | DSSGWSRYYMDV |
| 460 | VH CDR3 | KYSYVSGSPFGMDV |
| 461 | VH CDR3 | GRFRYFDWFLDY |
| 462 | VH CDR3 | DYFWSGFSAFDI |
| 463 | VL CDR3 | QQRSNWPRT |
| 464 | VL CDR3 | QQRSNWPT |
| 465 | VL CDR3 | QQYNSYPYT |
| 466 | VL CDR3 | QQYGSSPWT |
| 467 | VL CDR3 | QQYGSSP |
| 468 | VL CDR3 | QQFNSYPFT |
| 469 | VL CDR3 | QQSYSTPWT |
| 470 | VH CDR2 | WITAYNGNTNYAQKLQG |
| 471 | VH CDR2 | GIIPIFGKAHYAQKFQG |
| 472 | VH CDR2 | WLHADTGITKFSQKFQG |
| 473 | VH CDR2 | GIIPIFGTANHAQKFQG |
| 474 | VH CDR2 | GISGNSGNIGYADSVKG |
| 475 | VH CDR2 | GIIPIFGRAHYAQKFQG |
| 476 | VH CDR2 | GIIPIFGSANYAQKFQD |
| 477 | VH CDR2 | GIIPLFGIAHYAQKFQG |
| 478 | VH CDR2 | GISWNRGRIEYADSVKG |
| 479 | VL CDR2 | DASNRAT |
| 480 | VL CDR2 | GASSRAT |
| 481 | VL CDR2 | DASSLES |
| 482 | VL CDR2 | KASTLES |
| 483 | VH CDR1 | DYGFS |
| 484 | VH CDR1 | TYAIS |
| 485 | VH CDR1 | SYDVH |
| 486 | VH CDR1 | TYAIN |
| 487 | VH CDR1 | DYVVH |
| 488 | VH CDR1 | SYAIS |
| 489 | VH CDR1 | SYAIN |
| 490 | VH CDR1 | DYGMH |
| 491 | VL CDR1 | RASQSVSSYLV |
| 492 | VL CDR1 | RASQSVSSYLA |
| 493 | VL CDR1 | RASQGISSWLA |
| 494 | VL CDR1 | RASQSVSSSYLA |
| 495 | VL CDR1 | RASQGISSALA |
| 496 | VH CDR1 | TYSMN |
| 497 | VH CDR2 | SISSSGDYIYYADSVK |
| 498 | VH CDR3 | DLVTSMVAFDY |
| 499 | VL CDR1 | SGDALPQKYVF |
| 500 | VL CDR2 | EDSKRPS |
| 501 | VL CDR3 | YSTDRSGNHRV |
| 502 | VH CDR1 | RYWMS |
| 503 | VH CDR2 | NIKQDGSEKYYVDSVKG |
| 504 | VH CDR3 | EGGWFGELAFDY |
| 505 | VL CDR1 | RASQRVSSSYLA |
| 506 | VL CDR2 | DASSRAT |
| 507 | VL CDR3 | QQYGSLPWT |
| 508 | VH CDR1 | SWYMS |
| 509 | VH CDR2 | NIKQDGGEQYYVDSVK |
| 510 | VH CDR3 | DWNYGYYDMDV |
| 511 | VL CDR1 | RASQSVSSNYLA |
| 512 | VL CDR2 | GTSSRAT |
| 513 | VL CDR3 | QQYGSSIFT |
| 514 | VL CDR1 | RASQxxxPxxA |
| 515 | VL CDR2 | SASxLxS |
| 516 | VL CDR3 | QQxxxxPxT |
| 517 | VH CDR1 | GFTFSxSWIH |
| 518 | VH CDR2 | AWIxPYGGSxYYADSVKG |
| 519 | VH CDR3 | RHWPGGFDY |
| 520 | VL CDR1 | RASQDVSTAVA |
| 521 | VL CDR2 | SASFLYS |
| 522 | VL CDR3 | QQYLYHPAT |
| 523 | VH CDR1 | GFTFSDSWIH |
| 524 | VH CDR2 | AWISPYGGSTYYADSVKG |
| 525 | VL CDR1 | KSSQSLLxxxTRKNYLA |
| 526 | VL CDR2 | WASTRES |
| 527 | VL CDR3 | xQSYDVVT |
| 528 | VH CDR1 | SYWxH |
| 529 | VH CDR2 | YINPSSxYxEYxxKFxD |
| 530 | VH CDR3 | SGWLxHGDYYFDx |
| 531 | VL CDR1 | KSSQSLLNSRTRKNYLA |
| 532 | VL CDR3 | QQSYDVVT |
| 533 | VH CDR1 | SYWMH |
| 534 | VH CDR2 | YINPSSDYNEYSEKFMD |
| 535 | VH CDR3 | SGWLVHGDYYFDY |
| 536 | VL CDR1 | KSSQSLLHTSTRKNYLA |
| 537 | VL CDR3 | KQSYDVVT |
| 538 | VH CDR1 | GYIFTSYWMH |
| 539 | VH CDR2 | YINPSSGYHEYNQKFID |
| 540 | VH CDR3 | SGWLIHGDYYFDF |
| 541 | VH CDR1 | SYWIH |
| 542 | VH CDR1 | GTTFTSYWIH |
| 543 | VL CDR1 | TGTxxDVGxYNYVS |
| 544 | VL CDR2 | xVxxRPS |
| 545 | VL CDR3 | SSxTxxxxRV |
| 546 | VH CDR1 | xYxMx |
| 547 | VH CDR2 | SIYPSGGxTFYADxVK |
| 548 | VH CDR3 | IKLGTVTTVxY |
| 549 | VL CDR1 | TGTSSDVGGYNYVS |
| 550 | VL CDR2 | DVSNRPS |
| 551 | VL CDR3 | SSYTSSSTRV |
| 552 | VH CDR1 | SYIMM |
| 553 | VH CDR2 | SIYPSGGITFYADTVKG |
| 554 | VH CDR3 | IKLGTVTTVDY |
| 555 | VH CDR1 | MYMMM |
| 556 | VH CDR2 | SIYPSGGITFYADTVKG |
| 557 | VH CDR3 | TGTSSDVGAYNYVS |
| 558 | VL CDR1 | xxSxSLLYSSxxXxxxx |
| 559 | VL CDR2 | Xxxxxx |
| 560 | VL CDR3 | xQXxxxPxT |
| 561 | VH CDR1 | GxxxxxxxxN |
| 562 | VH CDR2 | XxXxxxxxTxxNxxKx |
| 563 | VH CDR3 | xxxXXXXx |
| 564 | VL CDR1 | RASSSVSYIY |
| 565 | VL CDR2 | ATFNLAS |
| 566 | VL CDR3 | HQRSSYPWT |
| 567 | VH CDR1 | GYTFPDYYMN |
| 568 | VH CDR2 | DIDPNYGGTTYNQKFKG |
| 569 | VL CDR1 | SASSSIRYMH |
| 570 | VL CDR2 | DTSKLTS |
| 571 | VL CDR3 | QQDSSYPLT |
| 572 | VH CDR1 | GYTFTSYDIN |
| 573 | VH CDR2 | WIFPRDNNTKYNENFKG |
| 574 | VH CDR3 | ENWVGDF |
| 575 | VL CDR1 | KASQDVGTAVA |
| 576 | VL CDR2 | WASTRHT |
| 577 | VL CDR3 | QQYYGYPLT |
| 578 | VH CDR1 | GYSITSDYWN |
| 579 | VH CDR2 | YISYTGSTYYNPSLKS |
| 580 | VH CDR3 | YGGWLSPF |
| 581 | VL CDR1 | KSSQSLLYSSNQKNSL |
| 582 | VH CDR1 | GYSIISDYWN |
| 583 | VH CDR3 | RGGWLLPF |
| 584 | VH CDR1 | GFSLTTYSIN |
| 585 | VH CDR2 | VMWAGGGTNSNSVLKS |
| 586 | VH CDR3 | YYGNSPYYAI |
| 587 | VL CDR1 | TRSSGSIGSNYVQ |
| 588 | VL CDR2 | EDNQRPS |
| 589 | VL CDR3 | QSYDSSTWVI |
| 590 | VH CDR2 | WISPIGGSTNYAQKVQG |
| 591 | VH CDR3 | GLXXXXXXXXXXXXXXXDV |
| 592 | VL CDR1 | TRSSGNIASNYVQ |
| 593 | VL CDR2 | GKNNRPS |
| 594 | VL CDR3 | QSYDSSNLWV |
| 595 | VH CDR1 | SYGIS |
| 596 | VH CDR2 | WISAYNGNTNYAQKLED |
| 597 | VH CDR3 | ALPSGTILNGGWFDP |
| 598 | VL CDR1 | QGDSLRSYYAS |
| 599 | VL CDR2 | SDRDRPS |
| 600 | VL CDR3 | NSRDSSGNHYV |
| 601 | VH CDR1 | SYALS |
| 602 | VH CDR2 | AISGGGGSTYYADSVKD |
| 603 | VH CDR3 | DVFPETFSMNYGMDV |
| 604 | VL CDR1 | GGSDIGRKSVH |
| 605 | VL CDR3 | QVWDNNSDHYV |
| 606 | VH CDR1 | DYAMH |
| 607 | VH CDR2 | LISGDGGSTYYADSVKD |
| 608 | VH CDR3 | VLLPCSSTSCYGSVGAFDI |
| 609 | VL CDR3 | SSYTSSTLP |
| 610 | VH CDR1 | NYDMS |
| 611 | VH CDR2 | RVNWNGGSTTYADAVKD |
| 612 | VH CDR3 | EFVGAYDL |
| 613 | VL CDR1 | RASQSIGNSLA |
| 614 | VL CDR2 | AASTLQS |
| 615 | VL CDR3 | QQHTIPTFS |
| 616 | VH CDR1 | GLYIH |
| 617 | VH CDR2 | WIIPIFGTANYAQKFED |
| 618 | VH CDR3 | GLRWGIWGWFDP |
| 619 | VL CDR1 | RASQGIGSYLA |
| 620 | VL CDR2 | QDIKRPS |
| 621 | VL CDR3 | QQLNNYPIT |
| 622 | VH CDR1 | DNAIS |
| 623 | VH CDR2 | WIIPIFGKPNYAQKFED |
| 624 | VH CDR3 | TMVRGFLGVMDV |
| 625 | VL CDR1 | SGDKLGNKYAY |
| 626 | VL CDR2 | EDYRRPS |
| 627 | VL CDR3 | QTWDNSVV |
| 628 | VH CDR2 | AISGSGGSTYYADSVKD |
| 629 | VH CDR3 | DQFVTIFGVPRYGMDV |
| 630 | VL CDR1 | TRSSGSIDSNYVQ |
| 631 | VL CDR2 | DDSDRPS |
| 632 | VL CDR3 | QSYDSNNRHVI |
| 633 | VH CDR1 | TYALN |
| 634 | VH CDR2 | RIVPLIGLVNYAHNFED |
| 635 | VH CDR3 | GRQMFGAGIDF |
| 636 | VL CDR1 | TRSSGNIGTNYVQ |
| 637 | VL CDR2 | EDNKRPS |
| 638 | VL CDR3 | QSYHSSGWE |
| 639 | VH CDR1 | SHGIT |
| 640 | VH CDR2 | WISAHNGHASNAQKVED |
| 641 | VH CDR3 | EVYGGNSDY |
| 642 | VL CDR1 | GGNNIGSKGVH |
| 643 | VL CDR2 | YKSDSNKQQAS |
| 644 | VL CDR3 | QVWDSSSDHWV |
| 645 | VH CDR1 | RHGMH |
| 646 | VH CDR2 | VISHDGSVKYYADSMKD |
| 647 | VH CDR3 | VHAALYYGMDV |
| 648 | VL CDR1 | TRSSGSIASNYVQ |
| 649 | VL CDR3 | QSYDSTTPSV |
| 650 | VH CDR2 | WTSPHNGLTAFAQILED |
| 651 | VH CDR3 | GLSYQVSGWFDP |
| 652 | VL CDR1 | TRSSGSIASHYVQ |
| 653 | VL CDR3 | QSYDGITVI |
| 654 | VH CDR2 | RIIPILGIANYAQKFED |
| 655 | VH CDR3 | VHPVFSYALDV |
| 656 | VL CDRQ | TLRSGLNVGSYRIY |
| 657 | VL CDR3 | QSYDSSNRWV |
| 658 | VH CDR1 | TYAFS |
| 659 | VH CDR2 | WISAYNGNTNYAQKVED |
| 660 | VH CDR3 | DGYGSDPVL |
| 661 | VL CDR3 | MIWYSSAVV |
| 662 | VH CDR1 | NYGIS |
| 663 | VH CDR3 | GDFRKPFDY |
| 664 | VL CDR1 | RATESVEYYGTSLVQ |
| 665 | VL CDR2 | AASSVDS |
| 666 | VL CDR3 | QQSRRVPYT |
| 667 | VH CDR1 | SYVMH |
| 668 | VH CDR2 | YVNPFNDGTKYNEMFKG |
| 669 | VH CDR3 | QAWGYP |
| 670 | VL CDR1 | QSISNW |
| 671 | VL CDR3 | QQYHSYSYT |
| 672 | VH CDR1 | GFTFSRFW |
| 673 | VH CDR2 | INQDGTEK |
| 674 | VH CDR3 | ANTYYDFWSGHFDY |
| 675 | VL CDR1 | QGIRND |
| 676 | VL CDR3 | LQHNSYPLT |
| 677 | VH CDR1 | GFTFSNFG |
| 678 | VH CDR2 | LWSDGSNK |
| 679 | VH CDR3 | ARGRGAPGIPIFGY |
| 680 | VL CDR3 | LQHNNYPYT |
| 681 | VH CDR1 | GFTFSNAW |
| 682 | VH CDR2 | IKRKTDGGTT |
| 683 | VH CDR3 | TTDDIVVVPAVMREYYGMDV |
| 684 | VL CDR1 | QTLVHGDGNTY |
| 685 | VL CDR3 | MQATHFPIT |
| 686 | VH CDR1 | GYSFTGYY |
| 687 | VH CDR2 | INPNSGTK |
| 688 | VH CDR3 | ARDEDWNFGSWFDS |
| 689 | VL CDR1 | PSLVHSDGNTY |
| 690 | VH CDR1 | GYTFTGYY |
| 691 | VH CDR2 | LNPNTGTT |
| 692 | VH CDR3 | ARDEDWNYGSWFDT |
| 693 | VL CDR1 | QSINSY |
| 694 | VL CDR3 | QQSYSTPPIT |
| 695 | VH CDR1 | GFTFDDYG |
| 696 | VH CDR2 | IHWHGKRT |
| 697 | VH CDR3 | VRGGMSTGDWFDP |
| 698 | VL CDR1 | QSISSY |
| 699 | VH CDR2 | IHWSGRST |
| 700 | VH CDR3 | ARGGMSTGDWFDP |
| 701 | VL CDR1 | QTINIY |
| 702 | VL CDR3 | HQSYSTPPIT |
| 703 | VH CDR1 | GFTVGSNY |
| 704 | VH CDR2 | IYSGGST |
| 705 | VH CDR3 | ARGIRGLDV |
| 706 | VL CDR1 | QSFNFNY |
| 707 | VL CDR3 | QQYESAPWT |
| 708 | VH CDR1 | GGIFSSST |
| 709 | VH CDR2 | IIPVFGTV |
| 710 | VH CDR3 | ARNWGLGSFYI |
| 711 | VL CDR3 | QQSYCTPPIT |
| 712 | VH CDR1 | GFPFDEYA |
| 713 | VH CDR2 | ISWSNNNI |
| 714 | VH CDR3 | AKSGIFDS |
| 715 | VH CDR1 | GFTFSSYG |
| 716 | VH CDR2 | ISYEGRNK |
| 717 | VH CDR3 | AKDRTLYGMDV |
| 718 | VL CDR1 | QVISNY |
| 719 | VL CDR3 | QKYNSAPRT |
| 720 | VH CDR1 | GFSLSTNRMC |
| 721 | VH CDR2 | IDWDGVK |
| 722 | VH CDR3 | ARSTSLTFYYFDY |
| 723 | VL CDR1 | QNINNY |
| 724 | VL CDR3 | QQSYNTPLT |
| 725 | VH CDR1 | EFTVGTNH |
| 726 | VH CDR2 | IYSGGNT |
| 727 | VH CDR3 | ARGLGGMDV |
| 728 | VL CDR1 | QTISTY |
| 729 | VL CHR3 | LQHNSYPYT |
| 730 | VH CDR1 | GFTFSKYW |
| 731 | VH CDR2 | IKGDGSEK |
| 732 | VH CDR3 | ARDYWGSGYYFDF |
| 733 | VL CDR3 | QQSYSTPFT |
| 734 | VH CDR1 | GFTFSSYW |
| 735 | VH CDR2 | IKQDGSEK |
| 736 | VH CDR3 | ARDDIVVVPAPMGYYYYYFGMDV |
| 737 | VH CDR1 | GFTFDDFA |
| 738 | VH CDR2 | ISWTGGNM |
| 739 | VH CDR3 | VKDIRGIVATGGAFDI |
| 740 | VH CDR1 | GFTVGTNY |
| 741 | VH CDR3 | ARGIRGFDI |
| 742 | VH CDR1 | GFTISTNY |
| 743 | VH CDR2 | IYSSGST |
| 744 | VH CDR1 | GFTIDDSA |
| 745 | VH CDR2 | ISWKSGSI |
| 746 | VH CDR3 | VKDIRGNWNYGGNWFDP |
| 747 | VH CDR1 | GFTVGVNH |
| 748 | VH CDR2 | IFSSGRT |
| 749 | VH CDR3 | ARGIGGLDI |
| 750 | VH CDR1 | GFTFDDYA |
| 751 | VH CDR2 | ISWTGGTI |
| 752 | VH CDR3 | TRDIRGNWKYGGWFDP |
| 753 | VH CDR1 | GYTFTAYY |
| 754 | VH CDR2 | ISPNSGFT |
| 755 | VH CDR3 | AREGSTHHNSFDP |
| 756 | VH CDR1 | GFTVGTNF |
| 757 | VH CDR2 | IIPILGAA |
| 758 | VH CDR3 | ARGGGMDV |
| 759 | VH CDR1 | GGTFNTYV |
| 760 | VH CDR2 | ISPYNGYT |
| 761 | VH CDR3 | ARDRTSGGFDP |
| 762 | VH CDR1 | GYIFTHYG |
| 763 | VH CDR3 | SRGRGPYWSFDL |
| 764 | VL CDR1 | QASESVYSNNYLS |
| 765 | VL CDR2 | LASTLAS |
| 766 | VL CDR3 | IGGKSSSTDGNA |
| 767 | VH CDR1 | SNGLT |
| 768 | VH CDR2 | TINKDASAYYASWAKG |
| 769 | VH CDR3 | IAFKTGTSI |
| 770 | VL CDR1 | RSSKSLLHSNGITYLY |
| 771 | VL CDR2 | QMSNLAS |
| 772 | VL CDR3 | AQNLEPPLT |
| 773 | VH CDR1 | DYYTH |
| 774 | VH CDR2 | WIDPENGKTAYAPKFQG |
| 775 | VH CDR3 | GGYDVYFLDY |
| 776 | VL CDR1 | KASQDVGIVVA |
| 777 | VL CDR2 | WASIRHT |
| 778 | VL CDR3 | QQYSNYPLYT |
| 779 | VH CDR1 | GFSLTSYGVH |
| 780 | VH CDR2 | VIWAGGSTNYNSALMS |
| 781 | VH CDR3 | AKPYGNSAMDY |
| 782 | VH CDR2 | VIWAGGSTNYVDSVKG |
| 783 | VH CDR3 | AKPYGTSAMDY |
| 784 | VH CDR3 | VIWAGGSTNYADSVKG |
| 785 | VL CDR1 | ASQSVSTSSSSFMH |
| 786 | VL CDR2 | YASNLES |
| 787 | VL CDR3 | QHSWEIPYT |
| 788 | VH CDR1 | SYGMS |
| 789 | VH CDR2 | SISSGGSTYYPDSVKG |
| 790 | VH CDR3 | GYDSGFAY |
| 791 | VL CDR1 | RASWSVSTSSSSYMH |
| 792 | VH CDR2 | SISSGGYTYYPDSVKG |
| 793 | VL CDR1 | KASQSVSNDVA |
| 794 | VL CDR2 | YAANRYT |
| 795 | VL CDR3 | QQDYTSPYT |
| 796 | VH CDR1 | TYGVH |
| 797 | VH CDR2 | VIWRGVYYDYNAAFMAS |
| 798 | VH CDR3 | LGFYAMDY |
| 799 | VL CDR1 | KASQSVSNDVG |
| 800 | VL CDR2 | YASNRYS |
| 801 | VH CDR1 | SYGVH |
| 802 | VH CDR2 | VIWSGGVTDYNAAFIS |
| 803 | VL CDR2 | RSSQIIVHSNANTYLE |
| 804 | VL CDR2 | KVSNRFS |
| 805 | VL CDR3 | FQGSHVPYT |
| 806 | VH CDR1 | TYWMH |
| 807 | VH CDR2 | QINPDSTTINTAPSLKD |
| 808 | VH CDR3 | PGDYGYDFDC |
| 809 | VL CDR1 | SASSSVSSSYLY |
| 810 | VL CDR2 | NTSNLAS |
| 811 | VL CDR3 | HQWRSYPPT |
| 812 | VH CDR1 | SGYWN |
| 813 | VH CDR2 | YISYSGSTYYNPSLKS |
| 814 | VH CDR3 | SLLWFSTGFAY |
| 815 | VL CDR1 | SANSSVSYMH |
| 816 | VL CDR2 | DTSKLAS |
| 817 | VL CDR3 | QQWSSNPWT |
| 818 | VH CDR2 | YYWSGGITDYNAAFKS |
| 819 | VL CDR1 | RASQSVSTSSYSYMH |
| 820 | VL CDR3 | QNSWEIPYT |
| 821 | VH CDR1 | STGMS |
| 822 | VH CDR2 | SISSGGTTYYLGSVQG |
| 823 | VH CDR3 | GYDAGFAY |
| 824 | VL CDR1 | KSSQSLLYSSNQKNSLA |
| 825 | VL CDR2 | WASNRES |
| 826 | VL CDR3 | QQYYSYPLT |
| 827 | VH CDR1 | SGYWT |
| 828 | VH CDR2 | YIYTGSLLYNPSLKS |
| 829 | VH CDR3 | QRDWLGFAY |
| 830 | VL CDR1 | RASQSVSTSSYSYVH |
| 831 | VH CDR2 | SISSGGSIYYPDSVKG |
| 832 | VH CDR3 | GYDAGFAF |
| 833 | VH CDR1 | GFTFSMYMMM |
| 834 | VH CDR1 | GFTFSAYAMA |
| 835 | VH CDR1 | GFTFSAYRMF |
| 836 | VH CDR1 | GFTFSAYLMV |
| 837 | VH CDR1 | GFTFSAYVMF |
| 838 | VH CDR1 | GFTFSAYVMS |
| 839 | VH CDR1 | GFTFSGYLMV |
| 840 | VH CDR1 | GFTFSGYQML |
| 841 | VH CDR1 | GFTFSGYSMF |
| 842 | VH CDR1 | GFTFSGYWMA |
| 843 | VH CDR1 | GFTFSQYLMY |
| 844 | VH CDR1 | GFTFSQYVMF |
| 845 | VH CDR1 | GFTFSQYYMY |
| 846 | VH CDR1 | GFTFSSYLMS |
| 847 | VH CDR1 | GFTFSSYLMT |
| 848 | VH CDR1 | GFTFSSYQMV |
| 849 | VH CDR1 | GFTFSSYSMA |
| 850 | VH CDR1 | GFTFSSYVMF |
| 851 | VH CDR1 | GFTFSSYVMS |
| 852 | VH CDR1 | GFTFSSYVMY |
| 853 | VH CDR1 | GFTFSSYYMF |
| 854 | VH CDR1 | GFTFSSYYMV |
| 855 | VH CDR1 | GFTFSYYSMV |
| 856 | VH CDR1 | GFTFSWYLMA |
| 857 | VH CDR1 | GFTFSWYQMS |
| 858 | Spacer + | CAAGGTCAGTCTGGATCCTATTGCGAGGTTAGTGAGC |
| | PL01-0003 | TGTTTGTTCTTCCTTGGTGCATGGGTGGAGGTGGCTCG |
| | LC | AGCGGTGGCAGCGGTGGCTCTGGTGGTACTAGCACCT |
| | (nucleotide | CTGGTCGTTCCGCTAACCCACGTGGCGGCGGTTCTGA |
| | sequence) | CATCCAGATGACCCAGAGCCCCAGCAGCCTGTCTGCT |
| | | AGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 859 | Spacer + | QGQSGSYCEVSELFVLPWCMGGGGSSGGSGGSGGTSTS |
| | PL01-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPTSTFGGGTKVEIKR |
| | sequence) | |
| 860 | PL01-0003 | TATTGCGAGGTTAGTGAGCTGTTTGTTCTTCCTTGGTG |
| | LC | CATGGGTGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 861 | PL01-0003 | YCEVSELFVLPWCMGGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 862 | Spacer + | CAAGGTCAGTCTGGATCCTCTTGCCTTATGCATCCGCA |
| | PL02-0003 | TTATGCTCATGATTATTGCTATGTTGGAGGTGGCTCGA |
| | LC | GCGGTGGCAGCGGTGGCTCTGGTGGTACTAGCACCTC |
| | (nucleotide | TGGTCGTTCCGCTAACCCACGTGGCGGCGGTTCTGAC |
| | sequence) | ATCCAGATGACCCAGAGCCCCAGCAGCCTGTCTGCTA |
| | | GCGTGGGCGACAGAGTGACCATCACCTGTAGAGCCA |
| | | GCCAGAGCATCAGCAGCTACCTGAACTGGTATCAGCA |
| | | GAAGCCCGGCAAGGCCCCCAAACTGCTGATCTACGCC |
| | | GCCAGCTCTCTGCAGTCTGGCGTGCCCAGCAGATTTT |
| | | CCGGCAGCGGCTCTGGCACCGACTTCACCCTGACCAT |
| | | CAGCTCCCTGCAGCCCGAGGACTTCGCCACCTACTAC |
| | | TGCCAGCAGGACAACGGCTACCCCAGCACCTTTGGCG |
| | | GAGGTACCAAGGTGGAAATCAAGCGTTGTGAGGCCA |
| | | CTCACAAGACATCAACTTCACCCATTGTCAAGAGCTT |
| | | CAACAGGAATGAGTGT |
| 863 | Spacer + | QGQSGSSCLMHPHYAHDYCYVGGGSSGGSGGSGGTSTS |
| | PL02-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVETKR |
| | sequence) | |
| 864 | PL02-0003 | TCTTGCCTTATGCATCCGCATTATGCTCATGATTATTG |
| | LC | CTATGTTGGAGGTGGCTCGAGCGGTGGCAGCGGTGGC |
| | nucleotide | TCTGGTGGTACTAGCACCTCTGGTCGTTCCGCTAACCC |
| | sequence | ACGTGGCGGCGGTTCTGACATCCAGATGACCCAGAGC |
| | | CCCAGCAGCCTGTCTGCTAGCGTGGGCGACAGAGTGA |
| | | CCATCACCTGTAGAGCCAGCCAGAGCATCAGCAGCTA |
| | | CCTGAACTGGTATCAGCAGAAGCCCGGCAAGGCCCCC |
| | | AAACTGCTGATCTACGCCGCCAGCTCTCTGCAGTCTG |
| | | GCGTGCCCAGCAGATTTTCCGGCAGCGGCTCTGGCAC |
| | | CGACTTCACCCTGACCATCAGCTCCCTGCAGCCCGAG |
| | | GACTTCGCCACCTACTACTGCCAGCAGGACAACGGCT |
| | | ACCCCAGCACCTTTGGCGGAGGTACCAAGGTGGAAAT |
| | | CAAGCGTTGTGAGGCCACTCACAAGACATCAACTTCA |
| | | CCCATTGTCAAGAGCTTCAACAGGAATGAGTGT |
| 865 | PL02-0003 | SCLMHPHYAHDYCYVGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 866 | Spacer + | CAAGGTCAGTCTGGATCCTTGTGCGAGGTTTTGATGTT |
| | PL03-0003 | GTTGCAGCATCCGTGGTGCATGGGGGGAGGTGGCTCG |
| | LC | AGCGGTGGCAGCGGTGGCTCTGGTGGTACTAGCACCT |
| | (nucleotide | CTGGTCGTTCCGCTAACCCACGTGGCGGCGGTTCTGA |
| | sequence) | CATCCAGATGACCCAGAGCCCCAGCAGCCTGTCTGCT |
| | | AGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 867 | Spacer + | QGQSGSLCEVLMLLQHPWCMGGGGSSGGSGGSGGTST |
| | PL03-0003 | SGRSANPRGGGSDIQMTQSPSSESASVGDRVITTCRASQS |
| | LC (amino | ISSYLNWYQQKPGKAPKLLIYAASSLQSGVPSRFSGSGS |
| | acid | GTDFTLTISSLQPEDFATYYCQQDNGYPSTFGGGTKVEI |
| | sequence) | KR |
| 868 | PL03-0003 | TTGTGCGAGGTTTTGATGTTGTTGCAGCATCCGTGGTG |
| | LC | CATGGGGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 869 | PL03-0003 | LCEVLMLLQHPWCMGGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATTYCQQDNGYPSTFGGGTKVEIKR |
| 870 | Spacer + | CAAGGTCAGTCTGGATCCATTGCGTGCCGGCATTTA |
| | PL04-0003 | TGGAGCAGTTGCCGTTTTGCCATCATGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 871 | Spacer + | QGQSGSIACRHFMEQLPFCHHGGGSSGGSGGSGGTSTSG |
| | PL04-0003 | RSANPRGGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 872 | PL04-0003 | ATTGCGTGCCGGCATTTTATGGAGCAGTTGCCGTTTTG |
| | LC | CCATCATGGAGGTGGCTCGAGCGGTGGCAGCGGTGGC |
| | nucleotide | TCTGGTGGTACTAGCACCTCTGGTCGTTCCGCTAACCC |
| | sequence | ACGTGGCGGCGGTTCTGACATCCAGATGACCCAGAGC |
| | | CCCAGCAGCCTGTCTGCTAGCGTGGGCGACAGAGTGA |
| | | CCATCACCTGTAGAGCCAGCCAGAGCATCAGCAGCTA |
| | | CCTGAACTGGTATCAGCAGAAGCCCGGCAAGGCCCCC |
| | | AAACTGCTGATCTACGCCGCCAGCTCTCTGCAGTCTG |
| | | GCGTGCCCAGCAGATTTTCCGGCAGCGGCTCTGGCAC |
| | | CGACTTCACCCTGACCATCAGCTCCCTGCAGCCCGAG |
| | | GACTTCGCCACCTACTACTGCCAGCAGGACAACGGCT |
| | | ACCCCAGCACCTTTGGCGGAGGTACCAAGGTGGAAAT |
| | | CAAGCGTTGTGAGGCCACTCACAAGACATCAACTTCA |
| | | CCCATTGTCAAGAGCTTCAACAGGAATGAGTGT |
| 873 | PL04-0003 | IACRHFMEQLPFCHHGGGSSGGSGGSGGTSTSGRSANPR |
| | LC amino | GGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 874 | Spacer + | CAAGGTCAGTCTGGATCCTTTGGTCCTAGGTGCGGTG |
| | PL05-0003 | AGGCTTCTACTGCGTTCCGTATGAGGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 875 | Spacer + | QGQSGSFGPRCGEASTCVPYEGGGSSGGSGGSGGTSTSG |
| | PL05-0003 | RSANPRGGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 876 | PL05-0003 | TTTGGTCCTAGGTGCGGTGAGGCTTCTACTTGCGTTCC |
| | LC | GTATGAGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 877 | PL05-0003 | FGPRCGEASTCVPYEGGGSSGGSGGSGGTSTSGRSANPR |
| | LC amino | GGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 878 | Spacer + | CAAGGTCAGTCTGGATCCATTCTTTATTGCGATAGTTG |
| | PL06-0003 | GGGGGCGGGGTGCTTGACGCGGCCGGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 879 | Spacer + | QGQSGSILYCDSWGAGCLTRPGGGSSGGSGGSGGTSTS |
| | PL06-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 880 | PL06-0003 | ATTCTTTATTGCGATAGTTGGGGGGCGGGGTGCTTGA |
| | LC | CGCGGCCGGGAGGTGGCTCGAGCGGTGGCAGCGGTG |
| | nucleotide | GCTCTGGTCTGTACTAGCACCTCTGGTCGTTCCGCTAAC |
| | sequence | CCACGTGGCGGCGGTTCTGACATCCAGATGACCCAGA |
| | | GCCCCAGCAGCCTGTCTGCTAGCGTGGGCGACAGAGT |
| | | GACCATCACCTGTAGAGCCAGCCAGAGCATCAGCAGC |
| | | TACCTGAACTGGTATCAGCAGAAGCCCGGCAAGGCCC |
| | | CCAAACTGCTGATCTACGCCGCCAGCTCTCTGCAGTC |
| | | TGGCGTGCCCAGCAGATTTTCCGGCAGCGGCTCTGGC |
| | | ACCGACTTCACCCTGACCATCAGCTCCCTGCAGCCCG |
| | | AGGACTTCGCCACCTACTACTGCCAGCAGGACAACGG |
| | | CTACCCCAGCACCTTTGGCGGAGGTACCAAGGTGGAA |
| | | ATCAAGCGTTGTGAGGCCACTCACAAGACATCAACTT |
| | | CACCCATTGTCAAGAGCTTCAACAGGAATGAGTGT |
| 881 | PL06-0003 | ILYCDSWGAGCLTRPGGGSSGGSGGSGGTSTSGRSANPR |
| | LC amino | GGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 882 | Spacer + | CAAGGTCAGTCTGGATCCGGGATTGCGTTGTGCCCGT |
| | PL07-0003 | CTCATTTTTGCCAGCTGCCTCAGACTGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | seqnouce) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 883 | Spacer + | QGQSGSGIALCPSHFCQLPQTGGGSSGGSGGSGGTSTSG |
| | PL07-0003 | RSANPRGGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 884 | PL07-0003 | GGGATTGCGTTGTGCCCGTCTCATTTTTGCCAGCTGCC |
| | LC | TCAGACTGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 885 | PL07-0003 | GIALCPSHFCQLPQTGGGSSGGSGGSGGTSTSGRSANPR |
| | LC amino | GGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 886 | Spacer + | CAAGGTCAGTCTGGATCCGATGGGCCGCGTTGCTTTG |
| | PL08-0003 | TGTCGGGGGAGTGCTCTCCGATTGGTGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 887 | Spacer + | QGQSGSDGPRCFVSGECSPIGGGGSSGGSGGSGGTSTSG |
| | PL08-0003 | RSANPRGGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 888 | PL08-0003 | GATGGGCCGCGTTGCTTTGTGTCGGGGGAGTGCTCTC |
| | LC | CGATTGGTGGAGGTGGCTCGAGCGGTGGCAGCGGTG |
| | nucleotide | GCTCTGGTGGTACTAGCACCTCTGGTCGTTCCGCTAAC |
| | sequence | CCACGTGGCGGCGGTTCTGACATCCAGATGACCCAGA |
| | | GCCCCAGCAGCCTGTCTGCTAGCGTGGGCGACAGAGT |
| | | GACCATCACCTGTAGAGCCAGCCAGAGCATCAGCAGC |
| | | TACCTGAACTGGTATCAGCAGAAGCCCGGCAAGGCCC |
| | | CCAAACTGCTGATCTACGCCGCCAGCTCTCTGCAGTC |
| | | TGGCGTGCCCAGCAGATTTTCCGGCAGCGGCTCTGGC |
| | | ACCGACTTCACCCTGACCATCAGCTCCCTGCAGCCCG |
| | | AGGACTTCGCCACCTACTACTGCCAGCAGGACAACGG |
| | | CTACCCCAGCACCTTTGGCGGAGGTACCAAGGTGGAA |
| | | ATCAAGCGTTGTGAGGCCACTCACAAGACATCAACTT |
| | | CACCCATTGTCAAGAGCTTCAACAGGAATGAGTGT |
| 889 | PL08-0003 | DGPRCFVSGECSPIGGGGSSGGSGGSGGTSTSGRSANPR |
| | LC amino | GGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 890 | Spacer + | CAAGGTCAGTCTGGATCCTTGTGCTATAAGCTGGATT |
| | PL09-0003 | ATGATGATAGGTCTTATTGCCATATTGGAGGTGGCTC |
| | LC | GAGCGGTGGCACTCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCCTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 891 | Spacer + | QGQSGSLCYKLDYDDRSYCHIGGGSSGGSGGSGGTSTS |
| | PL09-0003 | GRSANPRGGGSDIQMTQSRSSLSASVGDRVTITCRASQSI |
| | LC (amino | SSYLNWYQQKPGKAPKLLIYAASSLQSGVPSRFSGSGSG |
| | acid | TDFLLTISSLQPEDFATYYCQQDNGYPSTFGGGTKVEIKR |
| | sequence | |
| 892 | PL09-0003 | TTGTGCTATAAGCTGGATTATGATGATAGGTCTTATTG |
| | LC | CCATATTGGAGGTGGCTCGAGCGGTGGCAGCGGTGGC |
| | nucleotide | TCTGGTGGTACIAGCACCTCTGGTCGTTCCGCTAACCC |
| | sequence | ACGTGGCGGCGGTTCTGACATCCAGATGACCCAGAGC |
| | | CCCAGCAGCCTGTCTGCTAGCGTGGGCGACAGAGTGA |
| | | CCATCACCTGTAGAGCCAGCCAGAGCATCAGCAGCTA |
| | | CCTGAACTGGTATCAGCAGAAGCCCGGCAAGGCCCCC |
| | | AAACTGCTGATCTACGCCGCCAGCTCTCTGCAGTCTG |
| | | GCGTGCCCAGCAGATTTTCCGGCAGCGGCTCTGGCAC |
| | | CGACTTCACCCTGACCATCAGCTCCCTGCAGCCCGAG |
| | | GACTTCGCCACCTACTACTGCCAGCAGGACAACGGCT |
| | | ACCCCAGCACCTTTGGCGGAGGTACCAAGGTGGAAAT |
| | | CAAGCGTTGTGAGGCCACTCACAAGACATCAACTTCA |
| | | CCCATTGTCAAGAGCTTCAACAGGAATGAGTGT |
| 893 | PL09-0003 | LCYKLDYDDRSYCHIGGGSSGGSGGSGGTSTSGRSANPR |
| | LC amino | GGGSDIQMTQSPSSLSASVGDRVTITCRASQSISSYLNWY |
| | acid | QQKPGKAPKLLIYAASSLQSHVPSRFSGSGSGTDFFLTIS |
| | sequence | SLQPEDFATYYCQDNGYPSTFGGGTKVEIKR |
| 894 | Spacer + | CAAGGTCAGTCTGGATCCCCGTGCCATCCGCATCCTT |
| | PL10-0003 | ATGATGCTCGTCCTTATTGCAATGTGGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAACTGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 895 | Spacer + | QGQSGSPCHPHPYDARPYCNVGGGSSGGSGGSGGTSTS |
| | PL10-0003 | GRSANPRGGGSDIQMTQSPSSLSASVGDRVTITCRASQSI |
| | LC (amino | SSYLNWYQQKPGKAPKLLIYAASTLQSGVPSRFSGSGSG |
| | acid | TDFTLTISSLQPEDFATYYCQQDNGYPSTFGGGTKVEIKR |
| | sequence) | |
| 896 | PL10-0003 | CCGTGCCATCCGCATCCTTATGATGCTCGTCCTTATTG |
| | LC | CAATGTGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCACTCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 897 | PL10-0003 | PCHPHPYDARPYCNVGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SLQPEDFATYYCQQDNGYPSTFGGGTKVEIKR |
| 898 | Spacer + | CAAGGTCAGTCTGGATCCCCTTGCTATTGGCATCCTTT |
| | PL11-0003 | TTTTGCGTATAGGTATTGCAATACTGGAGGTGGCTCG |
| | LC | AGCGGTGGCAGCGGTGGCTCTGGTGGTACTAGCACCT |
| | (nucleotide | CTGGTCGTTCCGCTAACCCACGTGGCGGCGGTTCTGA |
| | sequence) | CATCCAGATGACCCAGAGCCCCAGCAGCCTGTCTGCT |
| | | AGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 899 | Spacer + | QGQSGSPCYWHPFFAYRYCNTGGGSSGGSGGSGGTSTS |
| | PL10-003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 900 | PL11-0003 | CCTTGCTATTGGCATCCTTTTTTTGCGTATAGGTATG |
| | LC | CAATACTGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 901 | PL11-0003 | PCYWHPFFAYRYCNTGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 902 | Spacer + | CAAGGTCAGTCTGGATCCGTTTGCTATTATATGGATTG |
| | PL12-0003 | GTTGGGGCGGAATTGGTGCTCFTCGGGAGGTGGCTCG |
| | LC | AGCGGTGGCAGCGGTGGCTCTGGTGGTACTAGCACCT |
| | (nucleotide | CTGGTCGTTCCGCTAACCCACGTGGCGGCGGTTCTGA |
| | sequence) | CATCCAGATGACCCAGAGCCCCAGCAGCCTGTCTGCT |
| | | AGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 903 | Spacer + | QGQSGSVCYYMDWLGRNWCSSGGGSSGGSGGSGGTST |
| | PL12-0003 | SGRSANPRGGGSDIQMTQSPSSLSASVGDRVTITCRASQS |
| | LC (amino | ISSYLNWYQQKPGKAPKLLIYAASSLQSGVPSRFSGSGS |
| | acid | GTDFTLTISSLQPEDFATYYCQQDNGYPSTFGGGTKVEI |
| | sequence) | KR |
| 904 | PL12-0003 | GTTTGCTATTATATGGATTGGTTGGGGCGGAATTGGT |
| | LC | GCTCTTCGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 905 | PL12-0003 | VCYYMDWLGRNWCSSGGGSSGGSGGSGGTSTSGRSAN |
| | LC amino | PRGGGSDIQMTQSPSSLSASVGDRVTITCRASQSISSYLN |
| | acid | WYQQKPGKAPKLLIYAASSLQSGVPSRFSGSGSGTDFTL |
| | sequence | TISSLQPEDFATYYCQQDNGYPSTFGGGTKVEIKR |
| 906 | Spacer + | CAAGGTCAGTCTGGATCCCTGTGCGATCTGTTTAAGTT |
| | PL13-0003 | GCGTGAGTTTCCTTATTGCATGGGGGGAGGTGGCTCG |
| | LC | AGCGGTGGCAGCGGTGGCTCTGGTGGTACTAGCACCT |
| | (nucleotide | CTGGTCGTTCCGCTAACCCACGTGGCGGCGGTTCTGA |
| | sequence) | CATCCAGATGACCCAGAGCCCCAGCAGCCTGTCTGCT |
| | | AGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 907 | Spacer + | QGQSGSLCDLFKLREFRYCMGGGGSSGGSGGSGGTSTS |
| | PL13-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 908 | PL13-0003 | CTGTGCGATCTGTTTAAGTTGCGTGAGTTTCCTTATTG |
| | LC | CATGGGGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 909 | PL13-0003 | LCDLFKLREFPYCMGGGGSSGGSGGSGGTSTSGRSANPR |
| | LC amino | GGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 910 | Spacer + | CAAGGTCAGTCTGGATCCTATCTTCCGTGCCATTTTGT |
| | PL14-0003 | TCCGATTGGGGCTTGCAATAATAAGGGAGGTGGCTCG |
| | LC | AGCGGTGGCAGCGGTGGCTCTGGTGGTACTAGCACCT |
| | (nucleotide | CTGGTCGTCCGCTAACCCACGTGGCGGCGGTTCTGA |
| | sequence) | CATCCAGATGACCCAGAGCCCCAGCAGCCTGTCTGCT |
| | | AGCGTGGGCGACAGAGTGACCATCACCTGTACGACGC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCACTTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGCTTGGAAATCAAGCGTTGTGAGGCC |
| | | ACTCACAAGACATCAACTTCACCCATTGTCAAGAGCT |
| | | TCAACAGGAATGAGTGT |
| 911 | Spacer + | QGQSGSYLPCHFVPIGACNNKGGGSSGGSGGSGGTSTSG |
| | PL14-0003 | RSANPRGGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 912 | P114-0003 | TATCTTCCGTGCCATTTTGTTCCGATTGGGGCTTGCAA |
| | LC | TAATAAGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GCCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 913 | PL14-0003 | YLPCHFVPIGACNNKGGGSSGGSGGSGGTSTSGRSANPR |
| | LC amino | GGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 914 | Spacer + | CAAGGTCAGTCTGGATCCATTTTTTGCCATATGGGTGT |
| | PL15-0003 | TGTGGTTCCTCAGTGCGCGAATTATGGAGGTGGCTCG |
| | LC | AGCGGTGGCAGCGGTGGCTCTGGTGGTACTAGCACCT |
| | (nucleotide | CTGGTCGTTCCGCTAACCCACGTGGCGGCGGTTCTGA |
| | sequence) | CATCCAGATGACCCAGAGCCCCAGCAGCCTGTCTGCT |
| | | AGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 915 | Spacer + | QGQSGIFCHMGVVVPQCANYGGGSSGGSGGSGGTSTS |
| | PL15-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 916 | PL15-0003 | ATTTTTTGCCATATGGGTGTTGTGGTTCCTCAGTGCGC |
| | LC | GAATTATGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 917 | PL15-0003 | IFCHMGVVVPQCANYGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 918 | Spacer + | CAAGGTCAGTCTGGATCCGCGTGCCATCCGCATCCTT |
| | PL16-003 | ATGATGCTCGTCCTTATTGCAATGTGGGAGGTGGCTC |
| | LC | GAGCGCTTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 919 | Spacer + | QGQSGSACHPHPYDARPYCNVGGGSSGGSGGSGGTSTS |
| | PL16-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 920 | PL16-0003 | GCGTGCCATCCGCATCCTTATGATGCTCGTCCTTATTG |
| | LC | CAATGTGGGAGGTGGCTCGAGCGGTGGCAGCGGTGG |
| | nucleotide | CTCTGGTGGTACTAGCACCTCTGGTCTTTCCGCTAACC |
| | sequence | CACGTGGCGGCGGTTCTGACATCCAGATGACCCAGAG |
| | | CCCCAGCAGCCIGTCTGCTAGCGTGGGCGACAGAGTG |
| | | ACCATCACCTGTAGAGCCAGCCAGAGCATCAGCAGCT |
| | | ACCTGAACTGCTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 921 | PL16-0003 | ACHPHPYDARPYCNVGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 922 | Spacer + | CAAGGTCAGTCTGGATCCCCGTGCCATCCGGCTCCTT |
| | PL17-0003 | ATGATGCTCGTLCTTATTGCAATGTGGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 923 | Spacer + | QGQSGSPCHPAPYDARPYCNVGGGSSGGSGGSGGTSTS |
| | PL17-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 924 | PL17-0003 | CCGTGCCATCCGGCTCCTTATGATGCTCGTCCTTATTG |
| | LC | CAATGTGGGAGGTGGCTCGAGCGGTGGCAGCGGTGG |
| | nucleotide | CTCTGGTCGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 925 | PL17-0003 | PCHPAPYDARPYCNVGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 926 | Spacer + | CAAGGTCAGTCTGGATCCCCGTGCCATCCGCATGCTT |
| | PL18-0003 | ATGATGCTCGTCCTTATTGCAATGTGGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACYTCACCCATTGTCAAGAGCT |
| | | TCAACAGGAATGAGTGT |
| 927 | Spacer + | QGQSGSPCHPHAYDARPYCNVGGGSSGGSGGSGGTSTS |
| | PL18-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 928 | PL18-0003 | CCGTGCCATCCGCATGCTTATGATGCTCGTCCTTATTG |
| | LC | CAATGTGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 929 | PL18-0003 | PCHPHAYDARPYCNVGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 930 | Spacer + | CAAGGTCAGTCTGGATCCCCGTGCCATCCGCATCCTG |
| | PL19-0003 | CTGATGCTCGTCCTTATTGCAATGTGGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 931 | Spacer + | QGQSGSPCHPHPADARPYCNVGGGSSGGSGGSGGTSTS |
| | PL19-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 932 | PL19-0003 | CCGTGCCATCCGCATCCTGCTGATGCTCGTCCTTATTG |
| | LC | CAATGTGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTACTCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 933 | PL19-0003 | PCHPHPADARPYCNVGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 934 | Spacer + | CAAGGTCAGTCTGGATCCCCGTGCCATCCGCATCCTT |
| | PL20-0003 | ATGCTGCTCGTCCTTATTGCAATGTGGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 935 | Spacer + | QGQSGSPCHPHPYAARPYCNVGGGSSGGSGGSGGTSTS |
| | PL20-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 936 | PL20-0003 | CCGTGCCATCCGCATCCTTATGCTGCTCGTCCTTATTG |
| | LC | CAATGTGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 937 | PL20-0003 | PCHPHPYAARPYCNVGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 938 | Spacer + | CAAGGTCAGTCTGGATCCCCGTGCCATCCGCATCCTT |
| | PL21-0003 | ATGATGCTGCTCCTTATTGCAATGTGGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 939 | Spacer + | QGQSGSPCHPHPYAARPYCNVGGGSSGGSGGSGGTSTS |
| | PL21-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 940 | PL21-003 | CCGTGCCATCCGCATCCTTATGATGCTGCTCCTTATTG |
| | LC | CAATGTGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 941 | PL21-0003 | PCHPHPYDAAPYCNVGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 942 | Spacer + | CAAGGTCAGTCTGGATCCCCGTGCCATCCGCATCCTT |
| | PL22-0003 | ATGATGCTCGTCCTGCTTGCAATGTGGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 943 | Spacer + | QGQSGSPCHPHPYAARPYCNVGGGSSGGSGGSGGTSTS |
| | PL22-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 944 | PL22-0003 | CCGTGCCATCCGCATCCTTATGATGCTCGTCCTGCTTG |
| | LC | CAATGTGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 945 | PL22-0003 | PCHPHPYDARPACNVGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 946 | Spacer + | CAAGGTCAGTCTGGATCCCCGTGCCATCCGCATCCTT |
| | PL23-0003 | ATGATGCTCGTCCTTATTGCGCTGTGGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 947 | Spacer + | QGQSGSPCHPHPYDARPYCAVGGGSSGGSGGSGGTSTS |
| | PL23-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 948 | PL23-0003 | CCGTGCCATCCGCATCCTTATGATGCTCGTCCTTATTG |
| | LC | CGCTGTGGGAGGTGGCTCGAGCGGTGGCAGCGGTGG |
| | nucleotide | CTCGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 949 | PL23-0003 | PCHPHPYDARPYCAVGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 950 | Spacer + | CAAGGTCAGTCTGGATCCCCGTGCCATGCGCATCCTT |
| | PL24-0003 | ATGATGCTCGTCCTTATTCCAATGIGGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCCGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 951 | Spacer + | QGQSGSPCHAHPYDARPYCNVGGGSSGGSGGSGGTSTS |
| | PL24-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 952 | PL24-0003 | CCGTGCCATGCGCATCCTTATGATGCTCGTCCTTATTG |
| | LC | CAATGTGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTCCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 953 | PL24-0003 | PCHAHPYDARPYCNVGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 954 | Spacer + | CAAGGTCAGTCTGGATCCCCGTGCCATCCGCATCCTT |
| | PL25-0003 | ATGATGCTCGTGCTTATTGCAATGTGGGAGGTGGCTC |
| | LC | GAGCGGTGGCAGCGGTGGCTCTGGTGGTACTAGCACC |
| | (nucleotide | TCTGGTCGTTCCGCTAACCCACGTGGCGGCGGTTCTG |
| | sequence) | ACATCCAGATGACCCAGAGCCCCAGCAGCCTGTCTGC |
| | | TAGCGTGGGCGACAGAGTGACCATCACCTGTAGAGCC |
| | | AGCCAGAGCATCAGCAGCTACCTGAACTGGTATCAGC |
| | | AGAAGCCCGGCAAGGCCCCCAAACTGCTGATCTACGC |
| | | CGCCAGCTCTCTGCAGTCTGGCGTGCCCAGCAGATTT |
| | | TCCGGCAGCGGCTCTGGCACCGACTTCACCCTGACCA |
| | | TCAGCTCCCTGCAGCCCGAGGACTTCGCCACCTACTA |
| | | CTGCCAGCAGGACAACGGCTACCCCAGCACCTTTGGC |
| | | GGAGGTACCAAGGTGGAAATCAAGCGTTGTGAGGCC |
| | | ACTCACAAGACATCAACTTCACCCATTGTCAAGAGCT |
| | | TCAACAGGAATGAGTGT |
| 955 | Spacer + | QGQSGSPCHPHPYDARAYCNVGGGSSGGSGGSGGTSTS |
| | PL25-0003 | GRSANPRGGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 956 | PL25-003 | CCGTGCCATCCGCATCCTTATGATGCTCGTGCTTATTG |
| | LC | CAATGTGGGAGGTGGCTCGAGCGGTGGCAGCGGTGG |
| | nucleotide | CTCTGGTGGTACTAGCACCTCTGGTCGTTCCGCTAACC |
| | sequence | CACGTGGCGGCGGTTCTGACATCCAGATGACCCAGAG |
| | | CCCCAGCAGCCTGTCTGCTAGCGTGGGCGACAGAGTG |
| | | ACCATCACCTGTAGAGCCAGCCAGAGCATCAGCAGCT |
| | | ACCTGAACTGGTATCAGCAGAAGCCCGGCAAGGCCCC |
| | | CAAACTGCTGATCTACGCCGCCAGCTCTCTGCAGTCT |
| | | GGCGTGCCCAGCAGATTTTCCGGCAGCGGCTCTGGCA |
| | | CCGACTTCACCCTGACCATCAGCTCCCTGCAGCCCGA |
| | | GGACTTCGCCACCTACTACTGCCAGCAGGACAACGGC |
| | | TACCCCAGCACCTTTGGCGGAGGTACCAAGGTGGAAA |
| | | TCAAGCGTTGTGAGGCCACTCACAAGACATCAACTTC |
| | | ACCCATTGTCAAGAGCTTCAACAGGAATGAGTGT |
| 957 | PL25-0003 | PCHPHPYDARAYCNVGGGSSGGSGGSGGTSTSGRSANP |
| | LC amino | RGGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 958 | Spacer + | CAAGGTCAGTCTGGATCCGGAGGTGGCTCGAGCGGTG |
| | PL03-2001 | GCAGCGGTGGCTCTGGTGGTATTAGCAGTGGTCTGTT |
| | LC | AAGCGGTCGTAGCGATAATCATGGCGGTTCTGACATC |
| | (nucleotide | CAGATGACCCAGAGCCCCAGCAGCCTGTCTGCTAGCG |
| | sequence) | TGGGCGACAGAGTGACCATCACCTGTAGAGCCAGCCA |
| | | GAGCATCAGCAGCTACCTGAACTGGTATCAGCAGAAG |
| | | CCCGGCAAGGCCCCCAAACTGCTGATCTACGCCGCCA |
| | | GCTCTCTGCAGTCTGGCGTGCCCAGCAGATTTTCCGG |
| | | CAGCGGCTCTGGCACCGACTTCACCCTGACCATCAGC |
| | | TCCCTGCAGCCCGAGGACTTCGCCACCTACTACTGCC |
| | | AGCAGGACAACGGCTACCCCAGCACCTTTGGCGGAG |
| | | GTACCAAGGTGGAAATCAAGCGTTGTGAGGCCACTCA |
| | | CAAGACATCAACTTCACCCATTGTCAAGAGCTTCAAC |
| | | AGGAATGAGTGT |
| 959 | Spacer + | QGQSGSLCEVLMLLQHPWCMGGGGSSGGSGGSGGISSG |
| | PL03-2001 | LLSGRSDNHGGSDIQMTQSPSSLSASVGDRVTITCRASQS |
| | LC (amino | ISSYLNWYQQKPGKAPKLLIYAASSLQSGVPSRFSGSGS |
| | acid | GTDFILTISSLQPEDFATYYCQQDNGYPSTFGGGTKVEI |
| | sequence) | KR |
| 960 | PL03-2001 | GGAGGTGGCTCGAFCGGTGGCAGCGGTGGCTCTGGTG |
| | LC | GTATTAGCAGTGGTCTGTTAAGCGGTCGTAGCGATAA |
| | nucleotide | TCATGGCGGTTCTGACATCCAGATGACCCAGAGCCCC |
| | sequence | AGCAGCCTGTCTGCTAGCGTGGGCGACAGAGTGACCA |
| | | TCACCTGTAGAGCCAGCCAGAGCATCAGCAGCTACCT |
| | | GAACTGGTATCAGCAGAAGCCCGGCAAGGCCCCCAA |
| | | ACTGCTGATCTACGCCGCCAGCTCTCTGCAGTCTGGC |
| | | GTGCCCAGCAGATTTTCCGGCAGCGGCTCTGGCACCG |
| | | ACTTCACCCTGACCATCAGCTCCCTGCAGCCCGAGGA |
| | | CTTCGCCACCTACTACTGCCAGCAGGACAACGGCTAC |
| | | CCCAGCACCTTTGGCGGAGGTACCAAGGTGGAAATCA |
| | | AGCGTTGTGAGGCCACTCACAAGACATCAACTTCACC |
| | | CATTGTCAAGAGCTTCAACAGGAATGAGTGT |
| 961 | PL03-2001 | LCEVLMLLQHPWCMGGGGSSGGSGGSGGISSGLLSGRS |
| | LC amino | DNHGGSDIQMTQSPSSLSASVGDRVTITCRASQSISSYLN |
| | acid | WYQQKPGKAPKLLIYAASSLQSGVPSRFSGSGSGTDFTL |
| | sequence | TISSLQPEDFATYYCQQDNGYPSTFGGGTKVEIKR |
| 962 | Spacer + | QGQSGSIACRHFMEQLPFCHHGGGSSGGSGGSGGISSGL |
| | PL04-2001 | LSGRSDNHGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 963 | PL04-2001 | IACRHFMEQLPFCHHGGGSSGGSGGSGGISSGLLSGRSD |
| | LC amino | NHGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 964 | Spacer + | CAAGGTCAGTCTGGATCCGGAGGTGGCTCGAGCGGTG |
| | PL06-2001- | GCAGCGGTGGCTCTGGTGGTATTAGCAGTGGTCTGTT |
| | mk LC | AAGCGGTCGTAGCGATAATCATGGCGGTTCTGACATC |
| | (nucleotide | CAGATGACCCAGAGCCCCAGCAGCCTGTCTGCTAGCG |
| | sequence) | TGGGCGACAGAGTGACCATCACCTGTAGAGCCAGCCA |
| | | GAGCATCAGCAGCTACCTGAACTGGTATCAGCAGAAG |
| | | CCCGGCAAGGCCCCCAAACTGCTGATCTACGCCGCCA |
| | | GCTCTCTGCAGTCTGGCGTGCCCAGCAGATTTTCCGG |
| | | CAGCGGCTCTGGCACCGACTTCACCCTGACCATCAGC |
| | | TCCCTGCAGCCCGAGGACTTCGCCACCTACTACTGCC |
| | | AGCAGGACAACGGCTACCCCAGCACCTTTGGCGGAG |
| | | GTACCAAGGTGGAAATCAAGCGTTGTGAGGCCACTCA |
| | | CAAGACATCAACTTCACCCATTGTCAAGAGCTTCAAC |
| | | AGGAATGAGTGT |
| 965 | Spacer + | QGQSGSILYCDSWGAGCLTRPGGGSSGGSGGSGGISSGL |
| | PL06-2001- | LSGRSDNHGGSDIQMTQSPSSLSASVGDRVTITCRASQSI |
| | mk LC | SSYLNWYQQKPGKAPKLLIYAASSLQSGVPSRFSGSGSG |
| | (amino acid | TDFTLTISSLQPEDFATYYCQQDNGYPSTFGGGTKVEIKR |
| | sequence) | |
| 966 | PL06-2001- | GGAGGTGGCTCGAGCGGTGGCAGCGGTGGCTCTGGTG |
| | mk LC | GTATTAGCAGTGGTCTGTTAAGCGGTCGTAGCGATAA |
| | nucleotide | TCATGGCGGTTCTGACATCCAGATGACCCAGAGCCCC |
| | sequence | AGCAGCCTGTCTGCTAGCGTGGGCGACAGAGTGACCA |
| | | TCACCTGTAGAGCCAGCCAGAGCATCAGCAGCTACCT |
| | | GAACTGGTATCAGCAGAAGCCCGGCAAGGCCCCCAA |
| | | ACTGCTGATCTACGCCGCCAGCTCTCTGCAGTCTGGC |
| | | GTGCCCAGCAGATTTTCCGGCAGCGGCTCTGGCACCG |
| | | ACTTCACCCTGACCATCAGCTCCCTGCAGCCCGAGGA |
| | | CTTCGCCACCTACTACTGCCAGCAGGACAACGGCTAC |
| | | CCCAGCACCTTTGGCGGAGGTACCAAGGTGGAAATCA |
| | | AGCGTTGTGAGGCCACTCACAAGACATCAACTTCACC |
| | | CATTGTCAAGAGCTTCAACAGGAATGAGTGT |
| 967 | PL06-2001- | ILYCDSWGAGCLTRPGGGSSGGSGGSGGISSGLLSGRSD |
| | mk LC | NHGGSDIQMTQSPSSLSASVGDRVTITCRASQSISSYLNW |
| | amino acid | YQQKPGKAPKLLIYAASSLQSGVPSRFSGSGSGTDFTLTI |
| | sequence | SSLQPEDFATYYCQQDNGYPSTFGGGTKVEIKR |
| 968 | Spacer + | CAAGGTCAGTCTGGATCCGGAGGTGGCTCGAGCGGTG |
| | PL07-2001 | GCAGCGGTGGCTCTGGTGGTATTAGCAGTGGTCTGTT |
| | LC | AAGCGGTCGTAGCGATAATCATGGCGGTTCTGACATC |
| | (nucleotide | CAGATGACCCAGAGCCCCAGCAGCCTGTCTGCTAGCG |
| | sequence) | TGGGCGACAGAGTGACCATCACCTGTAGAGCCAGCCA |
| | | GAGCATCAGCAGCTACCTGAACTGGTATCAGCAGAAG |
| | | CCCGGCAAGGCCCCCAAACTGCTGATCTACGCCGCCA |
| | | GCTCTCTGCAGTCTGGCGTGCCCAGCAGATTTTCCGG |
| | | CAGCGGCTCTGGCACCGACTTCACCCTGACCATCAGC |
| | | TCCCTGCAGCCCGAGGACTTCGCCACCTACTACTGCC |
| | | AGCAGGACAACGGCTACCCCAGCACCTTTGGCGGAG |
| | | GTACCAAGGTGGAAATCAAGCGTTGTGAGGCCACTCA |
| | | CAAGACATCAACTTCACCCATTGTCAAGAGCTTCAAC |
| | | AGGAATGAGTGT |
| 969 | Spacer + | QGQSGSGIALCPSHFCQLPQTGGGSSGGSGGSGGISSGLL |
| | PL07-2001 | SGRSDNHGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 970 | PL07-2001 | GGAGGTGGCTCGAGCGGTGGCAGCGGTGGCTCTGGTG |
| | LC | GTATTAGCAGTGGTCTGTTAAGCGGTCGTAGCGATAA |
| | nucleotide | TCATGGCGGTTCTGACATCCAGATGACCCAGAGCCCC |
| | sequence | AGCAGCCTGTCTGCTAGCGTGGGCGACAGAGTGACCA |
| | | TCACCTGTAGAGCCAGCCAGAGCATCAGCAGCTACCT |
| | | GAACTGGTATCAGCAGAAGCCCGGCAAGGCCCCCAA |
| | | ACTGCTGATCTACGCCGCCAGCTCTCTGCAGTCTGGC |
| | | GTGCCCAGCAGATTTTCCGGCAGCGGCTCTGGCACCG |
| | | ACTTCACCCTGACCATCAGCTCCCTGCAGCCCGAGGA |
| | | CTTCGCCACCTACTACTGCCAGCAGGACAACGGCTAC |
| | | CCCAGCACCTTTGGCGGAGGTACCAAGGTGGAAATCA |
| | | AGCGTTGTGAGGCCACTCACAAGACATCAACTTCACC |
| | | CATTGTCAAGAGCTTCAACAGGAATGAGTGT |
| 971 | PL07-2001 | GIALCPSHFCQLPQTGGGSSGGSGGSGGISSGLLSGRSDN |
| | LC amino | HGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 972 | Spacer + | CAAGGTCAGTCTGGATCCGGAGGTGGCTCGAGCGGTG |
| | PL10-2001 | GCAGCGGTGGCTCTGGTGGTATTAGCAGTGGTCTGTT |
| | LC | AAGCGGTCGTAGCGATAATCATGGCGGTTCTGACATC |
| | (nucleotide | CAGATGACCCAGAGCCCCAGCAGCCTGTCTGCTAGCG |
| | sequence) | TGGGCGACAGAGTGACCATCACCTGTAGAGCCAGCCA |
| | | GAGCATCAGCAGCTACCTGAACTGGTATCAGCAGAAG |
| | | CCCGGCAAGGCCCCCAAACTGCTGATCTACGCCGCCA |
| | | GCTCTCTGCAGTCTGGCGTGCCCAGCAGATTTTCCGG |
| | | CAGCGGCTCTGGCACCGACTTCACCCTGACCATCAGC |
| | | TCCCTGCAGCCCGAGGACTTCGCCACCTACTACTGCC |
| | | AGCAGGACAACGGCTACCCCAGCACCTTTGGCGGAG |
| | | GTACCAAGGTGGAAATCAAGCGTTGTGAGGCCACTCA |
| | | CAAGACATCAACTTCACCCATTGTCAAGAGCTTCAAC |
| | | AGGAATGAGTGT |
| 973 | Spacer + | QGQSGSPCHPHPYDARPYCNVGGGSSGGSGGSGGISSGL |
| | PL10-2001 | LSGRSDNHGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 974 | PL10-2001 | GGAGGTGGCTCGAGCGGTGGCAGCGGTGGCTCTGGTG |
| | LC | GTATTAGCAGTGGTCTGTTAAGCGGTCGTAGCGATAA |
| | nucleotide | TCATGGCGGTTCTGACATCCAGATGACCCAGAGCCCC |
| | sequence | AGCAGCCTGTCTGCTAGCGTGGGCGACAGAGTGACCA |
| | | TCACCTGTAGAGCCAGCCAGAGCATCAGCAGCTACCT |
| | | GAACTGGTATCAGCAGAAGCCCGGCAAGGCCCCCAA |
| | | ACTGCTGATCTACGCCGCCAGCTCTCTGCAGTCTGGC |
| | | GTGCCCAGCAGATTTTCCGGCAGCGGCTCTGGCACCG |
| | | ACTTCACCCTGACCATCAGCTCCCTGCAGCCCGAGGA |
| | | CTTCGCCACCTACTACTGCCAGCAGGACAACGGCTAC |
| | | CCCAGCACCTTTGGCGGAGGTACCAAGGTGGAAATCA |
| | | AGCGTTGTGAGGCCACTCACAAGACATCAACTTCACC |
| | | CATTGTCAAGAGCTTCAACAGGAATGAGTGT |
| 975 | PL10-2001 | PCHPHPYDARPYCNVGGGSSGGSGGSGGISSGLLSGRSD |
| | LC amino | NHGGSDIQMTQSPSSLSASVGDRVTITCRASGSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 976 | Spacer + | QGQSGSPCYWHPFFAYRYCNTGGGSSGGSGGSGGISSG |
| | PL11-2001 | LLSGRSDNHGGSDIQMTQSPSSLSASVGDRVTITCRASQS |
| | LC (amino | ISSYLNWYQQKPGKAPKLLIYAASSLQSGVPSRFSGSGS |
| | acid | GTDFTLTISSLQPEDFATYYCQQDNGYPSTFGGGTKVEI |
| | sequence) | KR |
| 977 | PL11-2001 | PCYWHPFFAYRYCNTGGGSSGGSGGSGGISSGLLSGRSD |
| | LC amino | NHGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 978 | Spacer + | QGQSGSVCYYMDWLGRNWCSSGGGSSGGSGGSGGISS |
| | PL12-2001 | GLLSGRSDNHGGSDIQMTQSPSSLSASVGDRVTITCRAS |
| | LC (amino | QSISSYLNWYQQKPGKAPKLLIYAASSLQSGVPSRFSGS |
| | acid | GSGTDFTLTISSLQPEDFATYYCQQDNGYPSTFGGGTKV |
| | sequence) | EIKR |
| 979 | PL12-2001 | VCYYMDWLGRNWCSSGGGSSGGSGGSGGISSGLLSGRS |
| | LC amino | DNHGGSDIQMTQSPSSLSASVGDRVTITCRASQSISSYLN |
| | acid | WYQQKPGKAPKLLIYAASSLQSGVPSRFSGSGSGTDFTL |
| | sequence | TISSLQPEDFATYYCQQDNGYPSTFGGGTKVEIKR |
| 980 | Spacer + | CAAGGTCAGTCTGGATCCGGAGGTGGCTCGAGCGGTG |
| | PL14-2001 | GCAGCGGTGGCTCTGGTGGTATTAGCAGTGGTCTGTT |
| | LC | AAGCGGTCGTAGCGATAATCATGGCGGTTCTGACATC |
| | (nucleotide | CAGATGACCCAGAGCCCCAGCAGCCTGTCTGCTAGCG |
| | sequence) | TGGGCGACAGAGTGACCATCACCTGTAGAGCCAGCCA |
| | | GAGCATCAGCAGCTACCTGAACTGGTATCAGCAGAAG |
| | | CCCGGCAAGGCCCCCAAACTGCTGATCTACGCCGCCA |
| | | GCTCTCTGCAGTCTGGCGTGCCCAGCAGATTTTCCGG |
| | | CAGCGGCTCTGGCACCGACTTCACCCTGACCATCAGC |
| | | TCCCTGCAGCCCGAGGACTTCGCCACCTACTACTGCC |
| | | AGCAGGACAACGGCTACCCCAGCACCTTTGGCGGAG |
| | | GTACCAAGGTGGAAATCAAGCGTTGTGAGGCCACTCA |
| | | CAAGACATCAACTTCACCCATTGTCAAGAGCTTCAAC |
| | | AGGAATGAGTGT |
| 981 | Spacer + | QGQSGSYLPCHFVPIGACNNKGGGSSGGSGGSGGISSGL |
| | PL14-2001 | LSGRSDNHGGSDIQMTQSPSSLSASVGDRVTITCRASQSI |
| | LC (amino | SSYLNWYQQKPGKAPKLLIYAASSLQSGVPSRFSGSGSG |
| | acid | TDFTLTISSLQPEDFATYYCQQDNGYPSTFGGGTKVEIKR |
| | sequence) | |
| 982 | PL14-2001 | GGAGGTGGCTCGAGCGGTGGCAGCGGTGGCTCTGGTG |
| | LC | GTATTAGCAGTGGTCTGTTAAGCGGTCGTAGCGATAA |
| | nucleotide | TCATGGCGGTTCTGACATCCAGATGACCCAGAGCCCC |
| | sequence | AGCAGCCTGTCTGCTAGCGTGGGCGACAGAGTGACCA |
| | | TCACCTGTAGAGCCAGCCAGAGCATCAGCAGCTACCT |
| | | GAACTGGTATCAGCAGAAGCCCGGCAAGGCCCCCAA |
| | | ACTGCTGATCTACGCCGCCAGCTCTCTGCAGTCTGGC |
| | | GTGCCCAGCAGATTTTCCGGCAGCGGCTCTGGCACCG |
| | | ACTTCACCCTGACCATCAGCTCCCTGCAGCCCGAGGA |
| | | CTTCGCCACCTACTACTGCCAGCAGGACAACGGCTAC |
| | | CCCAGCACCTTTGGCGGAGGTACCAAGGTGGAAATCA |
| | | AGCGTTGTGAGGCCACTCACAAGACATCAACTTCACC |
| | | CATTGTCAAGAGCTTCAACAGGAATGAGTGT |
| 983 | PL14-2001 | YLPCHFVPIGACNNKGGGSSGGSGGSGGISSGLLSGRSD |
| | LC amino | NHGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 984 | Spacer + | CAAGGTCAGTCTGGATCCGGAGGTGGCTCGAGCGGTG |
| | PL15-2001 | GCAGCGGTGGCTCTGGTGGTATTAGCAGTGGTCTGTT |
| | LC | AAGCGGTCGTAGCGATAATCATGGCGGTTCTGACATC |
| | (nucleotide | CAGATGACCCAGAGCCCCAGCAGCCTGTCTGCTAGCG |
| | sequence) | TGGGCGACAGAGTGACCATCACCTGTAGAGCCAGCCA |
| | | GAGCATCAGCAGCTACCTGAACTGGTATCAGCAGAAG |
| | | CCCGGCAAGGCCCCCAAACTGCTGATCTACGCCGCCA |
| | | GCTCTCTGCAGTCTGGCGTGCCCAGCAGATTTTCCGG |
| | | CAGCGGCTCTGGCACCGACTTCACCCTGACCATCAGC |
| | | TCCCTGCAGCCCGAGGACTTCGCCACCTACTACTGCC |
| | | AGCAGGACAACGGCTACCCCAGCACCTTTGGCGGAG |
| | | GTACCAAGGTGGAAATCAAGCGTTGTGAGGCCACTCA |
| | | CAAGACATCAACTTCACCCATTGTCAAGAGCTTCAAC |
| | | AGGAATGAGTGT |
| 985 | Spacer + | QGQSGSIFCHMGVVVPQCANYGGGSSGGSGGSGGISSG |
| | PL15-2001 | LLSGRSDNHGGSDIQMTQSPSSLSASVGDRVTITCRASQS |
| | LC (amino | ISSYLNWYQQKPGKAPKLLIYAASSLQSGVPSRFSGSGS |
| | acid | GTDFTLTISSLQPEDFATYYCQQDNGYPSTFHGGTKVEI |
| | Sequence) | KR |
| 986 | PL15-2001 | GGAGGTGGCTCGAGCGGTGGCAGCGGTGGCTCTGGTG |
| | LC | GTATTAGCAGTGGTCTGTTAAGCGGTCGTAGCGATAA |
| | nucleotide | TCATGGCGGTTCTGACATCCAGATGACCCAGAGCCCC |
| | sequence | AGCAGCCTGTCTGCTAGCGTGGGCGACAGAGTGACCA |
| | | TCACCTGTAGAGCCAGCCAGAGCATCAGCAGCTACCT |
| | | GAACTGGTATCAGCAGAAGCCCGGCAAGGCCCCCAA |
| | | ACTGCTGATCTACGCCGCCAGCTCTCTGCAGTCTGGC |
| | | GTGCCCAGCAGATTTTCCGGCAGCGGCTCTGGCACCG |
| | | ACTTCACCCTGACCATCAGCTCCCTGCAGCCCGAGGA |
| | | CTTCGCCACCTACTACTGCCAGCAGGACAACGGCTAC |
| | | CCCAGCACCTTTGGCGGAGGTACCAAGGTGGAAATCA |
| | | AGCGTTGTGAGGCCACTCACAAGACATCAACTTCACC |
| | | CATTGTCAAGAGCTTCAACAGGAATGAGTGT |
| 987 | PL15-2001 | IFCHMGVVVPQCANYGGGSSGGSGGSGGISSGLLSGRSD |
| | LC amino | NHGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 988 | Spacer + | CAAGGTCAGTCTGGATCCGGAGGTGGCTCGAGCGGTG |
| | PL18-2001 | GCAGCGGTGGCTCTGGTGGTATTAGCAGTGGTCTGTT |
| | LC | AAGCGGTCGTAGCGATAATCATGGCGGTTCTGACATC |
| | (nuucleotide | CAGATGACCCAGAGCCCCAGCAGCCTGTCTGCTAGCG |
| | sequence) | TGGGCGACAGAGTGACCATCACCTGTAGAGCCAGCCA |
| | | GAGCATCAGCAGCTACCTGAACTGGTATCAGCAGAAG |
| | | CCCGGCAAGGCCCCCAAACTGCTGATCTACGCCGCCA |
| | | GCTCTCTGCAGTCTGGCGTGCCCAGCAGATTTTCCGG |
| | | CAGCGGCTCTGGCACCGACTTCACCCTGACCATCAGC |
| | | TCCCTGCAGCCCGAGGACTTCGCCACCTACTACTGCC |
| | | AGCAGGACAACGGCTACCCCAGCACCTTTGGCGGAG |
| | | GTACCAAGGTGGAAATCAAGCGTTGTGAGGCCACTCA |
| | | CAAGACATCAACTTCACCCATTGTCAAGAGCTTCAAC |
| | | AGGAATGAGTGT |
| 989 | Spacer + | QGQSGSPCHPHAYDARPYCNVGGGSSGGSGGSGGISSG |
| | PL18-2001 | LLSGRSDNHGGSDIQMTQSPSSLSASVGDRVTITCRASQS |
| | LC (amino | ISSYLNWYQQKPGKAPKLLIYAASSLQSGVPSRFSGSGS |
| | acid | GTDFTLTISSLQPEDFATYYCQQDNGYPSTFGGGTKVEI |
| | sequence | KR |
| 990 | PL18-2001 | GGAGGTGGCTCGAGCGGTGGCAGCGGTGGCTCTGGTG |
| | LC | GTATTAGCAGTGGTCTGTTAAGCGGTCGTAGCGATAA |
| | nuucleotide | TCATGGCGGTTCTGACATCCAGATGACCCAGAGCCCC |
| | sequence | AGCAGCCTGTCTGCTAGCGTGGGCGACAGAGTGACCA |
| | | TCACCTGTAGAGCCAGCCAGAGCATCAGCAGCTACCT |
| | | GAACTGGTATCACTCAGAAGCCCGGCAAGGCCCCCAA |
| | | ACTGCTGATCTACGCCGCCAGCTCTCTGCAGTCTGGC |
| | | GTGCCCAGCAGATTTTCCGGCAGCGGCTCTGGCACCG |
| | | ACTTCACCCTGACCATCAGCTCCCTGCAGCCCGAGGA |
| | | CTTCGCCACCTACTACTGCCAGCAGGACAACGGCTAC |
| | | CCCAGCACCTTTGGCGGAGGTACCAAGGTGGAAATCA |
| | | AGCGTTGTGAGGCCACTCACAAGACATCAACTTCACC |
| | | CATTGTCAAGAGCTTCAACAGGAATGAGTGT |
| 991 | PL18-2001 | PCHPHAYDARPYCNVGGGSSGGSGGSGGISSGLLSGRSD |
| | LC amino | NHGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 992 | Spacer + | QGQSGSPCHPHPADARPYCNVGGGSSGGSGGSGGISSGL |
| | PL19-2001 | LSGRSDNHGGSDIQMTQSPSSLSASVGDRVTITCRASQSI |
| | LC (amino | SSYLNWYQQKPGKAPKLLIYAASSLQSGVPSRFSGSGSG |
| | acid | TDFLTISSLQPEDFATYYCQQDNGYPSTFGGGTKVEIKR |
| | sequence) | |
| 993 | PL19-2001 | PCHPHPADARPYCNVGGGSSGGSGGSGGISSGLLSGRSD |
| | LC amino | NHGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 994 | Spacer + | QGQSGSPCHPHPYAARPYCNVGGGSSGGSGGSGGISSGL |
| | PL20-2001 | LSGRSDNHGGSDIQMTQSPSSLSASVGDRVTITCRASQSI |
| | LC (amino | SSYLNWYQQKPGKAPKLLIYAASSLQSGVPSRFSGSGSG |
| | acid | TDFLTISSLQPEDFATYYCQQDNGYPSTFGGGTKVEIKR |
| | sequence) | |
| 995 | PL20-2001 | PCHPHPYAARPYCNVGGGSSGGSGGSGGISSGLLSGRSD |
| | LC amino | NHGGSDIQMTQSPSSLSASVGDRVTITCRASQSISSYLNW |
| | acid | YQQKPGKAPKLLIYAASSLQSGVPSRFSGSGSGTDFTLTI |
| | sequence | SSLQPEDFATYYCQQDNGYPSTFGGGTKVEIKR |
| 996 | Spacer + | CAAGGTCAGTCTGGATCCGGAGGTGGCTCGAGCGGTG |
| | PL03- | GCAGCGGTGCTGTGGGTCTCCTGGCTCCCCCGGGCGG |
| | 1004/GG/00 | CCTGTCCGGCCGCAGCGATAATCATGGCGGTTCTGAC |
| | 01 LC | ATCCAGATGACCCAGAGCCCCAGCAGCCTGTCTGCTA |
| | (nucleotide | GCGTGGGCGACAGAGTGACCATCACCTGTAGAGCCA |
| | sequence) | GCCAGAGCATCAGCAGCTACCTGAACTGGTATCAGCA |
| | | GAAGCCCGCsCAAGGCCCCCAAACTGCTGATCTACGCC |
| | | GCCAGCTCTCTGCAGTCTGGCGTGCCCAGCAGATTTT |
| | | CCGGCAGCGGCTCTGGCACCGACTTCACCCTGACCAT |
| | | CAGCTCCCTGCAGCCCGAGGACTTCGCCACCTACTAC |
| | | TGCCAGCAGGACAACGGCTACCCCAGCACCTTTGGCG |
| | | GAGGTACCAAGGTGGAAATCAAGCGTTGTGAGGCCA |
| | | CTCACAAGACATCAACTTCACCCATTGTCAAGAGCTT |
| | | CAACAGGAATGAGTGT |
| 997 | Spacer + | QGQSGSLCEVLMLLQHPWCMGGGGSSGGSGAVGLLAP |
| | PL03- | PGGLSGRSDNHGGSDIQMTQSPSSLSASVGDRVTITCRA |
| | 1004/GG/00 | SQSISSYLNWYQQKPGKAPKLLIYAASSLQSGVPSRFSGS |
| | 01 LC | GSGTDFTLTISSLQPEDFATYYCQQDNGYPSTFGGGTKV |
| | (amino acid | EIKR |
| | sequence | |
| 998 | PL03- | GGACTGTGGCTCGAGCGGTGGCAGCGGTGCTGTGGGTC |
| | 1004/GG/00 | TCCTGGCTCCCCCGGGCGGCCTGTCCGGCCGCAGCGA |
| | 01 LC | TAATCATGGCGGTTCTGACATCCAGATGACCCAGAGC |
| | nucleotide | CCCAGCAGCCTGTCTGCTAGCGTGGGCGACAGAGTGA |
| | sequence | CCATCACCTGTAGAGCCAGCCAGAGCATCAGCAGCTA |
| | | CCTGAACTGGTATCAGCAGAAGCCCGGCAAGGCCCCC |
| | | AAACTGCTGATCTACGCCGCCAGCTCTCTGCAGTCTG |
| | | GCGTGCCCAGCAGATTTTCCGGCAGCGGCTCTGGCAC |
| | | CGACTTCACCCTGACCATCAGCTCCCTGCAGCCCGAG |
| | | GACTTCGCCACCTACTACTGCCAGCAGGACAACGGCT |
| | | ACCCCAGCACCTTTGGCGGAGGTACCAAGGTGGAAAT |
| | | CAAGCGTTGTGAGGCCACTCACAAGACATCAACTTCA |
| | | CCCATTGTCAAGAGCTTCAACAGGAATGAGTGT |
| 999 | PL03- | LCEVLMLLQHPWCMGGGGSSGGSGAVGLLAPPGGLSG |
| | 1004/GG/00 | RSDNHGGSDIQMTQSPSSLSASVGDRVTITCRASQSISSY |
| | 01 LC | LNWYQQKPGKAPKLLIYAASSLQSGVPSRFSGSGSGTDF |
| | amino acid | TLTISSLQPEDFATYYCQQDNGYPSTFGGGTKVEIKR |
| | sequence | |
| 1000 | Spacer + | CAAGGTCAGTCTGGATCCGGAGGTGGCTCGAGCGGTG |
| | PL06- | GCAGCGGTGCTGTGGGTCTCCTGGCTCCCCCGGGCGG |
| | 1004/GG/00 | CCTGTCCGGCCGCAGCGATAATCATGGCGGTTCTGAC |
| | 01 LC | ATCCAGATGACCCAGAGCCCCAGCAGCCTGTCTGCTA |
| | (nucleotide | GCGTGGGCGACAGAGTGACCATCACCTGTAGAGCCA |
| | sequence) | GCCAGAGCATCAGCAGCTACCTGAACTGGTATCAGCA |
| | | GAAGCCCGGCAAGGCCCCCAAACTGCTGATCTACGCC |
| | | GCCAGCTCTCTGCAGTCTGGCGTGCCCAGCAGATTTT |
| | | CCGGCAGCGGCTCTGGCACCGACTTCACCCTGACCAT |
| | | CAGCTCCCTGCAGCCCGAGGACTTCGCCACCTACTAC |
| | | TGCCAGCAGGACAACGGCTACCCCAGCACCTTTGGCG |
| | | GAGGTACCAAGGTGGAAATCAAGCGTTGCGAAGTCA |
| | | CCCATCAGGGCCTGAGCTCGCCCGTCACAAAGAGCTT |
| | | CAACAGGGGAGAGTGT |
| 1001 | Spacer + | QGQSGSILYCDSWGAGCLTRPGGGSSGGSGAVGLLAPP |
| | PL06- | GGLSGRSDNHGGSDIQMTQSPSSLSASVGDRVTITCRAS |
| | 1004/GG/00 | QSISSYLNWYQQKPGKAPKLLIYAASSLQSGVPSRFSGS |
| | 01 LC | GSGTDFTLTISSLQPEDFATYYCQQDNGYPSTFGGGTKV |
| | (amino acid | EIKR |
| | sequence) | |
| 1002 | PL06- | GGAGGTGGCTCGAGCGGTGGCAGCGGTGCTGTGGGTC |
| | 1004/GG/00 | TCCTGCCTCCCCCGGCGGGCCTGTCCGGCCGCAGCGA |
| | 01 LC | TAATCATGGCGGTTCTGACATCCAGATGACCCAGAGC |
| | nucleotide | CCCAGCAGCCTGTCTGCTAGCGTGGGCGACAGAGTGA |
| | sequence | CCATCACCTGTAGAGCCAGCCAGAGCATCAGCAGCTA |
| | | CCTGAACTGGTATCAGCAGAAGCCCGGCAAGGCCCCC |
| | | AAACTGCTGATCTACGCCGCCAGCTCTCTGCAGTCTG |
| | | GCGTGCCCAGCAGATTTTCCGGCAGCGGCTCTGGCAC |
| | | CGACTTCACCCTGACCATCAGCTCCCTGCAGCCCGAG |
| | | GACTTCGCCACCTACTACTGCCAGCAGGACAACGGCT |
| | | ACCCCAGCACCTTTGGCGGAGGTACCAAGGTGGAAAT |
| | | CAAGCGTTGCGAAGTCACCCATCAGGGCCTGAGCTCG |
| | | CCCGTCACAAAGAGCTTCAACAGGGGAGAGTGT |
| 1003 | PL06- | ILYCDSWGAGCLTRPGGGSSGGSGAVGLLAPPGGLSGR |
| | 1004/GG/00 | SDNHGGSDIQMTQSPSSLSASVGDRVTITCRASQSISSYL |
| | 01 LC | NWYQQKPGKAPKLLIYAASSLQSGVPSRFSGSGSGTDFT |
| | amino acid | LTISSLQPEDFATYYCQQDNGYPSTFGGGTKVEIKR |
| | sequence | |
| 1004 | Spacer + | CAAGGTCAGTCTGGATCCGGAGGTGGCTCGAGCGGTG |
| | PL07- | GCAGCGGTGCTGTGGGTCTCCTGGCTCCCCCGGGCGG |
| | 1004/GG/00 | CCTGTCCGGCCGCAGCGATAATCATGGCGGTTCTGAC |
| | 01 LC | ATCCAGATGACCCAGAGCCCCAGCAGCCTGTCTGCTA |
| | (nucleotide | GCGTGGGCGACAGAGTGACCATCACCTGTAGAGCCA |
| | sequence) | GCCAGAGCATCAGCAGCTACCTGAACTGGTATCAGCA |
| | | GAAGCCCGGCAAGGCCCCCAAACTGCTGATCTACGCC |
| | | GCCAGCTCTCTGCAGTCTGGCGTGCCCAGCAGATTTT |
| | | CCGGCAGCGGCTCTGGCACCGACTTCACCCTGACCAT |
| | | CAGCTCCCTGCAGCCCGAGGACTICGCCACCTACTAC |
| | | TGCCAGCAGGACAACGGCTACCCCAGCACCTTTGGCG |
| | | GAGGTACCAAGGTGGAAATCAAGCGTTGCGAAGTCA |
| | | CCCATCAGGGCCTGAGCTCGCCCGTCACAAAGAGCTT |
| | | CAACAGGGGAGAGTGT |
| 1005 | Spacer + | QGQSGSGIALCPSHFCQLPQTGGGSSGGSGAVGLLAPPG |
| | PL07- | GLSGRSDNHGGSDIQMTQSPSSLSASVGDRVTITCRASQ |
| | 1004/GG/00 | SISSYLNWYQQKPGKAPKLLIYAASSLQSGVPSRFSGSGS |
| | 01 LC | GTDFTLTISSLQPEDFATYYCQQDNGYPSTFGGGTKVEI |
| | (amino acid | KR |
| | sequence) | |
| 1006 | PL07- | GGAGGTGGCTCGAGCGGTGGCAGCGGTGCTGTGGGTC |
| | 1004/GG/00 | TCCTGGCTCCCCCGGGCGGCCTGTCCGGCCGCAGCGA |
| | 01 LC | TAATCATGGCGGTTCTGACATCCAGATGACCCAGAGC |
| | nucleotide | CCCAGCAGCCTGTCTGCTAGCGTGGGCGACAGAGTGA |
| | sequence | CCATCACCTGTAGAGCCAGCCAGAGCATCAGCAGCTA |
| | | CCTGAACTGGTATCAGCAGAAGCCCGGCAAGGCCCCC |
| | | AAACTGCTGATCTACGCCGCCAGCTCTCTGCAGTCTG |
| | | GCGTGCCCAGCAGATTTTCCGGCAGCGGCTCTGGCAC |
| | | CGACTTCACCCTGACCATCAGCTCCCTGCAGCCCGAG |
| | | GACTTCGCCACCTACTACTGCCAGCAGGACAACGGCT |
| | | ACCCCAGCACCTTTGGCGGAGGTACCAAGGTGGAAAT |
| | | CAAGCGTTGCGAAGTCACCCATCAGGGCCTGAGCTCG |
| | | CCCGTCACAAAGAGCTTCAACAGGGGAGAGTGT |
| 1007 | PL07- | GIALCPSHFCQLPQTGGGSSGGSGAVGLLAPPGGLSGRS |
| | 1004/GG/00 | DNHGGSDIQMTQSPSSLSASVGDRVTITCRASQSISSYLN |
| | 01 LC | WYQQKPGKAPKLLIYAASSLQSGVPSRFSGSGSGTDFTL |
| | amino acid | TISSLQPEDFATYYCQQDNGYPSTFGGGTKVEIKR |
| | sequence | |
| 1008 | Spacer + | CAAGGTCAGTCTGGATCCGGAGGTGGCTCGAGCGGTG |
| | PL14- | GCAGCGGTGCTGTGGGTCTCCTGGCTCCCCCGGGCGG |
| | 1004/GG/00 | CCTGTCCGGCCGCAGCGATAATCATGGCGGTTCTGAC |
| | 01 LC | ATCCAGATGACCCAGAGCCCCAGCAGCCTGTCTGCTA |
| | (nucleotide | GCGTGGGCGACAGAGTGACCATCACCTGTAGAGCCA |
| | sequence) | GCCAGAGCATCAGCAGCTACCTGAACTGGTATCAGCA |
| | | GAAGCCCGGCAAGGCCCCCAAACTGCTGATCTACGCC |
| | | GCCAGCTCTCTGCAGTCTGGCGTGCCCAGCAGATTTT |
| | | CCGGCAGCGGCTCTGGCACCGACTTCACCCTGACCAT |
| | | CAGCTCCCTGCAGCCCGAGGACTTCGCCACCTACTAC |
| | | TGCCAGCAGGACAACGGCTACCCCAGCACCTTTGGCG |
| | | GAGGTACCAAGGTGGAAATCAAGCGTTGCGAAGTCA |
| | | CCCATCAGGGCCTGAGCTCGCCCGTCACAAAGAGCTT |
| | | CAACAGGGGAGAGTGT |
| 1009 | Spacer + | QGQSGSYLPCHFVPIGACNNKGGGSSGGSGAVGLLAPP |
| | PL 14- | GGLSGRSDNHGGSDIQMTQSPSSLSASVGDRVTTTCRAS |
| | 1004/GG/00 | QSISSYLNWYQQKPGKAPKLLIYAASSLQSGVPSRFSGS |
| | 01 LC | GSGTDFTLTISSLQPEDFATYYCQQDNGYPSTFGGGTKV |
| | (amino acid | EIKR |
| | sequence) | |
| 1010 | PL14- | GGAGGTGGCTCGAGCGGTGGCAGCGGTGCTGTGGGTC |
| | 1004/GG/00 | TCCTGGCTCCCCCGGGCGGCCTGTCCGGCCGCAGCGA |
| | 01 LC | TAATCATGGCGGTTCTGACATCCAGATGACCCAGAGC |
| | nucleotide | CCCAGCAGCCTGTCTGCTAGCGTGGGCGACAGAGTGA |
| | sequence | CCATCACCTGTAGAGCCAGCCAGAGCATCAGCAGCTA |
| | | CCTGAACTGGTATCAGCAGAAGCCCGGCAAGGCCCCC |
| | | AAACTGCTGATCTACGCCGCCAGCTCTCTGCAGTCTG |
| | | GCGTGCCCAGCAGATTTTCCGGCAGCGGCTCTGGCAC |
| | | CGACTTCACCCTGACCATCAGCTCCCTGCAGCCCGAG |
| | | GACTTCGCCACCTACTACTGCCAGCAGGACAACGGCT |
| | | ACCCCAGCACCTTTGGCGGAGGTACCAAGGTGGAAAT |
| | | CAAGCGTTGCGAAGTCACCCATCAGGGCCTGAGCTCG |
| | | CCCGTCACAAAGAGCTTCAACAGGGGAGAGTGT |
| 1011 | PL14- | YLPCHFVPIGACNNKGGGSSGGSGAVGLLAPPGGLSGRS |
| | 1004/GG/00 | DNHGGSDIQMTQSPSSLSASVGDRVTITCRASQSISSYLN |
| | 01 LC | WYQQKPGKAPKLLIYAASSLQSGVPSRFSGSGSGTDFTL |
| | amino acid | TISSLQPEDFATYYCQQDNGYPSTFGGGTKVEIKR |
| | sequence | |
| 1012 | Spacer + | CAAGGTCAGTCTGGATCCGGAGGTGGCTCGAGCGGTG |
| | PL15- | GCAGCGGTGCTGTGGGTCTCCTGGCTCCCCCGGGCGG |
| | 1004/GG/00 | CCTGTCCGGCCGCAGCGATAATCATGGCGGTTCTGAC |
| | 01 LC | ATCCAGATGACCCAGAGCCCCAGCAGCCTGTCTGCTA |
| | (nucleotide | GCGTGGGCGACAGAGTGACCATCACCTGTAGAGCCA |
| | sequence) | GCCAGAGCATCAGCAGCTACCTGAACTGGTATCAGCA |
| | | GAAGCCCGGCAAGGCCCCCAAACTGCTGATCTACGCC |
| | | GCCAGCTCTCTGCAGTCTGGCGTGCCCAGCAGATTTT |
| | | CCGGCAGCGGCTCTGGCACCGACTTCACCCTGACCAT |
| | | CAGCTCCCTGCAGCCCGAGGACTTCGCCACCTACTAC |
| | | TGCCAGCAGGACAACGGCTACCCCAGCACCTTTGGCG |
| | | GAGGTACCAAGGTGGAAATCAAGCGTTGTGAGGCCA |
| | | CTCACAAGACATCAACTTCACCCATTGTCAAGAGCTT |
| | | CAACAGGAATGAGTGT |
| 1013 | Spacer + | QGQSGSIFCHMGVVVPQCANYGGGSSGGSGAVGLLAPP |
| | PL15- | GGLSGRSDNHGGSDIQMTQSPSSLSASVGDRVTITCRAS |
| | 1004/GG/00 | QSISSYLNWYQQKPGKAPKLLIYAASSLQSGVPSRFSGS |
| | 01 LC | GSGTDFTLTISSLQPEDFATYYCQQDNGYPSTFGGGTKV |
| | (amino acid | EIKR |
| | sequence) | |
| 1014 | PL15- | GGAGGTGGCTCGAGCGGTGGCAGCGGTGCTGTGGGTC |
| | 1004/GG/00 | TCCTGGCTCCCCCGGGCGGCCTGTCCGGCCGCAGCGA |
| | 01 LC | TAATCATGGCGGTTCTGACATCCAGATGACCCAGAGC |
| | nucleotide | CCCAGCAGCCTGTCTGCTAGCGTGGGCGACAGAGTGA |
| | sequence | CCATCACCTGTAGAGCCAGCCAGAGCATCAGCAGCTA |
| | | CCTGAACTGGTATCAGCAGAAGCCCGGCAAGGCCCCC |
| | | AAACTGCTGATCTACGCCGCCAGCTCTCTGCAGTCTG |
| | | GCGTGCCCAGCAGATTTTCCGGCAGCGGCTCTGGCAC |
| | | CGACTTCACCCTGACCATCAGCTCCCTGCAGCCCGAG |
| | | GACTTCGCCACCTACTACTGCCAGCAGGACAACGGCT |
| | | ACCCCAGCACCTTTGGCGGAGGTACCAAGGTGGAAAT |
| | | CAAGCGTTGTGAGGCCACTCACAAGACATCAACCTCA |
| | | CCCATTGTCAAGAGCTTCAACAGGAATGAGTGT |
| 1015 | PLl5- | IFCHMGVVVPQCANYGGGSSGGSGAVGLLAPPGGLSGR |
| | 1004/GG/00 | SDNHGGSDIQMTQSPSSLSASVGDRVTITCRASQSISSYL |
| | 01 LC | NWYQQKPGKAPKLLIYAASSLQSGVPSRFSGSGSGTDFT |
| | amino acid | LTISSLQPEDFATYYCQQDNGYPSTFGGGTKVEIKR |
| | sequence | |
| 1016 | Spacer + | CAAGGTCAGTCTGGATCCGGAGGTGGCTCGAGCGGTG |
| | PL18- | GCAGCGGTGCTGTGGGTCTCCTGGCTCCCCCGGGCGG |
| | 1004/GG/00 | CCTGTCCGGCCGCAGCGATAATCATGGCGGTTCTGAC |
| | 01 LC | ATCCAGATGACCCAGAGCCCCAGCAGCCTGTCTGCTA |
| | (nucleotide | GCGTGGGCGACAGAGTGACCATCACCTGTAGAGCCA |
| | sequence) | GCCAGAGCATCAGCAGCTACCTGAACTGGTATCAGCA |
| | | GAAGCCCGGCAAGGCCCCCAAACTGCTGATCTACGCC |
| | | GCCAGCTCTCTGCAGTCTGGCGTGCCCAGCAGATTTT |
| | | CCGGCAGCGGCTCTGGCACCGACTTCACCCTGACCAT |
| | | CAGCTCCCTGCAGCCCGAGGACTTCGCCACCTACTAC |
| | | TGCCAGCAGGACAACGGCTACCCCAGCACCTTTGGCG |
| | | GAGGTACCAAGGTGGAAATCAAGCGTTGTGAGGCCA |
| | | CTCACAAGACATCAACTTCACCCATTGTCAAGAGCTT |
| | | CAACAGGAATGAGTGT |
| 1017 | Spacer + | QGQSGSPCHPHAYDARPYCNVGGGSSGGSGAVGLLAPP |
| | PL18- | GGLSGRSDNHGGSDIQMTQSPSSLSASVGDRVTITCRAS |
| | 1004/GG/00 | QSISSYLNWYQQKPGKAPKLLIYAASSLQSGVPSRFSGS |
| | 01 LC | GSGTDFTLTISSLQPEDFATYYCQQDNGYPSTFGGGTKV |
| | (amino acid | EIKR |
| | sequence) | |
| 1018 | PL18- | GGAGGTGGCTCGAGCGGTGGCAGCGGTGCTGTGGGTC |
| | 1004/GG/00 | TCCTGGCTCCCCCGGGCGGCCTGTCCGGCCGCAGCGA |
| | 01 LC | TAATCATGGCGGTTCTGACATCCAGATGACCCAGAGC |
| | nucleotide | CCCAGCAGCCTGTCTGCTAGCGTGGGCGACAGAGTGA |
| | sequence | CCATCACCTGTAGAGCCAGCCAGAGCATCAGCAGCTA |
| | | CCTGAACTGGTATCAGCAGAAGCCCGGCAAGGCCCCC |
| | | AAACTGCTGATCTACGCCGCCAGCTCTCTGCAGTCTG |
| | | GCGTGCCCAGCAGATTTTCCGGCAGCGGCTCTGGCAC |
| | | CGACTTCACCCTGACCATCAGCTCCCTGCAGCCCGAG |
| | | GACTTCGCCACCTACTACTGCCAGCAGGACAACGGCT |
| | | ACCCCAGCACCTTTGGCGGAGGTACCAAGGTGGAAAT |
| | | CAAGCGTTGTGAGGCCACTCACAAGACATCAACTTCA |
| | | CCCATTGTCAAGAGCTTCAACAGGAATGAGTGT |
| 1019 | PL18- | PCHPHAYDARPYCNVGGGSSGGSGAVGLLAPPGGLSGR |
| | 1004/GG/00 | SDNHGGSDIQMTQSPSSLSASVGDRVTITCRASQSISSYL |
| | 01 LC | NWYQQKPGKAPKLLIYAASSLQSGVPSRFSGSGSGTDFT |
| | amino acid | LTISSLQPEDFATYYCQQDNGYPSTFGGGTKVEIKR |
| | sequence | |
| 1020 | Spacer + | QGQSGSGIALCPSHFCQLPQTGGGSSGGSGGSGGLSGRS |
| | PL07-0001 | DNHGGSDIQMTQSPSSLSASVGDRVTITCRASQSISSYLN |
| | LC (amino | WYQQKPGKAPKLLIYAASSLQSGVPSRFSGSGSGTDFTL |
| | acid | TISSLQPEDFATYYCQQDNGYPSTFGGGTKVEIKR |
| | sequence) | |
| 1021 | PL07-0001 | GIALCPSHFCQLPQTGGGSSGGSGGSGGLSGRSDNHGGS |
| | LC amino | DIQMTQSPSSLSASVGDRVTITCRASQSISSYLNWYQQKP |
| | acid | GKAPKLLIYAASSLQSGVPSRFSGSGSGTDFTLTISSLQPE |
| | sequence | DFATYYCQIQDNGYPSTFGGGTKVEIKR |
| 1022 | Spacer + | QGQSGSGIALCPSHFCQLPQTGGGSSGGSGGSGGLSGRS |
| | PL07-0002 | DNHGGSDIQMTQSPSSLSASVGDRVTITCRASQSISSYLN |
| | LC (amino | WYQQKPGKAPKLLIYAASSLQSGVPSRFSGSGSGTDFTL |
| | acid | TISSLQPEDFATYYCQQDNGYPSTFGGGTKVEIKR |
| | sequence) | |
| 1023 | PL07-0002 | GIALCPSHFCQLPQTGGGSSGGSGGSGGLSGRSDNHGGS |
| | LC amino | DIQMTQSPSSLSASVGDRVTITCRASQSISSYLNWYQQKP |
| | acid | GKAPKLLIYAASSLQSGVPSRFSGSGSGTDFTLTISSLQPE |
| | sequence | DFATYYCQIQDNGYPSTFGGGTKVEIKR |
| 1024 | Spacer + | QGQSGSGIALCPSHFCQLPQTGGGSSGGSGGSGGISSGLL |
| | PL07-001 | SSGGSDIQMTQSPSSLSASVGDRVTITCRASQSISSYLNW |
| | LC (amino | YQQKPGKAPKLLIYAASSLQSGVPSRFSGSGSGTDFTLTI |
| | acid | SSLQPEDFATYYCQQDNGYPSTFGGGTKVEIKR |
| | sequence) | |
| 1025 | PL07-1001 | GIALCPSHFCQLPQTGGGSSGGSGGSGGISSGLLSSGGSD |
| | LC amino | IQMTQSPSSLSASVGDRVTITCRASQSISSYLNWYQQKPG |
| | acid | KAPKLLIYAASSLQSGVPSRFSGSGSGTDFTLTISSLQPED |
| | sequence | FATYYCQQDNGYPSTFGGGTKVEIKR |
| 1026 | Spacer + | QGQSGSGIALCPSHFCQLPQTGGGSSGGSGGSGGQNQAL |
| | PL07-1002 | RMAGGSDIQMTQSPSSLSASVGDRVTITCRASQSISSYLN |
| | LC (amino | WYQQKPGKAPKLLIYAASSLQSGVPSRFSGSGSGTDFTL |
| | acid | TISSLQPEDFATYYCQQDNGYPSTFGGGTKVEIKR |
| | sequence) | |
| 1027 | PL07-1002 | GIALCPSHFCQLPQTGGGSSGGSGGSGGQNQALRMAGG |
| | LC amino | SDIQMTQSPSSLSASVGDRVTITCRASQSISSYLNWYQQK |
| | acid | PGKAPKLLIYAASSLQSGVPSRFSGSGSGTDFTLTISSLQP |
| | sequence | EDFATYYCQQDNGYPSTFGGGTKVEIKR |
| 1028 | Spacer + | QGQSGSGIALCPSHFCQLPQTGGGSSGGSGGSGGVHMPL |
| | PL07-1003 | GFLGPGGSDIQMTQSPSSLSASVGDRVTITCRASQSISSY |
| | LC (amino | LNWYQQKPGKAPKLLIYAASSLQSGVPSRFSGSGSGTDF |
| | acid | TLTISSLQPEDFATYYCQQDNGYPSTFGGGTKVEIKR |
| | sequence) | |
| 1029 | PL07-003 | GIALCPSHFCQLPQTGGGSSGGSGGSGGVHMPLGFLGPG |
| | LC amino | GSDIQMTQSPSSLSASVGDRVTITCRASQSISSYLNWYQQ |
| | acid | KPGKAPKLLIYAASSLQSGVPSRFSGSGSGTDFTLTISSLQ |
| | sequence | PEDFATYYCQQDNGYPSTFGGGTKVEIKR |
| 1030 | Spacer + | QGQSGSGIALCPSHFCQLPQTGGGSSGGSGGSGGAVGLL |
| | PL07-1004 | APPGGSDIQMTQSPSSLSASVGDRVTITCRASQSISSYLN |
| | LC (amino | WYQQKPGKAPKLLIYAASSLQSGVPSRFSGSGSGTDFTL |
| | acid | TISSLQPEDFATYYCQQDNGYPSTFGGGTKVEIKR |
| | sequence) | |
| 1031 | PL07-1004 | GIALCPSHFCQLPQTGGGSSGGSGGSGGAVGLLAPPGGS |
| | LC amino | DIQMTQSPSSLSASVGDRVTITCRASQSISSYLNWYQQKP |
| | acid | GKAPKLLIYAASSLQSGVPSRFSGSGSGTDFTLTISSLQPE |
| | sequence | DFATYYCQQDNGYPSTFGGGTKVEIKR |
| 1032 | Spacer + | QGQSGSGIALCPSHFCQLPTGGGSSGGSGGSGGISSGLL |
| | PL07-2002 | SGRSGNHGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 1033 | PL07-2002 | GIALCPSHFCQLPQTGGGSSGGSGGSGGISSGLLSGRSGN |
| | LC amino | HGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 1034 | Spacer + | QGQSGSGIALCPSHFCQLPQTGGGSSGGSGGSGGISSGLL |
| | PL07-2003 | SGRSANPRGGGSDIQMTQSPSSLSASVGDRVTITCRASQS |
| | LC (amino | ISSYLNWYQQKPGKAPKLLIYAASSLQSGVPSRFSGSGS |
| | acid | GTDFTLTISSLQPEDFATYYCQQDNGYPSTFGGGTKVEI |
| | sequence) | KR |
| 1035 | PL07-2003 | GIALCPSHFCQLPQTGGGSSGGSGGSGGISSGLLSGRSAN |
| | LC amino | PRGGGSDIQMTQSPSSLSASVGDRVTITCRASQSISSYLN |
| | acid | WYQQKPGKAPKLLIYAASSLQSGVPSRFSGSGSGTDFTL |
| | sequence | TISSLQPEDFATYYCQQDNGYPSTFGGGTKVEIKR |
| 1036 | Spacer + | QGQSGSGIALCPSHFCQLPQTGGGSSGGSGGSGGAVGLL |
| | PL07-2004 | APPTSGRSANPRGGGSDIQMTQSPSSLSASVGDRVTITCR |
| | LC (amino | ASQSISSYLNWYQQKPGKAPKLLIYAASSLQSGVPSRFS |
| | acid | GSGSGTDFTLTISSLQPEDFATYYCQQDNGYPSTFGGGT |
| | sequence) | KVEIKR |
| 1037 | PL07-2004 | GIALCPSHFCQLPQTGGGSSGGSGGSGGAVGLLAPPTSG |
| | LC amino | RSANPRGGGSDIQMTQSPSSLSASVGDRVTITCRASQSIS |
| | acid | SYLNWYQQKPGKAPKLLIYAASSLQSGVPSRFSGSGSGT |
| | sequence | DFTLTISSLQPEDFATYYCQQDNGYPSTFGGGTKVEIKR |
| 1038 | Spacer + | QGQSGSGIALCPSHFCQLPQTGGGSSGGSGGSGGAVGLL |
| | PL07-2005 | APPSGRSANPRGGGSDIQMTQSPSSLSASVGDRVTITCRA |
| | LC (amino | SQSISSYLNWYQQKPGKAPKLLIYAASSLQSGVPSRFSGS |
| | acid | GSGTDFTLTISSLQPEDFATYYCQQDNGYPSTFGGGTKV |
| | sequence) | EIKR |
| 1039 | PL07-2005 | GIALCPSHFCQLPQTGGGSSGGSGGSGGAVGLLAPPSGR |
| | LC amino | SANPRGGGSDIQMTQSPSSLSASVGDRVTITCRASQSISS |
| | acid | YLNWYQQKPGKAPKLLIYAASSLQSGVPSRFSGSGSGTD |
| | sequence | FTLTISSLQPEDFATYYCQQDNGYPSTFGGGTKVEIKR |
| 1040 | Spacer + | QGQSGSGIALCPSHFCQLPQTGGGSSGGSGGSGGISSGLL |
| | PL07-2006 | SGRSDDHGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 1041 | PL07-2006 | GIALCPSHFCQLPQTGGGSSGGSGGSGGISSGLLSGRSDD |
| | LC amino | HGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 1042 | Spacer + | QGQSGSGIALCPSHFCQLPQTGGGSSGGSGGSGGISSGLL |
| | PL07-2007 | SGRSDIHGGSDIQMTQSPSSLSASVGDRVTITCRASQSISS |
| | LC (amino | YLNWYQQKPGKAPKLLIYAASSLQSGVPSRFSGSGSGTD |
| | acid | FTLTISSLQPEDFATYYCQQDNGYPSTFGGGTKVEIKR |
| | sequence) | |
| 1043 | PL07-2007 | GIALCPSHFCQLPQTGGGSSGGSGGSGGISSGLLSGRSDI |
| | LC amino | HGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 1044 | Spacer + | QGQSGSGIALCPSHFCQLPQTGGGSSGGSGGSGGISSGLL |
| | PL07-2008 | SGRSDQHGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 1045 | PL07-2008 | GIALCPSHFCQLPQTGGGSSGGSGGSGGISSGLLSGRSDQ |
| | LC amino | HGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 1046 | Spacer + | QGQSGSGIALCPSHFCQLPQTGGGSSGGSGGSGGISSGLL |
| | PL07-2009 | SGRSDTHGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 1047 | PL07-2009 | GIALCPSHFCQLPQTGGGSSGGSGGSGGISSGLLSGRSDT |
| | LC amino | HGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 1048 | Spacer + | QGQSGSGIALCPSHFCQLPQTGGGSSGGSGGSGGISSGLL |
| | PL07-2010 | SGRSDYHGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 1049 | PL07-2010 | GIALCPSHFCQLPQTGGGSSGGSGGSGGISSGLLSGRSDY |
| | LC amino | HGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 1050 | Spacer + | QGQSGSGIALCPSHFCQLPQTGGGSSGGSGGSGGISSGLL |
| | PL07-2011 | SGRSDNPGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 1051 | PL07-2011 | GIALCPSHFCQLPQTGGGSSGGSGGSGGISSGLLSGRSDN |
| | LC amino | PGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 1052 | Spacer + | QGQSGSGIALCPSHFCQLPQTGGGSSGGSGGSGGISSGLL |
| | PL07-2012 | SGRSDNPGGSDIQMTQSPSSLSASVGDRVTITCRASQSIS |
| | LC (amino | SYLNWYQQKPGKAPKLLIYAASSLQSGVPSRFSGSGSGT |
| | acid | DFTLTISSLQPEDFATYYCQQDNGYPSTFGGGTKVEIKR |
| | sequence) | |
| 1053 | PL07-2012 | GIALCPSHFCQLPQTGGGSSGGSGGSGGISSGLLSGRSAN |
| | LC amino | PGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 1054 | Spacer + | QGQSGSGIALCPSHFCQLPQTGGGSSGGSGGSGGISSGLL |
| | PL07-2013 | SGRSANIGGSDIQMTQSPSSLSASVGDRVTITCRASQSISS |
| | LC (amino | YLNWYQQKPGKAPKLLIYAASSLQSGVPSRFSGSGSGTD |
| | acid | FTLTISSLQPEDFATYYCQQDNGYPSTFGGGTKVEIKR |
| | sequence) | |
| 1055 | PL07-2013 | GIALCPSHFCQLPQTGGGSSGGSGGSGGISSGLLSGRSAN |
| | LC amino | IGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 1056 | Spacer + | QGQSGSGIALCPSHFCQLPQTGGGSSGGSGGSGGISSGLL |
| | PL07-2014 | SGRSDNIGGSDIQMTQSPSSLSASVGDRVTITCRASQSISS |
| | LC (amino | YLNWYQQKPGKAPKLLIYAASSLQSGVPSRFSGSGSGTD |
| | acid | FTLTISSLQPEDFATYYCQQDNGYPSTFGGGTKVEIKR |
| | sequence) | |
| 1057 | PL07-2014 | GIALCPSHFCQLPQTGGGSSGGSGGSGGISSGLLSGRSDN |
| | LC amino | IGGSDIQMTQSPSSLSASVGDRVTITCRASQSISSYLNWY |
| | acid | QQKPGKAPKLLIYAASSLQSGVPSRFSGSGSGTDFTLTIS |
| | sequence | SLQPEDFATYYCQQDNGYPSTFGGGTKVEIKR |
| 1058 | Spacer + | QGQSGSGIALCPSHFCQLPQTGGGSSGGSGGSGGAVGLL |
| | PL07- | APPGGTSTSGRSANPRGGGSDIQMTQSPSSLSASVGDRV |
| | 03 LC | TITCRASQSISSYLNWYQQKPGKAPKLLIYAASSLQSGVP |
| | (amino acid | SRFSGSGSGTDFTLTISSLQPEDFATYYCQQDNGYPSTFG |
| | sequence) | GGTKVEIKR |
| 1059 | PL07- | GIALCPSHFCQLPQTGGGSSGGSGGSGGAVGLLAPPGGT |
| | 100/GG/00 | STSGRSANPRGGGSDIQMTQSPSSLSASVGDRVTITCRAS |
| | 03 LC | QSISSYLNWYQQKPGKAPKLLIYAASSLQSGVPSRFSGS |
| | amino acid | GSGTDFTLTISSLQPEDFATYYCQQDNGYPSTFGGGTKV |
| | sequence | EIKR |
| 1060 | anti--PDL1 | GAGGTGCAGCTGTTGGAGTCTGGGGGAGGCTTGGTAC |
| | ScFvs | AGCCTGGGGGGTCCCTGAGACTCTCCTGTGCAGCCTC |
| | (nucleic acid | TGGATTCACCTTTAGCAGCTATGCCATGAGCTGGGTC |
| | sequence) | CGCCAGGCTCCAGGGAAGGGCTCTGGAGTGGGTCTCA |
| | | GATATTACTGCGTCGGGTTAGAGGACAACGTACGCAG |
| | | ACTCCGTGAAGGGCCGGTTCACCATCTCCAGAGACAA |
| | | TTCCAAGAACACGCTGTATCTGCAAATGAACAGCCTG |
| | | AGAGCCGAGGACACGGCCGTATATTACTGTGCGAGAT |
| | | CGAAGATTGCTTTTGACTACTGGGGCCAGGGAACCCT |
| | | GGTCACCGTCTCGAGCGGTGGAGGCGGTTCAGGCGGA |
| | | GGTGGCAGCGGCGGTGGCGGGTCGACGGACATCCAG |
| | | ATGACCCAGTCTCCATCCTCCCTGTCTGCATCTGTAGG |
| | | AGACAGAGTCACCATCACTTGCCGGGCAAGTCAGAGC |
| | | ATTAGCAGCTATTTAAATTGGTATCAGCAGAAACCAG |
| | | GGAAAGCCCCTAAGCTCCTGATCTATAAGGCATCCCG |
| | | TTTGCAAAGTGGGGTCCCATCAAGGTTCAGTGGCAGT |
| | | GGATCTGGGACAGATTTCACTCTCACCATCAGCAGTC |
| | | TGCAACCTGAAGATTTTGCAACTTACTACTGTCAACA |
| | | GCGTGCGCTTAAGCCTGTGACGTTCGGCCAAGGGACC |
| | | AAGGTGGAAATCAAACGG |
| 1061 | anti-PDL1 | EVQLLESGGGLVQPGGSLRLSCAASGFTFSSYAMSWVR |
| | ScFvs | QAPGKGLEWVSDITASGQRTTYADSVKGRFTISRDNSK |
| | (amino acid | NTLYLQMNSLRAEDTAVYYCARSKIAFDYWGQGTLVT |
| | sequence) | VSSGGGGSGGGGSGGGGSTDIQMTQSPSSLSASVGDRV |
| | | TITCRASQSISSYLNWYQQKPGRAPKLLIYKASRLQSGV |
| | | PSRFSGSGSGTDFTLTISSLQPEDFATYYCQQRALKPVTF |
| | | GQGTKVEIKR |
| 1062 | anti-PDL1 | GAGGTGCAGCTGTTGGAGTCTGGGGGAGGCTTGGTAC |
| | ScFvs | AGCCTGGGGGGTCCCTGAGACTATCCTGTGCAGCCTC |
| | (nucleic acid | TGGATTCACCTTTAGCAGCTATGCCATGAGCTGGGTC |
| | sequence) | CGCCAGGCTCCAGGGAAGGGGCTGGAGTGGGTCTCA |
| | | AGTATTAATAAGGATGGTCATTATACAAGTTACGCAG |
| | | ACTCCGTGAAGGGCCGGTTCACCATCTCCAGAGACAA |
| | | TTCCAAGAACACGCTGTATCTGCAAATGAACAGCCTG |
| | | AGAGCCGAGGACACGGCCGTATATTACTGTGCGAAA |
| | | AATCTTGATGAGTTTGACTACTGGGGCCAGGGAACCC |
| | | TGGTCACCGTCTCGAGCGGTGGAGGCGGTTCAGGCGG |
| | | AGGTGGCAGCGGCGGTGGCGGGTCGACGGACATCCA |
| | | GATGACCCAGTCTCCATCCTCCCTGTCTGCATCTGTAG |
| | | GAGACAGAGTCACCATCACTTGCCGGGCAAGTCAGA |
| | | GCATTAGCAGCTATTTAAATTGGTATCAGCAGAAACC |
| | | AGGGAAAGCCCCTAAGCTCCTGATCTATGCTGCATCC |
| | | AGTTTGCAAAGTGGGGTCCCATCAAGGTTCAGTGGCA |
| | | GTGGATCTGGGACAGATTTCACTCTCACCATCAGCAG |
| | | TCTGCAACCTGAAGATTTTGCAACTTACTACTGTCAAC |
| | | AGAGTTACAGTACCCCTAATACGTTCGGCCAAGGGAC |
| | | CAAGGTGGAAATCAAACGG |
| 1063 | anti-PDL1 | EVQLLESGGGLVQPGGSLRLSCAASGFTFSSYAMSWVR |
| | ScFvs | QAPGKGLEWVSSINKDGHYTSYADSVKGRFTISRDNSK |
| | (amino acid | NTLYLQMNSLRAEDTAVYYCAKNLDEFDYWGQGTLVT |
| | sequence) | VSSGGGGSGGGGSGGGGSTDIQMTQSPSSLSASVGDRV |
| | | TITCRASQSISSYLNWYQQKPGKAPKLLIYAASSLQSGVP |
| | | SRFSGSGSGTDFTLTISSLQPEDFATYYCQQSYSTPNTFG |
| | | QGTKVEIKR |
| 1064 | anti-PDL1 | GAGGTGCAGCTGTTGGAGTCTGGGGGAGGCTTGGTAC |
| | ScFvs | AGCCTGGGGGGTCCCTGAGACTCTCCTGTGCAGCCTC |
| | (nucleic acid | TGGATTCACCTTTAGCAGCTATGCCATGAGCTGGGTC |
| | sequence) | CGCCAGGCTCCAGGGAAGGGGCTGGAGTGGGTCTCAT |
| | | CTATTATGGCTACTGGTGCTGGTACATTGTACGCAGA |
| | | CTCCGTGAAGGGCCGGTTCACCATCTCCAGAGACAAT |
| | | TCCAAGAACACGCTGTATCTGCAAATGAACAGCCTGA |
| | | GAGCCGAGGACACGGCCGTATATTACTGTGCGAAAG |
| | | ATGGTGCGGGGTTTGACTACTGGGGCCAGGGAACCCT |
| | | GGTCACCGTCTCGAGCGGTGGAGGCGGTTCAGGCGGA |
| | | GGTGGCAGCGGCGGTGGCGGGTCGACGGACATCCAG |
| | | ATGACCCAGTCTCCATCCTCCCTGTCTGCATCTGTAGG |
| | | AGACAGAGTCACCATCACTTGCCGGGCAAGTCAGAGC |
| | | ATTAGCAGCTATTTAAATTGGTATCAGCAGAAACCAG |
| | | GGAAAGCCCCTAAGCTCCTGATCTATTCTGCATCCCA |
| | | GTTGCAAAGTGGGGTCCCATCAAGGTTCAGTGGCAGT |
| | | GGATCTGGGACAGATTTCACTCTCACCATCAGCAGTC |
| | | TGCAACCTGAAGATTTTGCAACTTACTACTGTCAACA |
| | | GGCGAATTCGCGGCCTTCTACGTTCGGCCAAGGGACC |
| | | AAGGTGGAAATCAAACGG |
| 1065 | anti-PDL1 | EVQLLESGGGLVQPGGSLRLSCAASGFTFSSYAMSWVR |
| | ScFvs | QAPGKGLEWVSSIMATGAGTLYADSVKGRFTISRDNSK |
| | (amino acid | NTLYLQMNSLRAEDTAVYYCAKDGAGFDYWGQGTLV |
| | sequence) | TVSSGGGGSGGGGSGGGGSTDIQMTQSPSSLSASVGDR |
| | | VTITCRASQSISSYLNWYQQKPGKAPKLLIYSASQLQSG |
| | | VPSRFSGSGSGTDFTLTISSLQPEDFATYYCQQANSRPST |
| | | FGQGTKVEIKR |
| 1066 | anti-PDL1 | GAGGTGCAGCTGTTGGAGTCTGGGGGAGGCTTGGTAC |
| | ScFvs | AGCCTGGGGGGTCCCTGAGACTCTCCTGTGCAGCCTC |
| | (nucleic acid | TGGATTCACCTTTAGCAGCTATGCCATGAGCTGGGTC |
| | sequence) | CGCCAGGCTCCAGGGAAGGGGCTGTAGTGGGTCTCAA |
| | | CTATTACTTCTTCTGGTGCTGCTACATATTACGCAGAC |
| | | TCCGTGAAGGGCCGGTTCACCATCTCCAGAGACAATT |
| | | CCAAGAACACGCTGTATCTGCAAATGAACAGCCTGAG |
| | | AGCCGAGGACACGGCCGTATATTACTGTGCGAAAAAT |
| | | TATACTGGTTTTGACTACTGGGGCCAGGGAACCCTGG |
| | | TCACCGTCTCGAGCGGTGGAGGCGGTTCAGGCGGAGG |
| | | TGGCAGCGGCGGTGGCGGGTCGACGGACATCCAGAT |
| | | GACCCAGTCTCCATCCTCCCTGTCTGCATCTGTAGGAG |
| | | ACAGAGTCACCATCACTTGCCGGGCAAGTCAGAGCAT |
| | | TAGCAGCTATTTAAATTGGTATCAGCAGAAACCAGGG |
| | | AAAGCCCCTAAGCTCCTGATCTATAATGCATCCTCCTT |
| | | GCAAAGTGGGGTCCCATCAAGGTTCAGTGGCAGTGGA |
| | | TCTGGGACAGATTTCACTCTCACCATCAGCAGTCTGC |
| | | AACCTGAAGATTTTGCAACTTACTACTGTCAACAGTA |
| | | TACTTATGGTCCTGGTACGTTCGGCCAAGGGACCAAG |
| | | GTGGAAATCAAACGG |
| 1067 | anti-PDL1 | EVQLLESGGGLVQPGGSLRLSCAASGFTFSSYAMSWVR |
| | ScFvs | QAPGKGLQWVSTITSSGAATYYADSVKGRFTISRDNSK |
| | (amino acid | NTLYLQMNSLRAEDTAVYYCAKNYTGFDYWGQGTLVT |
| | sequence) | VSSGGGGSGGGGSGGGGSTDIQMTQSPSSLSASVGDRV |
| | | TITCRASQSISSYLNWYQQKPGKAPKLLIYNASSLQSGVP |
| | | SRFSGSGSGTDFTLTISSLQPEDFATYYCQQYTYGPGTFG |
| | | QGTKVEIKR |
| 1068 | anti-PDL1 | GAGGTGCAGCTGTTGGAGTCTGGGGGAGGCTTGGTAC |
| | ScFvs | AGCCTGGGGGGTCCCTGAGACTCTCCTGTGCAGCCTC |
| | (nucleic acid | TGGATTCACCTTTAGCAGCTATGCCATGAGCTGGGTC |
| | sequence) | CGCCAGGCTCCAGGGAAGGGGCTGGAGTGGGTCTCA |
| | | AGTATTTATTCTACTGGTGGTGCTACAGCTTACGCAGA |
| | | CTCCGTGAAGGGCCGGTTCACCATCTCCAGAGACAAT |
| | | TCCAAGAACACGCTGTATCTGCAAATGAACAGCCTGA |
| | | GAGCCGAGGACACGGCCGTATATTACTGTGCGAAATC |
| | | TTCTGCTGGTTTTGACTACTGGGGCCAGGGAACCCTG |
| | | GTCACCGTCTCGAGCGGTGGAGGCGGTTCAGGCGGAG |
| | | GTGGCAGCGGCGGTGGCGGGTCGACGGACATCCAGA |
| | | TGACCCAGTCTCCATCCTCCCTGTCTGCATCTGTAGGA |
| | | GACAGAGTCACCATCACTTGCCGGGCAAGTCAGAGCA |
| | | TTAGCAGCTATTTAAATTGGTATCAGCAGAAACCAGG |
| | | GAAAGCCCCTAAGCTCCTGATCTATTATGCATCCACTT |
| | | TGCAAAGTGGGGTCCCATCAAGGTTCAGTGGCAGTGG |
| | | ATCTGGGACAGATTTCACTCTCACCATCAGCAGTCTG |
| | | CAACCTGAAGATTTTGCAACTTACTACTGTCAACAGG |
| | | ATAATGGTTATCCTTCTACGTTCGGCCAAGGGACCAA |
| | | GGTGGAAATCAAACGG |
| 1069 | anti-PDL1 | EVQLLESGGGLVQPGGSLRLSCAASGFTFSSYAMSWVR |
| | ScFvs | QAPGKGLEWVSSIYSTGGATAYADSVKGRFTISRDNSK |
| | (amino acid | NTLYLQMNSLRAEDTAVYYCAKSSAGFDYWGQGTLVT |
| | sequence) | VSSGGGGSGGGGSGGGGSTDIQMTQSPSSLSASVGDRV |
| | | TITCRASQSISSYLNWYQQKPGKAPKLLIYYASTLQSGVP |
| | | SRFSGSGSGTDFTLTISSLQPEDFATYYCQQDNGYPSTFG |
| | | QGTKVEIKR |
| 1070 | anti-PDL1 | GAAGTGCAGCTGCTCGAAAGCGGCGGAGGCTTGGTG |
| | heavy chain | CAGCCAGGAGGGAGCCTGCGACTGTCTTGCGCAGCCA |
| | (nucleic acid | GCGGATTCACTTTCTCTTCCTATGCCATGAGCTGGGTT |
| | sequence) | CGACAGGCACCCGGCAAAGGTCTCGAGTGGGTGTCTA |
| | | GCATCTGGCGAAACGGAATAGTTACAGTGTATGCCGA |
| | | TAGCGTGAAGGGTCGCTTTACTATTTCACGGGATAAT |
| | | TCTAAGAACACCCTCTACCTGCAAATGAATAGCCTTA |
| | | GGGCAGAAGATACCGCCGTGTACTACTGTGCCAAATG |
| | | GTCCGCAGCCTTTGACTACTGGGGCCAGGGGACACTG |
| | | GTGACCGTGTCCTCTGCATCAACCAAGGGGCCATCAG |
| | | TGTTCCCACTCGCCCCATCTTCCAAGAGTACTTCCGGC |
| | | GGAACCGCAGCCCTTGGCTGCCTTGTTAAGGACTATT |
| | | TCCCAGAACCCGTGACCGTAAGTTGGAACTCTGGCGC |
| | | CCTTACTTCTGGGGTGCACACCTTCCCAGCAGTGTTGC |
| | | AGTCCAGTGGCCTTTACTCTCTGTCTAGTGTAGTGACT |
| | | GTGCCTTCCTCTAGTCTCGGTACCCAGACCTATATTTG |
| | | TAATGTTAACCATAAGCCCAGCAATACAAAGGTTGAT |
| | | AAGAAAGTGGAACCCAAGAGCTGCGATAAGACACAT |
| | | ACCTGCCCACCTTGTCCAGCTCCCGAGCTGCTGGGCG |
| | | GACCCTCAGTCTTTCTCTTCCCACCTAAACCCAAGGAT |
| | | ACCCTTATGATCTCCAGGACTCCTGAGGTGACCTGCG |
| | | TTGTGGTCGACGTGTCACATGAGGACCCTGAGGTAAA |
| | | GTTTAACTGGTACGTGGACGGTGTGGAGGTACATAAC |
| | | GCTAAGACTAAGCCACGAGAGGAGCAATACGCTTCC |
| | | ACTTACAGGGTGGTCAGCGTCCTGACCGTTCTCCATC |
| | | AGGACTGGCTGAACGGGAAGGAATATAAGTGTAAGG |
| | | TTAGCAACAAAGCTCTCCCTGCACCAATCGAGAAGAC |
| | | AATCAGCAAGGCAAAAGGGCAGCCTCGGGAACCTCA |
| | | GGTCTACACCCTCCCTCCTAGCAGGGAAGAGATGACA |
| | | AAGAACCAGGTCTCTCTCACCTGCCTGGTGAAAGGCT |
| | | TCTATCCATCTGACATTGCTGTGGAGTGGGAATCCAA |
| | | CGGCCAGCCTGAAAATAATTATAAGACCACACCCCCC |
| | | GTCCTTGATTCCGATGGATCTTTCTTCCTGTACAGTAA |
| | | ACTCACCGTCGACAAATCACGGTGGCAGCAAGGTAAC |
| | | GTGTTCAGCTGTTCTGTCATGCATGAGGCTCTGCATAA |
| | | CCATTACACACAAAAGTCTTTGTCATTGTCTCCAGGAT |
| | | GATGAGAATTCATTGATCATAATCAGCCATACCAC |
| 1071 | anti-PDL1 | EVQLLESGGGLVQPGGSLRLSCAASGFTFSSYAMSWVR |
| | heavy chain | QAPGKGLEWVSSIWRNGIVTVYADSVKGRFTISRDNSK |
| | (amino acid | NTLYLQMNSLRAEDTAVYYCAKWSAAFDYWGQGTLV |
| | sequence) | TVSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCP |
| | | APELLGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPG |
| 1072 | anti-PDL1 | GAAGTGCAGCTGCTCGAAAGCGGCGGAGGCTTGGTG |
| | heavy chain | CAGCCAGGAGGGAGCCTGCGACTGTCTTGCGCAGCCA |
| | (nucleic acid | GCGGATTCACTTTCTCTTCCTATGCCATGAGCTGGGTT |
| | sequence) | CGACAGGCACCCGGCAAAGGTCTCGAGTGGGTGTCTA |
| | | GCATCTGGCGAAACGGAATAGTTACAGTGTATGCCGA |
| | | TAGCGTGAAGGGTCGCTTTACTATTTCACGGGATAAT |
| | | TCTAAGAACACCCTCTACCTGCAAATGAATAGCCTTA |
| | | GGGCAGAAGATACCGCCGTGTACTACTGTGCCAAATG |
| | | GTCCGCAGCCTTTGACTACTGGGGCCAGGGGACACTG |
| | | GTGACCGTGTCCTCTGCATCAACCAAGGGGCCATCAG |
| | | TGTTCCCACTCGCCCCATCTTCCAAGAGTACTTCCGGC |
| | | GGAACCGCAGCCCTTGGCTGCCTTGTTAAGGACTATT |
| | | TCCCAGAACCCGTGACCGTAAGTTGGAACTCTGGCGC |
| | | CCTTACTTCTGGGGTGCACACCTTCCCAGCAGTGTTGC |
| | | AGTCCAGTGGCCTTTACTCTCTGTCTAGTGTAGTGACT |
| | | GTGCCTTCCTCTAGTCTCGGTACCCAGACCTATATTTG |
| | | TAATGTTAACCATAAGCCCAGCAATACAAAGGTTGAT |
| | | AAGAAAGTGGAACCCAAGAGCTGCGATAAGACACAT |
| | | ACCTGCCCACCTTGTCCAGCTCCCGAGCTGCTGGGCG |
| | | GACCCTCAGTCTTTCTCTTCCCACCTAAACCCAAGGAT |
| | | ACCCTTATGATCTCCAGGACTCCTGAGGTGACCTGCG |
| | | TTGTGGTCGACGTGTCACATGAGGACCCTGAGGTAAA |
| | | GTTTAACTGGTACGTGGACGGTGTGGAGGTACATAAC |
| | | GCTAAGACTAAGCCACGAGAGGAGCAATACGCTTCC |
| | | ACTTACAGGGTGGTCAGCGTCCTGACCGTTCTCCATC |
| | | AGGACTGGCTGAACGGGAAGGAATATAAGTGTAAGG |
| | | TTAGCAACAAAGCTCTCCCTGCACCAATCGAGAAGAC |
| | | AATCAGCAAGGCAAAAGGGCAGCCTCGGGAACCTCA |
| | | GGTCTACACCCTCCCTCCTAGCAGGGAAGAGATGACA |
| | | AAGAACCAGGTCTCTCTCACCTGCCTGGTGAAAGGCT |
| | | TCTATCCATCTGACATTGCTGTGGAGTGGGAATCCAA |
| | | CGGCCAGCCTGAAAATAATTATAAGACCACACCCCCC |
| | | GTCCTTGATTCCGATGGATCTTTCTTCCTGTACAGTAA |
| | | ACTCACCGTCGACAAATCACGGTGGCAGCAAGGTAAC |
| | | GTGTTCAGCTGTTCTGTCATGCATGAGGCTCTGCATAA |
| | | CCATTACACACAAAAGTCTTTGTCATTGTCTCCAGGAT |
| | | GATGAGAATTCATTGATCATAATCAGCCATACCAC |
| 1073 | anti-PDL1 | EVQLLESGGGLVQPGGSLRLSCAASGFTFSSYAMSWVR |
| | heavy chain | QAPGKGLEWVSSIWRNGIVTVYADSVKGRFTISRDNSK |
| | (amino acid | NTLYLQMNSLRAEDTAVYYCAKWSAAFDYWGQGTLV |
| | sequence) | TVSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCP |
| | | APELLGGPSVFLFPPKPKDTLMISRTPEVTCVVVDVSHE |
| | | DPEVKFNWYVDGVEVHNAKTKPREEQYASTYRVVSVL |
| | | TVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPG |
| 1074 | anti-PDL1 | EVQLLESGGGLVQPGGSLRLSCAASGFTFSSYAMSWVR |
| | heavy chain | QAPGKGLEWVSSIWRNGIVTVYADSVKGRFTISRDNSK |
| | (amino acid | NTLYLQMNSLRAEDTAVYYCAKWSAAFDYWGQGTLV |
| | sequence) | TVSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCP |
| | | APELLGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPG |
| 1075 | CDR1, VL | QSMSSY |
| 1076 | CDR1, VH | GITVGTNY |
| 1077 | CDR2, VH | ISSGGNT |

While the foregoing invention has been described in some detail for purposes of clarity and understanding, it will be clear to one skilled in the art from a reading of this disclosure that various changes in form and detail can be made without departing from the true scope of the invention. It is understood that the materials, examples, and embodiments described herein are for illustrative purposes only and not intended to be limiting and that various modifications or changes in light thereof will be suggested to persons skilled in the art and are to be included within the spirit and scope of the appended claims.

We claim:

1. A method for detecting an in vivo distribution of an activated binding polypeptide in a mammalian subject, the method comprising:
administrating to a mammalian subject a tracer dose of a radiolabeled activatable binding polypeptide,
wherein the radiolabeled activatable binding polypeptide comprises a radionuclide and an activatable binding polypeptide,
wherein the activatable binding polypeptide comprises a prodomain and a binding moiety, wherein the prodomain comprises a masking moiety and a cleavable moiety,
wherein, when the radiolabeled activatable binding polypeptide is activated, a radiolabeled activated binding polypeptide is generated that is capable of specifically binding, in vivo, a biological target; and
imaging the mammalian subject using positron emission tomography (PET) at a time point following administration of the tracer dose.

2. The method of embodiment 1, wherein the radionuclide is selected from the group consisting of ¹¹¹In, ¹³¹I, ¹²³I, ^{99m}Tc, ¹⁷⁷Lu, ⁸⁹Zr, ¹²⁴I, ⁶⁴Cu, ⁸⁶Y, ⁷⁰Br, ¹⁸F, and ⁶⁸Ga.

3. The method of any of claims 1-2, wherein the radionuclide is Zr⁸⁹ and wherein the activatable binding polypeptide is a ⁸⁹Zr-conjugated activatable binding polypeptide.

4. The method of any of claims 1-3, wherein the radiolabeled activatable binding polypeptide comprises a chelation moiety.

5. The method of claim 4, wherein the chelation moiety comprises a structure corresponding to a chelation agent selected from the group consisting of diethylenetraminepentaacetic acid, ethylenediaminetetraacetic acid, 1,4,7,10-tetraacetic acid, and deferoxamine.

6. The method of claim 5, wherein the chelation moiety comprises a structure corresponding to deferoxamine.

7. The method of any of claims 4-6, wherein the chelation moiety further comprises a succinyl substituent.

8. The method of any of claims 1-2, wherein the radiolabeled activatable binding polypeptide comprises an N-succinimidyl deferoxamine activatable binding polypeptide.

9. The method of claim 8, wherein the radionuclide is ⁸⁹Zr whereby the radiolabeled activatable binding polypeptide comprises an ⁸⁹Zr-N-succinimidyl deferoxamine activatable binding polypeptide.

10. The method of any of claims 1-9, wherein radionuclide is present in the activatable binding polypeptide at a radionuclide:activatable binding polypeptide conjugation ratio in the range of from about 0.5 to about 3.0, or from about 0.5 to about 2.0, or from about 0.5 to about 1.5.

11. The method of any of claims 1-10, wherein the activatable binding polypeptide further comprises an additional moiety conjugated thereto that imparts an additional property to the corresponding radiolabeled activated binding polypeptide, wherein the additional property is selected from the group consisting of extended half-life and cytotoxicity.

12. The method of claim 11, wherein the additional property is extended half-life.

13. The method of claim 12, wherein the additional moiety is selected from the group consisting of a polyethylene glycol moiety and a human serum albumin moiety.

14. The method of claim 11, wherein the additional property is cytotoxicity.

15. The method of claim 14, wherein the additional moiety comprises all or part of a toxin.

16. The method of any of claims 1-15, wherein the tracer dose comprises a quantity of the radiolabeled activatable binding polypeptide corresponding to a radiation activity in the range of from about 1 MBq to about 5 MBq, or from about 1 MBq to about 4.5 MBq, or from about 1 MBq to about 4 MBq, or from about 2 MBq to about 4 MBq.

17. The method of claim 16, wherein the tracer dose comprises a quantity of the radiolabeled activatable binding polypeptide corresponding to a radiation activity of about 3.7 MBq.

18. The method of any of claims 1-17, wherein the tracer dose further comprises water.

19. The method of claim 18, wherein the tracer dose further comprises 0.9% NaCl in water.

20. The method of any of claims 1-19, wherein the tracer dose comprises a composition that is stable after storage at a time temperature in the range of from about 2 to about 8° C. stable after a time period of at least about 1 month, or at least about 3 months, or at least about 6 months, or at least about 12 months with respect to one or more properties selected from the group consisting of concentration of aggregates, concentration of radiolabeled activatable binding polypeptide, pH, and radiochemical purity.

21. The method of claim 20, wherein the property is concentration of aggregates.

22. The method of any of claims 20-21, wherein the property is concentration of radiolabeled activatable binding polypeptide.

23. The method of any of claims 20-22, wherein the property is pH.

24. The method of any of claims 20-23, wherein the property is radiochemical purity.

25. The method of any of claims 1-24, wherein the tracer dose comprises the radiolabeled activatable binding polypeptide at a concentration in the range of from about 1 mg/ml to about 20 mg/ml, or from about 5 mg/ml to about 20 mg/ml, or from about 5 mg/ml to about 15 mg/ml, or from about 6 mg/ml to about 14 mg/ml, or from about 7 mg/ml to about 13 mg/ml, or from about 8 mg/ml to about 12 mg/ml, or from about 9 mg/ml to about 11 mg/ml.

26. The method of any of claims 1-25, further comprising administering a blocking dose to the mammalian subject, wherein the blocking dose comprises a corresponding non-radiolabeled activatable binding polypeptide.

27. The method of claim 26, wherein administration of the blocking dose precedes administration of the tracer dose.

28. The method of claim 26, wherein the blocking dose and tracer dose are administered as a single composition comprising the radiolabeled activatable binding polypeptide and the corresponding non-radiolabeled activatable binding polypeptide.

29. The method of any of claims 26-28, wherein the blocking dose comprises a quantity of the corresponding non-radiolabeled activatable binding polypeptide in the range of from about 0.1 mg/Kg to about 10 mg/Kg, or in the range of from about 0.2 mg/Kg to about 10 mg/Kg, or from about 0.3 mg/Kg to about 10 mg/Kg, or from about 0.01 mg/Kg to about 0.3 mg/Kg or from about 0.01 mg/Kg to about 0.2 mg/Kg, or from about 0.1 mg/Kg to about 0.1 mg/Kg.

30. The method of any of claims 26-28, wherein the blocking dose comprises a fixed dose of about 5 mg.

31. The method of any of claims 26-28, wherein the blocking dose comprises a dose of about 0.07 mg/Kg.

32. The method of any of claims 26-28, wherein the blocking dose comprises about 0.1 mg/Kg, or about 0.2 mg/Kg, or about 0.3 mg/Kg, or about 1 mg/Kg, or about 3 mg/Kg, or about 10 mg/Kg of the corresponding non-radiolabeled activatable binding polypeptide.

33. The method of any of claims 26-28, wherein the blocking dose comprises the corresponding non-radiolabeled activatable binding polypeptide in a quantity that is less than about 0.3 mg/Kg, or less than about 0.2 mg/Kg, or less than about 0.1 mg/Kg, but greater than about 0.01 mg/Kg.

34. The method of claim 32, wherein the blocking dose comprises about 0.1 mg/Kg of the corresponding non-radiolabeled activatable binding polypeptide.

35. The method of claim 32, wherein the blocking dose comprises about 0.2 mg/Kg of the corresponding non-radiolabeled activatable binding polypeptide.

36. The method of claim 32, wherein the blocking dose comprises about 0.3 mg/Kg of the corresponding non-radiolabeled activatable binding polypeptide.

37. The method of claim 32, wherein the blocking dose comprises about 1 mg/Kg of the corresponding non-radiolabeled activatable binding polypeptide.

38. The method of claim 32, wherein the blocking dose comprises about 3 mg/Kg of the corresponding non-radiolabeled activatable binding polypeptide.

39. The method of claim 32, wherein the blocking dose comprises about 10 mg/Kg of the corresponding non-radiolabeled activatable binding polypeptide.

40. The method of any of claims 1-39, wherein the imaging step occurs at a time point in the period of from about 1 day to about 10 days post tracer dose administration, or at a time point in the period of from about 2 days to about 10 days post tracer dose administration, or in the period of from about 2 days to about 9 days post tracer dose administration, or in the period of from about 2 days to about 8 days post tracer dose administration, or in the period of from about 2 days to about 7 days post tracer dose administration, or in the period of from about 3 days to about 10 days post tracer dose administration, or in the period of from about 3 days to about 9 days post tracer dose administration, or in the period of from about 3 days to about 8 days post tracer dose administration.

41. The method of claim 40, wherein the imaging step occurs at a time point in the period of from about 1 day to about 10 days post tracer dose administration.

42. The method of claim 40, wherein the imaging step occurs at a time point in the period of from about 2 days to about 9 days post tracer dose administration.

43. The method of claim 40, wherein the imaging step occurs at a time point in the period of from about 2 days to about 8 days post tracer dose administration.

44. The method of claim 40, wherein the imaging step occurs at a time point in the period of from about 2 days to about 7 days post tracer dose administration.

45. The method of claim 40, wherein the imaging step occurs at a time point in the period of from about 3 days to about 10 days post tracer dose administration.

46. The method of claim 40, wherein the imaging step occurs at a time point in the period of from about 3 days to about 9 days post tracer dose administration.

47. The method of claim 40, wherein the imaging step occurs at a time point in the period of from about 3 days to about 8 days post tracer dose administration.

48. The method of any of claims 1-39, wherein the mammalian subject is subjected to PET scanning at a time point corresponding to day 2, and/or day 4, and/or day 7 post tracer dose administration.

49. The method of claim 48, wherein the mammalian subject is subjected to PET scanning at a time point corresponding to day 2 post tracer dose administration.

50. The method of claim 48, wherein the mammalian subject is subjected to PET scanning at a time point corresponding to day 4 post tracer dose administration.

51. The method of claim 48, wherein the mammalian subject is subjected to PET scanning at a time point corresponding to day 7 post tracer dose administration.

52. The method of any of claims 1-51, wherein the mammalian subject has been diagnosed as having a cancer.

53. The method of any of claims 1-52, wherein the mammalian subject has a tumor.

54. The method of any of claims 1-53, wherein the imaging step results in a resulting PET scan that covers an area that includes one or more organs or tissue corresponding to the heart, blood, lung, liver, kidney, pancreas, stomach, ilium, colon, muscle, bone, skin, brain, thymus, brown adipose tissue (BAT), spleen, and/or tumor.

55. The method of any of claims 53-54, wherein a resulting PET scan covers an area that includes all or a portion of a tumor.

56. The method of any of claims 1-55, wherein the imaging step comprises whole body imaging.

57. The method of any of claims 1-56, wherein the CM comprises a substrate for one or more proteases selected from the group consisting of ADAM, an ADAM-like, or ADAMTS; an aspartate protease; an aspartic cathepsin; a caspase; a cysteine proteinase; a kallikrein-related peptidase (KLK); a metallo proteinase, bone morphogenetic protein 1 (BMP-1), and the like); a matrix metalloproteinase (MMP); a serine protease, a coagulation factor protease; elastase, Granzyme B, Guanidinobenzoatase, HtrA1, Human Neutrophil Elastase, Lactoferrin, Marapsin, NS3/4A, PACE4, Plasmin, prostate-specific antigen (PSA), tissue plasminogen activator (tPA), Thrombin, Tryptase, urokinase (uPA), and a Type II transmembrane Serine Protease (TTSP).

58. The method of any of claims 1-56, wherein the CM is a substrate for one or more proteases selected from the group consisting of a matrix metalloprotease (MMP), a thrombin, a neutrophil elastase, a cysteine protease, a legumain, and a serine protease.

59. The method of any of claim 1-56, wherein the CM comprises an amino acid sequence corresponding to an amino acid sequence selected from the group consisting of SEQ ID NOS: 1-67.

60. The method of any of claims 1-59, wherein the radiolabeled activatable binding polypeptide is a radiolabeled activatable antibody.

61. The method of claim 60, wherein the radiolabeled activatable antibody is a radiolabeled activatable anti-PDL-1 antibody.

62. The method of claim 61, wherein the radiolabeled activatable anti-PDL-1 antibody comprises:
(a) a variable heavy chain complementarity determining region 1 (VH CDR1) comprising the amino acid sequence of SEQ ID NO:425;
(b) a variable heavy chain complementarity determining region 2 (VH CDR2) comprising an amino acid sequence selected from the group consisting of SEQ ID NOs: 436, 428, 430, 432, 434, 436, and 443-452; and
(c) a variable heavy chain complementarity determining region 3 (VH CDR3) comprising an amino acid sequence selected from the group consisting of SEQ ID NOs: 427, 429, 431, 433, 435, 437, and 438-442.

63. The method of claim 62, where the radiolabeled activatable anti-PDL-1 antibody further comprises:
(d) a variable light chain complementarity determining region 1 (VL CDR1) comprising the amino acid sequence of SEQ ID NO:414;
(e) a variable light chain complementarity determining region 2 (VL CDR2) comprising an amino acid sequence selected from the group consisting of SEQ ID NOs:415, 417, 419, 421, and 423; and
(f) a variable light chain complementarity determining region 3 (VL CDR3) comprising an amino acid sequence selected from the group consisting of SEQ ID NOs:416, 418, 420, 422, and 424.

64. The method of any of claims 62-64, wherein
the VL CDR2 comprises the amino acid sequence of SEQ ID NO:417,
the VL CDR3 comprises the amino acid sequence of SEQ ID NO:424,
the VH CDR2 comprises the amino acid sequence of SEQ ID NO: 451, and
the VH CDR3 comprises the amino acid sequence of SEQ ID NO: 440.

65. The method of any of claims 62-63, wherein
the VL CDR2 comprises the amino acid sequence of SEQ ID NO:423,
the VL CDR3 comprises the amino acid sequence of SEQ ID NO:424,
the VH CDR2 comprises the amino acid sequence of SEQ ID NO:451, and
the VH CDR3 comprises the amino acid sequence of SEQ ID NO:440.

66. The method of claim 61, wherein the radiolabeled activatable anti-PDL-1 antibody comprises a variable light chain comprising the amino acid sequence of SEQ ID NO:112 and a variable heavy chain comprising the amino acid sequence of SEQ ID NO:146.

67. The method of any of claims 59-66, wherein the MM comprises an amino acid sequence selected from the group consisting of SEQ ID NOs:84-108.

68. The method of claim 67, wherein the MM comprises the amino acid sequence of SEQ ID NO:90.

69. The method of any of claims 59-68, wherein the CM comprises the amino acid sequence of SEQ ID NO:24.

70. The method of any of claims 61-63, wherein the radiolabeled activatable anti-PDL-1 antibody comprises a light chain amino acid sequence comprising the amino acid sequence of SEQ ID NO:971.

71. The method of any of claims 61-63, wherein the radiolabeled activatable anti-PDL-1 antibody comprises a light chain amino acid sequence corresponding to SEQ ID NO:969.

72. The method of any of claims 61-63, wherein the radiolabeled activatable anti-PDL-1 antibody comprises a light chain amino acid sequence corresponding to SEQ ID NO:170.

73. The method of any of claims 61-63, wherein the radiolabeled activatable anti-PDL-1 antibody comprises a light chain amino acid sequence corresponding to SEQ ID NO:168.

74. The method of any of claims 60-73, wherein the radiolabeled activatable anti-PDL-1 antibody comprises a heavy chain amino acid sequence corresponding to SEQ ID NO:146.

75. The method of any of claims 60-73, wherein the radiolabeled activatable anti-PDL-1 antibody comprises a heavy chain amino acid sequence comprising the amino acid sequence of SEQ ID NO:172.

76. The method of claim 61, wherein the radiolabeled activatable anti-PDL-1 antibody comprises a light chain amino acid sequence comprising the amino acid sequence of SEQ ID NO:168 and a heavy chain amino acid sequence comprising the amino acid sequence of SEQ ID NO: 172.

77. The method of claim 61, wherein the radiolabeled activatable anti-PDL-1 antibody comprises a light chain amino acid sequence comprising the amino acid sequence of SEQ ED NO:169 and a heavy chain amino acid sequence comprising the amino acid sequence of SEQ ID NO:172.

78. A method for identifying a mammalian subject suitable for treatment with an activatable binding polypeptide, the method comprising:
detecting the in vivo distribution of an activated binding polypeptide in a mammalian subject in accordance with the method of any of claims 1-77, and
identifying the mammalian subject as being suitable for treatment with the activatable binding polypeptide if (a) the radionuclide is detectably present within the PET image of the tumor.

79. The method of claim 78, wherein the step of identifying the mammalian subject as being suitable for treatment with the activatable binding polypeptide further comprises (b) obtaining a tumor tissue sample from the subject.

80. A method of treating a mammalian subject with an activatable binding polypeptide, the method comprising:
identifying a mammalian subject suitable for treatment with an activatable binding polypeptide in accordance with any of claims 78-79; and
administering to the mammalian subject a therapeutically effective dose of the activatable binding polypeptide.

81. A ⁸⁹Zr-conjugated activatable binding polypeptide,
wherein the ⁸⁹Zr-conjugated activatable binding polypeptide comprises ⁸⁹Zr conjugated via a chelation moiety to an activatable binding polypeptide,
wherein the activatable binding polypeptide comprises a prodomain and a binding moiety, wherein the prodomain comprises a masking moiety and a cleavable moiety,
wherein, when the ⁸⁹Zr-conjugated activatable binding polypeptide is activated, a ⁸⁹Zr-conjugated activated binding polypeptide is generated that is capable of specifically binding, in vivo, a biological target.

82. The ⁸⁹Zr-conjugated activatable binding polypeptide of claim 81, wherein the radiolabeled activatable binding polypeptide comprises a deferoxamine moiety.

83. The ⁸⁹Zr-conjugated activatable binding polypeptide of claim 82, wherein the desferoxamine moiety comprises succinimidyl desferal and wherein the Zr⁸⁹-conjugated activatable binding polypeptide is an N-succinimidyl deferoxamine activatable binding polypeptide.

84. The ⁸⁹Zr-conjugated activatable binding polypeptide of any of claims 81-83, wherein the conjugation ratio is in the range of from about 0.5 to about 3.0, or from about 0.5 to about 2.0, or from about 0.5 to about 1.5.

85. The ⁸⁹Zr-conjugated activatable binding polypeptide of claim 84, wherein the conjugation ratio is in the range of from about 0.5 to about 2.0.

86. The ⁸⁹Zr-conjugated activatable binding polypeptide of any of claims 81-85, wherein the ⁸⁹Zr-conjugated activatable binding polypeptide further comprises an additional moiety conjugated thereto that imparts an additional property to the corresponding radiolabelled activated binding polypeptide, wherein the additional property is selected from the group consisting of extended half-life and cytotoxicity.

87. The ⁸⁹Zr-conjugated activatable binding polypeptide of claim 86, wherein the additional property is extended half-life.

88. The ⁸⁹Zr-conjugated activatable binding polypeptide of claim 87, wherein the additional moiety is selected from the group consisting of a polyethylene glycol moiety and a human serum albumin moiety.

89. The ⁸⁹Zr-conjugated activatable binding polypeptide of claim 86, wherein the additional property is cytotoxicity.

90. The ⁸⁹Zr-conjugated activatable binding polypeptide of claim 89, wherein the additional moiety comprises all or part of a toxin.

91. The ⁸⁹Zr-conjugated activatable binding polypeptide of any of claims 81-90, wherein the ⁸⁹Zr-conjugated activatable binding polypeptide is an ⁸⁹Zr-conjugated anti-PDL-1 activatable antibody.

92. The ⁸⁹Zr-conjugated activatable binding polypeptide of claim 91, wherein the ⁸⁹Zr-conjugated anti-PDL-1 activatable antibody comprises:
(a) a variable heavy chain complementarity determining region 1 (VH CDR1) comprising the amino acid sequence of SEQ ID NO:425;
(b) a variable heavy chain complementarity determining region 2 (VH CDR2) comprising an amino acid sequence selected from the group consisting of SEQ ID NOs:426, 428, 430, 432, 434, 436, and 438-442; and
(c) a variable heavy chain complementarity determining region 3 (VH CDR3) comprising an amino acid sequence selected from the group consisting of SEQ ID NOs: 429, 431, 433, 435, 437, and 438-442.

93. The ⁸⁹Zr-conjugated activatable binding polypeptide of claim 90, wherein the ⁸⁹Zr-conjugated anti-PDL-1 activatable antibody further comprises:
(d) a variable light chain complementarity determining region 1 (VL CDR1) comprising the amino acid sequence of SEQ ID NO: 414;
(e) a variable light chain complementarity determining region 2 (VL CDR2) comprising an amino acid sequence selected from the group consisting of SEQ ID NOs:415, 417, 419, 421, and 423; and
(f) a variable light chain complementarity determining region 3 (VL CDR3) comprising an amino acid sequence selected from the group consisting of SEQ ID NOs:416, 418, 420, 422, and 424.

94. The ⁸⁹Zr-conjugated activatable binding polypeptide of any of claims 92-93, wherein
the VL CDR2 comprises the amino acid sequence of SEQ ID NO:417,
the VL CDR3 comprises the amino acid sequence of SEQ ID NO:424
the VH CDR2 comprises the amino acid sequence of SEQ ID NO: 451, and
the VH CDR3 comprises the amino acid sequence of SEQ ID NO:440.

95. The ⁸⁹Zr-conjugated activatable binding polypeptide method of any of claims 92-93, wherein
the VL CDR2 comprises the amino acid sequence of SEQ ID NO:423,
the VL CDR3 comprises the amino acid sequence of SEQ ID NO:424,
the VH CDR2 comprises the amino acid sequence of SEQ ID NO:451, and
the VH CDR3 comprises the amino acid sequence of SEQ ID NO:440.

96. The ⁸⁹Zr-conjugated activatable binding polypeptide method of claim 91, wherein the radiolabeled activatable antibody comprises a variable light chain comprising the amino acid sequence of SEQ ID NO:112 and a variable heavy chain comprising the amino acid sequence of SEQ ID N):146.

97. The ⁸⁹Zr-conjugated activatable binding polypeptide method of any of claims 91-96, wherein the MM comprises an amino acid sequence selected from the group consisting of SEQ ID NOs:84-108.

98. The ⁸⁹Zr-conjugated activatable binding polypeptide method of claim 97, wherein the MM comprises an amino acid sequence corresponding to SEQ ID NO:90.

99. The ⁸⁹Zr-conjugated activatable binding polypeptide method of any of claims 91-98, wherein the CM comprises an amino acid sequence corresponding to SEQ ID NO:24.

100. The ⁸⁹Zr-conjugated activatable binding polypeptide method of any of claims 91-93, wherein the radiolabeled activatable antibody comprises a light chain amino acid sequence corresponding to SEQ ID NO:971.

101. The ⁸⁹Zr-conjugated activatable binding polypeptide method of any of claims 91-93, wherein the radiolabeled activatable antibody comprises a light chain amino acid sequence corresponding to SEQ ID NO:969.

102. The ⁸⁹Zr-conjugated activatable binding polypeptide method of any of claims 91-93, wherein the radiolabeled activatable antibody comprises a light chain amino acid sequence corresponding to SEQ ID NO:170.

103. The ⁸⁹Zr-conjugated activatable binding polypeptide method of any of claims 91-93, wherein the radiolabeled activatable antibody comprises a light chain amino acid sequence corresponding to SEQ ID NO:168.

104. The ⁸⁹Zr-conjugated activatable binding polypeptide method of any of claims 91-103, wherein the radiolabeled activatable antibody comprises a heavy chain amino acid sequence comprising the amino acid sequence of SEQ ID NO:146.

105. The ⁸⁹Zr-conjugated activatable binding polypeptide method of any of claims 91-93, wherein the radiolabeled activatable antibody comprises a heavy chain amino acid sequence comprising the amino acid sequence of SEQ ID NO:172.

106. The ⁸⁹Zr-conjugated activatable binding polypeptide of claim 89, wherein the radiolabeled activatable antibody comprises a light chain amino acid sequence comprising the amino acid sequence of SEQ ID NO:168 and a heavy chain amino acid sequence comprising the amino acid sequence of SEQ ID NO:172.

107. The ⁸⁹Zr-conjugated activatable binding polypeptide of claim 91, wherein the radiolabeled activatable antibody comprises a light chain amino acid sequence comprising the amino acid sequence of SEQ ID NO:169 and a heavy chain amino acid sequence comprising the amino acid sequence of SEQ ID NO:172.

108. A stable composition comprising the ⁸⁹Zr-conjugated activatable binding polypeptide of any of claims 91-107 and a liquid phase carrier, wherein the composition is stable after storage at a temperature in the range of from about 2′C to about 8° C. after a time period of at least about 1 month, or at least about 3 months, or at least about 6 months, or at least about 12 months, with respect to at least one property selected from the group consisting of percent (%) aggregates, concentration, pH, and radiochemical.

109. A tracer dose comprising the composition of claim 108, wherein the dose comprises a quantity of ⁸⁹Zr-conjugated activatable binding polypeptide corresponding to 37 MBq.

110. The tracer dose of claim 109, wherein the ⁸⁹Zr-conjugated activatable binding polypeptide is present at a concentration in the range of from about 1 mg/ml to about 20 mg/ml, or from about 5 mg/ml to about 20 mg/ml, or from about 5 mg/ml to about 15 mg/ml, or from about 6 mg/ml to about 14 mg/ml, or from about 7 mg/ml to about 13 mg/ml, or from about 8 mg/ml to about 12 mg/ml, or from about 9 mg/ml to about 11 mg/ml.

112. An ⁸⁹Zr-labeled activatable binding polypeptide for use as a tracer for positron emission tomography imaging a tumor in a mammalian subject.

113. The ⁸⁹Zr-labeled activatable binding polypeptide of claim 112, wherein the activatable binding polypeptide is an activatable antibody.

114. The ⁸⁹Zr-labeled activatable binding polypeptide of claim 113, wherein the activatable antibody is an activatable anti-PDL-1 antibody.

115. A composition comprising the ⁸⁹Zr-labeled activatable binding polypeptide of any of claims 112-114.
